SEQUENCE LISTING (229 sequences)

SEQ ID NO: 1 - DNA, 39 bp, synthetic construct
  source 1..39: mol_type = other DNA
attcgatcatgcatgttacgaaatcatcctgtggagctt

SEQ ID NO: 2 - DNA, 32 bp, synthetic construct
  source 1..32: mol_type = other DNA
caaggccttcgcgaggcgaaaaaaccccgccg

SEQ ID NO: 3 - DNA, 39 bp, synthetic construct
  source 1..39: mol_type = other DNA
attcgatcatgcatgctgcaggcctacggtatccaccgg

SEQ ID NO: 4 - DNA, 42 bp, synthetic construct
  source 1..42: mol_type = other DNA
caaggccttcgcgagaagctttataaacgcagaaaggcccac

SEQ ID NO: 5 - DNA, 38 bp, synthetic construct
  source 1..38: mol_type = other DNA
tgcgtttataaagcttttacgaaatcatcctgtggagc

SEQ ID NO: 6 - DNA, 33 bp, synthetic construct
  source 1..33: mol_type = other DNA
ccttcgcgagaagcttgcgaaaaaaccccgccg

SEQ ID NO: 7 - DNA, 39 bp, synthetic construct
  source 1..39: mol_type = other DNA
aagctaattcgatcatgcatttgacggctagctcagtcc

SEQ ID NO: 8 - DNA, 37 bp, synthetic construct
  source 1..37: mol_type = other DNA
cgtaggcctgcagcatataaacgcagaaaggcccacc

SEQ ID NO: 9 - DNA, 44 bp, synthetic construct
  source 1..44: mol_type = other DNA
gaagctaattcgatcatgcatgatttgtcctactcaggagagcg

SEQ ID NO: 10 - DNA, 33 bp, synthetic construct
  source 1..33: mol_type = other DNA
attgagtatttcttatccatatgtttttcctcc

SEQ ID NO: 11 - DNA, 42 bp, synthetic construct
  source 1..42: mol_type = other DNA
cggtttgcgtattgggcgcaatttgtcctactcaggagagcg

SEQ ID NO: 12 - DNA, 41 bp, synthetic construct
  source 1..41: mol_type = other DNA
acgtcttcgctactcgccatatgtttttcctcctaaccgcg

SEQ ID NO: 13 - DNA, 46 bp, synthetic construct
  source 1..46: mol_type = other DNA
ccagctggcaattccgacgtcgtcgaatttgctttcgaatttctgc

SEQ ID NO: 14 - DNA, 60 bp, synthetic construct
  source 1..60: mol_type = other DNA
aagaccggcggtcttaagttttttggctgaagaattcgcaaatattatacgcaaggcgac

SEQ ID NO: 15 - DNA, 36 bp, synthetic construct
  source 1..36: mol_type = other DNA
aacaggagtccaagcgcatggaagccatcacaaacg

SEQ ID NO: 16 - DNA, 22 bp, synthetic construct
  source 1..22: mol_type = other DNA
gacgtcggaattgccagctggg

SEQ ID NO: 17 - DNA, 39 bp, synthetic construct
  source 1..39: mol_type = other DNA
tatagggcgaattggagctcttgacagctagctcagtcc

SEQ ID NO: 18 - DNA, 49 bp, synthetic construct
  source 1..49: mol_type = other DNA
gggaacaaaagctggtctagcttaagagttcaccgacaaacaacagata

SEQ ID NO: 19 - DNA, 45 bp, synthetic construct
  source 1..45: mol_type = other DNA
tcggtgaactcttaacggatccttgacagctagctcagtcctagg

SEQ ID NO: 20 - DNA, 38 bp, synthetic construct
  source 1..38: mol_type = other DNA
gctggtctagcttaagagttcaccgacaaacaacagat

SEQ ID NO: 21 - DNA, 25 bp, synthetic construct
  source 1..25: mol_type = other DNA
gctcagtcctaggtataatactagt

SEQ ID NO: 22 - DNA, 60 bp, synthetic construct
  source 1..60: mol_type = other DNA
taggtataatactagtnnnnnnnnnnnnnnnnnnnngttttagagctagaaatagcaagt

SEQ ID NO: 23 - DNA, 40 bp, synthetic construct
  source 1..40: mol_type = other DNA
ttgcgtattgggcgcattacgaaatcatcctgtggagctt

SEQ ID NO: 24 - DNA, 33 bp, synthetic construct
  source 1..33: mol_type = other DNA
attacaacagtttttagcgaaaaaaccccgccg

SEQ ID NO: 25 - DNA, 32 bp, synthetic construct
  source 1..32: mol_type = other DNA
ttgcgtattgggcgcatggcaattccgacgtc

SEQ ID NO: 26 - DNA, 35 bp, synthetic construct
  source 1..35: mol_type = other DNA
attacaacagtttttatataaacgcagaaaggccc

SEQ ID NO: 27 - DNA, 60 bp, synthetic construct
  source 1..60: mol_type = other DNA
gcgttctggacacaattgggttccaccggatacctccggacttgacagctagctcagtcc

SEQ ID NO: 28 - DNA, 50 bp, synthetic construct
  source 1..50: mol_type = other DNA
ttgtgtccagaacgctccgtaggacaccgcaggatacctgaggtcgcccg

SEQ ID NO: 29 - DNA, 41 bp, synthetic construct
  source 1..41: mol_type = other DNA
ccaccgcggtggcggccgcttgacagctagctcagtcctag

SEQ ID NO: 30 - DNA, 40 bp, synthetic construct
  source 1..40: mol_type = other DNA
agctgggtaccgggcccagttcaccgacaaacaacagata

SEQ ID NO: 31 - DNA, 38 bp, synthetic construct
  source 1..38: mol_type = other DNA
gcggttaccaaaggcgtcctcgtcgtcttgaagttgcg

SEQ ID NO: 32 - DNA, 36 bp, synthetic construct
  source 1..36: mol_type = other DNA
gcctttggtaaccgcaggagaagtgaggagacgagc

SEQ ID NO: 33 - DNA, 20 bp, synthetic construct
  source 1..20: mol_type = other DNA
ttgggcgcatggcaattccg

SEQ ID NO: 34 - DNA, 20 bp, synthetic construct
  source 1..20: mol_type = other DNA
gcctggagatccttactcga

SEQ ID NO: 35 - DNA, 60 bp, synthetic construct
  source 1..60: mol_type = other DNA
gcgttctggacacaannnnnnnnnnnnnnnnnnnnnnnnnnttgacagctagctcagtcc

SEQ ID NO: 36 - DNA, 28 bp, synthetic construct
  source 1..28: mol_type = other DNA
ttgtgtccagaacgctccgtaggagaag

SEQ ID NO: 37 - DNA, 33 bp, synthetic construct
  source 1..33: mol_type = other DNA
cggtgcttaaaaactcgagtaaggatctccagg

SEQ ID NO: 38 - DNA, 35 bp, synthetic construct
  source 1..35: mol_type = other DNA
gctagcactatacctaggactgagctagccgtcaa

SEQ ID NO: 39 - DNA, 40 bp, synthetic construct
  source 1..40: mol_type = other DNA
cagtcctaggtatagtgctagcgaattcattaaagaggag

SEQ ID NO: 40 - DNA, 46 bp, synthetic construct
  source 1..46: mol_type = other DNA
atgatcgcaaatgctgagtacttataaacgcagaaaggcccacccg

SEQ ID NO: 41 - DNA, 39 bp, synthetic construct
  source 1..39: mol_type = other DNA
ttaaagaggagaaaggtaccatggcgagtagcgaagacg

SEQ ID NO: 42 - DNA, 20 bp, synthetic construct
  source 1..20: mol_type = other DNA
tgcgcccaatacgcaaaccg

SEQ ID NO: 43 - DNA, 40 bp, synthetic construct
  source 1..40: mol_type = other DNA
cggtttgcgtattgggcgcagttctcagggctcgccgaga

SEQ ID NO: 44 - DNA, 48 bp, synthetic construct
  source 1..48: mol_type = other DNA
ggtacctttctcctctttaatgaattctgatactggccacagacggct

SEQ ID NO: 45 - DNA, 28 bp, synthetic construct
  source 1..28: mol_type = other DNA
gcgcatggcaattccgatatcagcattt

SEQ ID NO: 46 - DNA, 45 bp, synthetic construct
  source 1..45: mol_type = other DNA
ggagatccttactcgagtttttattcgtcgtagaaatcaatgtgg

SEQ ID NO: 47 - DNA, 21 bp, synthetic construct
  source 1..21: mol_type = other DNA
ccagctggcaattccgacgtc

SEQ ID NO: 48 - DNA, 41 bp, synthetic construct
  source 1..41: mol_type = other DNA
agatccttactcgagtttttaattacgatagctacgcacgg

SEQ ID NO: 49 - DNA, 45 bp, synthetic construct
  source 1..45: mol_type = other DNA
ttaaagaggagaaaggtaccatgaaaaccgtggtgattattgatg

SEQ ID NO: 50 - DNA, 46 bp, synthetic construct
  source 1..46: mol_type = other DNA
tgcctgcaggtcgactctagatgaccgacctgatcgaagtgaagac

SEQ ID NO: 51 - DNA, 34 bp, synthetic construct
  source 1..34: mol_type = other DNA
atggcggatgcatgggctcggttctctactggcg

SEQ ID NO: 52 - DNA, 36 bp, synthetic construct
  source 1..36: mol_type = other DNA
cgagcccatgcatccgccattacgatctgacttgcc

SEQ ID NO: 53 - DNA, 46 bp, synthetic construct
  source 1..46: mol_type = other DNA
ataacagggtaatctgaatttcacttcactcgggaaaaatcagggg

SEQ ID NO: 54 - DNA, 39 bp, synthetic construct
  source 1..39: mol_type = other DNA
ccagtagagaaccgagcccagcatggcaattccgacgtc

SEQ ID NO: 55 - DNA, 39 bp, synthetic construct
  source 1..39: mol_type = other DNA
agtcagatcgtaatggcggatataaacgcagaaaggccc

SEQ ID NO: 56 - DNA, 46 bp, synthetic construct
  source 1..46: mol_type = other DNA
tgcctgcaggtcgactctagaccaggatgaataccttaaggacgcc

SEQ ID NO: 57 - DNA, 33 bp, synthetic construct
  source 1..33: mol_type = other DNA
acaccttatgcatgcgcgtgatgcgctgcacac

SEQ ID NO: 58 - DNA, 36 bp, synthetic construct
  source 1..36: mol_type = other DNA
cacgcgcatgcataaggtgtattcccccggcattca

SEQ ID NO: 59 - DNA, 41 bp, synthetic construct
  source 1..41: mol_type = other DNA
ataacagggtaatctgaatttccgctcggcgcgtgatccgc

SEQ ID NO: 60 - DNA, 44 bp, synthetic construct
  source 1..44: mol_type = other DNA
agcgcatcacgcgcatgcatttgacggctagctcagtcctaggt

SEQ ID NO: 61 - DNA, 46 bp, synthetic construct
  source 1..46: mol_type = other DNA
cgggggaatacaccttagtacttataaacgcagaaaggcccacccg

SEQ ID NO: 62 - DNA, 45 bp, synthetic construct
  source 1..45: mol_type = other DNA
cagcgcatcacgcgcatgcatagcatttgcgatcattcacgcagc

SEQ ID NO: 63 - DNA, 27 bp, synthetic construct
  source 1..27: mol_type = other DNA
ggtacctttctcctctttaatgaattc

SEQ ID NO: 64 - DNA, 27 bp, synthetic construct
  source 1..27: mol_type = other DNA
gaattcattaaagaggagaaaggtacc

SEQ ID NO: 65 - DNA, 20 bp, synthetic construct
  source 1..20: mol_type = other DNA
atggcgagtagcgaagacgt

SEQ ID NO: 66 - DNA, 60 bp, synthetic construct
  source 1..60: mol_type = other DNA
ttcgctactcgccatggtacctttctcctctttaatgaattctgaaattgttatccgctc

SEQ ID NO: 67 - DNA, 42 bp, synthetic construct; identical to SEQ ID NO: 11
  source 1..42: mol_type = other DNA

SEQ ID NO: 68 - DNA, 60 bp, synthetic construct
  source 1..60: mol_type = other DNA
ggtacctttctcctctttaatgaattcttgcataaagcctaaggggtaggccttactaga

SEQ ID NO: 69 - DNA, 103 bp, synthetic construct
  source 1..103: mol_type = other DNA
nnnnnnnnnnnnnnnnnnnngttttagagctagaaatagcaagttaaaataaggctagtccgttatcaacttgaaaaagtggcaccgagtcggtgcttttttt

SEQ ID NO: 70 - DNA, 111 bp, synthetic construct
  source 1..111: mol_type = other DNA
nnnnnnnnnnnnnnnnnnnngttttagagctagaaatagcaagttaaaataaggctagtccgttatcaacttgaaaaagtggcacatgaggatcacccatgtgcttttttt

SEQ ID NO: 71 - DNA, 20 bp, synthetic construct
  source 1..20: mol_type = other DNA
ggggccactagggacaggat

SEQ ID NO: 72 - DNA, 20 bp, synthetic construct
  source 1..20: mol_type = other DNA
tggaaccgtactggaactgc

SEQ ID NO: 73 - DNA, 20 bp, synthetic construct
  source 1..20: mol_type = other DNA
catctaattcaacaagaatt

SEQ ID NO: 74 - DNA, 20 bp, synthetic construct
  source 1..20: mol_type = other DNA
tgggttccaccggatacctc

SEQ ID NO: 75 - DNA, 20 bp, synthetic construct
  source 1..20: mol_type = other DNA
aggtatccggtggaacccaa

SEQ ID NO: 76 - DNA, 20 bp, synthetic construct
  source 1..20: mol_type = other DNA
aggcgtcctttgggttccac

SEQ ID NO: 77 - DNA, 20 bp, synthetic construct
  source 1..20: mol_type = other DNA
tggaacccaaaggacgcctt

SEQ ID NO: 78 - DNA, 20 bp, synthetic construct
  source 1..20: mol_type = other DNA
cggttaccaaaggcgtcctt

SEQ ID NO: 79 - DNA, 20 bp, synthetic construct
  source 1..20: mol_type = other DNA
aggacgcctttggtaaccgc

SEQ ID NO: 80 - DNA, 20 bp, synthetic construct
  source 1..20: mol_type = other DNA
cggtgtcctgcggttaccaa

SEQ ID NO: 81 - DNA, 20 bp, synthetic construct
  source 1..20: mol_type = other DNA
tggtaaccgcaggacaccgc

SEQ ID NO: 82 - DNA, 20 bp, synthetic construct
  source 1..20: mol_type = other DNA
aggtatcctgcggtgtcctg

SEQ ID NO: 83 - DNA, 20 bp, synthetic construct
  source 1..20: mol_type = other DNA
aggacaccgcaggatacctg

SEQ ID NO: 84 - DNA, 20 bp, synthetic construct
  source 1..20: mol_type = other DNA
gggcgacctcaggtatcctg

SEQ ID NO: 85 - DNA, 20 bp, synthetic construct
  source 1..20: mol_type = other DNA
aggatacctgaggtcgcccg

SEQ ID NO: 86 - DNA, 20 bp, synthetic construct
  source 1..20: mol_type = other DNA
gggccaccacgggcgacctc

SEQ ID NO: 87 - DNA, 20 bp, synthetic construct
  source 1..20: mol_type = other DNA
aggtcgcccgtggtggccca

SEQ ID NO: 88 - DNA, 20 bp, synthetic construct
  source 1..20: mol_type = other DNA
tggtgaccatgggccaccac

SEQ ID NO: 89 - DNA, 20 bp, synthetic construct
  source 1..20: mol_type = other DNA
tggtggcccatggtcaccat

SEQ ID NO: 90 - DNA, 20 bp, synthetic construct
  source 1..20: mol_type = other DNA
gggtgacctatggtgaccat

SEQ ID NO: 91 - DNA, 20 bp, synthetic construct
  source 1..20: mol_type = other DNA
tggtcaccataggtcaccct

SEQ ID NO: 92 - DNA, 20 bp, synthetic construct
  source 1..20: mol_type = other DNA
tggttgccaagggtgaccta

SEQ ID NO: 93 - DNA, 20 bp, synthetic construct
  source 1..20: mol_type = other DNA
aggtcacccttggcaaccaa

SEQ ID NO: 94 - DNA, 20 bp, synthetic construct
  source 1..20: mol_type = other DNA
aggacacctttggttgccaa

SEQ ID NO: 95 - DNA, 20 bp, synthetic construct
  source 1..20: mol_type = other DNA
ttgtgtccagaacgctccgt

SEQ ID NO: 96 - DNA, 20 bp, synthetic construct
  source 1..20: mol_type = other DNA
acttctcctacggagcgttc

SEQ ID NO: 97 - DNA, 20 bp, synthetic construct
  source 1..20: mol_type = other DNA
aacgctccgtaggagaagtg

SEQ ID NO: 98 - DNA, 20 bp, synthetic construct
  source 1..20: mol_type = other DNA
tcgtctcctcacttctccta

SEQ ID NO: 99 - DNA, 20 bp, synthetic construct
  source 1..20: mol_type = other DNA
ttcatgtagcttgtcccccg

SEQ ID NO: 100 - DNA, 20 bp, synthetic construct
  source 1..20: mol_type = other DNA
ccgactgaagatgcgctctc

SEQ ID NO: 101 - DNA, 20 bp, synthetic construct
  source 1..20: mol_type = other DNA
atgcgctctctggcgctcct

SEQ ID NO: 102 - DNA, 20 bp, synthetic construct
  source 1..20: mol_type = other DNA
tgcgctctctggcgctcctc

SEQ ID NO: 103 - DNA, 20 bp, synthetic construct
  source 1..20: mol_type = other DNA
gcgctctctggcgctcctcg

SEQ ID NO: 104 - DNA, 20 bp, synthetic construct
  source 1..20: mol_type = other DNA
cgctctctggcgctcctcgg

SEQ ID NO: 105 - DNA, 20 bp, synthetic construct
  source 1..20: mol_type = other DNA
actgggatttgtgtaggagc

SEQ ID NO: 106 - DNA, 20 bp, synthetic construct
  source 1..20: mol_type = other DNA
gggtttacccgcgaaagggc

SEQ ID NO: 107 - DNA, 20 bp, synthetic construct
  source 1..20: mol_type = other DNA
ggcagtgccggccctttcgc

SEQ ID NO: 108 - DNA, 20 bp, synthetic construct
  source 1..20: mol_type = other DNA
tggcagtgccggccctttcg

SEQ ID NO: 109 - DNA, 20 bp, synthetic construct
  source 1..20: mol_type = other DNA
aatgcgtggtcgcttaatcc

SEQ ID NO: 110 - DNA, 20 bp, synthetic construct
  source 1..20: mol_type = other DNA
atgcgtggtcgcttaatcct

SEQ ID NO: 111 - DNA, 20 bp, synthetic construct
  source 1..20: mol_type = other DNA
taatcctgggttaaccggac

SEQ ID NO: 112 - DNA, 20 bp, synthetic construct
  source 1..20: mol_type = other DNA
tgcgccggtccggttaaccc

SEQ ID NO: 113 - DNA, 20 bp, synthetic construct
  source 1..20: mol_type = other DNA
ggcccctgcgctgcgctccg

SEQ ID NO: 114 - DNA, 20 bp, synthetic construct
  source 1..20: mol_type = other DNA
cagcgcaggggccggatgat

SEQ ID NO: 115 - DNA, 20 bp, synthetic construct
  source 1..20: mol_type = other DNA
ccggagcgcagcgcaggggc

SEQ ID NO: 116 - DNA, 20 bp, synthetic construct
  source 1..20: mol_type = other DNA
tatcgatgaaatcgcagcat

SEQ ID NO: 117 - DNA, 20 bp, synthetic construct
  source 1..20: mol_type = other DNA
cagcataggcgatgcctatg

SEQ ID NO: 118 - DNA, 20 bp, synthetic construct
  source 1..20: mol_type = other DNA
ccttagacaatccacctcat

SEQ ID NO: 119 - DNA, 20 bp, synthetic construct
  source 1..20: mol_type = other DNA
aaagctgcgccagagtgtcg

SEQ ID NO: 120 - DNA, 20 bp, synthetic construct
  source 1..20: mol_type = other DNA
acactctggcgcagcttttg

SEQ ID NO: 121 - DNA, 20 bp, synthetic construct
  source 1..20: mol_type = other DNA
gacactctggcgcagctttt

SEQ ID NO: 122 - DNA, 20 bp, synthetic construct
  source 1..20: mol_type = other DNA
cgacactctggcgcagcttt

SEQ ID NO: 123 - DNA, 20 bp, synthetic construct
  source 1..20: mol_type = other DNA
ggcgtcctgggcaaagggta

SEQ ID NO: 124 - DNA, 20 bp, synthetic construct
  source 1..20: mol_type = other DNA
ctgtgtattgaagcatggcg

SEQ ID NO: 125 - DNA, 20 bp, synthetic construct
  source 1..20: mol_type = other DNA
cacagccataccctttgccc

SEQ ID NO: 126 - DNA, 20 bp, synthetic construct
  source 1..20: mol_type = other DNA
tatcccaccctcgccatttt

SEQ ID NO: 127 - DNA, 20 bp, synthetic construct
  source 1..20: mol_type = other DNA
ccctcgccattttcgggcac

SEQ ID NO: 128 - DNA, 20 bp, synthetic construct
  source 1..20: mol_type = other DNA
tgcccgaaaatggcgagggt

SEQ ID NO: 129 - DNA, 20 bp, synthetic construct
  source 1..20: mol_type = other DNA
gtgcccgaaaatggcgaggg

SEQ ID NO: 130 - DNA, 20 bp, synthetic construct
  source 1..20: mol_type = other DNA
tgcatgccagtgcccgaaaa

SEQ ID NO: 131 - DNA, 20 bp, synthetic construct
  source 1..20: mol_type = other DNA
ggtttttgtagtgcttgtgc

SEQ ID NO: 132 - DNA, 20 bp, synthetic construct
  source 1..20: mol_type = other DNA
gggtttttgtagtgcttgtg

SEQ ID NO: 133 - DNA, 20 bp, synthetic construct
  source 1..20: mol_type = other DNA
caatccagcgattactaaag

SEQ ID NO: 134 - DNA, 20 bp, synthetic construct
  source 1..20: mol_type = other DNA
acaatccagcgattactaaa

SEQ ID NO: 135 - DNA, 20 bp, synthetic construct
  source 1..20: mol_type = other DNA
tgggtatggcagggggattt

SEQ ID NO: 136 - DNA, 20 bp, synthetic construct
  source 1..20: mol_type = other DNA
gtgctgggaatgggtatggc

SEQ ID NO: 137 - DNA, 20 bp, synthetic construct
  source 1..20: mol_type = other DNA
ccacgtgctgggaatgggta

SEQ ID NO: 138 - DNA, 1095 bp, synthetic construct
  source 1..1095: mol_type = other DNA
gaattcaaaagatctaaataacctaaacgagaggaaagaataatgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcaacatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctggagagcggggagccgctggcctacggtaagagcatcaccgatgcctgcatcggctgggaagataccgatgctctgttacgtcaactggcgaatgcagtaaaagcgcgtcgcgggtaa

SEQ ID NO: 139 - DNA, 545 bp, synthetic construct
  source 1..545: mol_type = other DNA
ggatctaaaggaggccatccatgacacaacctctttttctgatcgggcctcggggctgtggtaaaacaacggtcggaatggcccttgccgattcgcttaaccgtcggtttgtcgataccgatcagtggttgcaatcacagctcaatatgacggtcgcggagatcgtcgaaagggaagagtgggcgggatttcgcgccagagaaacggcggcgctggaagcggtaactgcgccatccaccgttatcgctacaggcggcggcattattctgacggaatttaatcgtcacttcatgcaaaataacgggatcgtggtttatttgtgtgcgccagtatcagtcctggttaaccgactgcaagctgcaccggaagaagatttacggccaaccttaacgggaaaaccgctgagcgaagaagttcaggaagtgctggaagaacgcgatgcgctatatcgcgaagttgcgcatattatcatcgacgcaacaaacgaacccagccaggtgatttctgaaattcgcagcgccctggcacagacgatcaattgttaa

SEQ ID NO: 140 - DNA, 2110 bp, synthetic construct
  source 1..2110: mol_type = other DNA
gaattcaaaagatctaactggtaatttgaggaggtaatttatgaagatcctgctgatcgataactttgatagcttcacccagaatattgcccagtatctgtatgaagttaccggtatttgtgccgatattgttaccaataccgtgacctatgaacatctgcaaatcgaacagtatgatgccgttgttctgagccctggtccgggtcatccgggtgaatatctggattttggtgtttgtggtcaggtgattctgcatagtccggttccgctgctgggtatttgtctgggtcatcagggtattgcacagtttttaggtggcaccgttggtcatgcaccgacaccggttcatggttatcgtagcaaaattacccatagcggtagcggtctgtttcgtgatctgccggaacagtttgaagttgttcgttatcatagcctgatgtgtacccatctgccgcaagaactgcgttgtaccgcatggaccgaagagggtgttgttatggcaattgaacatgaaagccgtccgatttggggtgttcagtttcatccggaaagcattgatagcgaatatggtcatgccctgctgagcaactttattggtatggccatcgaacataatggcaatcatcgtaccagcgcaacccagaatccggatgcaagcgcaagcgccaatgaacattatcgtgcagttggtggtctgctgaatatgcagctggcctatcgtacctatccgggtccgtttgatccgctggcactgtttacccagcgttatgcacaggatcatcatgcattttggctggatagcgaaaaaagcgaacgtccgaatgcacgttatagcattatgggtagtggtcaggcacagggtagcattcgtctgacatatgatgttaatagcgaaagtctgaccctggcaggtccgaaaggtagccgtattgtgaccggtgattttttcaccctgtttagccagattgttgaaagcgttaatgttgcagttccgcagtatctgccgtttgagtttaaaggtggttttgtgggttatatgggctatgaactgaaagcactgaccggtggtaataaagtgtatcgtagcggtcagccggatgcaggttttatgtttgcaccgcatttttttgtgttcgatcatcacgatcagaccgtgtatgagtgcatgattagcgcaaccggtcagagtccgcagtggcctcagctgctgaccagcatgaccacactgaataatgcaaccgatcgtcgtccgtttgttcctggtgcagttgatgaactggaactgagcctggaagatggtccggatgattatattcgtaaagttaaacagagcctgcagtatattaccgatggtgaaagctatgaaatctgtctgaccaatcgtgcacgtatgagctatagcggtgaaccgctggcagcatatcgtcgtatgcgtgaagcatcaccggttccgtatggtgcatatctgtgttttgatagttttagcgttctgagcgcaagtccggaaacctttctgcgtattgatgaaggtggtctgattgaatcacgtccgattaaaggcacccgtgcgcgtagcaaagatccgagcgaagatcagcgtctgcgtagcgatctgcaggcaagcaccaaagatcgtgcagaaaatctgatgattgttgatctggttcgccatgatctgaatcaggtttgtcgtagtggtagcgttcatgttccgcatatttttgcagttgaaagctttagcagcgttcatcagctggttagcaccgttcgtggtcatctgcgtaatgatattagcaccatggaagcaattcgtgcctgttttcctggtggtagtatgacaggtgcaccgaaaaaacgtaccatggaaattattgatggtctggaaacctgtgcacgtggtgtttatagtggtgcattaggttggattagctttagcggtagtgcagaactgagcattgttattcgtaccgcagtgctgcataaacagcaggcagaatttggtattggtggtgcaattgttgcacatagcgatccgaatgaagagcttgaagagacgctggtcaaagccagcgtgccttattacagtttttacgcagggagcgaaaaatga

SEQ ID NO: 141 - DNA, 367 bp, synthetic construct
  source 1..367: mol_type = other DNA
atagtaatcagtaaggagataaagaatgaacatgaccgaacatcgtcatatgagcccgaccacaccgagcgcaattctgcagccgcagcgtgatcagctggatcgtattaacaatcatctggttgatctgctgggtgaacgtatgagcgtttgtatggatattgcagaactgaaagcagcacatgatattccgatgatgcagcctcagcgcattgttcaggttctggatcagctgaaagataaaagcagtaccgttggtctgcgtccggattatgttcagagcgtttttaaactgatcatcgaggaaacctgcatccaagaagaacagctgattcagcgtcgtcgtaatcagggtcagcgtagctaa

SEQ ID NO: 142 - DNA, 895 bp, synthetic construct
  source 1..895: mol_type = other DNA
cgtaaatataaggaggtcaaacatgaataccaataccgttgtggttttaggtggtgcaggtctgattggtagcatgattagccgtattctgaaacagtatggttattttgttcgtgtggttgatcgtcgtccggcagaatttgaatgtgaatatcatgaaatggacgtgaccaaaccgtttaatgataccggtgcagtttttcgtaatgcaaccgcagttgtttttgcactgccggaaagcgttgcagttagcgcaattccgtgggttaccacctttctgagcagcgaagttgttctgattccgacctgtagcgttcagggtccgttctataaagcactgaaagcagcagcaccgcgtcagccgtttgttggtgttaatccgatgtttagcccgaaactgagcgtgcagggtcgtagcgttgccgtttgtgttgaagatacccaggcagcacagacctttattgaacgtcatctgatggaagccggtatgaaaattcgtcgtatgaccccgagcgcacatgatgaactgatggcactgtgtcaggcactgccgcatgcagcaattttaggttttggtatggcactggcaaaaagcagcgttgatatggatattgttgcagaagttatgcctccgcctatgcgtaccatgatggccctgctgagtcgtattctggttaatccgcctgaagtttattgggatattcagctggaaaatgatcaggcaaccgcacagcgtgatgcactggttcatggtctggaacgtctgcaagaaaatattgtggaacaggattatgagcgcttcaaaagcgatctgcagagcgttagcaccgcactgggtaaacgtctgaatgcgggtgcagttgattgtcagcacctgtttagcctgctgaattaa

SEQ ID NO: 143 - DNA, 2168 bp, synthetic construct
  source 1..2168: mol_type = other DNA
atcgggctgccccagccaatcacaccataccataaggagcattttttatggcgccaagcgtcgactccatcgccacgtccgtggccaacagcttgagcaacggactggctggcgatctgcggaagaaaaccagcggcgcgggaagcctgctcccgacaaccgagactacccagatcgacatcgtcgagcggattctcgccgatgccggtgctactgaccagatcaagttggatggctataccttaacgctcggcgatgtggtgggcgccgcgcggcgagggaggaccgttaaagtcgccgactcgccgcagattcgcgaaaagatcgatgcgtcagtggagtttctgcgcactcaactggacaacagtgtgtacggcgtgaccaccggcttcggcggctcggccgacacccgcaccgaagacgccatttcgctgcaaaaggcgctgctggagcaccagctgtgcggcgttttgccaacctcgatggacggcttcgccttggggcgtggtctggagaactcgttgccactggaagtcgtgcggggcgccatgaccatcagagtcaatagcctgacccgcggccattcggctgtccgtatcgtcgtcctggaagccctgaccaacttcttgaaccacggcatcaccccgatcgtgccgctgcgcgggaccatcagcgcgtcgggcgacttgagcccgctcagttacatcgctgcgtcgatcaccgggcatccggacagcaaggtgcatgtagacgggcaaatcatgagcgcccaagaagctattgcactgaaaggcctgcaacccgtcgttcttggcccgaaggaaggcttgggcctggtcaacggcaccgccgtgtcggcctccatggccacgctcgccctgaccgacgcccacgtgctcagcctgctcgcgcaggccaataccgcactgacggtggaagcaatggtgggccatgccggctcattccacccgttcctgcatgatgtgactcgcccgcaccccacccagatcgaggtggcccgtaacattcgcacgctgctggagggcagcaagtatgccgtacaccacgaaaccgaagtaaaagtgaaagacgacgagggtatccttcgccaggaccgctacccgcttcgctgctcgcctcagtggctgggtccgctggtgagcgacatgatccacgcgcacagcgtgctgtcactggaagcaggccagagtacgaccgacaacccgctgattgacctagaaaacaagatgacccatcatggtggtgccttcatggccagcagcgtgggtaacaccatggagaagacccggctggccgtagcactgatgggcaaggtgtcgttcacccaattgacagaaatgctgaacgctggcatgaaccgcgccctgcccagctgcctggcagccgaggacccgtcactgagttaccactgcaagggccttgatatcgccgccgcagcctacaccagcgagctgggccacctggccaatcctgtcagcacgcatgtgcagccggccgagatgggtaaccaggcaatcaattccctggcgctcatatctgcccgccgtacagccgaggccaacgacgtgctgtctttgctgcttgcaactcacttgtactgtgtactgcaagccgttgatctgcgtgcgatggaatttgaacacaccaaggagttcgagccgatggtcaccgatttgctaaaacagcacttcggcgccctggccacagcggacgtggaggacaaggtgcgcaagagcatctacaagcgcttacagcagaacaattcctacgacctggagcaacgctggcacgacaccttcagcgtagcgaccggtgcggtcgtcgaggcgctggccggcaacgaagtgtctctggcctccctgaacgcctggaaggtggcgtgcgccgaaaaagctatcgccctgacccgtaccgtgcgcgacagcttctgggctgcgcctagctcggcctcgccggcgctcaagtacctgtcaccacggacccgcatcctgtacagctttgtccgcgaagatgtgggcgtcaaggccaggcgcggtgatgtttatctcggcaagcaggaagtgacgatcggtacgaacgtgtcccgtatctatgaggccatcaaagacgggcgcattgcacccgtgctggtcaaaatgatggcataa

SEQ ID NO: 144 - DNA, 993 bp, synthetic construct
  source 1..993: mol_type = other DNA
gaattcattaaagaggagaaaggtaccatggggcccatggcagaatacaaagataatttacttggtgaggcgaacagctttctcgaagtgctggaacaggtttcgcatctcgcaccgctggacaaaccggtgctcatcatcggcgaacgcggcaccggtaaagagctgattgccagccgcctgcattatctctcctcccgttggcaagggccgtttatttcccttaactgcgcggcgttaaatgaaaatctgctggattccgaactgtttggtcacgaagcgggggcgtttaccggtgcgcaaaaacgtcatccagggagatttgaacgtgccgacggcggtacgctatttcttgatgaactcgctacggcaccgatgatggtgcaggagaaattattgcgcgtgattgagtacggtgaactggagcgcgttggcggcagccaaccattgcaggtgaatgtgcggttggtatgcgcgacgaatgccgatctcccggcgatggtcaatgaaggcacttttcgcgctgacctgctcgaccgactggcttttgatgttgtacaactgccaccactgcgcgagcgcgaaagcgacataatgttgatggcagaatactttgccatccagatgtgtcgggaaatcaagctgcctctgttcccggggtttacggagcgcgccagagaaacattgctgaattatcgttggccgggaaatattcgtgaattgaaaaacgtggtggaacgttcagtgtatcgccacggcaccagcgattatccgcttgatgacatcattattgatccctttaaacggcgtccgcctgaagacgctatcgccgtttcagaaaccacctcgcttccaacactgccgctggatttacgtgagtttcagatgcagcaggaaaaagagttgctgcaactcagtttgcaaatgaatgcacgcacacgccgccgcgaacgtcgcgcagagaaacaggctcaatggaaagcagcaaattaa

SEQ ID NO: 145 - DNA, 36 bp, synthetic construct
  source 1..36: mol_type = other DNA
ataaaaaattggcacgcaaattgtattaacagttca

SEQ ID NO: 146 - DNA, 133 bp, synthetic construct
  source 1..133: mol_type = other DNA
nnnnnnnnnnnnnnnnnnnngtttgagagctagggccctgaagaagggccctagcaagttcaaataaggctagtccgttatcaacttgggccctgaagaagggcccaagtggcaccgagtcggtgcttttttt

SEQ ID NO: 147 - DNA, 4794 bp, synthetic construct
  source 1..4794: mol_type = other DNA
ttacgaaatcatcctgtggagcttagtaggtttagcaagatggcagcgcctaaatgtagaatgataaaaggattaagagattaatttccctaaaaatgataaaacaagcgttttgaaagcgcttgtttttttggtttgcagtcagagtagaatagaagtatcaaaaaaagcaccgactcggtgccactttttcaagttgataacggactagccttattttaacttgctatgctgttttgaatggttccaacaagattattttataacttttataacaaataatcaaggagaaattcaaagaaatttatcagccataaaacaatacttaatactatagaatgataacaaaataaactactttttaaaagaattttgtgttataatctatttattattaagtattgggtaatattttttgaagagatattttgaaaaagaaaaattaaagcatattaaactaatttcggaggtcattaaaactattattgaaatcatcaaactcattatggatttaatttaaactttttattttaggaggcaaaaatggataagaaatactcaataggcttagctatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacaggagattttttcaaatgagatggcgaaagtagatgatagtttctttcatcgacttgaagagtcttttttggtggaagaagacaagaagcatgaacgtcatcctatttttggaaatatagtagatgaagttgcttatcatgagaaatatccaactatctatcatctgcgaaaaaaattggtagattctactgataaagcggatttgcgcttaatctatttggccttagcgcatatgattaagtttcgtggtcattttttgattgagggagatttaaatcctgataatagtgatgtggacaaactatttatccagttggtacaaacctacaatcaattatttgaagaaaaccctattaacgcaagtggagtagatgctaaagcgattctttctgcacgattgagtaaatcaagacgattagaaaatctcattgctcagctccccggtgagaagaaaaatggcttatttgggaatctcattgctttgtcattgggtttgacccctaattttaaatcaaattttgatttggcagaagatgctaaattacagctttcaaaagatacttacgatgatgatttagataatttattggcgcaaattggagatcaatatgctgatttgtttttggcagctaagaatttatcagatgctattttactttcagatatcctaagagtaaatactgaaataactaaggctcccctatcagcttcaatgattaaacgctacgatgaacatcatcaagacttgactcttttaaaagctttagttcgacaacaacttccagaaaagtataaagaaatcttttttgatcaatcaaaaaacggatatgcaggttatattgatgggggagctagccaagaagaattttataaatttatcaaaccaattttagaaaaaatggatggtactgaggaattattggtgaaactaaatcgtgaagatttgctgcgcaagcaacggacctttgacaacggctctattccccatcaaattcacttgggtgagctgcatgctattttgagaagacaagaagacttttatccatttttaaaagacaatcgtgagaagattgaaaaaatcttgacttttcgaattccttattatgttggtccattggcgcgtggcaatagtcgttttgcatggatgactcggaagtctgaagaaacaattaccccatggaattttgaagaagttgtcgataaaggtgcttcagctcaatcatttattgaacgcatgacaaactttgataaaaatcttccaaatgaaaaagtactaccaaaacatagtttgctttatgagtattttacggtttataacgaattgacaaaggtcaaatatgttactgaaggaatgcgaaaaccagcatttctttcaggtgaacagaagaaagccattgttgatttactcttcaaaacaaatcgaaaagtaaccgttaagcaattaaaagaagattatttcaaaaaaatagaatgttttgatagtgttgaaatttcaggagttgaagatagatttaatgcttcattaggtacctaccatgatttgctaaaaattattaaagataaagattttttggataatgaagaaaatgaagatatcttagaggatattgttttaacattgaccttatttgaagatagggagatgattgaggaaagacttaaaacatatgctcacctctttgatgataaggtgatgaaacagcttaaacgtcgccgttatactggttggggacgtttgtctcgaaaattgattaatggtattagggataagcaatctggcaaaacaatattagattttttgaaatcagatggttttgccaatcgcaattttatgcagctgatccatgatgatagtttgacatttaaagaagacattcaaaaagcacaagtgtctggacaaggcgatagtttacatgaacatattgcaaatttagctggtagccctgctattaaaaaaggtattttacagactgtaaaagttgttgatgaattggtcaaagtaatggggcggcataagccagaaaatatcgttattgaaatggcacgtgaaaatcagacaactcaaaagggccagaaaaattcgcgagagcgtatgaaacgaatcgaagaaggtatcaaagaattaggaagtcagattcttaaagagcatcctgttgaaaatactcaattgcaaaatgaaaagctctatctctattatctccaaaatggaagagacatgtatgtggaccaagaattagatattaatcgtttaagtgattatgatgtcgatgccattgttccacaaagtttccttaaagacgattcaatagacaataaggtcttaacgcgttctgataaaaatcgtggtaaatcggataacgttccaagtgaagaagtagtcaaaaagatgaaaaactattggagacaacttctaaacgccaagttaatcactcaacgtaagtttgataatttaacgaaagctgaacgtggaggtttgagtgaacttgataaagctggttttatcaaacgccaattggttgaaactcgccaaatcactaagcatgtggcacaaattttggatagtcgcatgaatactaaatacgatgaaaatgataaacttattcgagaggttaaagtgattaccttaaaatctaaattagtttctgacttccgaaaagatttccaattctataaagtacgtgagattaacaattaccatcatgcccatgatgcgtatctaaatgccgtcgttggaactgctttgattaagaaatatccaaaacttgaatcggagtttgtctatggtgattataaagtttatgatgttcgtaaaatgattgctaagtctgagcaagaaataggcaaagcaaccgcaaaatatttcttttactctaatatcatgaacttcttcaaaacagaaattacacttgcaaatggagagattcgcaaacgccctctaatcgaaactaatggggaaactggagaaattgtctgggataaagggcgagattttgccacagtgcgcaaagtattgtccatgccccaagtcaatattgtcaagaaaacagaagtacagacaggcggattctccaaggagtcaattttaccaaaaagaaattcggacaagcttattgctcgtaaaaaagactgggatccaaaaaaatatggtggttttgatagtccaacggtagcttattcagtcctagtggttgctaaggtggaaaaagggaaatcgaagaagttaaaatccgttaaagagttactagggatcacaattatggaaagaagttcctttgaaaaaaatccgattgactttttagaagctaaaggatataaggaagttaaaaaagacttaatcattaaactacctaaatatagtctttttgagttagaaaacggtcgtaaacggatgctggctagtgccggagaattacaaaaaggaaatgagctggctctgccaagcaaatatgtgaattttttatatttagctagtcattatgaaaagttgaagggtagtccagaagataacgaacaaaaacaattgtttgtggagcagcataagcattatttagatgagattattgagcaaatcagtgaattttctaagcgtgttattttagcagatgccaatttagataaagttcttagtgcatataacaaacatagagacaaaccaatacgtgaacaagcagaaaatattattcatttatttacgttgacgaatcttggagctcccgctgcttttaaatattttgatacaacaattgatcgtaaacgatatacgtctacaaaagaagttttagatgccactcttatccatcaatccatcactggtctttatgaaacacgcattgatttgagtcagctaggaggtgactaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 148 - DNA, 802 bp, synthetic construct
  source 1..802: mol_type = other DNA
tttacggctagctcagccctaggtattatgctagcgaattcattaaagaggagaaaggtaccatggggcccgcttctaactttactcagttcgttctcgtcgacaatggcggaactggcgacgtgactgtcgccccaagcaacttcgctaacgggatcgctgaatggatcagctctaactcgcgttcacaggcttacaaagtaacctgtagcgttcgtcagagctctgcgcagaatcgcaaatacaccatcaaagtcgaggtgcctaaaggcgcctggcgttcgtacttaaatatggaactaaccattccaattttcgccacgaattccgactgcgagcttattgttaaggcaatgcaaggtctcctaaaagatggaaacccgattccctcagcaatcgcagcaaactccggcatctacggtggcggaggtagcatgtcccatcagaaaattattcaggatcttatcgcatggattgacgagcatattgaccagccgcttaacattgatgtagtcgcaaaaaaatcaggctattcaaagtggtacttgcaacgaatgttccgcacggtgacgcatcagacgcttggcgattacattcgccaacgccgcctgttactggccgccgttgagttgcgcaccaccgagcgtccgatttttgatatcgcaatggacctgggttatgtctcgcagcagaccttctcccgcgttttcgcgcggcagtttgatcgcactcccgcggattatcgccaccgcctgtaagcggccgccacgcaaaaaaccccgcttcggcggggttttttcgc

SEQ ID NO: 149 - DNA, 2117 bp, synthetic construct
  source 1..2117: mol_type = other DNA
atttgtcctactcaggagagcgttcaccgacaaacaacagataaaacgaaaggcccagtctttcgactgagcttttcgttttatttgatgcctttaattaaacgttcgtaatcaagccacttcctttttgcattgacgcagggtgtcggaaggcaactcgccgaacgcgctcctatagttttcagcgaagcgtcccaaatgtaagaagccgtagtctagggctatctcagttatactacgcacattggcactgggatcgttcaagcaggcgcggatgctttcgagcttgcggttgcggatgtagttcttcggcgtggtgccggcgtgcttctcgaacaaattgtagagcgagcgtggactcatcatcgccagctccgctaaccgctcaaggctgatattccgtttgagattctcctcaatgaattgaacgactcgctcgaaagacgggttacctttgctgaaaatttcacggctgacattgctgcccagcatttcgagcagcttggaagcgatgatccccgcatagtgctcttggacccgaggcatcgactttgtatgttccgcttcgtcacaaactaacccgagtagattgataaagccatcgagttgctggagattgtgtcgcgcggcgaaacggataccctccctcggcttgtgccaattgttgtcactgcacgcccgatcaaggaccactgagggcaatttaacgataaatttctcgcaatcttctgaataggtcaggtcggcttggtcatccggattgagcagcaatagttcgcccggcgcaaaatagtgctcctggccatggccacgccacaggcaatggcctttgagtattatttgcagatgataacaggtttctaatccaggcgagattaccctcacgctaccgccgtagctgattcgacacagatcgaggcatccgaagattctgtggtgcagcctgcctgccgggcgcccgcccttgggcaggcgaatagagtgcgtaccgacatactggttaacataatcggagactgcatagggctcggcgtggacgaagatctgacttttctcgttcaataagcaaaaatccatagttcacggttctcttattttaatgtgggctgcttggtgtgatgtagaaaggcgccaagtcgatgaaaatgcatctcgacgtgatgcgtatacgggttacccccattgccacgttgcgccatcctttttgcaatcagtgaccacttttccaagcaaaaataacgccaagcagaacgaagacgttctttttaagaagcgagaacaccagaagttcgtgctgtcggggcatggggcgacgaattggcggataaaggggatctgctggatattacggcctttttaaagaccgtaaagaaaaataagcacaagttttatccggcctttattcacattcttgcccgcctgatgaatgctcatccgtaattacgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaaccgcctatttccctaaagggtttattgagaatatgtttttcgtctcagccaatccctgggtgagtttcaccagttttgatttaaacgtggccaatatggacaacttcttcgcccccgttttcaccatgcatgggaattagcttgatctgaccaacgaccggtagcggagctatccaacggcggtataccaggaaaacacacagcaggtacatcagaacagtaccatgactgaagaacaaatagttttttcctgatccataaagcagaacggcctgctccatgacaaatctggctccccaactaatgccccatgcagccagcataaccagcataaagtgcagtgtccggtttgatagggataagtccagccttgcaagaagcggatacaggagtgcaaaaaatggctatctctagtaaggcctaccccttaggctttatgcaacagaaacaataataatggagtcatgaccatgcctaggccgcggttaggaggaaaaacatatg

SEQ ID NO: 150 - DNA, 949 bp, synthetic construct
  source 1..949: mol_type = other DNA
gcctacggtatccaccggagacctatggcagcctccggccgccataggacacctttggttgccaagggtgacctatggtgaccatgggccaccacgggcgacctcaggtatcctgcggtgtcctgcggttaccaaaggcgtcctttgggttccaccggatacctccggacttgacagctagctcagtcctagggattgtgctagcgaattcattaaagaggagaaaggtaccatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataa

SEQ ID NO: 151 - DNA, 910 bp, synthetic construct
  source 1..910: mol_type = other DNA
agcatttgcgatcattcacgcagcgcttattcagttgctcactgcgatgtcataatcatcgctacgagctgtgaaagatgcataaagctcgtacgacgcgttcgctcgtctcctcacttctcctacggagcgttctggacacaacgtcgtcttgaagttgcgattatagattgacggctagctcagtcctaggtatagtgctagcgaattcattaaagaggagaaaggtaccatggcgagtagcgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaa

SEQ ID NO: 152 - DNA, 919 bp, synthetic construct
  source 1..919: mol_type = other DNA
agcatttgcgatcattcacgcagcgcttattcagttgctcactgcgatgtcataatcatcgctacgagctgtgaaagatgcataaagctcgtacgacgcgttcgctcgtctcctcacttctcctacggagcgttctggacacaacgtcgtcttgaagttgcgattatagattgacagctagctcagtcctagggattgtgctagcgaattcattaaagaggagaaaggtaccatgcatcaccatcaccatcacccatcactcagtaaagaagcggccctggttcatgaagcgttagttgcgcgaggactggaaacaccgctgcgcccgcccgtgcatgaaatggataacgaaacgcgcaaaagccttattgctggtcatatgaccgaaatcatgcagctgctgaatctcgacctggctgatgacagtttgatggaaacgccgcatcgcatcgctaaaatgtatgtcgatgaaattttctccggtctggattacgccaatttcccgaaaatcaccctcattgaaaacaaaatgaaggtcgatgaaatggtcaccgtgcgcgatatcactctgaccagcacctgtgaacaccattttgttaccatcgatggcaaagcgacggtggcctatatcccgaaagattcggtgatcggtctgtcaaaaattaaccgcattgtgcagttctttgcccagcgtccgcaggtgcaggaacgtctgacgcagcaaattcttattgcgctacaaacgctgctgggcaccaataacgtggctgtctcgatcgacgcggtgcattactgcgtgaaggcgcgtggcatccgcgatgcaaccagtgccacgacaacgacctctcttggtggattgttcaaatccagtcagaatacgcgccacgagtttctgcgcgctgtgcgtcatcacaactaa

SEQ ID NO: 153 - DNA, 691 bp, synthetic construct
  source 1..691: mol_type = other DNA
agcatttgcgatcattcacgcagcgcttattcagttgctcactgcgatgtcataatcatcgctacgagctgtgaaagatgcataaagctcgtacgacgcgttcgctcgtctcctcacttctcctacggagcgttctggacacaacgtcgtcttgaagttgcgattatagattgacagctagctcagtcctagggattgtgctagcgaattcattaaagaggagaaaggtaccatgcatcaccaccatcaccatacgtcctcaactccagttagaactgcttacgttaccaggatcgaacacttctccgctgcgcacagattgaactccgtccacctctcgcctgctgagaacgtcaagctcttcggtaagtgcaaccacacttccggtcacggtcacaactacaaggtcgaggtgaccatcaagggtcagatcaacccacaatccggcatggtcatcaacatcaccgatcttaagaagactttgcaagtcgctgtcatggacccttgtgaccatagaaacttggatatagacgtcccatacttcgagtccagaccctccactactgagaacctcgctgtcttcttgtgggagaatatcaagagccacttgccaccttccgacgcgtacgatttgtacgagatcaagttgcacgaaaccgacaagaacgttgtcgtttacagaggtgaataa

SEQ ID NO: 154 - DNA, 1099 bp, synthetic construct
  source 1..1099: mol_type = other DNA
agcatttgcgatcattcacgcagcgcttattcagttgctcactgcgatgtcataatcatcgctacgagctgtgaaagatgcataaagctcgtacgacgcgttcgctcgtctcctcacttctcctacggagcgttctggacacaacgtcgtcttgaagttgcgattatagattgacagctagctcagtcctagggattgtgctagcgaattcattaaagaggagaaaggtaccatgcatcaccatcaccaccatagcagtaaagaacatcatttggttattattaacggtgttaatagaggtttcgggcactccgtcgcgttggattacataagacactcaggtgctcacgcggtgtcctttgtcttggttggtagaacccagcattccttggagcaagtcttaacggagctgcacgaggctgcatcccacgctggtgtcgtcttcaagggtgtcgttgtgtccgaggtcgacctggctcacttgaactccctcgactccaacctcgcgaggatacagtccgccgccgctgacctaagagacgaggcggcgcaaagcaccagaactatcactaagtcggtcctcttcaacaacgcgggtagcttgggtgacttgtccaagactgttaaggagttcacctggcaagaggctcgttcctacctcgatttcaacgtcgtgtccctcgttggtttgtgctccatgttcttgaaggataccctcgaagcattcccaaaggaacaatacccagatcatagaactgtggtcgtgtccatctcttccctattagctgttcaggctttcccaaactggggtttgtacgctgctggtaaggcagctagagatagactattaggtgttattgctctcgaagaagcagctaataacgtaaagaccttgaactacgctccaggtccattggataacgaaatgcaggctgacgtccgcagaactttgggtgataaggaacaactgaagatctacgacgacatgcataagtctggttccttggtgaagatggaggactcctctagaaagttgattcatttgttaaaggctgacaccttcacctccggtggccacattgatttctacgacgaataa

SEQ ID NO: 155 - DNA, 5102 bp, synthetic construct
  source 1..5102: mol_type = other DNA
ccagctggcaattccgacgtcgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagcgcgaattgatctggtttgacagcttatcatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatccggctcgtataatgtgtggaattgtgagcggataacaatttcagaattcaaaagatcttttaaggacgaaacgtacatatgaaaaccgtggtgattattgatgcactgcgtaccccgattggtaaatacaaaggtagcctgagccaggttagcgcagttgatctgggcacccatgttaccacccagctgctgaaacgtcatagcacattagcgaagaaattgatcaggtgatttttggcaatgttctgcaggcaggtaatggtcagaatccggcacgtcagattgcaattaatagcggtctgagccatgaaattccggcaatgaccgttaatgaagtttgtggtagcggtatgaaagcagttattctggcaaaacagctgatccagctgggcgaagccgaagttctgattgccggtggtattgaaaatatgagccaggcaccgaaactgcagcgtttcaattatgaaaccgaaagctatgatgcaccgtttagcagcatgatgtatgatggtctgaccgatgcatttagcggtcaggcaatgggtctgacagcagaaaatgttgcagaaaaatatcatgtgacccgtgaagaacaggatcagtttagcgttcatagccagctgaaagcagcacaggcacaggccgaaggtattttcgcagatgaaattgcaccgctggaagttagcggcaccctggttgaaaaagatgaaggtattcgtccgaatagcagcgttgaaaaactgggtacactgaaaacggtgtttaaagaagatggcaccgttaccgcaggcaatgcaagtaccattaatgatggtgcaagcgcactgattattgccagccaagaatatgccgaagcacatggtctgccgtatctggcaattattcgtgatagcgttgaagttggtattgatccggcatatatgggtattagcccgattaaagcaattcagaaactgctggcacgtaatcagctgaccaccgaagaaatcgacctgtacgaaattaatgaagcatttgccgcaaccagcattgttgttcagcgtgaactggcactgccggaagaaaaagttaacatttatggcggtggcatcagcctgggtcatgcaattggtgcaaccggtgcacgtctgctgaccagcctgagctatcagctgaatcagaaagagaaaaaatacggcgttgcaagcctgtgtattggtggtggcctgggtctggcaatgctgctggaacgccctcaacagaaaaaaaacagccgtttttatcagatgagtccggaagaacgtctggccagcctgctgaatgaaggtcagattagcgcagataccaaaaaagaatttgaaaacaccgcactgagcagccagattgccaaccacatgattgaaaatcagatcagcgaaaccgaagtgccgatgggtgttggtctgcatctgaccgtggatgaaacggattatctggttccgatggcaaccgaagaaccgagcgttattgcagccctgagcaatggtgcaaaaattgcacagggctttaaaaccgtgaatcagcagcgtctgatgcgtggtcagattgttttttatgatgttgccgatgcagaaagcctgattgatgaactgcaggttcgtgaaacagaaattttccagcaggcagaactgagttatccgagcattgttaaacgcggtggtggtctgcgtgatctgcagtatcgtgcatttgatgaaagttttgttagcgtggattttctggtggatgttaaagacgcaatgggtgccaatattgttaatgcaatgctggaaggtgttgccgaactgtttcgtgaatggtttgcagaacaaaaaatcctgtttagcatcctgagtaactatgccaccgaaagcgttgttaccatgaaaacagcaattccggttagccgtctgagcaaaggtagtaatggtcgtgaaattgccgaaaaaattgttctggcaagccgttatgccagcctggatccgtatcgtgccgttacccataataaaggtattatgaatggcattgaagcagttgtgctggccaccggtaatgatacccgtgcagttagcgcaagctgtcatgcatttgcagttaaagaaggtcgttatcagggtctgaccagctggaccctggatggtgagcagctgattggtgaaattagcgttccgctggcactggcaaccgttggtggtgccaccaaagttctgccgaaaagccaggcagcagccgatctgctggcagttaccgatgcaaaagaactgagccgtgttgttgcagcagttggtctggcacagaatctggcagcactgcgtgcactggttagcgaaggcattcagaaaggtcacatggcactgcaggcacgttcactggccatgaccgtgggtgcgaccggtaaagaagttgaagccgttgcacagcaactgaaacgccagaaaacaatgaatcaggatcgtgccctggcaattctgaatgatctgcgtaaacagtaatgattagcgacaaaatatgaggagtgcaaaaaatgaccattggcatcgacaaaatcagcttttttgttccgccttactatatcgacatgaccgcactggccgaagcacgtaatgttgatccgggtaaatttcatattggtattggtcaggatcagatggccgttaatccgattagccaggatattgttacctttgcagcaaatgcagcagaagcaattctgaccaaagaagataaagaagccatcgatatggttattgttggcaccgaaagcagcattgatgaaagcaaagcagccgcagttgttctgcatcgtctgatgggtattcagccgtttgcacgtagctttgaaattaaagaaggttgttacggcgcaaccgcaggtctgcagctggcaaaaaatcatgttgcactgcatccggataaaaaagttctggttgttgcagcagatatcgccaaatatggtctgaatagcggtggtgaaccgacccagggtgccggtgcagttgcaatgctggttgcaagcgaaccgcgtattctggcactgaaagaggataatgttatgctgacgcaggatatctatgatttttggcgtccgaccggtcatccgtatccgatggttgatggtccgctgagcaatgaaacctatattcagagctttgcacaggtgtgggatgaacataaaaaacgtaccggtctggatttcgcagattatgatgcactggcctttcatattccgtataccaaaatgggtaaaaaagcactgctggcgaaaattagcgatcagaccgaagccgaacaagaacgtatcctggcacgttatgaagaaagcattatctatagccgtcgtgtgggtaatctgtataccggtagcctgtatctgggtctgattagcctgctggaaaatgcaaccaccctgaccgctggtaatcagattggtctgtttagctatggtagcggtgccgttgcagaattctttaccggtgaactggttgcaggttatcagaatcatctgcagaaagaaacccatctggccctgctggataatcgtaccgaactgagcattgcagaatatgaagcaatgtttgcagaaaccctggataccgatattgatcagaccctggaagacgaattaaaatatagcattagcgccattaataacaccgtgcgtagctatcgtaattaa

SEQ ID NO: 156 - DNA, 3828 bp, synthetic construct
  source 1..3828: mol_type = other DNA
agcatttgcgatcattcacgcagcgcttattcagttgctcactgcgatgtcataatcatcgctacgagctgtgaaagatgcataaagctcgtacgacgcgttcgctcgtctcctcacttctcctacggagcgttctggacacaacgtcgtcttgaagttgcgattatagattgacggctagctcagtcctaggtatagtgctagcgaattcattaaagaggagaaaggtaccatgaaaaccgtggtgattattgatgcactgcgtaccccgattggtaaatacaaaggtagcctgagccaggttagcgcagttgatctgggcacccatgttaccacccagctgctgaaacgtcatagcaccattagcgaagaaattgatcaggtgatttttggcaatgttctgcaggcaggtaatggtcagaatccggcacgtcagattgcaattaatagcggtctgagccatgaaattccggcaatgaccgttaatgaagtttgtggtagcggtatgaaagcagttattctggcaaaacagctgatccagctgggcgaagccgaagttctgattgccggtggtattgaaaatatgagccaggcaccgaaactgcagcgtttcaattatgaaaccgaaagctatgatgcaccgtttagcagcatgatgtatgatggtctgaccgatgcatttagcggtcaggcaatgggtctgacagcagaaaatgttgcagaaaaatatcatgtgacccgtgaagaacaggatcagtttagcgttcatagccagctgaaagcagcacaggcacaggccgaaggtattttcgcagatgaaattgcaccgctggaagttagcggcaccctggttgaaaaagatgaaggtattcgtccgaatagcagcgttgaaaaactgggtacactgaaaacggtgtttaaagaagatggcaccgttaccgcaggcaatgcaagtaccattaatgatggtgcaagcgcactgattattgccagccaagaatatgccgaagcacatggtctgccgtatctggcaattattcgtgatagcgttgaagttggtattgatccggcatatatgggtattagcccgattaaagcaattcagaaactgctggcacgtaatcagctgaccaccgaagaaatcgacctgtacgaaattaatgaagcatttgccgcaaccagcattgttgttcagcgtgaactggcactgccggaagaaaaagttaacatttatggcggtggcatcagcctgggtcatgcaattggtgcaaccggtgcacgtctgctgaccagcctgagctatcagctgaatcagaaagagaaaaaatacggcgttgcaagcctgtgtattggtggtggcctgggtctggcaatgctgctggaacgccctcaacagaaaaaaaacagccgtttttatcagatgagtccggaagaacgtctggccagcctgctgaatgaaggtcagattagcgcagataccaaaaaagaatttgaaaacaccgcactgagcagccagattgccaaccacatgattgaaaatcagatcagcgaaaccgaagtgccgatgggtgttggtctgcatctgaccgtggatgaaacggattatctggttccgatggcaaccgaagaaccgagcgttattgcagccctgagcaatggtgcaaaaattgcacagggctttaaaaccgtgaatcagcagcgtctgatgcgtggtcagattgttttttatgatgttgccgatgcagaaagcctgattgatgaactgcaggttcgtgaaacagaaattttccagcaggcagaactgagttatccgagcattgttaaacgcggtggtggtctgcgtgatctgcagtatcgtgcatttgatgaaagttttgttagcgtggattttctggtggatgttaaagacgcaatgggtgccaatattgttaatgcaatgctggaaggtgttgccgaactgtttcgtgaatggtttgcagaacaaaaaatcctgtttagcatcctgagtaactatgccaccgaaagcgttgttaccatgaaaacagcaattccggttagccgtctgagcaaaggtagtaatggtcgtgaaattgccgaaaaaattgttctggcaagccgttatgccagcctggatccgtatcgtgccgttacccataataaaggtattatgaatggcattgaagcagttgtgctggccaccggtaatgatacccgtgcagttagcgcaagctgtcatgcatttgcagttaaagaaggtcgttatcagggtctgaccagctggaccctggatggtgagcagctgattggtgaaattagcgttccgctggcactggcaaccgttggtggtgccaccaaagttctgccgaaaagccaggcagcagccgatctgctggcagttaccgatgcaaaagaactgagccgtgttgttgcagcagttggtctggcacagaatctggcagcactgcgtgcactggttagcgaaggcattcagaaaggtcacatggcactgcaggcacgttcactggccatgaccgtgggtgcgaccggtaaagaagttgaagccgttgcacagcaactgaaacgccagaaaacaatgaatcaggatcgtgccctggcaattctgaatgatctgcgtaaacagtaatgattagcgacaaaatatgaggagtgcaaaaaatgaccattggcatcgacaaaatcagcttttttgttccgccttactatatcgacatgaccgcactggccgaagcacgtaatgttgatccgggtaaatttcatattggtattggtcaggatcagatggccgttaatccgattagccaggatattgttacctttgcagcaaatgcagcagaagcaattctgaccaaagaagataaagaagccatcgatatggttattgttggcaccgaaagcagcattgatgaaagcaaagcagccgcagttgttctgcatcgtctgatgggtattcagccgtttgcacgtagctttgaaattaaagaaggttgttacggcgcaaccgcaggtctgcagctggcaaaaaatcatgttgcactgcatccggataaaaaagttctggttgttgcagcagatatcgccaaatatggtctgaatagcggtggtgaaccgacccagggtgccggtgcagttgcaatgctggttgcaagcgaaccgcgtattctggcactgaaagaggataatgttatgctgacgcaggatatctatgatttttggcgtccgaccggtcatccgtatccgatggttgatggtccgctgagcaatgaaacctatattcagagctttgcacaggtgtgggatgaacataaaaaacgtaccggtctggatttcgcagattatgatgcactggcctttcatattccgtataccaaaatgggtaaaaaagcactgctggcgaaaattagcgatcagaccgaagccgaacaagaacgtatcctggcacgttatgaagaaagcattatctatagccgtcgtgtgggtaatctgtataccggtagcctgtatctgggtctgattagcctgctggaaaatgcaaccaccctgaccgctggtaatcagattggtctgtttagctatggtagcggtgccgttgcagaattctttaccggtgaactggttgcaggttatcagaatcatctgcagaaagaaacccatctggccctgctggataatcgtaccgaactgagcattgcagaatatgaagcaatgtttgcagaaaccctggataccgatattgatcagaccctggaagacgaattaaaatatagcattagcgccattaataacaccgtgcgtagctatcgtaattaa

SEQ ID NO: 157 - DNA, 150 bp, synthetic construct
  source 1..150: mol_type = other DNA
taaaaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttata

SEQ ID NO: 158 - DNA, 1152 bp, synthetic construct
  source 1..1152: mol_type = other DNA
gttctcagggctcgccgagaaacgcataacccatgctttgaggtaattattcctgaataaagcgggttggccattgaacgttcacgcgcgcagttgtctcaaacctgccatttgagtttcgccgcccgacggtgcagttgctaaaacggcggttgaacagccgactgaagatgcgctctctggcgctcctcgggggacaagctacatgaaaaaaactctggtattctacgagcctcagctcgggtctggagcctggatggccaggaattgcctcgctgggcgcgtatatttattgctgctgcacccgaccgcagcggctgtcagatattaagatacatgcaggtttcctgaggtttgaaacttcaagtggccgttaaggactcaaatatggaattgatcccggtaattttatccggtggcgttggtagccgtctgtggccagtatcagaattcattaaagaggagaaaggtaccatggcgagtagcgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagttaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaa

SEQ ID NO: 159 - DNA, 455 bp, synthetic construct
  source 1..455: mol_type = other DNA
ggaagcctcacgggcagcgcgacccaaacgggtcatatagtcaagaacggactcagtcatgggttcggtgtcttggcgaaggggaaatcggctgattataactgccgcgcaggtgtacgcccagcggcgggtggcggatggtagaaaatggatggggcaatgtgtaggaaagatgtaaccggggttatcgagatttccatctcctgtcgcggccctttagcgggcgcgcccgctcccactgggatttgtgtaggagcgggtttacccgcgaaagggccggcactgccaacatcaccgtccaattcagcctcgattaagccattcattgctatcatccccgcctctccagccacgaactgccccgcatgccagccctgcccgacagctttttcgaccgcgacgcccagaccctggccaaggccctggaattcattaaagaggagaaaggtaccatg

SEQ ID NO: 160 - DNA, 585 bp, synthetic construct
  source 1..585: mol_type = other DNA
aaattcgtggtcaagcaggtgatcgtgctcgggttcgtgcaggtcgtcttcgtcgcgcatgctggctcctgggataatggcaggcctgtataggctgatcagggtgccgggtcaatgcgtggtcgcttaatcctgggttaaccggaccggcgcaacctgcaggctctcccttcaaacgtcttttgcccgccttccatggcgggcttttttatgaccctgcgcacttcgttgcagatgatgacagcctcatgacctgaccttcacgaaatgtcgacatccggatgggcacactggccctgctccgtatgttcttgcagcccgccgcatccttgccgcgggcttttcttttttccgcaaaggccagccaggcatacgcaggaattttgtggaagcgcccaccttgaccatgacctgaagcagttttgcctgggccggcaaggtctatgctatcccgacgatcacccaagctcacatcggatagacacggaggctctcatgaaagctgctgtcgttgcaccaggccgtcgcgtggacgtgatagagaaaagcctgcgcgaattcattaaagaggagaaaggtaccatg

SEQ ID NO: 161 - DNA, 541 bp, synthetic construct
  source 1..541: mol_type = other DNA
gtacatgctcatgtttcgacggatcgggtcatcaaagccgccaaaaccaaaagaccttgaaagggatggcccttacggcgagtcactttttgtcaaacgcgacaaaaagtaaccaaaaaacgctgcgctcccatcatccggcccctgcgctgcgctccggggtcccctcactccggccttgctcccggcaggaccgcgccgaaggccccatcctggggcctcagcgcttgacgggcatccatgcccgtcacctgcctccgcaaggcctgcgttcggcctcctgaagtcgcgaagatcaagatcaagatcaagatcaagatcaagatcaagatcaacagcaacagcaacagcaatagctacagcaataggcaacgctgggggaggcacagacaaaaccctacaatccgagtaaagtgccgcccccgccgtttacagcaaaaggatcccttccatgacacaccctttggatatcgccgtcgtcggcgccaccggcagcgtcggtgaagccttggaattcattaaagaggagaaaggtaccatg

SEQ ID NO: 162 - DNA, 480 bp, synthetic construct
  source 1..480: mol_type = other DNA
cagcaccagtttcaggccttcgcgcaggtattgggggccagagaggacaggggcttgcatctagagactccgcagagaaggaaaacgcgctgaccttaccgagtttgccgcgccgacgaaaggcacgccgtgggcgggaaacgggatgtaacaaaagcgcccggggcttgtttatcgatgaaatcgcagcataggcgatgcctatgaggtggattgtctaaggatatttccttaatctttgcgacctcgctacagtgcgctcaacttttcgctttgcgggcctgcgcgttttagccttccccaagtgctgcgtgggtcctttttaatttcttggctggcgcagccggtacaccgatgccggccctgcggcccgctcgacaggagttcgacatgtctgaagtacgtcattcgcgcgtcatcattctcggttccggccctgccggttacagcgaattcattaaagaggagaaaggtaccatg

SEQ ID NO: 163 - DNA, 434 bp, synthetic construct
  source 1..434: mol_type = other DNA
gatagcctcgggctggtcgccagccggctggaaacgtgtgacgagctggaactcggacatgaaggacctcgcggtgcagcagctgtaaatttagccagtagtctatacccaaatgcgcccgttcgggggcgctttcaaggcacaggtgtgggcgcctgcaacggtggacacggcatttagaccaatggtcgaaaaatatttgcgcaaatagccccaaaagctgcgccagagtgtcgcggtgaccggtcggtatcactatactgactccccgtttgtgcaccgcttcagtgcattcggctggagcgtgtacgccctatcacactccaatcagagccaaggtaacaatgagcctgttttccgctgtcgagctggcaccccgcgaccctattctgggcctcaacgaagaattcattaaagaggagaaaggtaccatg

SEQ ID NO: 164 - DNA, 536 bp, synthetic construct
  source 1..536: mol_type = other DNA
cagggcttcgaacagcaccagcaggttcatgtcgacgcggcgcaggtcgttgcggttcatgagggctcggcgtcctgggcaaagggtatggctgtgtattgaagcatggcgcggggccttgtgccttgtatggatgtgccggcctcatcgcgggcttgaccgcgatgaggccgatacaggcctgcgcctgacagagccagcctatcaggctccaacagccactctattagacctctgcccaagctcggctagtctttctcgtggccccgcgaattccgcgacagggcagcgcgtcagcacctgcgtgcaagaccgtgccccctcgccgtgacagcttcgcaagcccagtgtacacctgatgaggggtagtacgagcccacccgctcggctgaaagaacactggcatagaccggaaatctggataaccgacccaaaggtacccgcagatgtcgaacgaatcgaaatgcccgttccatcaaaccgcaggtggcggcaccaccaaccgtgaattcattaaagaggagaaaggtaccatg

SEQ ID NO: 165 - DNA, 620 bp, synthetic construct
  source 1..620: mol_type = other DNA
cttgcgcaccggccggcttttgatggtgatttctgggaagactttgacgataagtttcattggttaacagcgcgcgcagggcctgccgaaaatgaggggcgcgaattatatcggaaattgctcaggatttgaccaacttttgatcagaagctttgagataaatgcagaggccaggttttgcagcgcctgttccggccctttcgcgggtaaacccgcgcctacaggcggtgcaaagcccgtaggagcgggtttacccgcgaagaggccctagaacctggcacaccactcacgccacgcaccctattggtgcgacacgatcaaaaaacgcatcgtaagggtgcactttcacccgctaacccaacgccaaatgcacgcaaacgcccccttttatcccaccctcgccattttcgggcactggcatgcaatttgctcccttgtgaggcaggtaagcttggccgactatccgcgcccggcaacaccctttttccagggcagcggcccaccgcgctctagaccatccggaggacaacatgtcgaagtcggttcaactcatcaaagatcatgacgtcaagtggattgatctgcgtttcgaattcattaaagaggagaaaggtaccatg

SEQ ID NO: 166 - DNA, 424 bp, synthetic construct
  source 1..424: mol_type = other DNA
gttgaagaatttgccatgttcatggcggcgcatctgggggccctgtcgccgcaggggtgatggtttctcctgttgcggcctcttcgcgggtgaacccgctcctacgaggatttcacagtgtaggggcgggttcacccgcgaaggggcccgcacaagcactacaaaaacccctttagtaatcgctggattgtctgtagccttcggcctccgataataatccgcgcccgcagagggccgtggcggtcaacatgccgtccggcacccttagccgatctgccagggccgggtgatacactcgatttgacgcgcaagcgcgcctgcaggtccagcgatcatgacccagatttccgaacgccttttggttcaggcccacctcgacgccaagcagcccaacgaattcattaaagaggagaaaggtaccatg

SEQ ID NO: 167 - DNA, 356 bp, synthetic construct
  source 1..356: mol_type = other DNA
ctgggtaacacgcggttgatcgacaacctctacctgcatttggaagagaagaccgcataacggtcttgggctgccttgcagcccattcgcgggcaagcccgctcctgcacgtttacctgtaggggcgggcttgcttgcaatgcccccaaaatccccctgccatacccattcccagcacgtggcctttgcctataatggtgccagcctgaacccggcaacgactgccgtgtccaagccctcaccacgcaccaagggaaccccgcgcaatggcgtattaccgtacaccccacgatgtgacggccctgcccgcctggcaggcgcttcaggaattcattaaagaggagaaaggtaccatg

SEQ ID NO: 168 - DNA, 902 bp, synthetic construct
  source 1..902: mol_type = other DNA
gtcgaatttgctttcgaatttctgccattcatccgcttattatcacttattcaggcgtagcaaccaggcgtttaagggcaccaataactgccttaaaaaaattacgccccgccctgccactcatcgcagtacggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctccaccgcggtggcggccgctctagaactagtggatcccccgggctgcaggaattcgatatcaagcttatcgataccgtcgacctcgagggggggcccggtacccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgc

SEQ ID NO: 169 - DNA, 3077 bp, synthetic construct
  source 1..3077: mol_type = other DNA
atgaccgacctgatcgaagtgaagacggcagacctggtgggcgaggcgcttgggtgggccgtgggcacggcggaaggcctggacctgttcatggcgccgccggagtacggcaacccacaccgagtgttcgcccgctaccagggccaggccatcgagcacaccaagcgcttcaacccgtgggaagactgggcggttggcgggccgatcatgcagaagcacaacgtcagcctgcactgcccgcagccagagtgggactactgggcagcctggataaccgataacggcaaggacgtcgcccagggcgctgatctgccgttgccggcggcgtgccgggccatagtcgcccaccagctcggcgataccgtccaggtgccgaaggagctgatgccatgaccgtgatccttcccctcgcctacatggcctacctgatctacagggggcttctcggtgagggaggcgcctgcaagcaaagggcacgacatgacctgacgacagcacggcaaaaaacaaactcgaaaggatcatccacaagatcaagcgctgcctggcgctattcaaaagctcgaatgaatatgagagagtctaggcccacccgccgattacgaaggtcttcgctcggagcacaccccagaccaaggctcgactcatagtttcgcttggtctggtgctgtaagcctcttctacaattcggtccccgcttttggagtacaccccgatgaagagctgcgtttcgcctgtccgcgaaagacgggtttgcacgtcgatactcctgccgtcctcaaggatttcgtcgtgatgacgaaggtgaagcgctgggtctgcccaggtccagaatttttcgccgcgacatctcatatcaatctcctcttacttatcccagtaggcgcggtaaagagagggatagatatccatttcgcttaaatgcgaccggtggaaaatgatcggccctaatccttgctgatagatatcagcgggacagcgccagtagagaaccgagcccagcatggcaattccgacgtcagcatttgcgatcattcacgcagcgcttattcagttgctcactgcgatgtcataatcatcgctacgagctgtgaaagatgcataaagctcgtacgacgcgttcgctcgtctcctcacttctcctacggagcgttctggacacaacgtcgtcttgaagttgcgattatagattgacagctagctcagtcctagggattgtgctagcgaattcattaaagaggagaaaggtaccatggcgagtagcgaagacgttatcaaagagttcatgcgtttcaaagttcgtatggaaggttccgttaacggtcacgagttcgaaatcgaaggtgaaggtgaaggtcgtccgtacgaaggtacccagaccgctaaactgaaagttaccaaaggtggtccgctgccgttcgcttgggacatcctgtccccgcagttccagtacggttccaaagcttacgttaaacacccggctgacatcccggactacctgaaactgtccttcccggaaggtttcaaatgggaacgtgttatgaacttcgaagacggtggtgttgttaccgttacccaggactcctccctgcaagacggtgagttcatctacaaagtaaactgcgtggtaccaacttcccgtccgacggtccggttatgcagaaaaaaaccatgggttgggaagcttccaccgaacgtatgtacccggaagacggtgctctgaaaggtgaaatcaaaatgcgtctgaaactgaaagacggtggtcactacgacgctgaagttaaaaccacctacatggctaaaaaaccggttcagctgccgggtgcttacaaaaccgacatcaaactggacatcacctcccacaacgaagactacaccatcgttgaacagtacgaacgtgctgaaggtcgtcactccaccggtgcttaaaaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttatatccgccattacgatctgacttgccactttggccgttatccctatctctggtcatacgcacctctcaatgctcagaagtcctgagctaaattgagtgtgcgcctacagagactagagaagatttatatcagaccgacgggtacaaacgaaaaaatcaataaccggctcctgtcagcacctcgcagagaaaacagatcgcagatattcagcatgcgtagctgatgctcgaccgagatcgcacctgacatgcaccgctgcgcggcacatcacactgagtagagagcgttccgcgacgccgatggcgtgaagcgcgagctggaaaacgtcgtgccgttcaagggcgcctaacccctccccatacaactcaagcccgccgacatgcgcgggcgaggattacctatgtccaatttcctgactcgctggctcaagcgcaagaagaagcccgggccgcgtccaacactggcaccaactggatttgctcgcggccacagcccggcagtcggcaggctagatccgatgcttgatccgctcaacccgttgagccccgtcagcccgttgcatcccgcctaccaggccgacagctacgaaccaccgcgcagcaccagcagttcctgctccagccgtgattacagcagctacgactgcggcagcagctactcgtcgagcgacagcagcagttccagcgatagcggatccagctccagcagctgcgactgaccaccaacctgccgccaccggcggcgtggagaccatcccatggaaaccgaaatcctttcggacgaagagctggtggcgatcaccggctacaaaccccgggcgtggcagcgccgttggctaacagaaaaaggctggcacttcgtcgagagccgcggcggccggccactggttggccgccagtacgcccgccagaagctcagcggcgtggtgatcgacaccttgccggtcgcaccagccccaccaccaacgcccgcctggacccctgatttttcccgagtgaagtga

SEQ ID NO: 170 - DNA, 3114 bp, synthetic construct
  source 1..3114: mol_type = other DNA
accaggatgaataccttaaggacgccaccggtaaccggcgttattggccggtcgcttgcgtcaaggtggaccttgaagcattgcgtcgcgctcgtgaccagctgtgggctgaggccatgttctgctaccaggccggtgatatctggtgggtgacccgtgaggaggaagaactgttcactgcagagcaggaagagcgcttcgtggtagatgaatgggaggggccgatcctgaaatggttggaggaatcccaggccggcgagacggtcaccggaagcgaagtgttggggcaggcattgaaccttgaccctggccactggggcaagcctgagcagatgcgggtgggatcgatcatgcaccgcctaggttggcggcgtcgcaggctggctgcgctgccgaagagcggtaagcgcccttgggcatatcagaagcctgatggttgggggcgcagcgccttggagcagtccacgcagccgaaggaggagtgcttttgatcaaacacatagatgagatgctgaaactgtgggctcaagagctccatgcgccggagccctgtcattcggcgggcggtgttggtagcatgctcggcctgttgatcgagtgcaagggtgaccttgtgcgtggcacccgaggcagcaaggtgctactggacgagtctgcggacatcgagatcattgtgaataagcatctggcaccggagctctacctggtggttcgtgagcactactgtaacgccgacagcgagctgtaccaaaagtaccggcactgtgggtgtagccgggatacctattacaagcgcttacatgaggcgcacgtctgtatcgcaggcttgctcttggggcgagcggcatgattcgccatccacggtcctaccgtcccgctgccgtcctgccgcgtttgatgcaggtcagcctagctcaagcccgcgcaggtcgcgggctgtcccaccgtcccaccgtacacacgaaggcgcgcacataggcgtgtgcagcgcatcacgcgcatgcatagcatttgcgatcattcacgcagcgcttattcagttgctcactgcgatgtcataatcatcgctacgagctgtgaaagatgcataaagctcgtacgacgcgttcgctcgtctcctcacttctcctgcggttaccaaaggcgtcctcgtcgtcttgaagttgcgattatagattgacggctagctcagtcctaggtatagtgctagcgaattcattaaagaggagaaaggtaccatgagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaataaggatccaaactcgagtaaggatctccaggcatcaaataaaacgaaaggctcagtcgaaagactgggcctttcgttttatctgttgtttgtcggtgaacgctctctactagagtcacactggctcaccttcgggtgggcctttctgcgtttataagtactaaggtgtattcccccggcattcaacagacattccccggacgtttaccacttattgccgggtggcattaaaactcgcttgctgccaccggaatcgacctgtaaaaagtacccatcttcgagacgtgcgggcgcacaaagcggcccgccaaacactgaaaaccccggccctggcgccggggtttttgcctttgggggattcgatgaacagcgagcagcaagcgttagttgagatgccaatctggttggtgatcttcctgtccctggtcggcggggtgtcaggcgagatgtggcgggccgacatggccggcgttagcggctggttcattttccgccaggtgctgctgcgctccggtgcctgcgtcgtatgcggactgtcgaccatcatgctgctgtactcggcgggcatgtcgatgtggtcggccagtgccattggttgtctcactgccactgccggtgcggatgtggccatagggttgtacaagcgttgggtcgccaagcggctgggcgtctgcgatgtcacgtcccgtagcggcgaacctggacagtgacccgatcgccagcctcggtggggtcggggaccctggcgatatggccgggttacggggcaggaaacccgcggctcttcgctagcggacagttcgccagcttactgaaattcaacctgttgaaattgaaaggttgttggttgaaataccattgaaatggagggctcatgacggattcgaacttcttgtcaaagagcgccttcgctgctcgcatagggagatcacccagctacatcacctggttgaaagacaacggccgcctggtgctttcacccgatggaaaattggtggatgtgctggccaccgaggccaagattcaggagacagctgatccggccaaagcagccgtcgcggctcggcatgaagaaaaccgcatcgagcgggacgtccgggcccacatccagcctagcgccgacacacctgcggtgcagccagcggatcacgcgccgagcgga

SEQ ID NO: 171 - DNA, 62 bp, synthetic construct
  source 1..62: mol_type = other DNA
tttacagctagctcagtcctagggactgtgctagcgaattcattaaagaggagaaaggtacc

SEQ ID NO: 172 - DNA, 36 bp, synthetic construct
  source 1..36: mol_type = other DNA
ctgatggctagctcagtcctagggattatgctagcg

SEQ ID NO: 173 - DNA, 36 bp, synthetic construct
  source 1..36: mol_type = other DNA
ttgacagctagctcagtcctagggattgtgctagcg

SEQ ID NO: 174 - DNA, 36 bp, synthetic construct
  source 1..36: mol_type = other DNA
tttatggctagctcagtcctaggtacaatgctagcg

SEQ ID NO: 175 - DNA, 36 bp, synthetic construct
  source 1..36: mol_type = other DNA
tttatagctagctcagcccttggtacaatgctagcg

SEQ ID NO: 176 - DNA, 36 bp, synthetic construct
  source 1..36: mol_type = other DNA
tttacggctagctcagccctaggtattatgctagcg

SEQ ID NO: 177 - DNA, 36 bp, synthetic construct
  source 1..36: mol_type = other DNA
tttacggctagctcagtcctaggtactatgctagcg

SEQ ID NO: 178 - DNA, 36 bp, synthetic construct
  source 1..36: mol_type = other DNA
tttacggctagctcagtcctaggtatagtgctagcg

SEQ ID NO: 179 - DNA, 36 bp, synthetic construct
  source 1..36: mol_type = other DNA
ctgacagctagctcagtcctaggtataatgctagcg

SEQ ID NO: 180 - DNA, 36 bp, synthetic construct
  source 1..36: mol_type = other DNA
tttacggctagctcagtcctaggtacaatgctagcg

SEQ ID NO: 181 - DNA, 36 bp, synthetic construct
  source 1..36: mol_type = other DNA
ttgacggctagctcagtcctaggtatagtgctagcg

SEQ ID NO: 182 - DNA, 36 bp, synthetic construct
  source 1..36: mol_type = other DNA
ttgacagctagctcagtcctaggtataatgctagcg

SEQ ID NO: 183 - DNA, 27 bp, synthetic construct
  source 1..27: mol_type = other DNA
gaattcattaaagaggagaaaggtacc

SEQ ID NO: 184 - DNA, 26 bp, synthetic construct
  source 1..26: mol_type = other DNA
caatatgacgtgttgttaatttggtt

SEQ ID NO: 185 - DNA, 26 bp, synthetic construct
  source 1..26: mol_type = other DNA
ttgggttccaccggatacctccggac

SEQ ID NO: 186 - DNA, 26 bp, synthetic construct
  source 1..26: mol_type = other DNA
gtcgtaaataagtaagtcactcccac

SEQ ID NO: 187 - DNA, 26 bp, synthetic construct
  source 1..26: mol_type = other DNA
gttgtccttctagtcgcccatgactc

SEQ ID NO: 188 - DNA, 26 bp, synthetic construct
  source 1..26: mol_type = other DNA
acaccgactacccctgctgggcccag

SEQ ID NO: 189 - DNA, 506 bp, synthetic construct
  source 1..506: mol_type = other DNA
ttgacagctagctcagtcctaggtataatactagtnnnnnnnnnnnnnnnnnnnngttttagagctagaaatagcaagttaaaataaggctagtccgttatcaacttgaaaaagtggcaccgagtcggtgctttttttgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaact

SEQ ID NO: 190 - DNA, 308 bp, synthetic construct
  source 1..308: mol_type = other DNA
ttgacagctagctcagtcctaggtataatactagtnnnnnnnnnnnnnnnnnnnngttttagagctagaaatagcaagttaaaataaggctagtccgttatcaacttgaaaaagtggcacatgaggatcacccatgtgctttttttgaagcttgggcccgaacaaaaactcatctcagaagaggatctgaatagcgccgtcgaccatcatcatcatcatcattgagtttaaacggtctccagcttggctgttttggcggatgagagaagattttcagcctgatacagattaaatcagaacgcagaagc

SEQ ID NO: 191 - DNA, 358 bp, synthetic construct
  source 1..358: mol_type = other DNA
gacgccgagcagcgggtacgggtagcacagggtctgttcgggcatgtgcccaaccgctgaaatacaggggctgccttgcagcccattcgcagcacaaggttgctcttgcaagcactgcagttgattcaagcgcttcgctcgacctgcaggaactgccttgtgctgcgtatgggccaaacacaatgttgaatgtcgtacccagcgctaggcgcttttcctgcacaggtctacaatcgccctcctcgcttttacatcgccgtccatactgatgccgacctgcacgctacaccccctgccctaccagcccgaccctgccgcctatttcgccgaattcattaaagaggagaaaggtaccatg

SEQ ID NO: 192 - DNA, 384 bp, synthetic construct
  source 1..384: mol_type = other DNA
ggcggcggcctgggccaggccagtgcacagcaggaagagcaaggcgacaaggcgcgacatcatgcggtactacatggggaggtgtgggtttgacccaatgtttgcgggaaggttctatgcagggcaggtctggccctttcgcgggtgaacccgctcctacgggggctgcacaccattggaacctggtggtgaccgtgtaggagcgggtttacccgcgaagaggccagcacaggccacccaaccatctgtatactgctgctttcgtccatttcaggcagaactcgcggagcgtccatgtccggcaatacctacggcaagctgttcactgtcaccaccgctggcgagagccatggcgaattcattaaagaggagaaaggtaccatg

SEQ ID NO: 193 - DNA, 401 bp, synthetic construct
  source 1..401: mol_type = other DNA
tttgcacagggctgccaatgtttcgcgttgctcggggatcgattgcgcacgcagattcatggggcggcagtctagacaggcatcgatgccctagcaaggccaatatcaaaaagttttgatattggcgatgagctgcaagccgaaagcctcaagttacacgctttggggcgccccgagtcgcgctctgcttcttcagttgcggcttaaagccggcgcttggcgcttaaatgtttgccccaagcgcccccagtgggcgaaaatggccgcctttttcgtgacaccacctattcaagccccctaggagatcagcgatgcccagccgtcgtgaacgtgccaacgccattcgtgccctcagcatggatgccgtgcaagaattcattaaagaggagaaaggtaccatg

SEQ ID NO: 194 - DNA, 341 bp, synthetic construct
  source 1..341: mol_type = other DNA
gcccagggtttcggcagtgataccggtgccgtcggagatgaagaacgcggttcgtttcatttgcgatctgggccttaagctgatgacgattcttggatatgataagttcggtttgccgaatgcggctgtcgacattctgccacatttgcagcgcctattttccaggtacaggccacaaacgcccggccaagtcatagaagacaggcgggcgccttttgagcttttccaacacagttagtggagagatcaccttggtagagtacgtagtttccctcgataagctcggcgtccatgatgtggagcatgtgggggaattcattaaagaggagaaaggtaccatg

SEQ ID NO: 195 - DNA, 306 bp, synthetic construct
  source 1..306: mol_type = other DNA
aaggcggctgaacccttggcggtgtttgaccaggccgtggagttgttgcaggggcggtgacctcagagggagggaacctcgggcttgcttgcgactcgaatcttcacctttcgtacagccctgtgcgggccagcgggtccgcagggtttcggcggcccttgagcggctgccggggctcgggtaatgtcgagtaaatgcacaggacagagcacgcccatgacctccaagctggaacaactcaagcagttcaccaccgtggtcgccgacaccggggacgaattcattaaagaggagaaaggtaccatg

SEQ ID NO: 196 - DNA, 353 bp, synthetic construct
  source 1..353: mol_type = other DNA
tacgagcgcgcagatgactgcgacggaagaccaccagaagaacttcagcagacgtatcaaggcttttcggtgtccaggttgggagtggattgcacgcaggtccggtggaccaaaaaacgctgggcattataagcatttttcgcctttccgggtgaccggccaggctactcgtcgagtcgatgggtagccctggcccagtgttgccgcggggttgcgacaggtggctgcgcttggcagctgtaacagacagcaaaggagtcgcgatgtttggacgcttcggcaaggatgccggttcactcgtgggggtggaaattacgcccgacgaattcattaaagaggagaaaggtaccatg

SEQ ID NO: 197 - DNA, 407 bp, synthetic construct
  source 1..407: mol_type = other DNA
gcccgccaccagtgcggcattcaccaggctgacggcgactttcaggttaagaacgttcatggaaaggtcctcttcttgttgtgagaagccctaagggcagttgaaaaccgatcgaaaaaatagaacacaacgagcgatatttttgtatacaatattttgaaacgatcgtatgcgatggcgcaaacctccgttttcacgggcgctctgacgaaagccagcttagccgatgaaaacccattgacctaagccgtcaggcgtgaatacactctgtcgcaaagcaagttgtatacaattacaaaatcgatgaggcacaaaccatgagcaaaatgagagcaatcgatgcagccgttctggtcatgcgccgtgaaggtgtagatgaattcattaaagaggagaaaggtaccatg

SEQ ID NO: 198 - DNA, 1355 bp, synthetic construct
  source 1..1355: mol_type = other DNA
ataactgtgcacacgatgctcggcacggtgcttcttcaacaggtctagggcgggggtcatgcaagggctccagtcagggacgactcggcggctactttaggacaattcccacatcccagccaccgcaccgcaatcgcgcgcgtctagtacagggatcagcagctgattctagcgaccatatcgtgactgaccgttcactttcgacctttgacatcagcgtttcttgtctatattttttcgtttctgaataagcggtgcacttataaggtgcatttcctacatcacccggggtcaatggggatagacaccgggttttgctgtcgagcgccacgcgcctcacaacaaaaaaaacgaggtcatacatgacgactgctctgcgccaacccacactgtccagccaatgcctggccgagtttctcggcaccgccctgctcatcttcttcggtaccggctgcgtcgccgcgctcaaggtggccggcgccagcttcgggctttgggaaatcagtatcatctggggcgtgggcgtcagcatggccatctacctcaccgccggtatttccggtgcgcacctgaacccagccgtgagtatcgccctcacactgttcgcagggttcgacaagcgcaagctgcccttctacatgctggcccaggtatgcggcgcattctgtggcgcagcactggtctacacgctgtacagcaatctgttcttcgatttcgaacaagcccacgccatgctgcgcggtagcgaaggcagcctggagctggcctcggtgttctccacctacccgcacccgtcgctgtccaccagccaggcgtttctggttgaagtggtgatcactgccattctgatggccgtgatcatggccctgaccgacgacaacaacggcctgccgcgcggcgccatggccccgctgctgatcggcctgctgattgcagtgattggcagcgccatgggcccattgactggctttgcgatgaacccggcccgcgatttcgggccaaaactcatgaccttcctggccggttggggcgaaatcgccttcactggcgggcgggatattccctatttcctggttccggtatttgcaccgatccttggcgcctgcttgggtgcagcgagctatcgcggcctgattgcacgcaacctgccaatggcgcccgccgcgacccctgagacaaatgacattcgccagggcgatactcaagccaattgatgccaccatcacgcacagccctgcccccctattcttgcaaggcctacgaccatgacagacacccaggataagaactacatcatcgccctggaccagggtaccaccagttcggaattcattaaagaggagaaaggtaccatg

SEQ ID NO: 199 - DNA, 377 bp, synthetic construct
  source 1..377: mol_type = other DNA
accgccgagcagggcggtcaataacagcaggcgcgggtacgggggcgtgccagggaacacggagcggcctttgtttcaggattgaaacaggcatggtaactgaccgtgggggttttgccagtgggtgtcgggcgtacgtcttgaaaagtaccgcgtggcggtgctcgatctcgacggctccgaaaatgccatggcaagtaccgcaaacgtccagacactggttctgtagcaggcgagccgtataatggccgggtcgccacttaacttttggctttaatggattggatatgactgaacagcaacctgttgcggttctgggcggcggcagcttcggcaccgccgtggcagaattcattaaagaggagaaaggtaccatg

SEQ ID NO: 200 - DNA, 626 bp, synthetic construct
  source 1..626: mol_type = other DNA
ttccgtgacggcatcgacaatgcagctaaagtgggtatcagcgccgtgatccagccgggtggttcgatgcgtgatgctgaagtcatcgctgctgccgacgaggccggcatcgcgatggtcttcactggcatgcgccacttccgccactaattacgcggatcccgaggcaagtcgagatcctgtgggagcgggcttgcccgcgaatacgatggtggattcaccgccgcattcgcgggcaagcccgctcccacagtgttcggcgcaagccacgggatcacttgaatcgaggttttgacatgaaagttttgatcatcggcagcggtggccgtgagcacgccctggcctggaaagtcgcccaggacccacgcgtcgagaaagtcttcgttgccccgggcaacgccggtaccgccattgaagccaagtgcgagaacgtcgccatcgacgtgtgcgccctggagcaattggccgacttcgccgagaaaaacgtcgacctgaccatcgtcggcccggaagcaccgctggttatcggtgtggtcgacctgttccgcagccgcggcctggactgcttcggcccaaccaagggcgcggcccagctggaattcattaaagaggagaaaggtaccatg

SEQ ID NO: 201 - DNA, 617 bp, synthetic construct
  source 1..617: mol_type = other DNA
aacaatgcctcgaagatccgcgccctgctgctggccggcatccgcgccgcgcgactgtggcggcagctgggcgggcaccgttggcagctggtgttcagccggcgcaagttgctgaacgaactgtacgacatgatgcgcagccccaactgacgggctgggcagcccgcttccacagggtctgccaccagatccaaattgggccgacctttggtcagccacccgacccaggcgcatttttcatgtatgatatgcgcccttccaaaagcctgactgtccgagaacaccccatgcagctttcttcgctcactgcggtttcccctgtagacggccgttatgccggcaaaacccaggccttgcgccccattttcagcgaattcggcctgatccgtttccgcgccctggtcgaagtgcgctggctgcagcgcctggccgcccacccgcaaatcggcgaagtgccggcgttctccgccgaagccaacgccctgctggacagcctggccaccgatttcaagctggagcacgccgagcgcgtcaaggaaatcgagcgcaccaccaaccacgacgtcaaggccattgaatacctcctcgaattcattaaagaggagaaaggtaccatg

SEQ ID NO: 202 - DNA, 681 bp, synthetic construct
  source 1..681: mol_type = other DNA
ggcccagtgcacaatggcttgcacctgttctatcgttttgggaaagacgatggcgctgggcgcaggcgggtaatgcttggtccagtccttgccatacgcttcgagggaaaccgggtcagtcaggaccttgccagggtcgacaagggtcatcagttcttcaataacagcggggtgggtcatcgctggaactctcgacttattcatggtcaccctgagcactcttcacctgtcgggataagctcagattgtgtcgcgtatgctagcatagccctcccgctgttcatgctaaggctgccctcgcgcctggcatcacccctcgccaatttatctccgggatacaggtttacgcagatgagcaagacttctctcgacaagagcaagatccggttccttcttcttgaaggtgtgcaccagaacgcggtcgataccctcaaggccgccggctacaccaacatcgaatacctcactggttcgttgccggaagccgagctgaaggaaaagatcgccgatgcccacttcatcggcatccgttcgcgtacccagctcaccgaagagatcttcgactgtgccaagaaactggtcgcagttggctgcttctgcatcggcaccaaccaggttgacctggaagctgcccgcgcgcgcggtatcgccgaattcattaaagaggagaaaggtaccatg

SEQ ID NO: 203 - DNA, 750 bp, synthetic construct
  source 1..750: mol_type = other DNA
aacatcctgctgaccagtgcccgccacctggaccgcaacatgcagctgctcgagcgaggcggtcagggcccggatcacccggtacacccggccatcgccgaaacccgctacatcaaaagcatcacctgccggttactgccaaacagctgatatctgccaaaaagggccgcccagcggccctttcctgccgcaatccccccgccaaccatccacatctgcattccctcaccgcgccagcggtgtagaatcggcctattcatcgccagtcatccccggcgggtttatgagctctggtcaagcacgcggcgatcccgcgcggtcttggccccatccgtgccaatggcaaccggcctgcggcgcaaaggacaagagaagctcactcccctatttgtgacctgattaagccgccaggagtgtttcatgcctgattatcgttccaagacttccacccaaggccgcaacatggccggcgcccgtgccctgtggcgcgccaccgggatgaaggacgaagacttcaagaaaccgatcatcgccatcgccaactcgttcacccagttcgtaccgggccatgtgcacctgaaggacctgggccagctggtggctcgcgaaatcgaacgcgccggtggcgtggccaaggaattcaacaccatcgcggtcgatgacggcatcgccatgggccacgacggcatgctgtactcgctgccaagccgcgaaatcattgaattcattaaagaggagaaaggtaccatg

SEQ ID NO: 204 - DNA, 582 bp, synthetic construct
  source 1..582: mol_type = other DNA
aaggctggcgatctgatgaaacaagccgctgcggcggtgggtggcaagggcggcggccgtccggacatggcccagggtggtggcgtcgacgtcgctgccctggaccaggccctggcgctggccgtgccattcgcagagcagggactttgagatgagggggtggagggtctagtggccacacaggccactcccgtccaccccttcatgttggattgttttttggggccctgtatgggctgaggcaccattgaaatggcgttgatcgtacagaaatttggcggcacctctgtcggttccatcgagcggatcgagcaggtagccgaaaaggtcaagaaacaccgtgaagcgggcgacgacctggtggttgtgctgtcggccatgagcggtgaaaccaatcgcctgatcgacctggccaagcagatcaccgatcagccggttcctcgtgaactggacgtgatcgtgtcgacgggtgagcaggtcaccattgccctgctgaccatggccttgatcaagcgtggtgtgccagcggtgtcctacaccggcaaccaggtggaattcattaaagaggagaaaggtaccatg

SEQ ID NO: 205 - DNA, 170 bp, synthetic construct
  source 1..170: mol_type = other DNA
agcatttgcgatcattcacgcagcgcttattcagttgctcactgcgatgtcataatcatcgctacgagctgtgaaagatgcataaagctcgtacgacgcgttcgctcgtctcctcacttctcctacggagcgttctggacacaacgtcgtcttgaagttgcgattataga

SEQ ID NO: 206 - DNA, 170 bp, synthetic construct
  source 1..170: mol_type = other DNA
tatacatcgcatcactacactattgattatcattgtgtacgtaacgagcttgcacaacgtgaagttcttcgagcacttcagctcgcaacgtaaatgacagttgctgttaagtgacgtgaatccttcaatgctgctcatgctgctgtcgtaaataagtaagtcactcccac

SEQ ID NO: 207 - DNA, 170 bp, synthetic construct
  source 1..170: mol_type = other DNA
agcatttgcgatcattcacgcagcgcttattcagttgctcactgcgatgtcataatcatcgctacgagctgtgaaagatgcataaagctcgtacgacgcgttcgctcgtctcctcacttctcctacggagcgttctggacacaaccggccccccccgctgccgcgggccg

SEQ ID NO: 208 - DNA, 170 bp, synthetic construct
  source 1..170: mol_type = other DNA
tatacatcgcatcactacactattgattatcattgtgtacgtaacgagcttgcacaacgtgaagttcttcgagcacttcagctcgcaacgtaaatgacagttgctgttaagtgacgtgaatccttcaatgctgctcatgctgctcgccgcccggcccgtgcccgcgcccg

SEQ ID NO: 209 - DNA, 170 bp, synthetic construct
  source 1..170: mol_type = other DNA
ctgcacgagttcgctgtcgagacaagtctcttagcgacgtattacgaagatcacatagtcagatgaagctatagagcacgacgctaacgattacgtcacgcttgacacaacagtttcgctacctagtgctcgcgcgactgcgacccggccccccccgctgccgcgggccg

SEQ ID NO: 210 - DNA, 2179 bp, synthetic construct
  source 1..2179: mol_type = other DNA
aactggtaatttgaggaggtaatttatggatcaaatcgaagcaatgttgtgcggcggaggagagaagacaaaagtggcggttactacgaagactttggcagatccattgaattggggtttagcagcggatcaaatgaaaggaagtcatttagatgaagtgaagaagatggtcgaagagtatcgtagaccagtcgtgaatcttggcggagaaacactgacgatcggacaagttgctgccatctccaccgtaggaggcagcgttaaggttgagttagcggagacttcaagagccggtgtgaaagctagcagtgattgggttatggagagcatgaacaaaggtactgacagttacggagtcaccaccggctttggtgctacttctcaccggagaaccaaaaacggcaccgcattacaaacagaactcattagatttttgaacgccggaatattcggaaacacgaaggagacatgtcacacactgccgcaatccgccacaagagccgccatgctcgtcagagtcaacactcttctccaaggatactccgggatccgattcgagatcctcgaagcgattacaagtctcctcaaccacaacatctctccgtcactacctctccgtggaaccattaccgcctccggcgatctcgttcctctctcttacatcgccggacttctcaccggccgtcctaattccaaagccaccggtcccgacggtgaatcgctaaccgcgaaagaagcttttgagaaagccggaatcagtactggattcttcgatttacaacctaaggaaggtttagctctcgttaatggcacggcggttggatctggaatggcgtcgatggttctattcgaagcgaatgtccaagcggtgttagcggaggttttatcagcgatcttcgcggaggttatgagcgggaaacctgagtttaccgatcatctgactcatcgtttaaaacatcatcccggacaaatcgaagcggcggcgataatggagcacatactcgacggaagctcatacatgaaattagctcaaaaggttcacgagatggatccattgcagaaaccaaaacaagatcgttacgctcttcgtacatctcctcaatggctaggtcctcaaattgaagtaatccgtcaagctacgaaatcgatagagcgtgaaatcaactccgttaacgataatccgttgatcgatgtttcgaggaacaaggcgattcacggtggtaacttccaaggaacaccaatcggagtttctatggataacacgagattggcgattgctgcgattgggaagctaatgtttgctcaattctctgagcttgttaatgatttctacaacaatggacttccttcgaatctaactgcttcgagtaatccaagtttggattatggattcaaaggagcagagattgctatggcttcttattgttctgagcttcaatacttggctaatccagtcacaagccatgttcaatcagctgagcaacataatcaagatgtgaactctcttggtttgatctcgtctcgtaaaacatctgaagctgtggatattcttaagctaatgtcaacaacgttccttgtggggatatgtcaagctgttgatttgagacatttggaggagaatctgagacaaactgtgaagaacacagtttctcaagttgctaagaaagtgttaaccactggaatcaacggtgagttacatccgtcaaggttttgcgagaaggacttgcttaaggttgttgatcgtgagcaagtgttcacgtatgtggatgatccttgtagcgctacgtacccgttgatgcagagactaagacaagttattgttgatcacgctttgtccaacggtgagactgagaagaatgcagtgacttcgatctttcaaaagattggagcttttgaagaggagcttaaggctgtgcttccaaaggaagttgaagcggctagagcggcttatgggaatggaactgcgccgattcctaaccggattaaggaatgtaggtcgtatccgttgtataggttcgtgagggaagagcttggaacgaagttgttgactggagaaaaggttgtgtctccgggagaggagtttgataaggtcttcactgctatgtgtgaaggtaaacttattgatccgttgatggattgtctcaaggaatggaacggagctccgattccgatttgctaa

SEQ ID NO: 211 - DNA, 53 bp, synthetic construct
  source 1..53: mol_type = other DNA
aacgcatgagaaagcccccggaagatcaccttccgggggcttttttattgcgc

SEQ ID NO: 212 - DNA, 305 bp, synthetic construct
  source 1..305: mol_type = other DNA
accgaagcgctggcggggcggggtcgtgtgctgttgcgcaagtccggtaccgagccgttggtgcgggtcatggttgagggcgaggacgaaagccaggtgcgggcccatgctgaagcgctggccaaactggtcggcgaagtttgtgtctgaaggcgcttgccagcgcagatctggttgggtaagatctgcgcccactttgaccgacgaggtaaagcatgcgtcgccctatggtagctggtaactggaaaatgcacggtacccgcgctagcgtcgctgaattcattaaagaggagaaaggtaccatg

SEQ ID NO: 213 - DNA, 976 bp, synthetic construct
  source 1..976: mol_type = other DNA
tatacatcgcatcactacactattgattatcattgtgtacgtaacgagcttgcacaacgtgaagttcttcgagcacttcagctcgcaacgtaaatgacagttgctgttaagtgacgtgaatccttcaatgctgctcatgctgctgtcgtaaataagtaagtcactcccacttgacagctagctcagtcctagggattgtgctagcacccgttttttgggctaacaggaggaattaaccatggggagccaccatcaccatcaccatggcagatctatgagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacgccaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaa

SEQ ID NO: 214 - DNA, 20 bp, synthetic construct
  source 1..20: mol_type = other DNA
aagcgcttcgctcgacctgc

SEQ ID NO: 215 - DNA, 20 bp, synthetic construct
  source 1..20: mol_type = other DNA
caatggtgtgcagcccccgt

SEQ ID NO: 216 - DNA, 20 bp, synthetic construct
  source 1..20: mol_type = other DNA
cttcagttgcggcttaaagc

SEQ ID NO: 217 - DNA, 20 bp, synthetic construct
  source 1..20: mol_type = other DNA
ggttcgtttcatttgcgatc

SEQ ID NO: 218 - DNA, 20 bp, synthetic construct
  source 1..20: mol_type = other DNA
cggacccgctggcccgcaca

SEQ ID NO: 219 - DNA, 20 bp, synthetic construct
  source 1..20: mol_type = other DNA
gaacttcagcagacgtatca

SEQ ID NO: 220 - DNA, 20 bp, synthetic construct
  source 1..20: mol_type = other DNA
cgtcagagcgcccgtgaaaa

SEQ ID NO: 221 - DNA, 20 bp, synthetic construct
  source 1..20: mol_type = other DNA
aatcgcgcgcgtctagtaca

SEQ ID NO: 222 - DNA, 20 bp, synthetic construct
  source 1..20: mol_type = other DNA
tcttgaaaagtaccgcgtgg

SEQ ID NO: 223 - DNA, 20 bp, synthetic construct
  source 1..20: mol_type = other DNA
ggccgaagcagtccaggccg

SEQ ID NO: 224 - DNA, 20 bp, synthetic construct
  source 1..20: mol_type = other DNA
ggccgaattcgctgaaaatg

SEQ ID NO: 225 - DNA, 20 bp, synthetic construct
  source 1..20: mol_type = other DNA
ggcacagtcgaagatctctt

SEQ ID NO: 226 - DNA, 20 bp, synthetic construct
  source 1..20: mol_type = other DNA
gatgccgtcatcgaccgcga

SEQ ID NO: 227 - DNA, 20 bp, synthetic construct
  source 1..20: mol_type = other DNA
ggccaggtcgatcaggcgat

SEQ ID NO: 228 - DNA, 20 bp, synthetic construct
  source 1..20: mol_type = other DNA
agcagcatgagcagcattga

SEQ ID NO: 229 - DNA, 20 bp, synthetic construct
  source 1..20: mol_type = other DNA
gtcgcagtcgcgcgagcact

(19) United States
(12) Patent Application Publication
(10) Pub. No.: US 2023/0340440 A1
(43) Pub. Date: Oct. 26, 2023

(54) GENETIC MANIPULATION METHOD IN BACTERIA

(71) Applicant: University of Washington, Seattle, WA (US)

(72) Inventors: Cholpisit Kiattisewee, Seattle, WA (US); James M. Carothers, Seattle, WA (US); Jesse Zalatan, Seattle, WA (US); Ian D. Faulkner, Seattle, WA (US); Jason Fontana, Seattle, WA (US); Chen Dong, Seattle, WA (US)

(73) Assignee: University of Washington, Seattle, WA (US)

(21) Appl. No.: 18/306,960

(22) Filed: Apr. 25, 2023

Related U.S. Application Data
(60) Provisional application No. 63/335,143, filed on Apr. 26, 2022.

(51) Int. Cl. C12P 13/00; C12P 13/22; C12N 9/22; C12N 15/11

(52) U.S. Cl. CPC C12N 9/22; C12N 15/11; C12P 13/005; C12P 13/222; C12N 2310/20

(57) ABSTRACT

The present disclosure relates to bacterium engineered to produce aromatic compounds or compounds with aromatic metabolites or intermediates using the CRISPR-CAS transcriptional activation (CRISPRa) and/or transcriptional repression (CRISPRi). Accordingly, in an aspect the present disclosure relates to an engineered bacterium comprising genetic elements supporting programmable transcriptional activation and/or repression. The present disclosure also provides methods and systems for producing aromatic compounds or compounds with aromatic metabolites or intermediates using the engineered bacterium disclosed herein.

### CROSS REFERENCE TO RELATED APPLICATION

This application claims the benefit of U.S. Provisional Application No. 63/335143, filed Apr. 26, 2022, the disclosure of which is incorporated herein by reference in its entirety.

### STATEMENT OF GOVERNMENT LICENSE RIGHTS

This invention was made with government support under Grant Nos. CBET 1844152 and EF-1935087 and MCB 1817623, awarded by the National Science Foundation and Grant No. EERE DE-EE0008927, awarded by the U.S. Department of Energy. The government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 3915-P1247USPNP_Seq_List_20230424.xml. The XML file is 258,462 bytes; was created on Apr. 24, 2023; and is being submitted electronically via Patent Center with the filing of the specification.

### BACKGROUND

The development of microbial platforms for industrial chemical production frequently requires optimizing the expression levels of multiple genes. The advent of CRISPR-Cas provides tools that can be used to rapidly program gene expression promises to accelerate pathway engineering for the efficient production of high-value compounds. The application of CRISPR-Cas tools for transcriptional repression (CRISPRi) in bacterial metabolic engineering is well-established. By comparison, the development of CRISPR-Cas tools for programmable transcriptional activation (CRISPRa) has lagged due to the paucity of effective transcriptional activators, and the complexity of the rules governing CRISPRa-directed transcription from bacterial promoters. Despite these challenges, the potential for using CRISPRa to program gene expression has been demonstrated through the successful implementation in E. coli, M. xanthus, K. oxytoca, and S. enterica. Determining how to strategically port CRISPRa systems into other microbes could significantly improve available metabolic engineering capabilities.

Pseudomonas putida is a gram-negative soil bacterium that has recently received attention as a potential chassis for bioproduction due to desirable metabolic capabilities and the capacity to survive harsh bioprocessing conditions. P. putida has high reducing power and the ability to metabolize a broad range of feedstocks, from glucose to the toxic products of aromatic lignin degradation. The successful implementation of CRISPR genome editing and CRISPRi in P. putida shows that CRISPR gene targeting can be effective in P. putida and provides a starting point to assess whether gene activation with a CRISPRa system can be achieved.

CRISPR-Cas transcriptional control typically uses the catalytically inactive Cas9 protein (dCas9) with programmable guide RNAs that recognize DNA targets through Watson-Crick base pairing. Recently, a variant of the transcriptional activator SoxS (R93A/S101A) that can be linked to a programmable CRISPR-Cas DNA binding domain to activate gene expression in E. coli was identified and optimized. SoxS interacts with an interface on the α-subunit of RNA polymerase (RpoA) that is widely conserved throughout bacterial species, including in P. putida, suggesting that the CRISPRa system that was developed in E. coli should also be effective in P. putida and other bacteria. However, in contrast to the relative permissiveness of CRISPRi (and CRISPRa in eukaryotes), CRISPRa in bacteria is known to be sensitive to several features of target promoters, including the precise distance from the transcription start site and the intervening sequence composition. Accordingly, it is not known to what extent the rules characterized in one bacterial species are generalizable in others.

Despite the advances in the art of CRISPR-based modification of gene expression, there remains a need for efficient systems and methods for implementing programmed transcriptional activation in desirable bacterial species. The present disclosure addresses these and related needs.

### SUMMARY

Accordingly, in an aspect of the present disclosure there is provided an engineered bacterium comprising genetic elements supporting programmable transcriptional activation and/or repression. In some embodiments, the genetic elements comprise at least one heterologous nucleic acid construct. In certain embodiments, the at least one heterologous nucleic acid construct comprises a first nucleic acid sequence encoding an endonuclease that lacks endonuclease activity. In some embodiments, the endonuclease is selected from dCas9, dCas12, dCasX, dCasPhi, dCas3 (Cascade), and the like. In an embodiment, the at least one heterologous nucleic acid construct comprises a second nucleic acid sequence encoding a transcriptional activator. In some embodiments, the transcriptional activator comprises an RNA-binding protein (RBP) fused to an effector domain. In certain embodiments, the effector domain is selected from SoxS, TetD, PspF, AsiA, N-terminus of RpoA (aNTD), and SoxS-family activators. In some embodiments of the present disclosure, the RNA-binding protein is selected from MCP, PCP, Com, LambdaN22Plus, Qbeta. In certain embodiments, the effector domain comprises SoxS. In an embodiment, the SoxS is engineered to reduce or abolish DNA-binding capacity. In some embodiments, the SoxS is engineered to comprise a mutation. In certain embodiments, the mutation in SoxS is at R93 and/or S101. In an embodiment, the SoxS mutation comprises R93A and/or S101A.

In some embodiments of the present disclosure, the at least one heterologous nucleic acid construct comprises a third nucleic acid sequence encoding a scaffold RNA (scRNA). In an embodiment, the scRNA comprises a 3′ MS2 hairpin loop that interacts with a transcriptional activator. In some embodiments, the scRNA comprises a 5′ domain comprising a guide sequence that hybridizes to a target sequence. In an embodiment, the target sequence is proximal to a PAM and/or a promoter sequence of an endogenous gene of the engineered bacterium.

In another embodiment of the present disclosure, the at least one heterologous nucleic acid construct comprises a fourth nucleic acid sequence. In some embodiments, the fourth nucleic acid sequence comprises an open reading frame of at least one gene of interest. In some embodiments, the at least one gene of interest is operatively linked to a promoter sequence. In an embodiment, the at least one gene of interest is linked to a PAM sequence. In some embodiments, the at least one gene of interest is operatively linked to a promoter sequence and a PAM sequence. In some embodimets, the target sequence is proximal to the promoter sequence and/or the PAM sequence. In certain embodiments of the present disclosure, the open reading frame encodes a gene product that results in production of an aromatic compound.

In an aspect of the present disclosure, the at least one heterologous nucleic acid construct comprises the first, second, third, and fourth sequences distributed in any combination on two vectors. In another aspect, the at least one heterologous nucleic acid construct comprises the first, second, third, and fourth sequences distributed on a single vector. In some embodiments, the vector is optionally pBBR1, pRK2, pRSF1010, pBAV1, and the like, or is a derivative thereof. In some embodiments, the at least one heterologous nucleic acid construct is integrated into the genome of the engineered bacterium. In some embodiments, the first, second, third, and fourth sequences each comprise or are operatively linked to a promoter operable in the engineered bacterium. In some embodiments, the engineered bacterium is Pseudomonas putida or Acinetobacter baylyi.

In an embodiment of the present disclosure, the engineered bacterium is Pseudomonas putida, and wherein the target sequence is between about 60 to about 120 bases upstream (5′) of a transcriptional start site (TSS) of the endogenous gene or open reading frame. In an embodiment, the target sequence is about 15 to about 25 bases upstream (5′) of a transcriptional start site (TSS) of the endogenous gene or open reading frame. In some embodiments, the target sequence corresponds with the J1, J3, J5, or J6 promoter, or portions thereof. In some embodiments of the present disclosure, the promoter sequence resides in the intervening sequence between the target sequence and the transcriptional start site (TSS) of the endogenous genes or open reading frame. In certain embodiments, the promoter sequence is a synthetic 5′-upstream sequences containing appropriate NGG PAM at an optimal position, wherein the optimal position is selected from about 75 to 85 nucleotides, about 78 to 83 nucleotides, and about 81 nucleotides upstream of the TSS.

In certain aspects of the present disclosure, the genetic elements are under control of a small-molecule inducible promoter. In some embodiment, the small molecule inducer is selected from m-toluic acid, salicylic acid, benzoic acid, and related compounds. In some embodiments, the small-molecule inducible promoter is XylS/Pm, derived from P. putida mt-2.

In an aspect of the present disclosure, there is provided a bacterium engineered to produce p-aminophenylalanine (p-AF) or p-aminocinnamic acid (p-ACA). In some embodiments, the bacterium comprises an open reading frame encoding PAL. In certain embodiments, the PAL is derived from Arabinobsis thaliana. In some embodiments, the PAL is derived from or Rhodotorula glutinis. In some embodiments, the bacterium comprises an open reading frame encoding PapABC. In some embodiments, the open reading frame encoding PapABC is derived from Pseudomonas fluorescens. In some embodiments, the bacterium comprises an open reading frame encoding AroGL. In an embodiment, the open reading frame encoding AroGL is derived from E. coli.

In yet another aspect of the present disclosure there is provided a bacterium engineered to produce tetrahydrobiopterin (BH4) or derivatives thereof. In some embodiments, the bacterium comprises an open reading frame encoding GTPCH. In some embodiments, the open reading frame encoding GTPCH is derived from E. coli. In some embodiments, the bacterium comprises an open reading frame encoding PTPS/SR. In an embodiment, the open reading frame encoding PTPS/SR is derived from M. alpina.

In yet another aspect, of the present disclosure, there is provided a system for production of aromatic compounds or compounds with aromatic metabolites or intermediates. In some embodiments, the system comprises an engineered bacterium comprising genetic elements supporting programmable transcriptional activation and/or repression. In some embodiments, the system further comprises a suitable growth medium.

In a related aspect, the present disclosure also pertains to a method of producing aromatic compounds or compounds with aromatic metabolites or intermediates. In some embodiments, the method comprises providing an engineered bacterium comprising genetic elements supporting programmable transcriptional activation and/or repression; and a suitable substrate permitting production of the compounds. In some embodiments, the compound is p-AF, and/or p-ACA. In some embodiments, the substrate is selected from glucose, glycerol, p-coumaric acid, and other substrates from lignocellulosic biomass.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1C show configuring CRISPRa in P. putida. CRISPRa components (i-iii) are necessary to activate the gene of interest (iv). The CRISPRa ternary complex recruits and stabilizes RNA polymerase at the promoter region (FIG. 1A). Available gene expression tools in P. putida include pBBR1 plasmid, pRK2 plasmid, and genome integration (FIG. 1B). Two antibiotic selection markers, Gentamicin (GmR) and/or Kanamycin (KmR) were used. Testing CRISPRa in different expression systems. The CRISPRa fold-activation is highest when dCas9/MCP-SoxS were integrated into the genome and the scRNA/reporter genes were expressed on pBBR1-GmR plasmid (FIG. 1C). The J109 scRNA was used for activation and hAAVS1 was used as an off-target scRNA. Values in FIG. 1C represent the mean ± standard deviation calculated from n = 3 independent biological replicates.

FIGS. 2A-2D show sensitivity of CRISPRa to distance from the TSS and promoter sequence composition in P. putida. Factors known to affect CRISPRa efficiency in E. coli include: i) distance to TSS; ii) scRNA target sequence; iii) minimal promoter strength; and (iv) 5′-proximal sequence between target sequence and minimal promoter (FIG. 2A). Effect of distance to TSS on CRISPRa efficiency at 10 bp resolution (FIG. 2B). The J1 synthetic sequence upstream of the minimal promoter includes target sites every 10 bp in both the template strand (filled), and the non-template strand (unfilled). scRNAs J101-J121 were expressed in the pBBR1-GmR backbone. The observed peaks of activation are slightly offset on the template and non-template strands because the distance is defined from the TSS to the PAM sites, which is proximal to the TSS on template strand targets and distal to the TSS on non-template strands. The most effective sites at -91 on the template strand (J108) and -80 on the non-template strand (J109) target overlapping 20-base sites. Effect of distance to TSS on CRISPRa efficiency at single bp resolution (FIG. 2C). N bases were added upstream of the minimal promoter (N = 1 - 12), and the J106 scRNA was used to target sites at -81 to -93 upstream of the TSS. The J3 upstream sequence has lower basal expression (11-fold) and higher fold-activation by CRISPRa than the J1 sequence. When the 20 bp target sequence J106 was inserted into the J3 promoter, the basal expression remains low. When the 20 bp target sequence J306 was inserted into the J1 promoter, basal expression remains high (FIG. 2D). Values in FIGS. 2B-2D represent the mean ± standard deviation calculated from n = 3 independent biological replicates.

FIGS. 3A-3D show sensitivity of CRISPRa to promoter strength and 5′ upstream sequence in P. putida. CRISPRa is sensitive to basal promoter strength. Variants of pPPC021.J231XX were constructed by changing the BBa_J23117 promoter into ten other minimal promoters (FIG. 3A). The promoters weaker than BBa_J23117 exhibited low CRISPRa efficiency while the fold-activation was maximized at BBa_J23117 and decreased as the promoter strength increased beyond that point. CRISPRa is sensitive to the sequence composition of the 26 bp 5′-proximal sequence between the scRNA target site and the minimal BBa_J23117 promoter (FIG. 3B). Comparison of CRISPRa-induced expression with different 5′-proximal sequences characterized in E. coli and P. putida (FIG. 3C). Correlation between CRISPRa-induced mRFP expression levels from different promoter contexts in E. coli and P. putida (R² = 0.80) (FIG. 3D). Values in FIG. 3A and FIGS. 3C-3D represent the mean ± standard deviation calculated from n = 3 independent biological replicates. Bars in panel B represent the value of one (n = 1) sample.

FIG. 4 shows multi-gene CRISPRa/CRISPRi in plasmid-borne dual reporters. A multi-gene CRISPRa/CRISPRi reporter with weakly expressed mRFP (J3-BBa_J23117) and highly expressed sfGFP (J3(106)-BBa_J23111) shows simultaneous activation and repression when an activator scRNA for mRFP and a repressor sgRNA for sfGFP are delivered. The strong sfGFP reporter can also be further activated ~2-3-fold if targeted by an upstream activating scRNA. This strain exhibits noticeably slower growth in both agar and liquid media (data not shown). Values represent the mean ± standard deviation calculated from n = 3 independent biological replicates.

FIGS. 5A-5B show CRISPRa with endogenous promoters and inducible CRISPRa/CRISPRi in P. putida. The putative promoter sequences between two open reading frames (ORFs) with 60 bp flanking sequences were incorporated into the mRFP reporter (FIG. 5A). scRNAs were introduced for all potentially activable target sites corresponding to the effective distances as tested in FIGS. 2B & 2C. Precise distances from the target site to the TSS are listed in Table 5. hAAVS1 was used as an off-target scRNA for all ten promoters. Tunable activation of mRFP expression with CRISPRa and tunable repression of mRFP expression with CRISPRi were achieved with different inducer concentrations (0-5 mM m-toluic acid) in the inducible-dCas9 strain (FIG. 5B, right panel). The inducible-dCas9 strain yielded 3-fold activation with CRISPRa or 7-fold repression with CRISPRi at 1 mM m-toluic acid compared to the no-inducer control. Fold-changes compared to the off-target control were 4-fold and 5-fold, respectively. The constitutively expressed dCas9 strain (FIG. 5B, left panel) showed little to no response to inducer concentration. Values represent the mean ± standard deviation calculated from n = 3 independent biological replicates.

FIGS. 6A-6D show multi-target CRISPR activation on a biopterin pathway. Graphical depiction shows CRISPRa implementation to any gene of interest by integrated dCas9/MCP-SoxS strains (PPC001) where scRNA(s) and heterologous genes were delivered on pBBR1-GmR plasmid (FIG. 6A). Biosynthetic and spontaneous oxidation scheme from GTP into tetrahydrobiopterin (BH4) and its oxidized variants. The three-enzyme pathway consisted of E. coli gtpch, M. alpina ptps, and M. alpina sr. Tetrahydrobiopterin is reactive towards ambient oxygen and is readily oxidized into dihydrobiopterin (BH2) and biopterin, respectively (FIG. 6B). Graphical depiction of CRISPRa activating three genes with a single scRNA (FIG. 6C). Dihydrobiopterin (BH2) levels observed by HPLC-MS of PPC01 strains bearing pPPC024 (3-gene pathway) or pPPC025 (absence of sr gene) (FIG. 6D). HPLC-MS data of three biopterin species are shown in FIGS. 20A-20D. Values represent the mean ± standard deviation calculated from n = 3 technical replicates.

FIGS. 7A-7C show CRISPR activation on mevalonate production operon. Biosynthetic pathway for D-mevalonic acid production from acetoacetyl-CoA with heterologous mvaS and mvaE genes from Enterococcus faecalis (FIG. 7A). Graphical depiction showing comparison of CRISPR activation complex (pPPC030) with the LacI-Ptrc inducible system (pPPC029) for gene activation (FIG. 7B). Titer of mevalonate calculated based on GC-MS detection of cyclized mevalonolactone (m/z = 71). The J306 scRNA was used as an on-target CRISPRa scRNA while hAAVS1 was used as an off-target scRNA. Up to 5.0 mM IPTG was used for induction of LacI-Ptrc and up to 1 mM m-toluic acid was used for induction of XylS-Pm (FIG. 7C). The off-target scRNA produced a mevalonate titer indistinguishable from the no plasmid control (less than 10 mg/L, see FIGS. 21A-21B). Values in FIG. 7C represent the mean ± standard deviation calculated from n = 3 independent biological replicates, n = 5 for the no plasmid control and off-target scRNA, n = 7 for the constitutively expressed dCas9/MCP-SoxS strain, and n = 10 for the LacI-Ptrc strain.

FIG. 8 shows basal mRFP expression on pBBR1 and pRK2 plasmids. Basal expression of mRFP reporter gene from J1-BBa_J23117-mRFP in different plasmid backbones (pBBR1 or pRK2 origins), with either GmR or KmR antibiotics. The plasmids expressing an off-target scRNA were tested side-by-side to the no-gRNA control and exhibited indistinguishable expression levels. See Table 3 for plasmid constructs used here. Values represent the mean ± standard deviation calculated from n = 3 independent biological replicates. independent samples.

FIGS. 9A-9B show expression cassettes affect CRISPRa efficiency and growth of P. putida. CRISPRa from different expression methods for dCas9/MCP-SoxS, scRNA, and reporter. P. putida compatible plasmids are pBBR1 and pRK2 in which GmR and KmR can be used as antibiotic selection markers (FIG. 9A). Both basal expressions and fold-activation varied with different expression systems. The genomically-integrated dCas9/MCP-SoxS cassettes give the highest fold-activation in pBBR1-GmR (5-fold compared to that of an off-target scRNA control). Growth curve (OD₆₀₀ vs. time) of P. putida strains in liquid culture with dCas9/MCP-SoxS cassette either on plasmid or integrated into the genome (FIG. 9B). Every strain expresses an off-target scRNA. Expressing dCas9/MCP-SoxS on plasmid systems (dashed or dotted lines) significantly reduces the growth rate compared to that of 2-plasmid strains with integrated dCas9/MCP-SoxS (dotted line). Qualitatively similar growth defects were observed when colonies were grown on agar plates. Values represent the mean ± standard deviation calculated from n = 3 independent biological replicates.

FIGS. 10A-10B shows an additional plasmid decreases CRISPRa efficiency. P. putida (PPC01) strains bearing J1-mRFP and scRNA plasmids with different origin of replications and antibiotic markers (Table 2B, FIG. 10A) were further transformed with a second empty plasmid of different origin of replication and antibiotic marker. Expressing a second empty plasmid led to significant drops in both basal expression levels and fold-activation (FIG. 10B). Values represent the mean ± standard deviation calculated from n = 3 independent biological replicates.

FIG. 11 shows the effect of distance on CRISPRa using the J3 promoter in comparison to the J1 promoter. Comparison of optimal target sites of the J1 promoter (J106-J109) and the J3 promoter (J306-J309). The highest fold-activation was obtained with the J306 scRNA. Values represent the mean ± standard deviation calculated from n = 3 independent biological replicates.

FIGS. 12A-12B shows correlation plot of fold-activation between P. putida and E. coli. Randomized 5′-proximal sequences (5′-PS) were transformed into either a no-CRISPR (KT2440) strain or a CRISPRa (PPC01) strain (FIG. 12A). Differences in basal expression with variable 5′-PS are detectable but are relatively small compared to the effects on CRISPRa fold-activation. Correlation plot of fold-activation between CRISPRa strains with cognate scRNAs and off-target scRNAs (R² = 0.69) (FIG. 12B). E. coli data are from (Fontana et al., 2020). Bars in FIG. 12A represent the value of n = 1 independent biological replicate. Values in FIG. 12B represent the mean ± standard deviation calculated from n = 3 independent biological replicates.

FIG. 13 shows dual CRISPR activation on the plasmid-borne dual reporter. A multi-gene CRISPRa reporter with weakly expressed mRFP (J3-BBa_J23117) and weakly expressed sfGFP (J3(106)-BBa_J23117) can be simultaneously activated by targeting the reporters with two cognate scRNAs. The observed fold-activations with two scRNAs expressed are weaker than with only one scRNA expressed, possibly due to competition for a limited pool of dCas9. A similar effect is observed with one on-target and one off-target scRNA. Values represent the mean ± standard deviation calculated from n = 3 independent biological replicates.

FIGS. 14A-14B show simultaneous CRISPRa/CRISPRi in strains with integrated dual reporters. Highly expressed mRFP (BBa_J23111) and weakly expressed sfGFP (J1-BBa_J23117) were integrated together with a dCas9/MCP-SoxS construct to produce strain PPC04 (FIG. 14A). Simultaneous CRISPRa on sfGFP and CRISPRi on mRFP are detectable, but the CRISPRa fold-activation is modest compared to that observed with the plasmid reporter (see FIG. 4). Weakly expressed mRFP (J3-BBa_J23117) and highly expressed sfGFP (BBa_J23111) were integrated into the PPC01 strain at the pp1 and pp2 sites, respectively, to produce strain PPC05 (FIG. 14B). The fold-change from CRISPRa and CRISPRi marginally improved compared to the PPC04 strain (FIG. 14A). The magnitude of CRISPRa fold-activation in simultaneous CRISPRa/CRISPRi was weaker than that observed if just a single scRNA was delivered to activate the mRFP reporter, possibly due to competition between multiple scRNA/gRNA cassettes for a limited pool of dCas9. A similar effect was observed with one activating scRNA and one off-target scRNA. Values represent the mean ± standard deviation calculated from n = 3 independent biological replicates.

FIGS. 15A-15B show multi-gene CRISPRa/CRISPRi regulation in the integrated dual reporters. An integrated dual reporter with weakly expressed mRFP (J3-BBa_J23117) and weakly expressed sfGFP (J3(106)-BBa_J23117) can be activated with two scRNAs, one targeting each reporter (FIG. 15A). The presence of the second sgRNA/scRNA reduced CRISPRa efficiency compared to single gene activation. An integrated dual reporter with weakly expressed mRFP (J3-BBa_J23117) and a highly expressed sfGFP (J3(106)-BBa_J23111) can be activated or repressed at the sfGFP reporter (FIG. 15B). Simultaneous activation of mRFP and repression of sfGFP occurs with a J306 scRNA for mRFP activation and an sgRNA that targets within the sfGFP ORF for repression. Activation of both mRFP and sfGFP occurs a J306 scRNA for mRFP and a J106 scRNA for sfGFP. An unexpected improvement in CRISPRa mRFP expression from the second off-target sgRNA was observed, while sfGFP CRISPRa suffered from the presence of the second guide-RNA, similar to previous conditions. Values represent the mean ± standard deviation calculated from n = 3 independent biological replicates.

FIGS. 16A-16C show CRISPR activation of P. putida endogenous promoters. Ten P. putida endogenous promoters were selected based on available scRNA target sites at the appropriate phase and distance from reported transcription start sites (TSSs) and were coupled with mRFP reporter (FIG. 16A). Each gene was given an abbreviated code (A-J). Activation profiles of CRISPRa on the endogenous promoters with relatively low basal expression (promoters A-G except promoter C) were plotted with the corresponding scRNAs (A1-A6, for example) (FIG. 16B). Fold-changes were provided for instances where >1.5-fold activation was observed compared to an off-target scRNA (hAAVS1). Activation profiles of CRISPRa on the endogenous promoters with relatively high basal expression promoters (promoters C and H-J) revealed no significant CRISPRa activity with the scRNAs that were tested (FIG. 16C). The J3-BBa_J23117 promoter with J306 scRNA was included as a positive CRISPRa control. Values represent the mean ± standard deviation calculated from n = 3 independent biological replicates.

FIGS. 17A-17C show inducible promoters in P. putida (FIG. 17A). Inducible LacI-Ptrc-mediated activation of an mRFP reporter gene in P. putida KT2440 with 0-1 mM IPTG. A 13-fold activation at 1 mM IPTG was observed compared to the no inducer condition. Inducible XylS-Pm-mediated activation of an mRFP reporter gene in P. putida KT2440 with 0-5 mM m-toluic acid (FIG. 17B). A >300-fold activation was observed at 1 mM m-toluic acid compared to the no inducer condition. The XylS-Pm promoter provides a better dynamic range compared to LacI-Ptrc promoter mainly due to its relatively low basal expression. mRFP reporter gene fluorescence distributions measured by flow cytometry (FIG. 17C). At every inducer concentration, the LacI-Ptrc promoter demonstrated higher variability within the population than XylS-Pm. A bimodal population distribution with LacI-Ptrc was observed, suggesting that this promoter is unstable in P. putida. No bimodal distributions were detected with constitutive CRISPRa, and only a small bimodal population was observed with XylS-Pm inducible mRFP or dCas9. The strains shown for constitutive CRISPRa are on-target (J306) and off-target (OT) in the PPC01 background. For inducible CRISPRa, the strain background is PPC08. Values in FIG. 17A represent the mean ± standard deviation calculated from n = 6 independent biological replicates. Values in FIG. 17B represent the mean ± standard deviation calculated from n = 3 independent biological replicates. Values in FIG. 17C represent the data (n =1) from different inducer concentrations or scRNAs.

FIGS. 18A-18B show inducible CRISPRa by XylS-Pm on CRISPRa machinery. Inducible dCas9/MCP-SoxS constructs were integrated into the P. putida genome with the mini-Tn7 method (FIG. 18A). XylS-Pm promoters were introduced in place of the Sp.pCas9 promoter regulating dCas9 and/or the BBa_J23107 promoter for MCP-SoxS. Strains were transformed with a vector (pPPC020) carrying either an off-target scRNA or a J306 scRNA (FIG. 18B). The fold-activation in the presence of m-toluic acid as an inducer are 9-fold, 5-fold, and 10-fold from PPC08, PPC09, and PPC10, respectively. Strains with inducible dCas9 only, or both dCas9 and MCP-SoxS inducible, showed minimal leaky activation in the absence of inducer. If only MCP-SoxS is under control of the inducible promoter, leaky activation is detectable. In a strain with constitutively expressed CRISPRa machinery that should not be responsive to inducer, high inducer concentrations (5 mM) also led to a modest increase in mRFP expression (1.3-fold). Values represent the mean ± standard deviation calculated from n = 3 independent biological replicates.

FIGS. 19A-19C show Biopterin production in P. putida with CRISPRa tool. OD₃₄₀ absorption of P. putida supernatant, which corresponds to absorption of dihydrobiopterin and biopterin (FIG. 19A). The HPLC-MS signals of three biopterin derivatives with pathway genes regulated by different CRISPRa programs (FIG. 19B). Comparison of biopterin and dihydrobiopterin (BH2) production from a CRISPR-activated tetrahydrobiopterin pathway in E. coli MG1655 (transformed with pCK015 and pCK005.AAV/pCD581 bearing hAAVS1/J306 scRNA) and P. putida PPC01 (transformed with pPPC027 bearing either hAAVS1 or J306 scRNA) (FIG. 19C). The ratio of signal between BH2 and biopterin produced from P. putida (32:1) is higher than that of E. coli (7:1). Values in FIG. 19A represent the mean ± standard deviation calculated from n = 3 independent biological replicates. The no pathway control in FIG. 19A represents one (n = 1) sample. Values in FIGS. 19B-19C represent the mean ± standard deviation calculated from n = 3 technical replicates.

FIGS. 20A-20D show HPLC-MS Spectra of Biopterin products in P. putida. Overlaid chromatograms of commercial standards for biopterin, BH2, and BH4 normalized to maximum signal of each corresponding ion count (FIG. 20A). Biopterin pathway outputs from a P. putida strain with CRISPRa-mediated activation of the metabolic pathway (FIGS. 20B-20D). Panels correspond to m/z ion count signals for Biopterin (FIG. 20B), BH2 (FIG. 20C), and BH4 (FIG. 20D). The parental strain KT2440 (solid line) was used as a negative control (no heterologous pathway). PPC01 carrying pPPC027 with a J306 scRNA (dashed line) showed significant improvement in BH2 product compared to that of an off-target scRNA (dash-dotted line). BH4 (r.t. ~ 3 min) was not observed in any tested condition.

FIGS. 21A-21B show GC-MS detection of Mevalonic Acid. A representative standard curve of mevalonolactone in ethyl acetate at different concentrations (0, 25, 50, 100, 200, 400, and 500 mg/mL) measured by GC-MS at m/z = 71 (FIG. 21A). CRISPRa-mediated mevalonate production with additional off-target controls (see also FIG. 7) (FIG. 21B). Strains with an off-target scRNA yielded mevalonate levels that were indistinguishable from that of an empty plasmid control (less than 10 mg/L). Values in panel A represent the mean ± standard deviation calculated from n = 3 technical replicates. Values in panel B represent the mean ± standard deviation calculated from n = 3 independent biological replicates, n = 5 for the no plasmid control and off-target scRNA of constitutively expressed dCas9/MCP-SoxS strain, and n = 7 for the J306 scRNA.

FIG. 22 shows mini-Tn7T Plasmid Maps. Selected examples of integration plasmid maps with labeled important parts and restriction sites of pPPC001, pPPC002, and pPPC005.

FIG. 23 shows replicable Plasmid Maps. Selected examples of replicable plasmid maps with labeled important parts and restriction sites of pBBR1-GmR, pRK2-GmR, pPPC016, and pPPC030.

FIG. 24 illustrates overlaid HPLC chromatograms of p-ACA production and low-concentration spike in. Initial, low-concentration production of p-ACA was verified by superimposition of a larger peak after spiking a small amount of known p-ACA (2 µM) into the same supernatant. p-ACA is observed at 10.05 minutes.

FIGS. 25A-25B shows typical standard curves for HPLC quantification of p-AF(0 - 1000 µM) (FIG. 25A) and p-ACA (0 - 1000 µM) (FIG. 25B).

FIG. 26 shows CRISPRa-controlled p-AF production in E. coli. are arranged under the control of J3 and J2 synthetic promoters and are controlled by constitutive CRISPRa (removing the induction delay) (left panel). HPLC-detected p-AF production increases when papABC is activated, especially when aroGL is activated as well (right panel). This experiment was performed in MG1655 cells to be sure of CRISPRa function.

FIG. 27 shows production of p-ACA from the two-plasmid system in P. putida. Despite the small amount of p-AF substrate, activating At-PAL2 on the second plasmid results in a very small, but detectable, amount of p-ACA. Detection of p-ACA by this HPLC method is very sensitive due to the lack of interfering peaks in this area of the chromatogram; detecting even this amount of p-ACA is highly reproducible. The reduction in p-AF when PAL2 is activated could be in part due to substrate consumption but is probably mostly due to increased burden from the additional enzyme expression. This graph is presented with peak area on the y-axis, to better distinguish the presence or absence of p-ACA. Noted fold-changes are relative to the no-activation p-AF value. Error bars represent standard deviation of n=3 biological replicates.

FIG. 28 shows effects of second-plasmid burden, and genomic copy number, on p-AF production. The burden of carrying the second plasmid is substantial in P. putida, especially using a kanamycin resistance marker, reducing HPLC-measured p-AF production 16-fold in this experiment. Likely, this reduction is a result of lower heterologous enzyme expression due to lower global expression capacity. Also, probably due to low enzyme levels, the same promoters used in plasmid-based production fail to produce any p-AF when integrated in the genome. In all cases, only PapABC and AroGL are activated. Error bars represent standard deviation of n=3 biological replicates.

FIG. 29 shows one-plasmid p-ACA production using Rg-PAL. With the entire pathway contained on one large plasmid driven by CRISPR-activated J23117 promoters (left), p-ACA production is maximal at 480 µM, but drops off considerably when TyrB is included in an operon with PAL. This is presumably because of effects on PAL enzyme levels because it seems that conversion of p-AF to p-ACA is the most diminished factor. When PapABC and AroGL are integrated and driven by CRISPR-activated J23105 promoters (right), p-AF production exceeds that of a two-plasmid system by about two-fold, and p-ACA production is on par with the two-plasmid system when Rg-PAL is added. When PapABC and AroGL are integrated and driven by CRISPR-activated J23110 promoters (middle), genetic instability is observed, resulting in minimal and variable production across replicates. Seemingly the extra burden of the J23110 promoter strength overwhelms the stability of the strain’s production, but it is unclear whether this instability manifests at the scRNA plasmid or at the integration site. Error bars represent standard deviation of n=3 biological replicates.

FIG. 30 shows p-ACA production (right y-axis) from strains with various configurations of pathway enzymes integrated into the P. putida genome. Highest p-ACA production is observed using a fully plasmid-based system (left), due to the higher copy number. When only PAL is integrated (center left), excess p-AF (left y-axis) accumulates due to the plasmid’s higher copy number, increasing PapABC activity relative to PAL activity. When all pathway genes are integrated, with low-off-state (LL) CRISPRa promoters driving AroGL and PapABC expression (center right), maximal p-ACA production from an integrated strain is observed. Higher production results from a lower number of scRNAs expressed from the plasmid, and at a weaker promoter strength (3 × 105), than from a higher number of scRNAs at a stronger promoter strength (4 × 110). This potentially could be due to fewer scRNA transcripts competing to bind dCas9 or MCP-SoxS, or due to slight differences in termination efficiency between the two plasmids. When PAL is also expressed from a low-off-state CRISPRa promoter in an integrated strain (right), p-ACA production is decreased relative to a normal-off-state promoter. In all fully-integrated strains, no p-AF accumulation is observed, suggesting ample PAL activity relative to PapABC activity. Error bars represent standard deviation of n=3 biological replicates assessed by HPLC.

FIGS. 31A-31F show CRISPRa efficiency can be tuned by expression level of scRNA. The optimized expression level of scRNA from pBBR1-GmR is under J23110 promoter. Higher level (BBa_J23119) or lower level (BBa_J23106) led to decrease in CRISPRa effects (FIG. 31A). CRISPRa efficiency can be tuned by scRNA composition (FIG. 31B). Truncating the spacer sequence from 20 nucleotides to 8 nucleotides led to titratable expression level by CRISPRa. Desired product p-ACA is toxic to E. coli but not P. putida (FIG. 31C). In this kinetic growth experiment, extracellular p-ACA is supplied in the media at the indicated concentrations, and cultures are grown in a plate reader with periodic density measurements. E. coli growth is severely limited above 20 mM p-ACA (dash-dotted line). Early timepoints are sometimes obscured by p-ACA precipitate, which eventually resolubilizes. P. putida growth is relatively unaffected (dashed line), inspiring some confidence that p-ACA-producing cells will remain prominent in the culture. Error bars represent standard deviation of n=3 biological replicates. Multiple scRNAs investigation demonstrates that CRISPRa can regulate multiple genes using multiple scRNAs input (FIG. 31D). An example of CRISPRa with 3 orthogonal scRNAs activating 3 fluorescent reporters (FIG. 31E). Multiple scRNAs construction strategy was based on Golden-Gate Assembly (FIG. 31F).

FIGS. 32A-32D illustrate pAF/pACA production in P. putida. Schematic illustration of construct design used for pAF/pACA production in P. putida (FIG. 32A). p-ACA production using R. glutinis PAL (FIG. 32B). Changing the At-PAL2 ORF to that of RgPAL in the two-plasmid system results in dramatically increased p-ACA production. TyrB is only sometimes included in heterologous expression because there is also an endogenous copy. Its overexpression does seem to boost p-AF production, but its net effect on p-ACA production is less clear. A heterologous TyrB might be best expressed by a separate promoter than J6, as its inclusion in that operon could reduce PAL expression. Error bars represent standard deviation of n=3 biological replicates. A schematic depiction of the genome integration and plasmid design for expression strategy in i) all in the genome, ii) two separate plasmids, and iii) one big plasmid (FIG. 32C). Summary of one-plasmid p-ACA production using Rg-PAL (FIG. 32D). Compared to the modest production by the two-plasmid Rg-PAL system (left), the one-plasmid Rg-PAL systems can produce one- to four-fold higher p-ACA titer (middle). The entire pathway on one plasmid produces relatively high p-ACA titer and leaves little p-AF unconverted, suggesting that enzyme stoichiometry is well-balanced at these expression levels. On the right, PapABC and AroGL are integrated and driven by CRISPR-activated J23105 promoters, resulting in p-ACA production on par with the two-plasmid system with full conversion of p-AF. This finding suggested that PAL enzyme concentration is well-supplied in this condition and PapABC/AroGL are limiting. Error bars represent standard deviation of n=3 biological replicates.

FIGS. 33A-33C illustrate CRISPRa and CRISPRi on endogenous P. putida genes. CRISPRa is effective for 7 out of 11 targets with at least 1.5-fold activation (FIG. 33A). tpiA results are not shown as they cannot be transformed. CRISPRi is applicable for all 5 targets tested (FIG. 33B). CRISPRi-repression at sfGFP gene on the single-copy genome is more efficient than the high-copy plasmid condition (FIG. 33C).

FIGS. 34A-34D show the effect of increasing number of gRNA in pACA production. 3 sets of pACA metabolic pathway genes were incorporated either on a plasmid or on a genome while gRNAs were delivered on a plasmid (FIGS. 34A and 34B). CRISPRa is still functional when the number of gRNA increases from 3 to 6 when pACA pathway genes were delivered on the plasmid, a minimal decrease in pACA level, where production at 6 scRNAs equal to ~75% of pACA production was observed (FIG. 34C). The pACA production decreases by half with increasing number of gRNAs was increased from 3 to 6, when the pACA pathway was moved to the genome, but remains functional. (FIG. 34D).

FIGS. 35A-35C show CRISPRa in Acinetobacter baylyi ADP1. ADP1 was engineered into CRISPR enabled strain (CKAB029, FIG. 35A) which can consistently activate heterologous gene in different plasmid vectors (FIG. 35B). A. baylyi yielded higher fold-change at weak basal expression level (FIG. 35C).

FIGS. 36A-36B show PspF CRISPRa in P. putida and simultaneous functionality with SoxS. PspF-λN22 was integrated into dCas9/MCP-SoxS bearing strain to enable PspF CRISPRa (CKPP038, FIG. 36A). P. putida CKPP038 strain is functional for both SoxS-CRISPRa and PspF-CRISPRa (FIG. 36A). A dual fluorescent reporter was used to demonstrate the orthogonal programmability of two CRISPRa systems working simultaneously (FIG. 36B). sfGFP was activated with MCP-SoxS recruited by scRNA (J306) with MS2 hairpin while mRFP was activated with PspF-λN22 recruited by scRNA (J102) with BoxB hairpin.

FIG. 37 depicts P. putida genes targeting using PAM-expanded dCas9 variants.

### DETAILED DESCRIPTION

CRISPR-Cas transcriptional programming in bacteria is an emerging tool to regulate gene expression for metabolic pathway engineering. The present disclosure provides methods of CRISPR-Cas transcriptional activation (CRISPRa) in P. putida using a system previously developed in E. coli. The present disclosure provides a methodology to transfer CRISPRa to a new host by first optimizing expression levels for the CRISPRa system components, and then applying rules for effective CRISPRa based on a systematic characterization of promoter features. Using the optimized system disclosed herein, the inventors regulated biosynthesis in the biopterin and mevalonate pathways. The present disclosure demonstrates that multiple genes can be activated simultaneously by targeting multiple promoters or by targeting a single promoter in a multi-gene operon. The optimized CRISPRa approach provided herein can activate endogenous promoters for P. putida and inducible CRISPRa can be obtained by expressing dCas9 from inducible promoters. The present disclosure facilitates new metabolic engineering strategies in P. putida and paves the way for CRISPR-Cas transcriptional programming in other bacterial species.

In accordance with the foregoing, in one aspect the disclosure provides an engineered Pseudomonas bacterium containing genetic elements supporting programmable transcriptional activation and/or repression. In some embodiments, the engineered Pseudomonas bacterium comprises at least one heterologous nucleic acid construct.

In some embodiments, the at least one heterologous nucleic acid construct comprises a first sequence encoding an endonuclease that lacks endonuclease activity. In some embodiments, the endonuclease is dCas9, dCas12, dCasX, dCasPhi, dCas3 (Cascade), and the like.

In some embodiments, the at least one heterologous nucleic acid construct comprises a second sequence encoding a transcriptional activator. In some embodiments, the transcriptional activator comprises an RNA-binding protein (RBP) fused to an effector domain of a transcriptional activator. In some embodiments, the transcriptional activator is selected from SoxS, TetD, PspF, AsiA, N-terminus of RpoA (aNTD), and Soxs-family activators (e.g., AraC-XylS superfamily), and the like. In some embodiments, the RNA-binding protein can be selected from MCP, PCP, Com, LambdaN22Plus, Qbeta. In some embodiments, the SoxS is derived from E. coli. In some embodiments, the SoxS is engineered to reduce or abolish DNA-binding capacity. In some embodiments, the SoxS is engineered to contain a mutation, e.g., substitution, at reside R93 and/or S101, e.g., R93A and/or S101A, and the like.

In some embodiments, the at least one heterologous nucleic acid construct comprises a third sequence encoding a scaffold RNA (scRNA). In some embodiments, the scRNA comprises a 3′ MS2 hairpin loop that interacts with a transcriptional activator. In some embodiments, the scRNA comprises a 5′ domain comprising a guide sequence that hybridizes to a target sequence. In some embodiments, the target sequence is proximal to a protospacer adjacent motif (PAM) and/or promoter sequence of an endogenous gene of the Pseudomonas bacterium. In some embodiments, the at least one heterologous nucleic acid construct comprises a fourth sequence comprising an open reading frame of a gene of interest (GOI) operatively linked to a promoter sequence and/or PAM sequence, and wherein the target sequence is proximal to the promoter sequence and/or PAM sequence.

In some embodiments, the at least one heterologous nucleic acid construct comprises the first, second, third, and fourth sequences distributed in any combination on two vectors. In some embodiments, the at least one heterologous nucleic acid construct comprises the first, second, third, and fourth sequences distributed on a single vector. In some embodiments, the vector is optionally pBBR1, pRK2, pRSF1010, and the like, or is derived from.

In some embodiments, the at least one heterologous nucleic acid construct is integrated into the genome of the Pseudomonas bacterium. In some embodiments, the first, second, third, and fourth sequences each comprise or are operatively linked to a promoter operable in the Pseudomonas bacterium. In some embodiments, the Pseudomonas bacterium is P. putida. In some embodiments, the target sequence is between 60 and 120 bases upstream (5′ to) the transcriptional start site of the endogenous gene or open reading frame. In some embodiments, the target sequence is 15-25 bases. In some embodiments, the target sequence corresponds with the J1 or J3 promoter, or portion thereof.

In some embodiments, a promoter sequence resides in the intervening sequence between the target sequence and the transcriptional start site (TSS) of the endogenous genes or open reading frame. In some embodiments, the promoter sequence is a synthetic 5′-upstream sequence containing appropriate NGG PAM at an optimal position (e.g., 75-85 nt, e.g, 78-83 nt, e.g., 81 nt upstream of the TSS). In some embodiments, the genetic elements are under control of a small-molecule inducible promoter. In some embodiments, the small molecule inducer is selected from m-toluic acid, salicylic acid, benzoic acid, and related compounds. In some embodiments, the small-molecule inducible promoter is XylS/Pm, e.g., derived from P. putida mt-2. In some embodiments, the at least one heterologous nucleic acid construct comprises a fourth sequence comprising an open reading frame of a gene of interest, wherein the open reading frame encodes gene product that results in production of an aromatic compound.

In some embodiments, the Pseudomonas bacterium is engineered to produce p-aminocinnamic acid (pACA) from glucose. In some embodiments, the Pseudomonas bacterium comprises an open reading frame encoding PAL, optionally wherein the PAL is derived from Arabinobsis thaliana or Rhodotorula glutinis. In some embodiments, the Pseudomonas bacterium comprises an open reading frame encoding PapABC (4-amino-4-deoxychorismate synthase (PapA), 4-amino-4-deoxychorismate mutase (PapB) and 4-amino-4-deoxyprephenate dehydrogenase (PapC)), e.g., derived from Pseudomonas fluorescens, to facilitate p-AF synthesis. In some embodiments, the Pseudomonas bacterium comprises an open reading frame encoding AroGL, e.g., derived from E. coli, to facilitate chorismite flux upcycling.

In some embodiments, the Pseudomonas bacterium is engineered to produce tetrahydrobiopterin (BH4) or derivatives thereof. In some embodiments, the Pseudomonas bacterium comprises an open reading frame encoding GTP cyclohydrolase I (GTPCH), e.g., derived from E. coli. In some embodiments, the Pseudomonas bacterium comprises an open reading frame encoding PTPS (pyruvoyltetrahydropterin synthase) /SR (sepiapterin reductase), e.g., derived from M. alpina.

In another aspect, the disclosure provides a system for production of aromatic compounds or compounds with aromatic metabolites or intermediates, comprising the engineered Pseudomonas bacterium disclosed herein and a growth medium.

In another aspect, the disclosure provides a method of producing aromatic compounds or compounds with aromatic metabolites or intermediates, comprising providing the engineered Pseudomonas bacterium disclosed herein and a suitable substrate and permitting production of the compounds. In some embodiments, the compound is p-AF and/or p-ACA and the substrate is glucose.

### Additional Definitions

Unless specifically defined herein, all terms used herein have the same meaning as they would to one skilled in the art of the present disclosure. Practitioners are particularly directed to Sambrook J., et al. (eds.), Molecular Cloning: A Laboratory Manual, 3rd ed., Cold Spring Harbor Press, Plainsview, New York (2001); Ausubel, F.M., et al. (eds.), Current Protocols in Molecular Biology, John Wiley & Sons, New York (2010); Coligan, J.E., et al. (eds.), Current Protocols in Immunology, John Wiley & Sons, New York (2010); Mirzaei, H. and Carrasco, M. (eds.), Modern Proteomics - Sample Preparation, Analysis and Practical Applications in Advances in Experimental Medicine and Biology, Springer International Publishing, 2016; Comai, L, et al., (eds.), Proteomic: Methods and Protocols in Methods in Molecular Biology, Springer International Publishing, 2017; Mali P, Esvelt KM, and Church GM. Cas9 as a versatile tool for engineering biology. Nat Methods. 2013 Oct;10(10):957-63; and Dominguez AA, Lim WA, and Qi LS. Beyond editing: repurposing CRISPR-Cas9 for precision genome regulation and interrogation. Nat Rev Mol Cell Biol. 2016 Jan;17(1):5-15, for definitions and terms of art.

For convenience, certain terms employed herein, in the specification, examples and appended claims are provided here. The definitions are provided to aid in describing particular embodiments and are not intended to limit the claimed invention, because the scope of the disclosure is limited only by the claims.

A nucleic acid is a polymer of monomer units or “residues”. The monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5 -carboxycytosine b-glucosyl-5-hydroxy-methylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

The five-carbon sugar to which the nucleobases are attached can vary depending on the type of nucleic acid. For example, the sugar is deoxyribose in DNA and is ribose in RNA. In some instances, herein, the nucleic acid residues can also be referred with respect to the nucleoside structure, such as adenosine, guanosine, 5-methyluridine, uridine, and cytidine. Moreover, alternative nomenclature for the nucleoside also includes indicating a “ribo” or deoxyribo” prefix before the nucleobase to infer the type of five-carbon sugar. For example, “ribocytosine” as occasionally used herein is equivalent to a cytidine residue because it indicates the presence of a ribose sugar in the RNA molecule at that residue. A nucleic acid polymer can be or comprise a deoxyribonucleotide (DNA) polymer, a ribonucleotide (RNA) polymer. The nucleic acids can also be or comprise a PNA polymer, or a combination of any of the polymer types described herein (e.g., contain residues with different sugars).

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

As used herein, the term “polypeptide” or “protein” refers to a polymer in which the monomers are amino acid residues that are joined together through amide bonds. When the amino acids are alpha-amino acids, either the L-optical isomer or the D-optical isomer can be used, the L-isomers being preferred. The term polypeptide or protein as used herein encompasses any amino acid sequence and includes modified sequences such as glycoproteins. The term polypeptide is specifically intended to cover naturally occurring proteins, as well as those that are recombinantly or synthetically produced.

One of skill will recognize that individual substitutions, deletions or additions to a peptide, polypeptide, or protein sequence which alters, adds, or deletes a single amino acid or a percentage of amino acids in the sequence is a “conservatively modified variant” where the alteration results in the substitution of an amino acid with a chemically similar amino acid. Conservative amino acid substitution tables providing functionally similar amino acids are well known to one of ordinary skill in the art. The following six groups are examples of amino acids that are considered to be conservative substitutions for one another:
(1) Alanine (A), Serine (S), Threonine (T),
(2) Aspartic acid (D), Glutamic acid (E),
(3) Asparagine (N), Glutamine (Q),
(4) Arginine (R), Lysine (K),
(5) Isoleucine (I), Leucine (L), Methionine (M), Valine (V), and
(6) Phenylalanine (F), Tyrosine (Y), Tryptophan (W).

Reference to sequence identity addresses the degree of similarity of two polymeric sequences, such as protein or nucleic acid sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

“Promoter” as used herein means a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which may be located as much as several thousand base pairs, or anywhere in the genome, from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, hormones, toxins, drugs, pathogens, metal ions, or inducing agents.

“Protospacer sequence” or “protospacer segment” as used interchangeably herein refers to a DNA sequence targeted by the Cas9 nuclease or Cpf1 nuclease in the CRISPR bacterial adaptive immune system. In the CRISPR/Cas9 system, the protospacer sequence is typically followed by a protospacer-adjacent motif (PAM); the PAM is at the 5′-end. In the CRISPR/Cpf1 system, PAM is followed by the protospacer sequence; the PAM is at the 3′-end.

“Protospacer adjacent motif” or “PAM” as used herein refers to a DNA sequence immediately following the DNA sequence targeted by the Cas9 or immediately before the DNA sequence targeted by the Cpf1 nuclease in the CRISPR bacterial adaptive immune system.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

CRISPR-Cas system has been repurposed for several applications in the field of synthetic biology including transcriptional modifications based on catalytically-dead Cas9 (dCas9), guided-RNA, and other prosthetic machinery. CRISPR interference (CRISPRi) can be achieved by having dCas9 physically blocking the RNA polymerase function. On the other hand, CRISPR activation (CRISPRa) requires an auxiliary component to recruit/stabilize RNA polymerase to the proper position to elevate the expression of designated genes. Combined CRISPR activation/repression (CRISPRa/i) circuits can be programmed at the engineered guide-RNA(s) containing complementary sequences to the DNA target, single-guide-RNA (sgRNA) for CRISPRi, and scaffold-RNA (scRNA) for CRISPRa. Therefore, CRISPRa/i can provide a programmable environment towards genome-scale engineering to accelerate chemical production optimization.

Moreover, the CRISPRa/i tool was recently demonstrated to be applicable in Pseudomonas putida, the emerging bacterial chassis that has different industrially relevant traits suitable for metabolic engineering applications. Hence, the accelerated genetic engineering platform will be applied to P. putida and other microbes of interest to explore biosynthesis space beyond a benchmark E. coli host. The biosynthetic pathway of p-aminocinnamic acid was chosen for manipulation due to difficulties observed in the E. coli system.

Successful acceleration of genetic manipulation tools will provide a novel platform for strains engineering towards bioproduction of any desired chemicals in multiple organisms of choice. By reducing the time from individual gene engineering at the genome level to multiple gene perturbations based on CRISPRa/i program, the accessibility to novel strains will be significantly boosted.

### Port CRISPRa Tool to Pseudomonas Putida

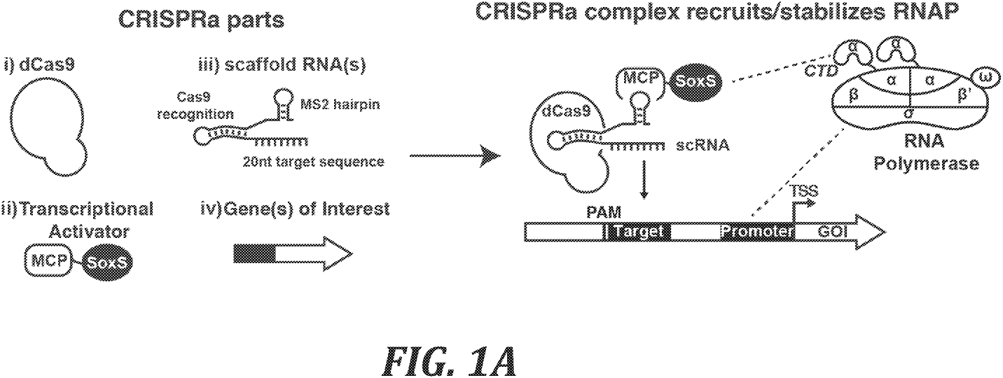

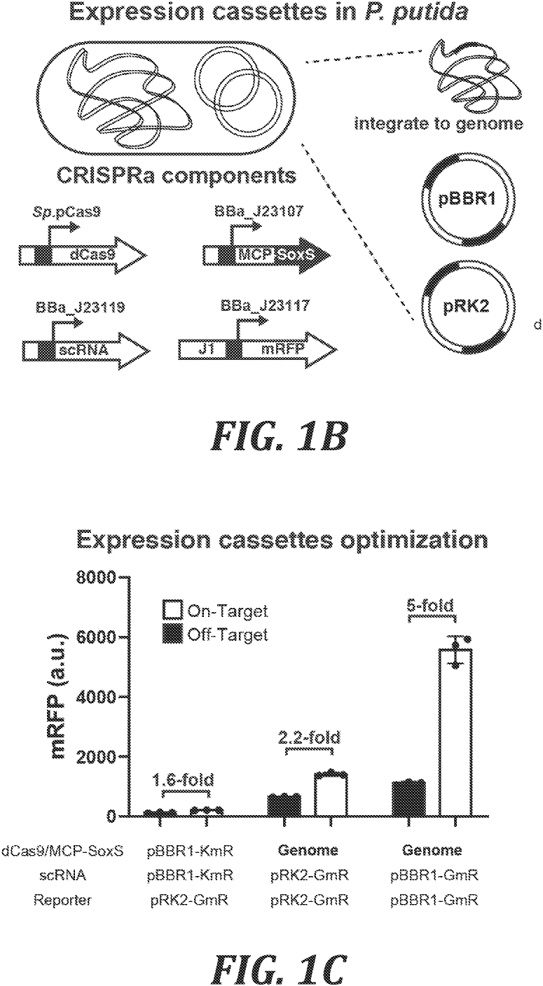

CRISPR-based transcriptional activation is enabled by appending the transcription factor that recruits/stabilizes RNA polymerase to the bacterial promoter and increases transcription rate. The initial screening covered diverse protein candidates and SoxS was found to be the best performer among several candidates. The novel CRISPRa tool was also demonstrated simultaneously with CRISPRi to activate/repress multiple fluorescent reporters. Further, the engineering of SoxS with R93A and S101A mutations led to significant improvement in activation which also allowed upregulation of endogenous promoters with moderate success rate. The present disclosure demonstrates successful use of CRISPRa tool in P. putida with thorough methodology to enable bioproduction of biopterins and mevalonic acid. Even though the genetic tools available in E. coli were shown to be somewhat compatible in P. putida, key limitations existed in the plasmid-borne expression system used in E. coli, which is incompatible with other bacterial strains distantly related to Escherichia genus. Therefore, broad-host-range plasmids were used instead, and an initial CRISPRa activity was demonstrated, and the expression system was optimized in both plasmid-borne and genome-integrated manners in which the latter was demonstrated to be superior in comparison (Example 1, FIG. 1C).

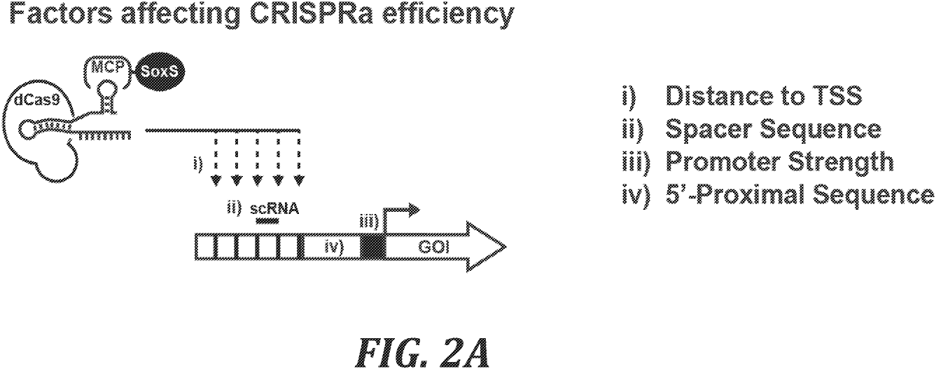

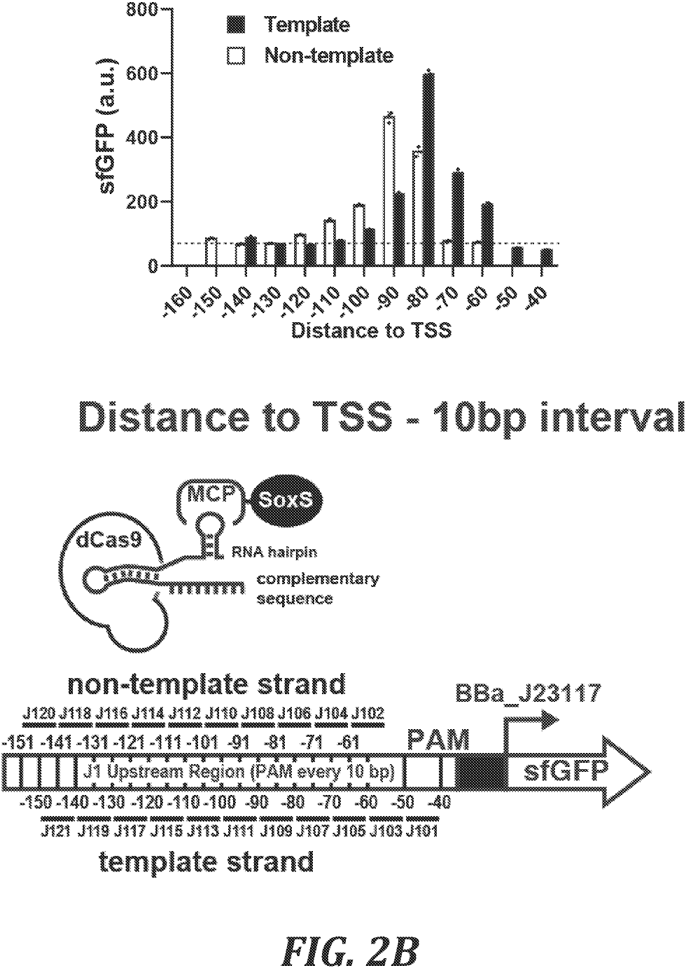

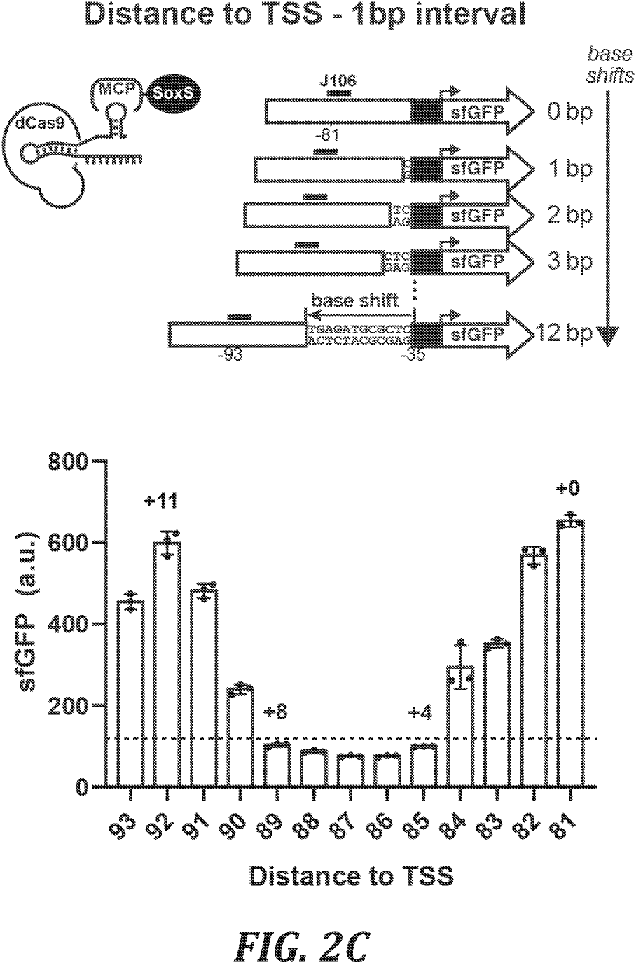

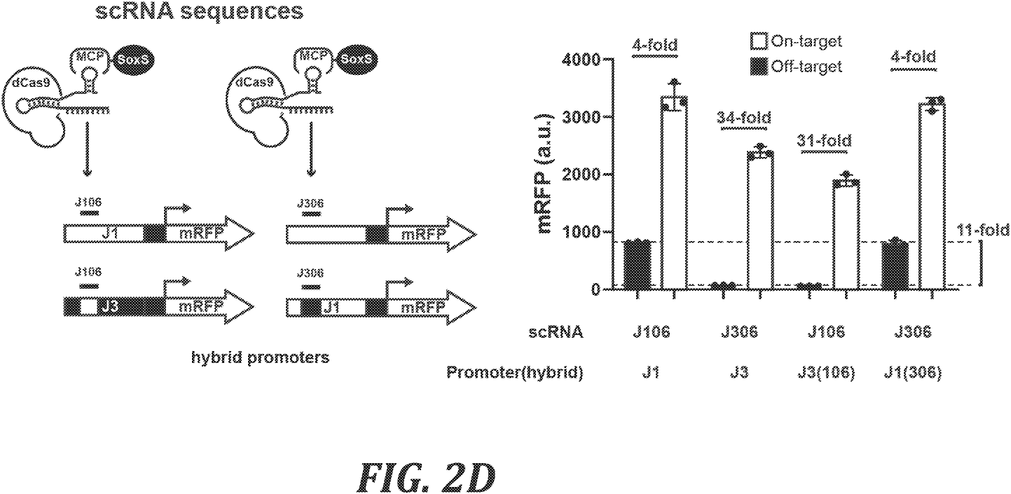

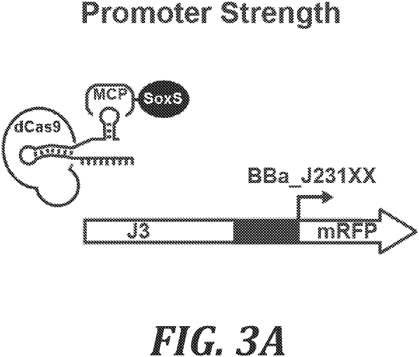

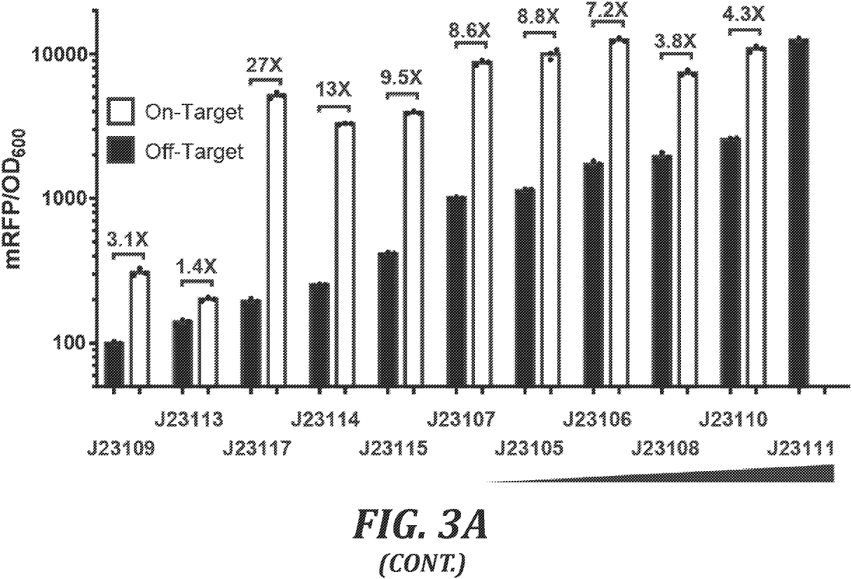

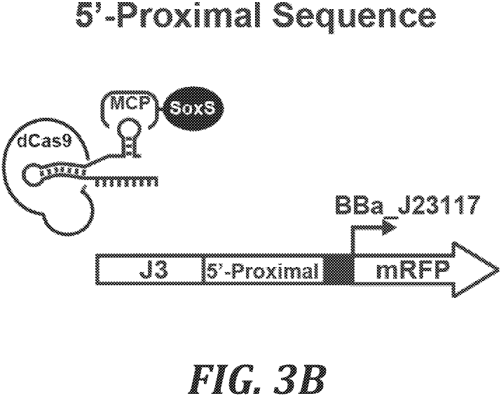

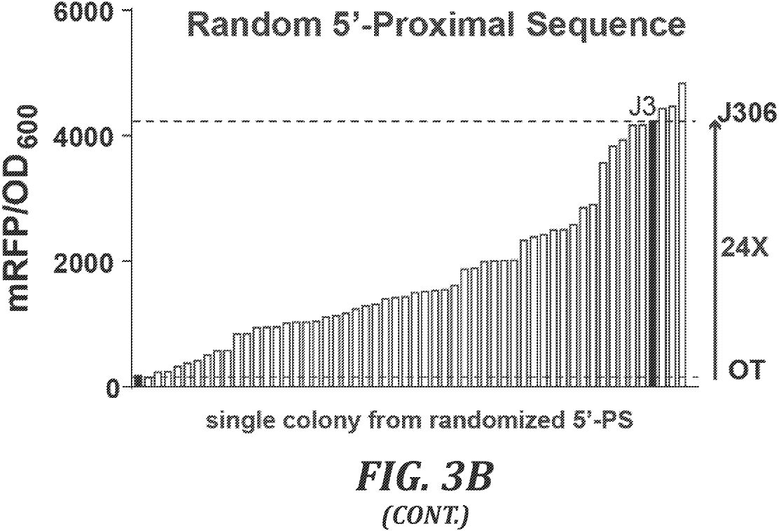

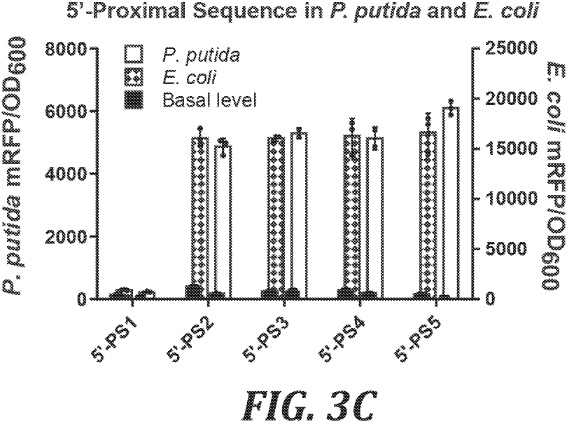

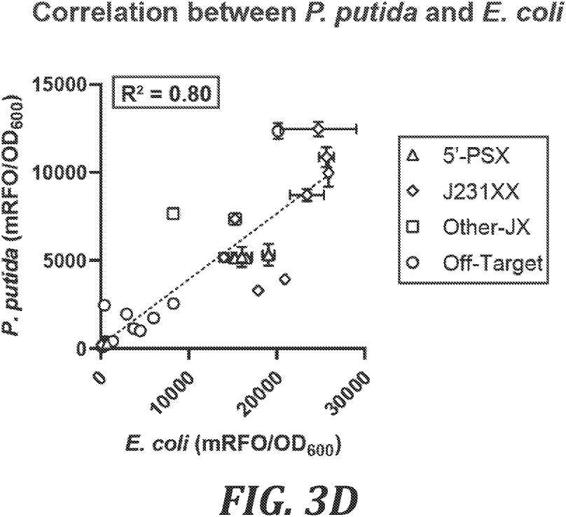

As CRISPRa recruits/stabilizes RNA polymerase to the designated promoter region, there are specific requirements for CRISPRa to work effectively. Several factors, e.g., distance to the transcriptional start site (TSS) and promoter strengths, influence the efficiency of CRISPRa, and these factors were investigated in the P. putida system for optimization (Example 1, FIGS. 2A-2D and FIGS. 3A-3C).

Despite the CRISPRa activity of synthetic reporters observed, the activation of endogenous promoters remains challenging. The inability to engineer the endogenous promoter led to unoptimized traits for CRISPRa, e.g., limited availability of the PAM sites and out-of-range promoter strengths.

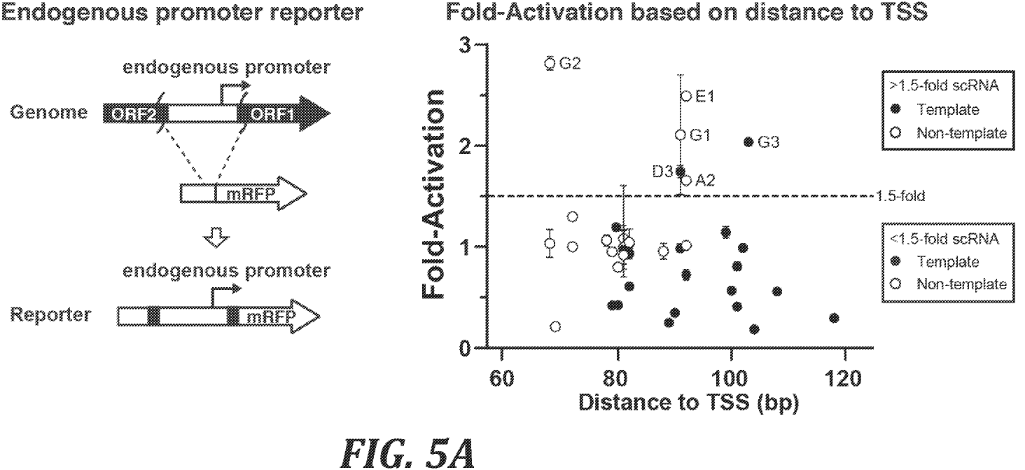

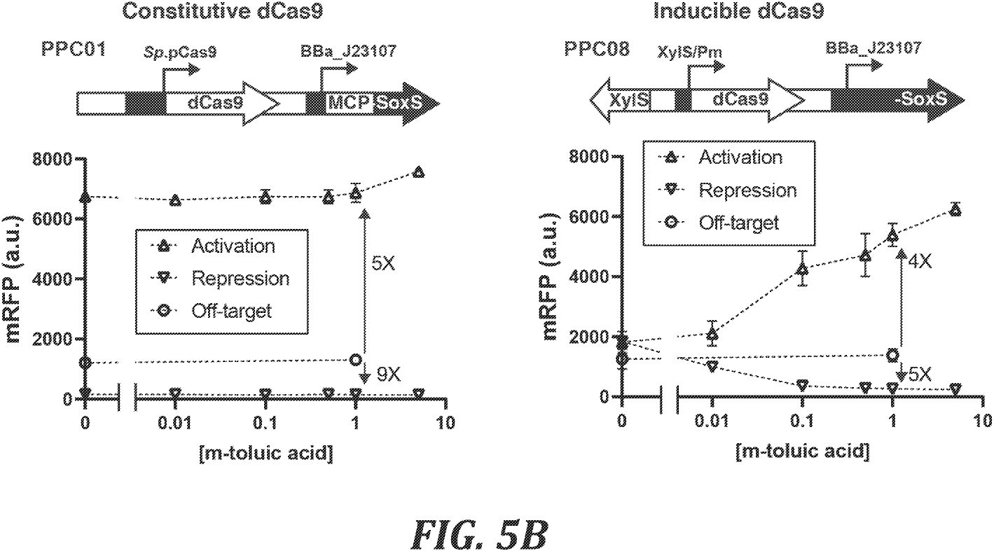

To examine the endogenous CRISPRa capability in P. putida, fluorescent protein fusion reporters were constructed with the native/endogenous promoters and CRISPRa activity tested with all available scRNAs filtered by working distance-to-TSS. Out of 10 promoters tested, 4 can be activated with at least 1.5-fold increase in the fluorescent output (FIGS. 5A-5B). These findings support the idea that endogenous CRISPRa is accessible but that preliminary validation is necessary. Based on these data, a predictive model to filter the set of activatable genes from the genome-wide transcriptome is contemplated.

The inventors have successfully ported CRISPRa and characterized effective CRISPRa in P. putida. Further, the data presented herein establish that endogenous genes can be activated in P. putida using fluorescent protein fusion reporter.

### Characterize CRISPRa/i Program for the Strains Engineering Acceleration

Although characterization of CRISPRa in P. putida was an important step, it was equally critical to characterize the repression counterpart (CRISPRi). CRISPRi in P. putida was previously reported to be accessible and can be used in various applications. To this end the inventors tested CRISPRi efficiency with 17 different sgRNAs targeting a sfGFP reporter integrated into the P. putida genome. It was observed that distance is not the only factor governing the CRISPRi efficiency and that RNA folding energetics play an important role in the efficiency of CRISPRi. To further investigate the polar effect from interfering with the adjacent gene in the operon, CRISPRi was also tested in the sfGFP-mRFP and mRFP-sfGFP operon. It is evident that targeting the adjacent gene will likely affect the expression level of the adjacent gene regardless of the orientation. (Data not shown)

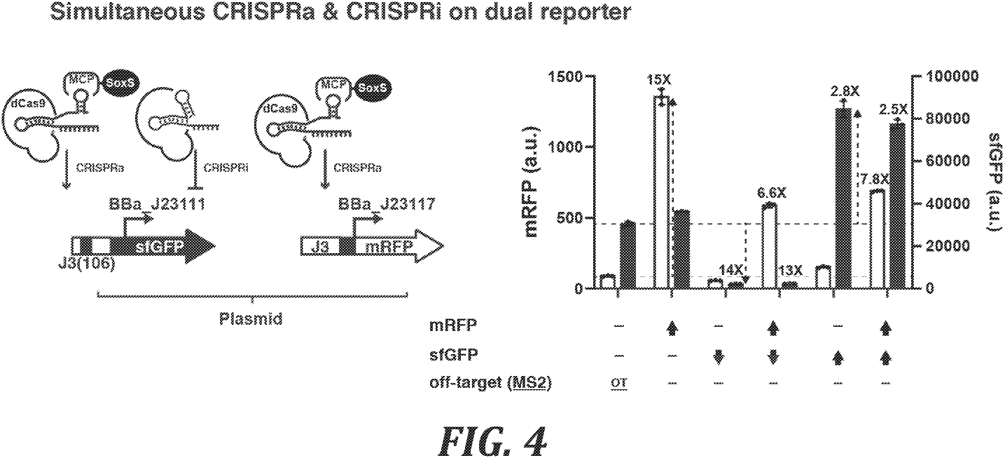

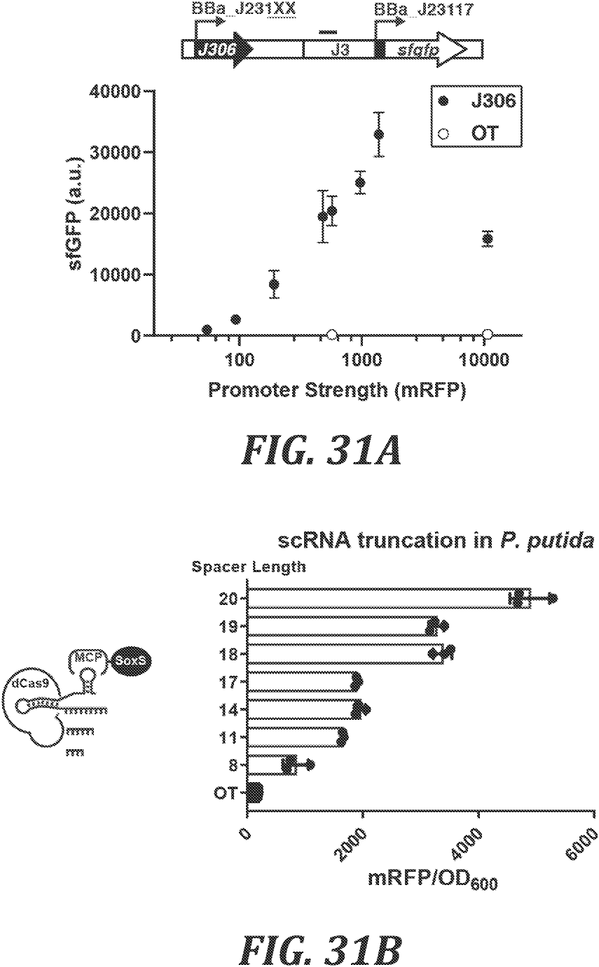

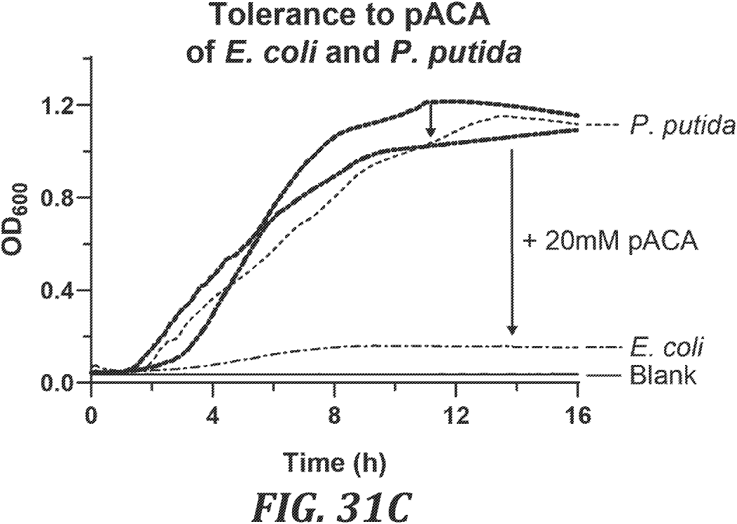

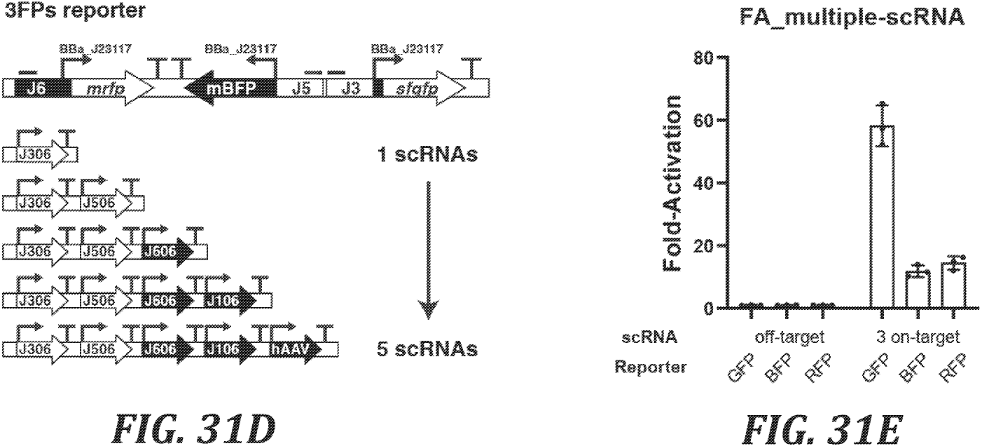

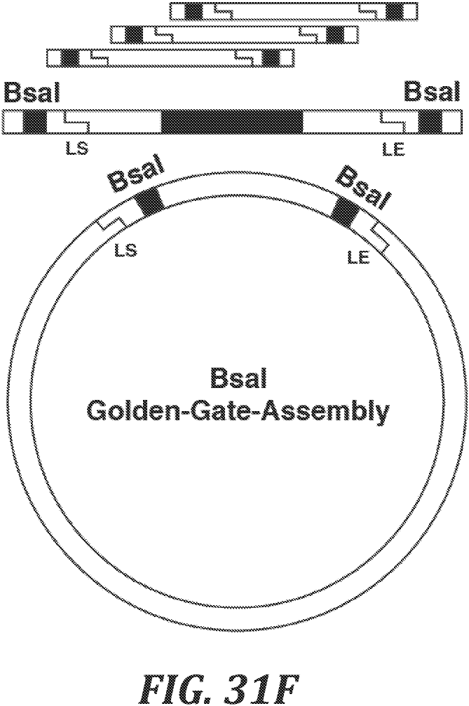

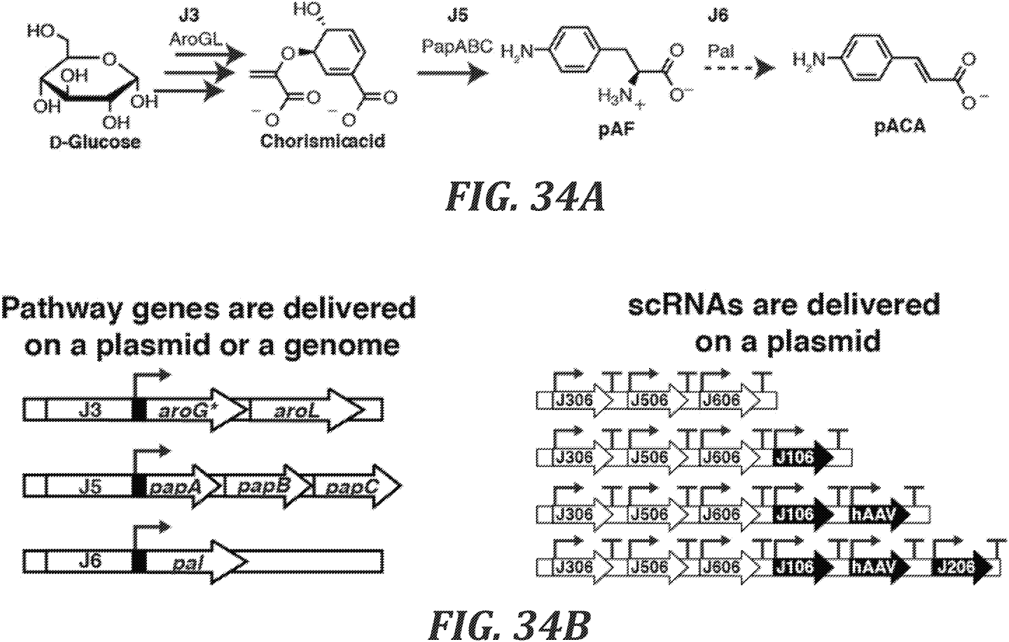

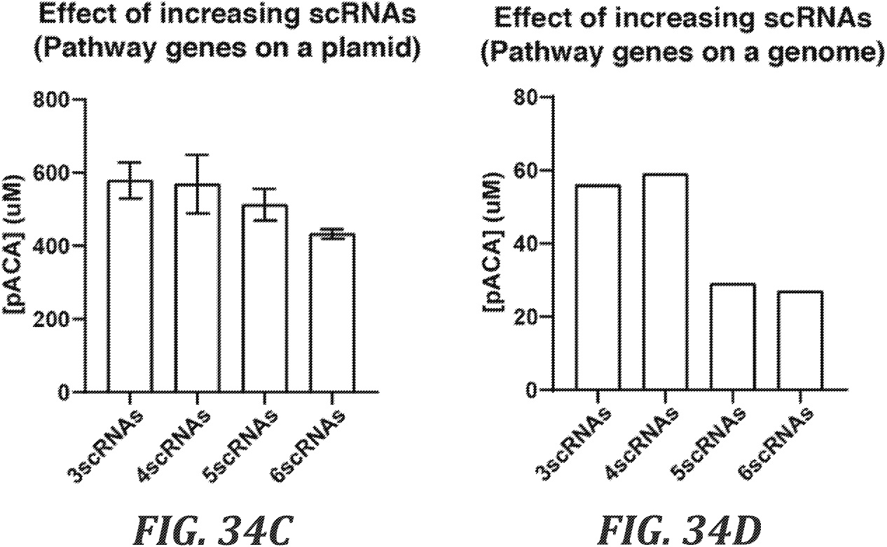

Furthermore, to demonstrate the simultaneous activity of CRISPRa and CRISPRi, the inventors constructed and transfected, plasmid-borne and integrated dual-fluorescent proteins reporters, in P. putida. The CRISPRa and CRISPRi circuits were shown to be functional both individually and simultaneously on these promoters (FIG. 4). However, even though multiple scRNAs can be used to activate multiple targets simultaneously, it was observed that additional scRNA significantly impair the fold-activation. Similar effects were observed in the triple fluorescent protein reporter controlled by three orthogonal scRNAs tested in E. coli (Example 1). To validate this limitation, multi-gRNA expression plasmid based on Golden-Gate Assembly inspired by the BioBrick cloning method was designed. Plasmids expressing 1-6 scRNA(s) were constructed and tested accordingly (FIG. 31F). Using multiple fluorescent proteins as reporters, we found that 3 scRNAs were effective for activation of 3 reporter genes (FIGS. 31D-31E). Further evaluation in the chemical production showed slight decreased efficiency when pathway genes are on the plasmid (FIG. 34C) but significant decrease could be found when pathway genes were integrated into the genome (FIG. 34D).

Other than its ability to activate and repress genes of interest, the CRISPRa/i program also encompasses fine-tuning of the perturbation levels using various strategies. The degree of activation and repression can be tuned in respect to concentration of each CRISPRa/i machinery (FIGS. 31A-31B). The titration of dCas9 protein via inducible XylS/Pm promoter can lead to different CRISPRa and CRISPRi levels of target genes. Tuning the concentration of the activator protein under control of aTc-inducible promoter can also regulate the degree of CRISPRa. Titrating the sg/scRNA concentration with different promoter strength can also lead to variation in CRISPRa/i efficiency. However, the change in the concentration of CRISPR components will affect the overall efficiency of every target which makes fine-tuning at the specific gene difficult. DNA-RNA energetics also affect the efficiency of CRISPRalI, which can be achieved through truncation or mismatch of the sg/scRNA. The truncation of scRNA was tested in P. putida and was found to provide different levels of CRISPRa. With the strategies disclosed herein, the genome-scale CRISPRa/i can be designed and tuned to achieve desired expression levels of GOIs using computational models.

Based on these studies and the data presented herein, the inventors have characterized CRISPRi in P. putida, including the polar effect by targeting the multi-gene operon, demonstrated that CRISPRa and CRISPRi work simultaneously both in the plasmid and genome-integrated platforms, CRISPRa/i level can be tuned in various aspects, CRISPR components affect level of CRISPRa/i, and scRNA truncation led to decreased CRISPRa magnitude.

### Applying CRISPRa/i Program to Accelerate Strains Engineering of pACA Production

To prove the ability to accelerate strain engineering processes in bacteria, p-aminocinnamic acid (pACA) production pathway was chosen as an exemplary pathway. pACA is a non-native chemical in biologically-derived chemical repertoire which can be achieved by coupling the p-aminophenylalanine (pAF) biosynthetic pathway, retrieved from Pseudomonas fluorescens, with phenylalanine ammonia lyase, available in plant and fungal chemistry. With further chemical modification or bioconversions, pACA can be converted into p-aminostyrene (pAS) which is a precursor of derivatized polystyrene, containing a functional group for further modification or functionalization with other polymers. The biosynthesis of pACA directly from common feedstocks, e.g., glucose, has not been reported which may suggest that production of pACA in bacteria is problematic. Assuming that high concentration of pACA could be deleterious to the host, a growth experiment using E. coli, the standard chassis, and P. putida, known for resistance to aromatic compounds was performed. It is obvious that E. coli cannot tolerate high concentrations of pACA while P. putida growth is less affected. See FIG. 31C. Thus, the solution to microbial production of pACA might be achievable with P. putida as an alternative chassis.

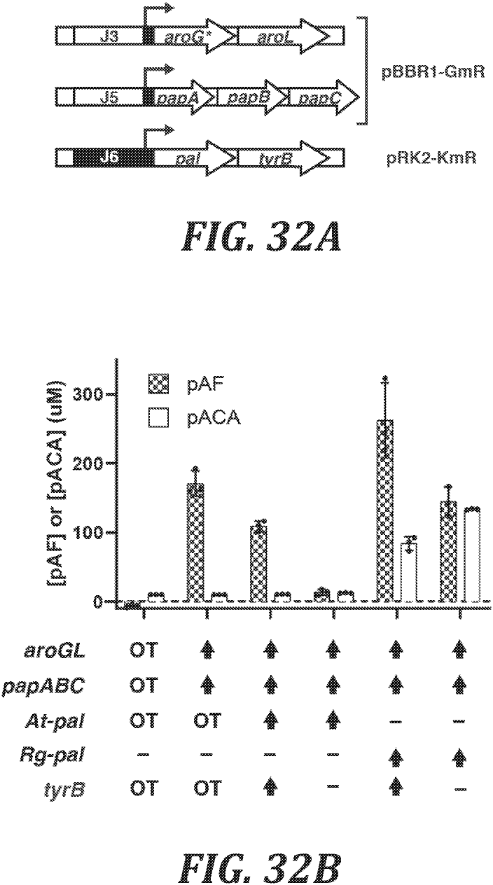

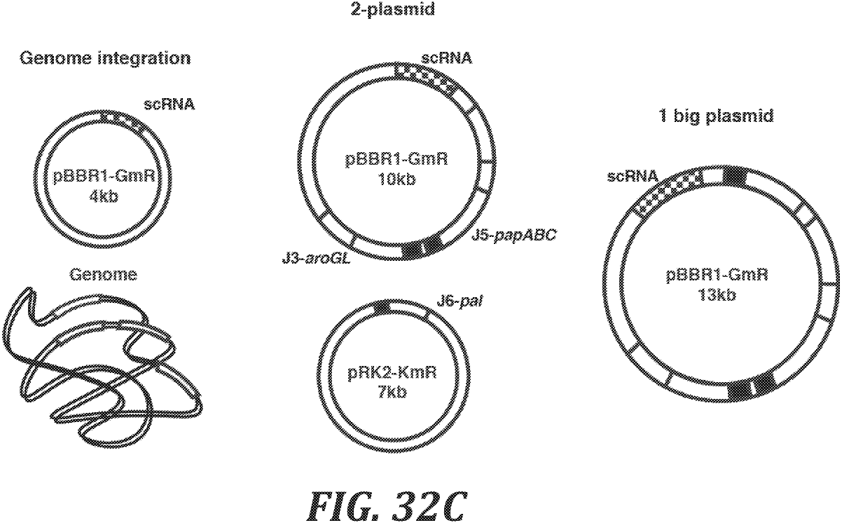

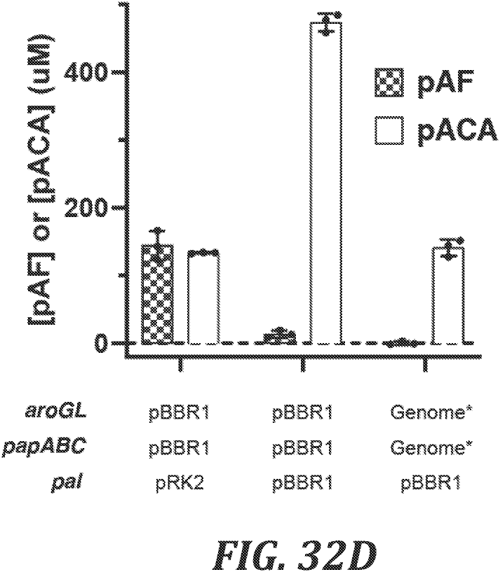

In E. coli, pAF can be produced by CRISPRa control of two operons: aroG*L from E. coli and papABC from P. fluorescens. The whole cassette was successfully ported into P. putida compatible plasmid, and it was shown that pAF can be produced efficiently. Next, the pal-tyrB operon, Pal from Arabidopsis thaliana and tyrB from E. coli, was supplied to enable the pACA production from pAF on the second plasmid and trace amounts of pACA in the supernatant were observed. See FIG. 32B. The high concentration of pAF remaining in the system with the presence of Pal enzyme suggested that the conversion of pAF to pACA might be inefficient. Phenylalanine ammonia lyase (Pal) has phenylalanine as an original substrate which might not be optimal for an amino-group containing derivative. Thus, other variants of Pal that could be more compatible with pACA production were sought. Rhodotorula glutinis Pal, reported to provide higher compatibility with pAF, was tested and a significant improvement in pACA production was found. Further, it was found that exclusion of tyrB led to improvement in pACA level. See FIG. 32B.

Prior to genome-wide perturbations, the heterologous gene expressions were optimized to ease the downstream engineering. Three approaches, to both express pACA biosynthetic pathway and perturb P. putida endogenous metabolism, were developed (FIG. 32C). First is the two-plasmid set-up described above where three heterologous operons were expressed from two different plasmids. Even though this approach has proved to be successful in pACA production, it was observed that P. putida bearing two plasmids suffer from higher degree of burden compared to one-plasmid strain. It was also observed that having the second empty plasmid significantly affects the pAF production.

Therefore, a second approach was taken to reduce the number of plasmids down to just one with three operons incorporated with a multi-gRNA program. Accordingly, a big plasmid (13kb) incorporating the three operons, was successfully constructed, which demonstrated elevated production of pACA. However, additional morphology, with larger colony size, in the P. putida transformation was observed, which may suggest instability of oversized plasmid. Restriction digest and sequencing suggested that part of the plasmid was deleted plausibly by transient recombination activity of P. putida. To solve this problem, a smaller backbone architecture for this broad-host-range pBBR1 plasmid was used to mitigate the plasmid size problem.

The third approach utilized by the present disclosure was to move the whole heterologous gene cassettes into the P. putida genome which leaves only the compact gRNA program on the plasmid. Initially, the first two operons for producing pAF were moved into the genome and it was observed that the pAF being produced by this approach is drastically reduced. By comparing the protein expression from multi-copy plasmid and single-copy genome-integrated cassette, it was observed that the expression capacity of the genome integrated cassette is several magnitudes lower than that of plasmid ones. The weak promoter of integrated cassettes was altered to one with moderate strength and it was found that pAF level significantly increases and is enough for pACA production. However, pACA production levels by this approach were lower than that of the big plasmid approach. To this point, both approaches are suitable for pACA production.

With the pACA production platform established, the inventors used genome-scale manipulation to optimize the process. A modified genome-scale model (GSM) that includes the pACA production pathway (papABC and pal reactions from chorismate to pACA) into the available P. putida KT2440 GSM using MetaCyc database was used. With iterations of change in chemical reactions corresponding to single-gene perturbations, the upregulation or downregulation of gene candidates that lead to higher production of pACA were recommended. 12 recommended genes for upregulation were mainly related to aromatic amino acid biosynthesis or central carbon metabolism. 31 recommended genes for downregulation were mostly nucleic acid biosynthesis and amino acid biosynthesis. 8 additional reactions were also identified that potentially compete with the pACA biosynthesis and these were included into downregulation candidates.

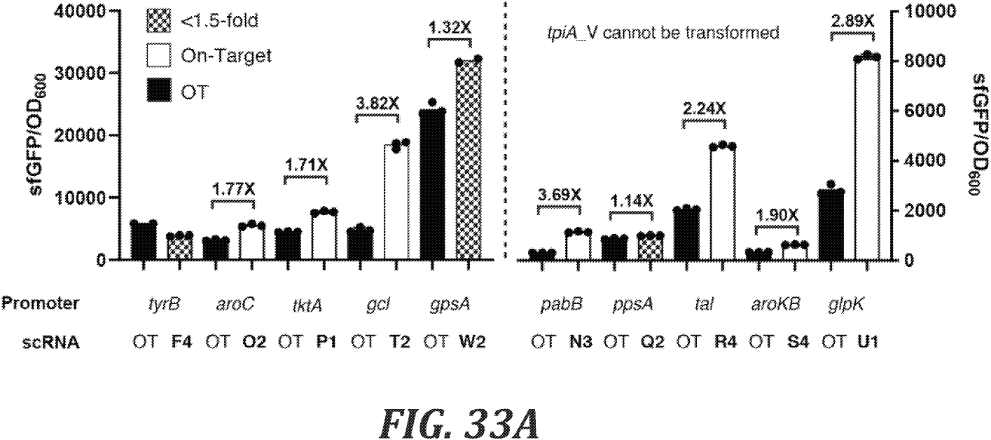

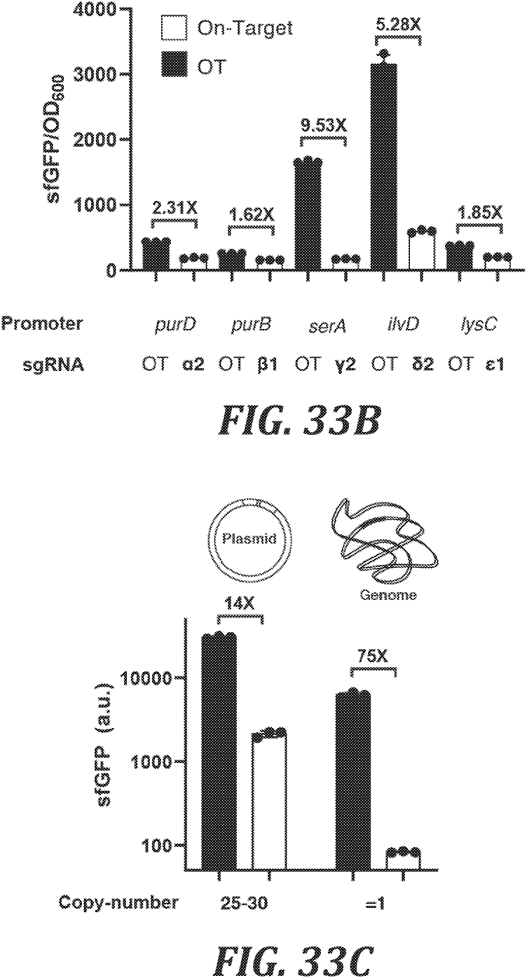

To test the ability to perturb endogenous gene candidates, the CRISPRa/i activity was investigated using GFP-fusion reporters by appending the sfGFP gene to the coding sequence of potential targets. sfGFP sequence 60bp was tagged after the start codon for CRISPRa similar to reported literature and 300bp for CRISPRi to accommodate space for CRISPRi target in the coding sequence. All scRNA with proper distance-to-TSS with reported and predicted TSS were screened. Out of 11 promoters (PP_0578 and PP_0579 are under the same promoter), 7 promoters were activated with >1.5-fold activation (FIG. 33A).

For CRISPRi candidates, all sgRNA were analyzed through the Wayfinder algorithm and screened based on RNA folding energetics. Two best sgRNA candidates for each promoter will be experimentally tested for both CRISPRi efficiency and growth defect. Out of 38 CRISPRi target promoters (PP_0420 and PP_0421 are under the same promoter), the first 5 promoters were tested to have > 1.5-fold repression (FIG. 33B).

In summary, pACA was selected to demonstrate strain engineering acceleration. The direct bioproduction of pACA from glucose in bacteria was demonstrated in P. putida CRISPRa platform. Phenylalanine ammonia lyase (Pal) from R. glutinis was observed to outperform A. thaliana Pal in pACA conversion.

Further, different approaches in heterologous genes and multi-gRNA cassettes delivery were tested. The two-plasmid system appeared to be relatively burdensome, whereas use of the big, single plasmid system provided the highest pACA production but suffered from instability. Genome integration of the pAF/pACA pathway were tested. The CRISPR-control expression of pAF pathway yielded significantly decreased amount of pAF compared to the plasmid version plausibly due to change in copy-number. Changing the promoter strength elevates the pAF production, and pACA production is enabled with plasmid-borne Pal expression. Finally, adjusted Genome-Scale Model (GSM) was utilized to recommend the target for CRISPRa/i perturbations. Twelve CRISPRa targets and 31 CRISPRi targets were identified using GSM. Eight additional genes were identified to be potential competing pathways for pACA production. Seven out of the eleven CRISPRa candidates were found to be activatable. Five CRISPRi candidates tested were all found to be repressible.

Production of valuable chemical compounds using engineered biological hosts is a promising route with many chemical advantages, but accommodating, avoiding, or taking advantage of endogenous metabolism and its accompanying regulation can be a major obstacle to industrially relevant bioproduction. Often, overcoming this obstacle requires wide-ranging alterations of endogenous metabolism, and new tools have emerged to understand the effects of such changes. Large-scale observation of strain engineering effects using -omics technologies, combined with genome-scale modeling, and design-build-test-learn (DBTL) approaches enhanced by machine learning, hold great promise for rapid improvement of production strains through well-targeted changes to endogenous metabolism.

The present disclosure demonstrates that the combinatorial, orthogonal, and tunable features of CRISPR-based expression control can be effectively leveraged and is well-matched with the framework of DBTL cycles for incremental strain improvement.

Aromatic compounds are a promising but challenging class of bioproducts due to their connection to the host’s central metabolism through the aromatic amino acid precursor chorismate. Development of aromatic compound-producing strains is particularly attractive when using renewable, non-edible lignocellulosic feedstocks. The products and intermediates can pose challenges, however, due to toxicity and solubility concerns. For example, p-aminocinnamic acid (p-ACA) can be used as a precursor for p-aminostyrene, but p-ACA production in E. coli is accompanied by toxic effects, even though its immediate precursor p-aminophenylalanine (p-AF) can safely accumulate in that host. The present disclosure demonstrates that Pseudomonas putida is a more suitable host, free from these toxic effects.

The heterologous contributions to the p-ACA production pathway start with a feedback-resistant AroG and overexpression of AroL aimed at boosting levels of the endogenous precursor chorismate. From there, the Pseudomonas fluorescens enzymes PapA, PapB, and PapC produce p-aminophenyl pyruvate, which becomes p-AF through endogenous transaminase activity, sometimes supplemented by additional expression of E. coli TyrB. Finally, a phenylalanine ammonia lyase enzyme (PAL), either from Arabidopsis thaliana or Rhodotorula glutinis, converts p-AF to p-ACA. Production of p-ACA from this strain is likely to be enhanced by boosting metabolic flux into the pathway and by limiting loss of flux to side products, and we aim to rapidly design this enhancement using the machine-learning-based DBTL approach.

The modularity and orthogonality of not only heterologous enzyme expression by CRISPRa, but of a whole array of endogenous CRISPRa/i interventions, can be used to introduce the variation driving such DBTL improvement, especially in a host like P. putida. Additionally, the data presented herein suggest substantial freedom in an ability to expand this array of scRNAs/gRNAs to arbitrary size. This expansion relates less to the autoregulatory architectures and more to a wide-ranging, single-layered control circuit-but if circuit expansion becomes overly burdensome at some point, either through expression burden or through changes to endogenous metabolism, autoregulation can be added to the circuit as easily as adding another gRNA. The present disclosure thus describes a non-model bacterial strain producing p-ACA under the control of CRISPR-based expression.

### Heterologous Genetics

As originally implemented in E. coli, the p-AF production pathway consisted of the P. fluorescens papABC operon78 under tet-inducible control, along with a feedback-resistant E. coli aroG74 and aroL in their own operon, which is also tet-inducible. Inducing this pathway in the DH10B strain routinely produced up to 800 µM p-AF, but trying to use an A. thaliana PAL2 enzyme to extend this pathway flux to the products downstream of p-AF proved difficult, probably due to the toxic effect of p-ACA on E. coli arresting the growth of any cells producing it. Toxic amounts of extracellular p-ACA, the result of a spike into the media, are shown in FIG. 31B, with 50% reduction of E. coli growth occurring above 10 mM extracellular.

In contrast to E. coli, p-ACA has little effect on P. putida growth, even up to 20 mM extracellular (close to its solubility limit). Therefore, the inventors chose to port the existing pAF pathway into P. putida to extend it to more valuable downstream aromatic products.

From this baseline of p-AF production, p-ACA, was produced first with the A. thaliana PAL2, and later replacing it with R. glutinis PAL75. The challenging aspect of this process was to balance the burden contributed by plasmid-based genetics versus the need to express enough enzyme to produce detectable amounts of metabolite. The pathway including PAL was large enough to present difficulties fitting onto one stable 76 plasmid, P. putida is severely burdened by a second plasmid, and strengthening the base CRISPRa promoters in preparation for genomic integration (and its reduction in copy number relative to plasmid) proved difficult. Despite these challenges, surprisingly, even suboptimal expression was enough to produce small amounts of p-ACA, a first from a bacterial host.

### Output Copy Number, Burden, and Integration

While the plasmid-based, CRISPRa-controlled p-AF pathway was producing up to 1.3 mM p-AF extracellularly, the inventors sought to expand the circuit, through both: additional enzymes (namely, PAL); and additional scRNAs and gRNAs with endogenous targets. Since the pBBR1 plasmid is already large, initially the inventors focused on integrating the enzyme genes, driven by their synthetic CRISPRa promoters, while keeping the arbitrarily large scRNA/gRNA array on the plasmid for ease of adjustment. Given the eventual goal of several DBTL cycles optimizing the effects of these adjusted CRISPRa/i interventions, this ease of adjustment was an important design factor balancing the substantial burden of carrying a plasmid in P. putida. This burden is mitigated somewhat by limiting the size of the plasmid, and the inventors integrated as much of the heterologous genes as possible, aided by the trans-acting nature of scRNAs.

Because integration would lead to a reduction in DNA copy number from the medium-copy plasmid to the single-copy genome, and this reduction in gene dosage would reduce overall expression levels, even when activated by CRISPRa, the inventors sought to use stronger base promoters within the synthetic CRISPRa promoters. As anticipated, upon integration of papABC and aroGL, the lower expression level was found to be not producing enough enzyme to produce measurable p-AF in the culture supernatant.

Guided by a small promoter strength library controlling RFP expression by CRISPRa, J23110, J23106, and J23105 base promoters were cloned, combined with low leak upstream sequences (between the spacer target and the base promoter). The challenge with this methodology was that the integration process required an initial step of plasmid cloning in E. coli. E. coli does not tolerate even low-copy plasmids like pSC101**, pBBR1, and pGNW, specifically, when combined with the stronger base promoters and the substantial size of the output genes. This challenge was also not resolved by cloning-specific E. coli strains.

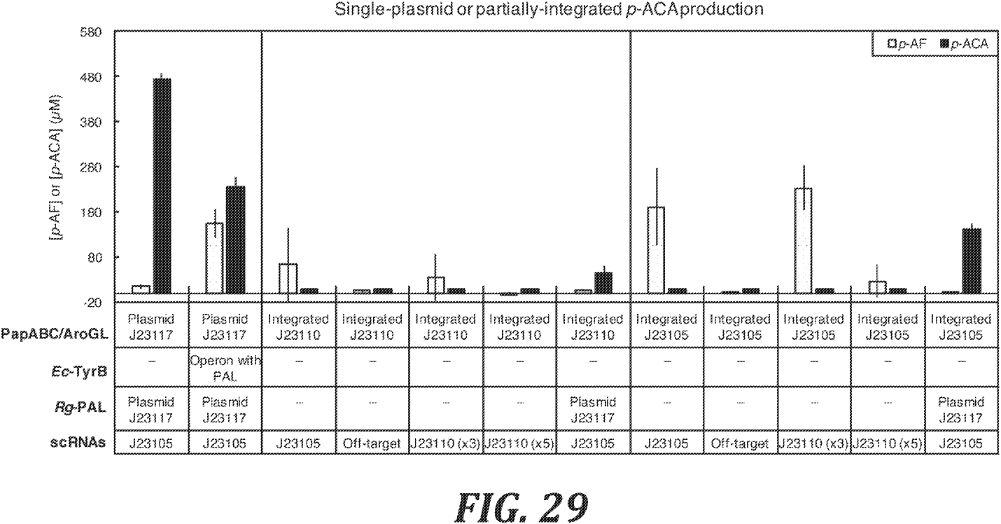

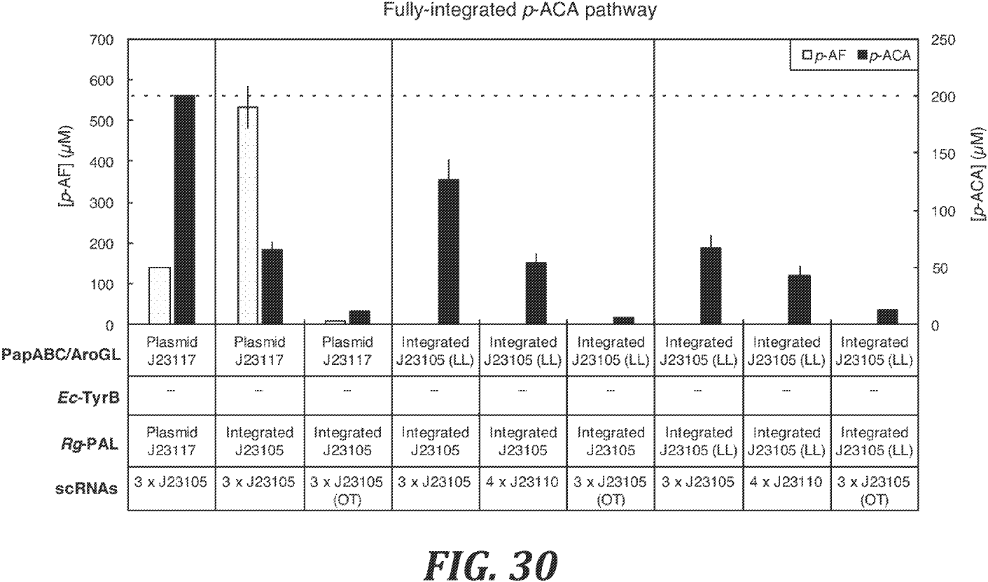

To address this problem, a cloning workflow was devised in which In-Fusion reactions were co-transformed with a “helper” plasmid consisting of dCas9 and a gRNA targeted to repress any output of a J23110 promoter. Due to sequence similarities between J23110, J23106, and J23105, it was reasoned that the same helper plasmid would sufficiently repress any of these promoters. This helper plasmid was maintained throughout the plasmid-cloning phase of integration, until eventual transformation into P. putida, before which it was restriction digested into nonreplicable linear fragments. Combined with a highly competent pir+ cloning strain, this strategy resulted in successful cloning of the plasmid-based phase of the integration workflow, and successful transformation of the integration plasmid into the P. putida recipient strain. Production of p-AF and p-ACA by strains with J23110 and J23105 base promoters driving papABC and aroGL expression is shown in FIG. 29 and FIG. 30, though even the integration of these promoter strengths seems to be accompanied by genetic instability in some cases.

An alternative strategy using a second (pRK2 origin) plasmid, into which A. thaliana PAL2 under control of the J6 synthetic promoter was cloned, with an optional inclusion of E. coli TyrB in the same operon. The burden of the second plasmid was not well-tolerated by P. putida, resulting in diminished growth rate and greatly reduced p-AF production. Despite the low concentration of its substrate, activation of PAL2 in this system resulted in a miniscule amount of p-ACA production, demonstrating the viability of even this suboptimal strategy. Because the extracellular p-ACA titers were so low, however, optimization of the base pathway was continued before implementing endogenous CRISPRa/i or iterating DBTL cycles, aiming to have more certainty in the quantification of p-ACA production differences arising from these interventions.

### Pathway Engineering for p-ACA Production Improvement

Within the two-plasmid system, the A. thaliana PAL2 was replaced with a PAL enzyme from the yeast Rhodotorula glutinis, despite the system’s low production of pAF. It has been reported that Rg-PAL shows more promiscuous activity than A. thaliana’s similar enzyme PAL4, which is more specific to the native substrate phenylalanine. It was rationalized, therefore, that Rg-PAL might have more activity on the heterologous substrate p-AF than At-PAL2. Even with low amounts of that substrate, Rg-PAL indeed produced a substantial increase in p-ACA.

Assuming a proportional increase in p-ACA production as in p-AF production, this new, Rg-PAL-containing pathway worked into a one-plasmid system would theoretically predict p-ACA titers reaching into the millimolar range, even before flux optimization by endogenous CRISPRa/i. Upon building multiple versions of one-plasmid p-ACA production strains, it was found that p-ACA titers were not quite so dramatic, but still an improvement over the two-plasmid system.

The strategies for constructing this system were either a large plasmid containing scRNAs, papABC, aroGL, and Rg-PAL, but excluding tyrB; or a smaller plasmid containing only scRNAs and Rg-PAL, used in one of the strains with papABC and aroGL integrated, driven by either the J23110 and J23105 base promoters. Avoiding the burden of maintaining and replicating the second plasmid resulted in up to four-fold improvement of p-ACA titer. Hence, the one-plasmid strain was chosen as the basis for production improvement through endogenous CRISPRa/i during DBTL cycles.

To confirm that the identified p-ACA was the product, and that its accumulation in culture supernatant was stable, numerous follow-up experiments were performed verifying the peak location within the chromatogram, the lack of consumption of extracellular p-ACA by growing P. putida cultures, and the lack of p-ACA toxicity even in a strain with the putative catabolic pathway knocked out.

Not only does HPLC indicate p-ACA production, but it also reveals several side products whose titers are increased by CRISPR activation of papABC/aroGL or of either PAL. These metabolites are produced by heterologous enzymes acting on endogenous substrates, or by endogenous enzymes acting on heterologous substrates, especially the pathway intermediates. Independent activation of each promoter was utilized to determine which side products are associated with each heterologous gene expression. Metabolites with peaks occurring at 4.8 minutes and 6.1 minutes in the HPLC chromatogram are produced by PapABC and/or AroGL, while a metabolite with a 17.7-minute peak is produced by PAL. Interestingly, they all undergo significant CRISPR activation even in the weak (J23117) integrated strain, leading to the conclusion that even in that system there is enough enzyme to affect overall metabolism, and suggesting that reducing side pathway activity through endogenous CRISPRa/i could result in p-ACA production even from that nonproducing strain. Regardless of whether one can achieve production from the J23117 integrated strain, the aim was to use endogenous CRISPRa/i to improve a strain that has already demonstrated production: either using the two-plasmid system or one of the one-plasmid alternatives.

### Endogenous CRISPRa/i for p-ACA Production Improvement

Once a base strain was optimized (to the point where it’s stable, produces a reasonably-quantifiable amount of p-ACA, and easily accepts changes to the scRNA/gRNA program), the inventors sought to improve production through iterative improvement of the CRISPR program responsible for modulating endogenous metabolism. The detection of side products in the heterologous-only pathway suggests the potential for substantial improvement because there is metabolic flux adjacent to the desired pathway. Whether the detected side products arise from endogenous enzymes or endogenous substrates, they can provide clues for rational selection of endogenous CRISPRa/i targets. For example, it was determined through spike-in experiments that the 6.1-minute peak corresponds to paminobenzoic acid, likely resulting from endogenous PapC acting on a heterologous intermediate and competing with PapB for that substrate. It is reasonable to expect, then, that PapC is a high-priority target for CRISPRi. Such a knockdown would relieve some of the competition with PapB and redirect metabolic flux into the heterologous pathway. Thus, a wide array of endogenous CRISPRa/i that will work in combination to improve product titer are envisioned herein.

### Circuit Size Considerations

The present disclosure provides systems and methods for predicting the size of endogenous CRISPRa/i scRNAs/gRNAs for optimizing p-ACA production. Other factors that are likely to limit size include competition for dCas9 binding and the metabolic effects of the interventions themselves. To investigate the former’s effect, and perhaps to quantify the burden limited size of a CRISPRa/i circuit, the effects of an arbitrary number of off-target scRNAs/gRNAs on both a CRISPR-activated reporter gene and a different constitutive reporter gene will be determined. The former reporter will determine the circuit size’s effect on CRISPR functionality, while the latter will determine the circuit size’s effect on overall expression capacity (and normalize this effect out of the CRISPR-specific effect).

The present disclosure provides the utility and portability of CRISPR-based control with pathway enzymes as outputs instead of reporter proteins. The present disclosure not only demonstrates an on par production of p-AF with tet-inducible control, it also demonstrates p-ACA production in a bacterial host, made possible by porting the circuit to a host better-suited to the pathway chemistry. Compared to TF control, orthogonal CRISPR-activatable promoters allow for more independent control of individual operons and endogenous targets, while still retaining the ability to use dCas9 or MCP-SoxS expression as a master regulator. The independent control of individual operons and endogenous targets, while still retaining the ability to use dCas9 or MCP-SoxS expression as a master regulator, can be used to tune enzyme stoichiometry within heterologous pathways, and to rationally prioritize endogenous CRISPRa/i targets based on observed side products.

Another potential pitfall of large CRISPR-controlled circuits is competition between RNAs for binding to dCas9, with a recent report noting a ten-fold reduction in CRISPRi efficacy when co-expressing 5-10 gRNAs, though CRISPRa efficacy may be more resistant. To try to boost this circuit size, autoregulation of CRISPR activity is an option, and importantly can be controlled by CRISPR itself, dCas9 (and MCP) affinity between different scRNAs/gRNAs can be equalized. Observations of very low CRISPRa fold-activation at high base promoter strengths could form the basis of a small autoregulatory boost to unbound shared component levels when reduced by binding competition.

Publications cited herein and the subject matter for which they are cited are hereby specifically incorporated by reference in their entireties. A listing of bacterial strains and plasmids used in the present disclosure can be found in Tables 1 &2. Sequence identifiers for all the sequences disclosed herein are listed in Tables 4, 5, 7, and 8.

### EXAMPLES

The following examples are set forth so as to provide those of ordinary skill in the art with a complete disclosure and description of how to make and use the present invention and are not intended to limit the scope of what the inventors regard as their invention nor are they intended to represent that the experiments below are all or the only experiments performed.

The examples disclose the inventors’ development of CRISPRa for programming heterologous gene expression in a Pseudomonas bacterial strain, e.g., P. putida KT2440. These efforts establish a framework for the further development of CRISPRa tools for programming gene expression in industrially promising bacteria.

Elements of the disclosure are included in Kiattisewee, C., Dong, et al., (2021) Portable bacterial CRISPR transcriptional activation enables metabolic engineering in Pseudomonas putida. Metabolic engineering, 66, 283-295, incorporated herein by reference in its entirety. Briefly, genetic components were constructed and experimental approaches established to permit CRISPRa machinery to be expressed and utilized in P. putida. By investigating promoter features that impact CRISPRa, such as guide RNA target sites and promoter strengths, designs permitting 30- to 100-fold activation of heterologous reporter gene expression were identified. CRISPRa was coupled with CRISPRi for multi-gene programming and endogenous gene activation. Using an inducible system derived from P. putida, an inducible CRISPRa/CRISPRi platform with low leakage in the uninduced state was developed. Further it was demonstrated that CRISPRa can drive the expression of heterologous genes to produce desirable metabolic products including biopterin derivatives and mevalonate. Using this approach, the inventors demonstrated that the inducible CRISPRa system can generate 40-fold increases in mevalonate production, achieving titers comparable to those from a previously reported IPTG-inducible system. Taken together, this work and the data generated herein provide a toolbox of components and validated workflows for implementing CRISPRa to program heterologous gene expression in P. putida.

### Example 1

Plasmids pBBR1-MCS2(pBBR1-KmR), pBBR1-MCS5(pBBR1-GmR) (Kovach et al., 1995), pTNS1, pUC18T-miniTn7T-GmR (Choi and Schweizer, 2006. mini-Tn7 insertion in bacteria with single attTn7 sites: example Pseudomonas aeruginosa. Nat. Protoc. 1, 153-161), pRK2013, pFLP2, and P. putida KT2440 were a gift from the Harwood lab at the University of Washington. pRK2-AraE (Cook et al., 2018 Genetic tools for reliable gene expression and recombineering in Pseudomonas putida. J. Ind. Microbiol. Biotechnol. 45, 517-527) was a gift from the Pfleger lab at the University of Wisconsin-Madison (Addgene #110141). pMVA2RBS035 (Jervis et al., 2019. Machine Learning of Designed Translational Control Allows Predictive Pathway Optimization in Escherichia coli. ACS Synth. Biol. 8, 127-136) was a gift from the Scrutton lab at the University of Manchester (Addgene #121051). S. pyogenes dCas9 (Sp-dCas9) was expressed from the endogenous Sp.pCas9 promoter and the MCP-SoxS (R93A, S101A) (abbreviated MCP-SoxS) transcriptional activator fusion protein was expressed from the BBa_J23107 promoter (Fontana et al., 2020a. Effective CRISPRa-mediated control of gene expression in bacteria must overcome strict target site requirements. Nat. Commun. 11, 1618) (http://parts.igem.org). The modified single guide RNAs (sgRNA) (Dong et al., 2018. Synthetic CRISPR-Cas gene activators for transcriptional reprogramming in bacteria. Nat. Commun. 9, 2489), scaffold RNAs b2.1xMS2 (scRNAs), were expressed from the BBa_J23119 promoter in the pBBR1-GmR plasmid, unless specified. 20 bp scRNA/sgRNA target sequences are provided in Table 5. mRFP1 and sfGFP reporters were expressed from the weak BBa_J23117 minimal promoter (http://parts.igem.org), unless specified, either by integrating into the genome or in the pBBR1-GmR plasmid together with the scRNA(s). All plasmids were constructed and propagated in E. coli NEB turbo cells (New England Biolabs). All P. putida strains were constructed from the wild-type strain KT2440. See Tables 1 and 2 for a complete list of bacterial strains and plasmid constructs used in the present disclosure. Exemplary plasmids used in the present disclosure are listed in Table 3.

TABLE 1

Bacterial strains and plasmids used in the present disclosure.

| Strains/Plasmids | Features | Sources* |
|---|---|---|
| Strains | | |
| P. putida KT2440 | Wildtype strain | Harwood lab |
| PPC01 | KT2440 with integrated Sp.pCas9-dCas9 and BBa_J23107-MCP-SoxS made from pPPC001 (also known as CKPP002) | This study |
| PPC02 | KT2440 with integrated J1-BBa_J23117-sfGFP, Sp.pCas9-dCas9, and BBa_J23107-MCP-SoxS, made from pPPC002 | This study |
| PPC03.N | KT2440 with integrated J1(+N)-BBa_J23117-sfGFP, Sp.pCas9-dCas9, and BBa_J23107-MCP-SoxS, made from pPPC003.N | This study |
| PPC04 | KT2440 with integrated BBa_J23111-mRFP, BBa_J1-J23117-sfGFP, Sp.pCas9-dCas9, and BBa_J23107-MCP-SoxS, made from pPPC004 | This study |
| PPC05 | PPC01 with integrated J3-BBa_J23117-mRFP and BBa_J23111-sfGFP, made from pPPC031 and pPPC032 | This study |
| PPC06 | PPC01 with integrated J3-BBa_J23117-mRFP and J3(106)- BBa_J23117-sfGFP, made from pPPC031 and pPPC033 | This study |
| PPC07 | PPC01 with integrated J3-BBa_J23117-mRFP and 13(106)- BBa_J23111-sfGFP, made from pPPC031 and pPPC034 | This study |
| PPC08 | KT2440 with integrated XylS-Pm-dCas9, BBa_J23107-MCP-SoxS made from pPPC005 | This study |
| PPC09 | KT2440 with integrated Sp.pCas9-dCas9, XylS-Pm-MCP-SoxS made from pPPC006 | This study |
| PPC10 | KT2440 with integrated XylS-Pm-dCas9, XylS-Pm-MCP-SoxS made from pPPC007 | This study |

Plasmids

| Strains/Plasmids | Features | Sources* |
|---|---|---|
| pUC18T- miniTn7T-Gm | Plasmid backbone for integration into P. putida genome, GmR/AmpR | (Choi and Schweizer, 2006) |
| pTNS1 | Tr7 transposase (tnsABCD) expressing plasmid, R6K origin of replication, AmpR | (Choi and Schweizer, 2006) |
| pRK2013 | Helper plasmid for triparental mating, KmR | (Choi and Schweizer, 2006) |
| pFLP2 | S. cerevisiae Flippase expression plasmid for marker deletion, AmpR | (Choi and Schweizer, 2006) |
| pBBR1-MCS5 (pBBR1-GmR) | Broad-host-range plasmid backbone with multiple cloning site, GmR | (Kovach et al., 1995) |
| pBBR1-MCS2 | Broad-host-range plasmid backbone with multiple cloning site, KmR | (Kovach et al., |
| (pBBR1-KmR) | | 1995) |
| pRK2-AraE | Broad-host-range plasmid backbone with AraE expressing cassette, GmR | (Cook et al., 2018) |
| pRK2-GmR | Broad-host-range plasmid backbone with multiple cloning site, GmR | This study |
| pRK2-KmR | Broad-host-range plasmid backbone with multiple cloning site, KmR | This study |
| pGNW2 | Integrative vector carrying P14g-msfGFP, KmR | (Wirth et al., 2019) |
| pS448-CsR | CRISPR/Cas9 counterselection in Gram-negative bacteria with XylS/Pm promoter, SmR | (Wirth et al., 2019) |
| pSEVA1213S | pRK2, PEM7-I-SceI; AmpR | (Wirth et al., 2019) |
| pGNW2-ppl | pGNW2 derivative with integration site at prophage1, KmR | This study |
| pGNW2-pp2 | pGNW2 derivative with integration site at prophage2, KmR | This study |
| pCK241 | pBBR1 bearing LacI-Ptrc-mRFP, GmR | This study |
| pCK243 | pBBR1 bearing XylS-Pm-mRFP, GmR | This study |
| pCK255 | pBBR1 bearing I-SceI and sacB genes, GmR | This study |
| pMVA2RBS035 | pl5A, LacI-Ptrc mvaE, mvaS, mvak1, mvaK2, and mvaD from E. faecalis, and idi gene from E. coli, KmR | (Jervis et al., 2019) |
| pCD442 | pl5A, Sp.pCas9-dCas9, BBa_J23107-MCP-SoxS, CmR | (Fontana et al., 2020a) |
| pPPC001 | pUC18T-miniTn7T, Sp.pCas9-dCas9, BBa_J23107-MCP-SoxS, AmpR/GmR | This study |
| pPPC002 | pUC18T-miniTn7T, J1-BBa_J23117-sfGFP, Sp.pCas9-dCas9, and BBa_J23107-MCP-SoxS, AmpR/GmR | This study |
| pPPC003.N | pUC18T-miniTn7T, J1(+N)-BBa_J23117-sfGFP, Sp.pCas9-dCas9, and BBa_J23107-MCP-SoxS, AmpR/GmR | This study |
| pPPC004 | pUC18T-miniTn7T, BBa_J23111-mRFP, J1-BBa_J23117-sfGFP, Sp.pCas9-dCas9, and BBa_J23107-MCP-SoxS, AmpR/GmR | This study |
| pPPC005 | pUC18T-miniTn7T, XylS-Pm-dCas9, BBa_J23107-MCP-SoxS, AmpR/GmR | This study |
| pPPC006 | pUC18T-miniTn7T, Sp.pCas9-dCas9, XylS-Pm-MCP-SoxS, AmpR/GmR | This study |
| pPPC007 | pUC18T-miniTn7T, XylS-Pm-dCas9, XylS-Pm-MCP-SoxS, AmpR/GmR | This study |
| pPPC008 | pBBR1, sgRNA or scRNA, GmR | This study |
| pPPC009 | pBBR1, sgRNA or scRNA, KmR | This study |
| pPPC010 | pBBR1, Sp.pCas9-dCas9, BBa_J23107-MCP-SoxS, scRNA, KmR | This study |
| pPPC011 | pRK2, Sp.pCas9-dCas9, BBa_J23107-MCP-SoxS, KmR | This study |
| pPPC012 | pBBR1, J1-BBa_J23117-mRFP, GmR | This study |
| pPPC013 | pBBR1, J1-BBa_J23117-mRFP, KmR | This study |
| pPPC014 | pRK2, J1-BBa_J23117-mRFP, GmR | This study |
| pPPC015 | pRK2, J1-BBa_J23117-mRFP, KmR | This study |
| pPPC016 | pBBR1, J1-BBa_J23117-mRFP, scRNA, GmR | This study |
| pPPC016(306) | pBBR1, J1-BBa_J23117-mRFP where J106 was replaced with J306, scRNA, GmR | This study |
| pPPC017 | pBBR1, J1-BBa_J23117-mRFP, scRNA, KmR | This study |
| pPPC018 | pRK2, J1-BBa_J23117-mRFP, scRNA, GmR | This study |
| pPPC019 | pRK2, J1-BBa_J23117-mRFP, scRNA, KmR | This study |
| pPPC020 | pBBR1, J3-BBa_J23117-mRFP, scRNA, GmR | This study |
| pPPC020(106) | pBBR1, J3-BBa_J23117-mRFP where J306 was replaced with J106, scRNA, GmR | This study |
| pPPC021.J231XX | pBBR1, J3-BBa_J231XX-mRFP, scRNA, GmR | This study |
| pPPC022.5PS | pBBR1, J3-Random-5PS-BBa_J23117-mRFP, scRNA-J306, GmR | This study |
| pPPC023.5PSN | pBBR1, J3-Ec-5PS-BBa_J23117-mRFP, scRNA, GmR | This study |
| pPPC024 | pBBR1, J3(106)-BBa_J23111-sfGFP, J3-BBa_J23117-mRFP, scRNA, GmR | This study |
| pPPC025 | pBBR1, J3(106)-BBa_J23117-sfGFP, J3-BBa_J23117-mRFP, scRNA, GmR | This study |
| pPPC026.XN | pBBR1, PP_NNNN-mRFP, scRNA, GmR where PP_NNNN is an endogenous promoter | This study |
| pPPC027 | pBBR1, J3-BBa_J23117-GTPCH, J3-J23117-PTPS, J3-J23117-SR, scRNA, GmR | This study |
| pPPC028 | pBBR1, J3-BBa_J23117-GTPCH, J3-J23117-PTPS, scRNA, GmR | This study |
| pPPC029 | pBBR1, LacI-Ptrc-mvaES, GmR | This study |
| pPPC030 | pBBR1, J3-BBa_J23117-mvaES, scRNA, GmR | This study |
| pPPC031 | pGNW2 derivative with integration site at prophage1 for integration of J3-BBa_J23117-mRFP cassette, KmR | This study |
| pPPC032 | pGNW2 derivative with integration site at prophage2 for integration of BBa_J23111-sfGFP, KmR | This study |
| pPPC033 | pGNW2 derivative with integration site at prophage2 for integration of J3(106)-BBa_J23117-sfGFP, KmR | This study |
| pPPC034 | pGNW2 derivative with integration site at prophage2 for integration of J3(106)-BBa_J23111-sfGFP, KmR | This study |
| A. baylyi ADP1- ISx | A. baylyi ADP1 with deletions of transposable insertion sequence (IS) | Jeffrey Barrick lab, Suarez-2017 |
| CKPP038 | PP_5409::Sp.pCas9-dCas9_BBa_J23107-MCP-SoxS_BBa_J23107- PspF-λN22 made from PPP01 and pCK302 | This study |
| pJF229B | J3-aroGL and J5-papABC in pSC101-AmpR plasmid | This study |
| pJF234.X-X-X | dCas9/MCP-SoxS expression with 3 scRNA expressions (J306, J506, and J606 analogs) in p15A-CmR plasmid | This study |
| pIDFP003.117.X- X-X | J3-BBa_J23117-aroGL, J5-BBa_J23117-papABC, and 3 scRNAs in pBBR1-GmR | This study |
| pCK425.X | J6-BBa_J23117-pal on pRK2-KmR where pal can be either At-pal or Rg-pal | This study |
| pCK426.X | J6-BBa_J23117-pal-tyrB on pRK2-KmR where pal can be either At-pal or Rg-pal | This study |
| plDFP003-int.105 | J3_LL-BBa_J23105-aroGL and J5_LL-BBa_J23105-papABC for integration at prophage1 site | This study |
| pCK439.X-X-X | J6-BBa_J23117-pal and 3 scRNAs in pBBR1-GmR | This study |
| pCK440.X-X-X | J3-BBa_J23117-aroGL, J5-BBa_J23117-papABC, J6-BBa_J23117-Rg- pal, and 3 scRNAs in pBBR1-GmR | This study |
| pCK443.X-X-X | J3-BBa_J23117-aroGL, J5-BBa_J23117-papABC, J6-BBa_J23117-Rg- pal-tyrB, and 3 scRNAs in pBBR1-GmR | This study |
| pCK520.105.X | J6-BBa_J23105-Rg-pal for integration at prophage2 site | This study |
| IFPP002 | CKPP002 with integration of J3_LL-BBa_J23105-aroGL and J5_LL- BBa_J23105-papABC at prophage1 site made by pIDFP003-int.105 | This study |
| IFPP008 | IFPP002 with integration of J6-BBa_J23105-Rg-pal at prophage2 site made by pCK520 | This study |
| pCK365.J231XX | J3-BBa_J23117-sfGFP and J306 scRNA under different promoter strengths (BBa_J231XX) | This study |
| pCK190.N | J3-BBa_J23117-mRFP and truncated J306 scRNA (19bp to 8bp) on pBBR1-GmR | This study |
| pCK422 | 3 fluorescent proteins (sfGFP, mTagBFP, and mRFP) under J3/J5/J6- BBa_J23117 promoters in pRK2-KmR | This study |
| pCK537.N | pBBR1-GmR plasmid with N scRNAs | This study |
| pCK343.P.X | PP_NNNN-sfGFP fusion reporter for CRISPRa investigation with scRNA(X) on pBBR1-GmR | This study |
| pCK348.P.X | PP_NNNN-sfGFP fusion reporter for CRISPRi investigation with scRNA(X) on pBBR1-GmR | This study |
| CKAB029 | ACIAD2184::FRT-SmR-FRT-J23107-dCas9_J23107-MCP-SoxS made from ADP1-ISx and pCK653 | This study |
| pCK302 | Integration of BBa_J23107-PspF-λN22 to P. putida genome | This study |
| pCK653 | Integration of BBa_J23107-dCas9 and BBa_J23107-MCP-SoxS to A. baylyi genome | This study |
| pCK509 | pBBR1-GmR plasmid expressing pACA pathway with J306, J506, and J606 scRNAs | This study |
| pCK511.X | pBBR1-GmR plasmid expressing pACA pathway with J306, J506, J606, and the 4th gRNA targeting nothing or endogenous targets | This study |
| pCK683 | pBBR1-GmR plasmid expressing pACA pathway with J306, J506, J606, and 2 non-targeting scRNAs (J106 and hAAV) | This study |
| pCK684 | pBBR1-GmR plasmid expressing pACA pathway with J306, J506, J606, and 3 non-targeting scRNAs (J106, hAAV, and J206) | This study |
| pCK396.X | pBAV1-KmR plasmid with J23106-sfGFP and scRNA | This study |
| pCK681.X | ColE1-GmR plasmid with J231XX-sfGFP and scRNA, J231XX is either J23114 or J23117 | This study |
| pCK682.X | pRSF1010-GmR plasmid with J231XX-sfGFP and scRNA, J231XX is either J23114 or J23117 | This study |
| pCK279.X | pBBR1-GmR plasmid with J1-pspAp-mRFP and 2x-BoxB scRNA | This study |
| pCK729.X | pBBR1-GmR plasmid with J1-pspAp-mRFP and J3-BBa_J23117- sfGFP dual reporter and scRNAs | This study |

### Example 2

### Plasmid Construction

All PCR fragments were amplified with Phusion DNA Polymerase (Thermo-Fisher Scientific) for Infusion Cloning (Takara Bio). Transformants were cultured or selected either on Lysogeny Broth (LB) or agar plates, with appropriate antibiotics, used in the following concentrations: 100 µg/mL Carbenicillin, 25 µg/mL Chloramphenicol, 30 µg/mL Kanamycin, 30 µg/mL Gentamicin. Successful constructs were confirmed by Sanger sequencing (GENEWIZ). Details for cloning strategies are well known in the art. The various constructs used for the cloning strategies are described in Tables 2-4, below. sgRNA/scRNA target sequences are provided in Table 5.

### Example 3

### Pseudomonas Putida Strain Construction

Pseudomonas putida genome integrations were performed using the tri-parental conjugation for the mini-Tn7 method (Choi and Schweizer, 2006. mini-Tn7 insertion in bacteria with single attTn7 sites: example Pseudomonas aeruginosa. Nat. Protoc. 1, 153-161) or electroporation for the pGNW2 method (Wirth et al., 2019 Wirth, N.T., Kozaeva, E., Nikel, P.I., 2019). Accelerated genome engineering of Pseudomonas putida by I-SceI-mediated recombination and CRISPR-Cas9 counter selection. Microb Biotechnol). Plasmid transformations into P. putida were performed either by electroporation (Choi and Schweizer, 2006. mini-Tn7 insertion in bacteria with single attTn7 sites: example Pseudomonas aeruginosa. Nat. Protoc. 1, 153-161) or heat-shock of CaCl₂ chemically competent cells (Zhao et al., 2013. [CaCl2-heat shock preparation of competent cells of three Pseudomonas strains and related transformation conditions]. Ying Yong Sheng Tai Xue Bao 24, 788-94).

### Example 4

### Fluorescence Measurements of Reporter Gene Expression

Fluorescence measurements of reporter gene expression were carried out either by flow cytometry or plate reader. Single colonies from LB plates were inoculated in 500 µL of EZ-RDM (Teknova) supplemented with the appropriate antibiotics and grown in 96-deep-well plates at 30° C. with shaking overnight 225 rpm. For small-molecule induction, overnight cultures were diluted 100-fold into a new culture with appropriate antibiotics and inducers, then shaken overnight at 30° C., 225 rpm. For flow cytometry, overnight cultures were diluted 1:50 in Dulbecco’s phosphate-buffered saline (PBS) and analyzed on a MACSQuant VYB flow cytometer with the MACSQuantify 2.8 software (Miltenyi Biotec) using the methods and instruments settings as described (Dong et al., 2018. Synthetic CRISPR-Cas gene activators for transcriptional reprogramming in bacteria. Nat Commun 9, 2489). For plate reader measurements, 150 µL of overnight culture were transferred into a flat, clear-bottomed black 96-well plate. OD₆₀₀ and fluorescence values were measured in a Biotek Synergy HTX plate reader and analyzed using the BioTek Gen5 2.07.17 software. For mRFP1 detection, the excitation wavelength was 540 nm and emission wavelength was 600 nm. For sfGFP detection, the excitation wavelength was 485 nm and the emission wavelength was 528 nm. Data were plotted using Prism (GraphPad).

### Example 5

### Mevalonate Production and Quantitation by GC-MS

For mevalonate production experiments, the GC-MS method was adapted from prior methods (Pfleger et al., 2007. Microbial sensors for small molecules: Development of a mevalonate biosensor. Metab. Eng. 9, 30-38). Single colonies from LB plates were inoculated in 500 µL of EZ-RDM (Teknova) supplemented with the appropriate antibiotics and grown in 96-deep-well plates at 30° C. with shaking overnight at 225 rpm. Overnight cultures were subcultured by 1:100 dilution into 3 mL of EZ-RDM media with 1% glucose as the carbon source, supplemented with the appropriate antibiotics, and shaken at 225 rpm for 72 hours at 30° C. After 72 hours, 560 µL of cell suspension was acidified with 140 µL of 0.5 M HCl and vortexed. 700 µL ethyl acetate was added and samples were then vortexed again vigorously for 3 minutes and centrifuged at maximum speed in a benchtop centrifuge (15,000 rcf) for 10 min. The organic phase was then transferred into GC-MS vials for analysis. GC-MS analysis was performed using an Agilent 5973 instrument with a temperature program as follows. The inlet temperature was 250° C. (splitless mode). The column flow was kept at 1 mL/min in HP-5MS (Agilent). The temperature cycle started at 80° C. and was followed by a gradient of 20° C./min to 260° C., a second gradient of 40° C./min to 300° C., and a hold at 300° C. for 2 min. m/z = 71, the second most abundant peak corresponding to mevalonolactone, was used for quantitation (Pfleger et al., 2007. Microbial sensors for small molecules: Development of a mevalonate biosensor. Metab. Eng. 9, 30-38). A calibration curve was generated using freshly-prepared D,L-mevalonolactone (Sigma) dissolved in ethyl acetate. The calculated concentration was adjusted by the addition of HCl. Data were plotted using Prism (GraphPad).

### Example 6

### Biopterin Production and Measurement

For the biopterin production experiments, single colonies from LB plates were inoculated in 500 µL of EZ-RDM supplemented with the appropriate antibiotics and grown in 96-deep-wellplates at 30° C. with shaking overnight. Each sample was then sub-cultured at 100-fold dilution in 5 mL of EZ-RDM supplemented with the appropriate antibiotics and grown in 14 mL culture tubes at 30° C. and shaking for 24 hours. The overnight cultures were spun down and pteridine concentrations were determined by measuring the OD₃₄₀ and comparing the results to a standard calibration curve prepared with purchased reagents (Cayman Chemical). The HPLC-MS measurements were performed as described (Ehrenworth et al., 2015. Pterin-Dependent Mono-oxidation for the Microbial Synthesis of a Modified Monoterpene Indole Alkaloid. ACS Synth. Biol. 4, 1295-1307). A detailed HPLC-MS protocol is provided in the Supplementary Methods. Data were plotted using Prism (GraphPad).

### Example 7

### Enabling CRISPRa in P. Putida

The first challenge to enable a CRISPRa system in P. putida is to express the components from E. coli in P. putida. The bacterial CRISPRa system developed in E. coli consists of three components, dCas9, MCP-SoxS, and scRNA (Dong et al., 2018. Synthetic CRISPR-Cas gene activators for transcriptional reprogramming in bacteria. Nat Commun 9, 2489), delivered in a p15A plasmid that is present at ~10 copies/cell (Shetty et al., 2008. Engineering BioBrick vectors from BioBrick parts. J. Biol. Eng. 2, 5) (FIG. 1A). The scRNA is a modified sgRNA with a 3′ MS2 hairpin to recruit the MCP-SoxS activator. The reporter gene(s) were delivered in a pSC101** plasmid which is present at ~5 copies/cell (Lee et al., 20111. BglBrick vectors and datasheets: A synthetic biology platform for gene expression. J. Biol. Eng. 5, 12).

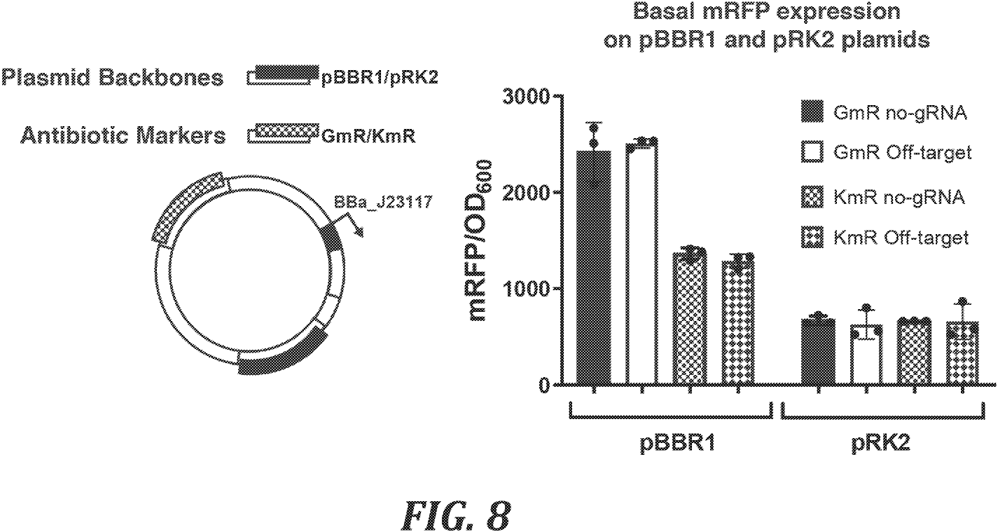

E. coli SoxS activator domain was used because it recognizes a motif on RpoA that is conserved between E. coli and P. putida (Dong et al., 2018. Synthetic CRISPR-Cas gene activators for transcriptional reprogramming in bacteria. Nat Commun 9, 2489), and there is no direct homolog of SoxS in P. putida (Park et al., 2006. Regulation of superoxide stress in Pseudomonas putida KT2440 is different from the SoxR paradigm in Escherichia coli. Biochem. Biophys. Res. Commun. 341, 51-56). To test this system in P. putida, the three CRISPRa components need to be expressed at levels sufficient to activate the target gene without dCas9 expression being so high that cellular functions are inhibited (Depardieu and Bikard, 2020. Gene silencing with CRISPRi in bacteria and optimization of dCas9 expression levels. Methods 172, 61-75; Zhang and Voigt, 2018. Engineered dCas9 with reduced toxicity in bacteria: implications for genetic circuit design. Nucleic Acids Res. 46, 11115-11125). Components from two E. coli plasmid constructs, a CRISPRa system plasmid and a reporter plasmid, were first moved directly into two P. putida expression plasmids, pBBR1 and pRK2 (each present at 25-30 copies/cell according to (Cook et al., 2018. Genetic tools for reliable gene expression and recombineering in Pseudomonas putida. J. Ind. Microbiol. Biotechnol. 45, 517-527)) (FIG. 1B). Reporter gene expression that depends on the presence of an on-target scRNA was observed (FIG. 1C). Reporter gene expression in the presence of an off-target scRNA was indistinguishable from a strain without scRNA/sgRNA present (FIG. 8).

### Example 8

### Growth-Defect Mitigation Elevates CRISPRa Efficiency

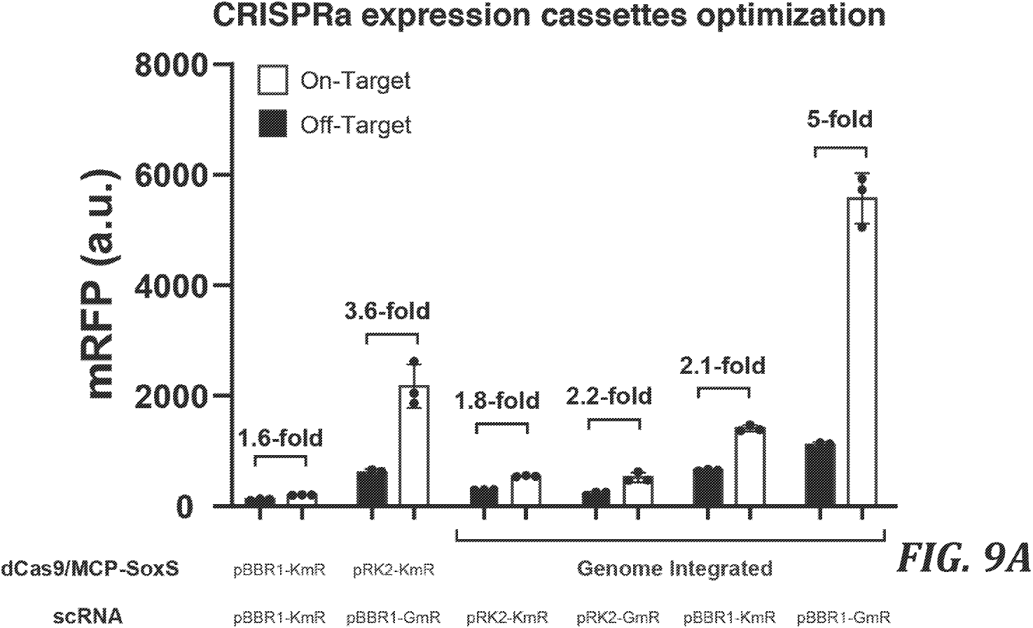

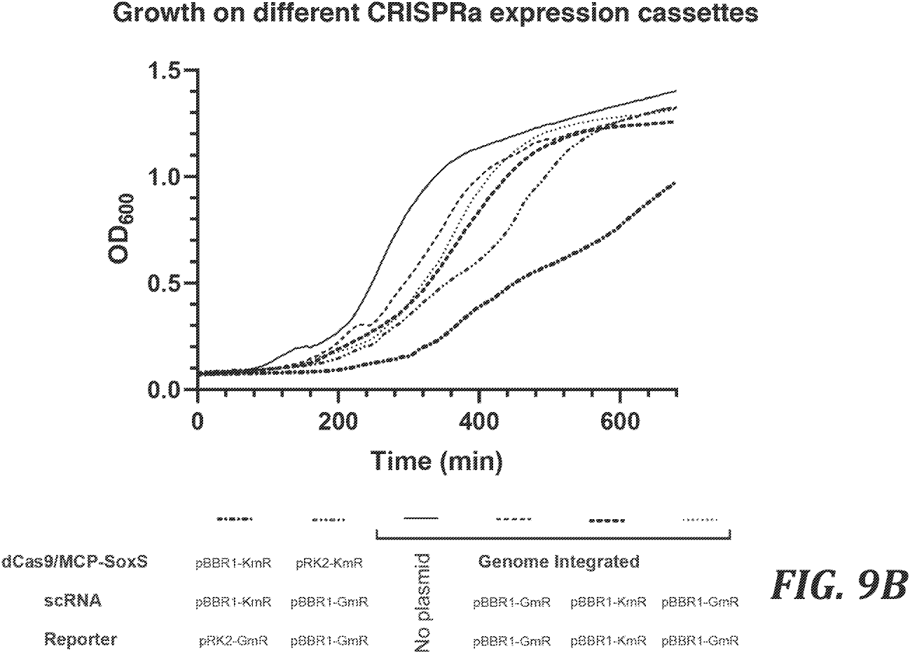

P. putida strains with the initial implementation of the CRISPRa system grew poorly on both agar and liquid media (FIG. 9B). To mitigate the growth defect, multiple different plasmid and genome-integrated delivery methods for the CRISPRa components were tested. The expression levels of dCas9 and MCP-SoxS were reduced first by moving these genes from the pBBR1 plasmid to the pRK2 plasmid, which expresses transgenes at a lower level in P. putida (Damalas et al., 2020. SEVA 3.1: enabling interoperability of DNA assembly among the SEVA, BioBricks and Type IIS restriction enzyme standards. Microb Biotechnol) (FIG. 8). This change partially mitigated the growth defect and improved the CRISPRa reporter gene expression (FIGS. 9A-9B). The expression levels of dCas9 and MCP-SoxS were further reduced by integrating the dCas9/MCP-SoxS cassette into the P. putida KT2440 genome (generating strain PPC01). The scRNA and reporter gene cassettes on plasmids with different combinations of two origins of replication (pBBR1 and pRK2) and two antibiotic markers (GmR and KmR) were then delivered to test whether variations in the plasmid backbones impart different metabolic burdens (Mi et al., 2016).

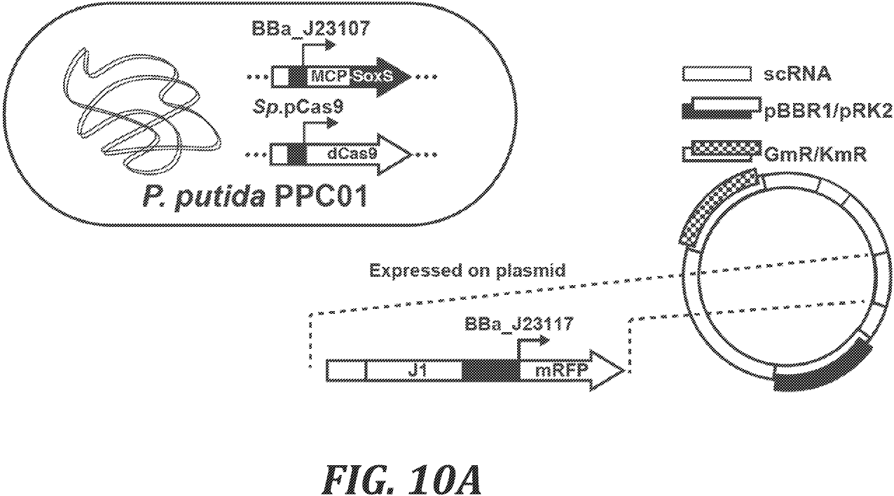

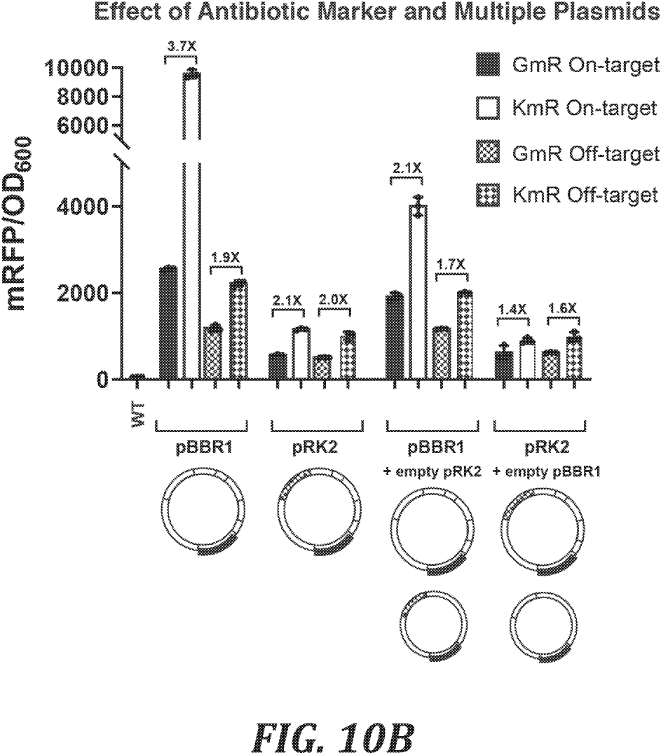

The highest level of activation (~5-fold) were observed with the scRNA and reporter both expressed from a single pBBR1-GmR backbone, while the plasmid with either pRK2 origin or KmR marker yielded weaker activation (~2-fold) (FIG. 1C and FIG. 9A). The presence of the second plasmid reduced both fold-activation by CRISPRa and basal expression of mRFP significantly (FIG. 10B). In general, both CRISPRa fold-activation and the corresponding basal mRFP expression (off-target control) increased in strains that grew faster (FIGS. 9A -9B and FIG. 10B), suggesting that there are different metabolic burdens associated with different delivery methods and plasmid expression systems. Taken together, these results suggest that optimizing expression levels will be important for implementing CRISPRa in new bacterial species. To improve P. putida CRISPRa beyond the five-fold activation obtained in FIG. 1C, the genomically integrated dCas9/MCP-SoxS strain (PPC01) was selected for further optimization. While there is no specific target value for fold-activation, the largest dynamic range possible was used to provide the highest possible tunable range in future applications.

### Example 9

### Characterization of Promoter Elements for Optimal CRISPRa Efficiency in P. Putida

To improve the fold-activation of CRISPRa in P. putida, the criteria for effective CRISPRa observed for E. coli (Fontana et al., 2020a. Effective CRISPRa-mediated control of gene expression in bacteria must overcome strict target site requirements. Nat. Commun. 11, 1618) were investigated. Specifically, factors known to affect CRISPRa efficiency in E. coli include i) the distance of target sequence to transcription start-site (TSS), ii) the sequence composition of the 20 bp scRNA targeting sequence, iii) the basal minimal promoter strength, and iv) the 5′-proximal sequence composition between target sequence and minimal promoter (FIG. 2A).

### Example 10

### Distance to Transcription Start-Site (TSS)

In E. coli, the most effective CRISPRa target sites are in the region of -60 to -100 bp before the TSS, with sharp peaks of activity every 10 bases, separated by regions of inactivity (Fontana et al., 2020a. Effective CRISPRa-mediated control of gene expression in bacteria must overcome strict target site requirements. Nat. Commun. 11, 1618). An integrated reporter that can be targeted at multiple sites (J1-sfGFP, previously characterized in E. coli (Fontana et al., 2020a. Effective CRISPRa-mediated control of gene expression in bacteria must overcome strict target site requirements. Nat. Commun. 11, 1618) was constructed and used to deliver plasmids with scRNAs targeting different sites as shown in FIG. 2B. With target sites spaced 10 bp apart, the optimal sites in P. putida were located in the -60 to -100 bp range before the TSS, similar to that in E. coli. When sites were tested at single base resolution between -81 to -93 bp, peaks of activity ~10-11 bases apart, similar to what was observed in E. coli (FIG. 2C) were observed. The efficiency of CRISPRa diminished after a 4-5 bp shift and was recovered at 10-12 bp. This finding suggests that CRISPRa has a periodic dependence on distance from the TSS.

### Example 11

### scRNA Target Sequences

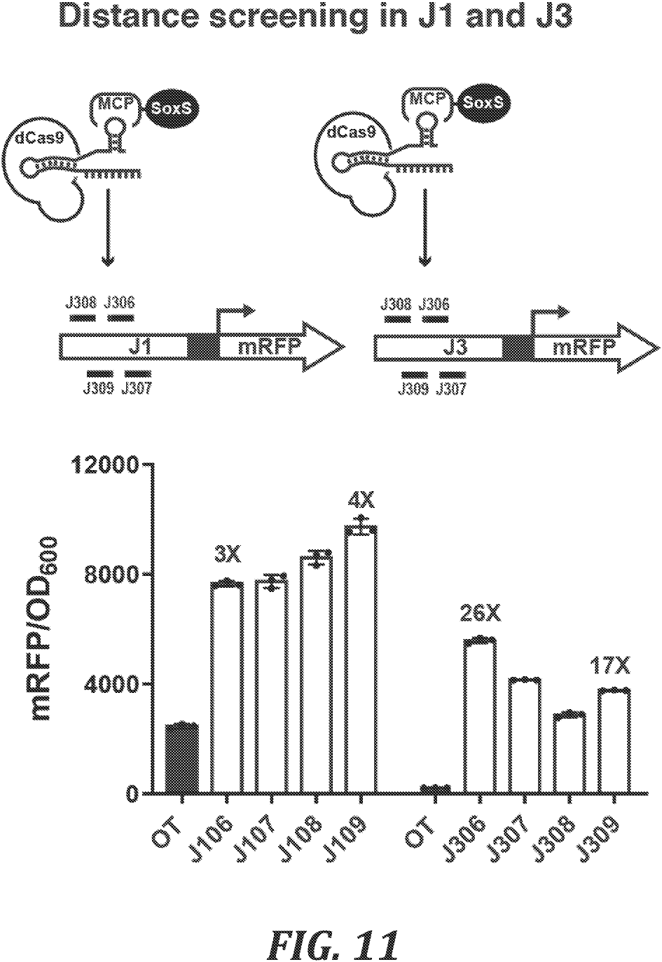

Next, the 20 bp target sequence that is recognized by the scRNA was examined. The experiments described above were performed with the J1 promoter, which contains an array of 20 base target sites. An alternative promoter, termed J3, that has a different set of 20 base target sites was tested. Multiple target sites in the J3 promoter were tested and it was found that the J306 site, located 81 bases upstream of the TSS, yielded the highest fold-activation (FIG. 11). Compared to the J1 promoter, where a 4-fold activation (J106 target site) was observed, the fold-activation with the J3 promoter increased to 34-fold (J306 target site) (FIG. 2D). For both J1 and J3, the CRISPRa-induced expression levels were similar. The large difference in fold-activation results from an unexpected difference in basal expression levels. The basal expression of J3-mRFP is 11-fold lower than that of J1-mRFP, which leads to much higher fold-activation. This difference in basal expression was not observed in E. coli, where J1 and J3 reporters produced 27-fold and 36-fold activation, respectively (Fontana et al., 2020a. Effective CRISPRa-mediated control of gene expression in bacteria must overcome strict target site requirements. Nat. Commun. 11, 1618).

To test whether the different basal expression levels were due to differences in the 20 base target sites or to other features of the promoters, hybrid promoters where the 20 base J106 target site in J1 was replaced by J306 (J1(306)) and vice versa (J3(106)) were constructed and tested similarly. A low basal expression only with the hybrid J3(106) promoter (FIG. 2D) was observed, suggesting that other sequence features of the J3 promoter besides the 20 base target site are responsible for the low basal expression of the J3 promoter. These sequence features could be upstream of the target sequence or between the target sequence and the minimal BBa_J23117 promoter. The J3 upstream sequence was selected for further optimization of CRISPRa, as it yielded the best dynamic range from the promoter sequences tested.

### Example 12

### Minimal Promoter Strength

The promoters tested comprise a 35 base minimal promoter that binds the sigma subunit of RNA polymerase and an upstream 170 base sequence region with scRNA target sites. The 35 bp minimal promoter sequence is also a key factor that governs the dynamic range of CRISPRa. In E. coli, it was observed that minimal promoter strength and the sigma factor regulating the promoter have large effects on CRISPRa (Fontana et al., 2020a. Effective CRISPRa-mediated control of gene expression in bacteria must overcome strict target site requirements. Nat. Commun. 11, 1618). However, the alternative sigma factor regulons in P. putida are less characterized compared to those in E. coli. Therefore, the sigma-70 regulon, the house-keeping sigma factor, that covers the majority of E. coli and P. putida endogenous promoters (Fujita et al., 1995) was selected for further investigation.

To test the effects of promoter strength, 11 minimal 35 base promoters from the Anderson promoter collection (BBa_J231XX, parts.igem.org) were introduced into the J3-mRFP reporter (FIG. 3A). The variations in promoter strength arise from point mutations in the -10 and -35 sites that tune transcriptional activity; no significant changes in the transcription start sites (TSS) were detected when these promoters were experimentally characterized (Kosuri et al., 2013. Composability of regulatory sequences controlling transcription and translation in Escherichia coli. Proc. Natl. Acad. Sci. 110, 14024) (see Supplemental Information for annotated sequences).

CRISPRa-mediated expression from the Anderson promoter series followed trends similar to that previously observed in E. coli (Fontana et al., 2020a. Effective CRISPRa-mediated control of gene expression in bacteria must overcome strict target site requirements. Nat. Commun. 11, 1618). When the promoter strengths are extremely weak (BBa_J23109 and BBa_J23113), the CRISPRa fold-activation dropped significantly to 3.1-fold and 1.4-fold compared to 27-fold with the moderately weak BBa_J23117 minimal promoter. As promoter strength increases from BBa_J23117 to the strong BBa_J23110 promoter, CRISPRa fold-activation decreases because basal expression increases ~10-fold, while the maximal CRISPRa output varies by <4-fold (FIG. 3A). CRISPRa with the strongest promoter tested (BBa_J23111) could not be measured because no colonies were obtained when the CRISPR machinery was delivered to P. putida with this reporter, possibly due to the metabolic burden of expressing high levels of mRFP and the CRISPR system at the same time. The minimal BBa_J23117 promoter yields the highest fold-activation in both E. coli and P. putida (36-fold and 27-fold, respectively) presumably because basal expression is weak enough that significant activation is possible, but not so weak that the promoter is difficult to activate. Thus, reporters with the BBa_J23117 minimal promoter were selected for further characterization and application. Notably, if a higher absolute expression level is preferred, the stronger BBa_J23106 promoter yielded the highest absolute expression level (2.4-fold higher than CRISPRa-mediated activation of BBa_J23117), although the fold-activation was smaller (FIG. 3A).

### Example 13

### 5′-Proximal Sequences

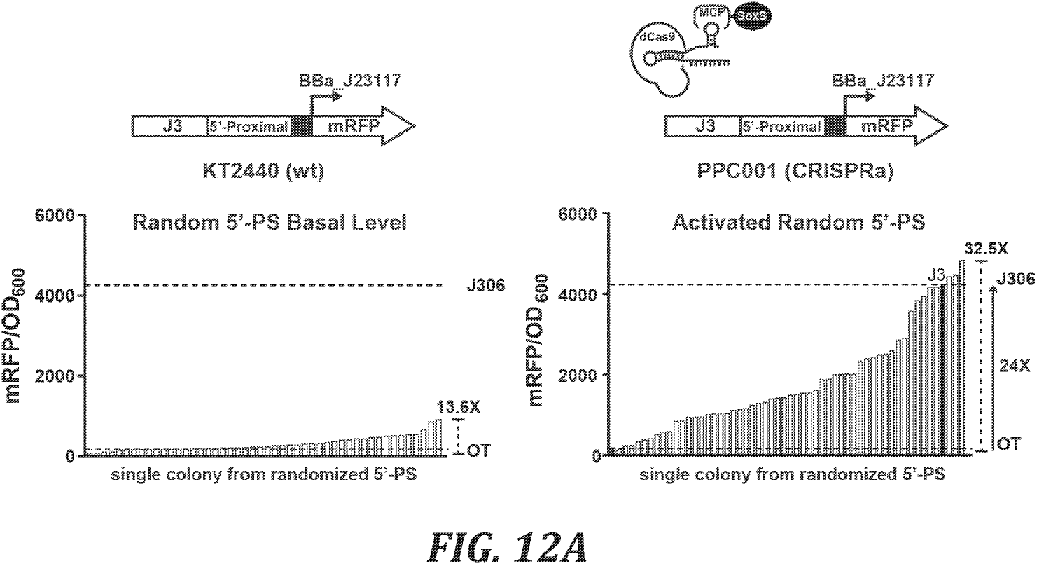

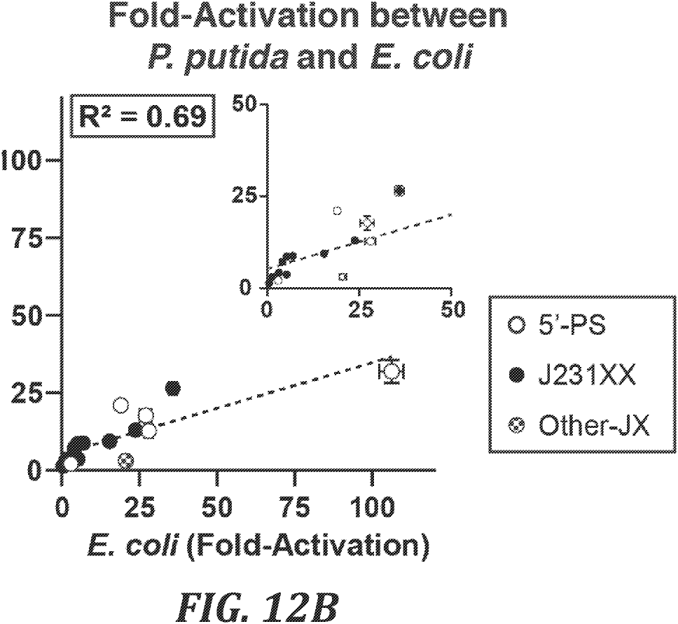

The last factor tested was the intervening sequence between the 20 base target site and the 35 base minimal promoter, termed the 5′-proximal sequence. This sequence is 26 bp long when using an optimal target site located at -81 bp from the TSS. A pooled library of reporter gene plasmids with variable 26 base 5′ proximal sequences was constructed using a randomized oligo pool. Each reporter retains the same 20 base J306 scRNA target site and the BBa_J23117 minimal promoter. This library was transformed into a P. putida reporter strain and a large number of single colonies were functionally characterized without sequencing each colony. The random 5′-proximal sequences led to a broad range of mRFP levels from CRISPRa (FIG. 3B), similar to what has been observed in E. coli (Fontana et al., 2020a. Effective CRISPRa-mediated control of gene expression in bacteria must overcome strict target site requirements. Nat. Commun. 11, 1618). Random 5′ proximal sequences also affect basal expression levels in the absence of CRISPRa, although these effects are relatively small (FIG. 12A). To determine if 5′-proximal sequence preferences are correlated between E. coli and P. putida, several known sequences previously characterized in E. coli were tested. It was observed that high-efficiency 26 bp sequences from E. coli yield high CRISPRa efficiency in P. putida while a weak sequence from E. coli remains weak in P. putida (FIG. 3C). Across the set of sequences analyzed, one of these (5′-PS5) exhibited a higher fold activation (32-fold) compared to the J3-BBa_J23117 promoter (27-fold). The basal expression in 5′-PS5 is 15% lower than J3-BBa_J23117, and both sequences gave similar activated levels. Further, whether the 26 bp 5′ proximal sequence from the J1 promoter was responsible for the high basal activity of the J1 promoter (FIG. 2D) was also tested. When the 26 bp 5′ proximal sequence from J1 was inserted into the J3 promoter, relatively low basal expression (5′-PS2 in FIG. 3C), similar to the J3 promoter was observed. This result suggests that the 5′ proximal sequence of J1 is not the cause of the high basal activity of the complete J1 promoter, and that sequence features upstream of the 5′ proximal sequence and the 20 bp target site could be responsible.

The variation in CRISPRa outputs with different promoter features suggests that a set of distinct and orthogonal heterologous promoters could be developed for tunable control of gene expression. Promoters with orthogonal 20 base target sequences, together with different 5′ proximal sequences, minimal promoters, and target site positions could be used to access a broad range of CRISPRa-mediated gene expression levels. Further, systematically varying the 5′-proximal sequence could allow for the identification of promoters with lower basal expression and higher dynamic range of activation, similar to the case of the 5′-PS5 sequence mentioned above. The present disclosure contemplates constructing combinatorial libraries of multi-gene programs to explore how independently tuning gene expression levels in metabolic pathways affects product titers.

### Example 14

### Correlation of CRISPRa Efficiency Between Organisms

To compare CRISPRa in P. putida to that in E. coli, a correlation plot of mRFP expression from CRISPRa strains with different promoter sequence variations was constructed (FIG. 3D and FIG. 12B). This plot indicates that the expression level induced by CRISPRa in E. coli correlates well with CRISPRa in P. putida (R² = 0.80). The fold-activation is also correlated (R² = 0.69 Figure S5B), although the fold-activation of P. putida CRISPRa tends to be lower than that of E. coli. The discrepancies across organisms might arise from variations in genetic context, transcription machinery, or cellular compositions between bacterial species. Despite these modest discrepancies, CRISPRa behaves largely similarly in E. coli and P. putida, suggesting that optimized CRISPRa circuits will be portable between species and that further modifications and improvements to CRISPRa systems should be readily transferable. While these trends are not expected to be generalizable across all bacterial species, the metrics described herein can be systematically evaluated in alternative bacterial hosts to assess whether design principles and optimized CRISPRa circuits can be easily ported to new hosts.

### Example 15

### Using P. Putida CRISPRa for Sophisticated Transcriptional Control Strategies

With an optimized CRISPRa system in P. putida, several strategies to enable more sophisticated control over gene expression programs were explored. Multi-gene CRISPRa/CRISPRi programs were constructed, and endogenous gene activation was demonstrated. Further, an inducible CRISPRa system for tunable, dynamically regulated expression was developed. These strategies will enable the construction of multi-gene programs to rewire metabolic networks for optimal biosynthesis in P. putida.

### Example 16

### Multi-Gene Regulation by CRISPRa and CRISPRi

With optimized expression levels and a delivery strategy for the CRISPRa system in P. putida in place, whether CRISPRa and CRISPRi can be used together to activate and repress multiple genes was tested. This strategy has been previously successful in E. coli (Dong et al., 2018. Synthetic CRISPR-Cas gene activators for transcriptional reprogramming in bacteria. Nat Commun 9, 2489). A dual-reporter plasmid with weakly expressed mRFP (J3-BBa_J23117-mRFP) and highly expressed sfGFP (J3(106)-BBa_J23111-sfGFP) was constructed. A dual scRNA/sgRNA cassette was inserted in this plasmid with a J306 scRNA for mRFP activation and an sgRNA that targets within the sfGFP open reading frame (ORF) for repression. This plasmid was delivered to a P. putida strain with integrated dCas9/MCP-SoxS and simultaneous activation of mRFP (6.6-fold) and repression of sfGFP (13-fold) (FIG. 4) was observed. The magnitude of CRISPRa fold-activation in simultaneous CRISPRa/i was weaker than the 15-fold activation that was observed if just a single scRNA was delivered to activate the mRFP reporter, possibly due to competition between multiple scRNA/sgRNA cassettes for a limited pool of dCas9.

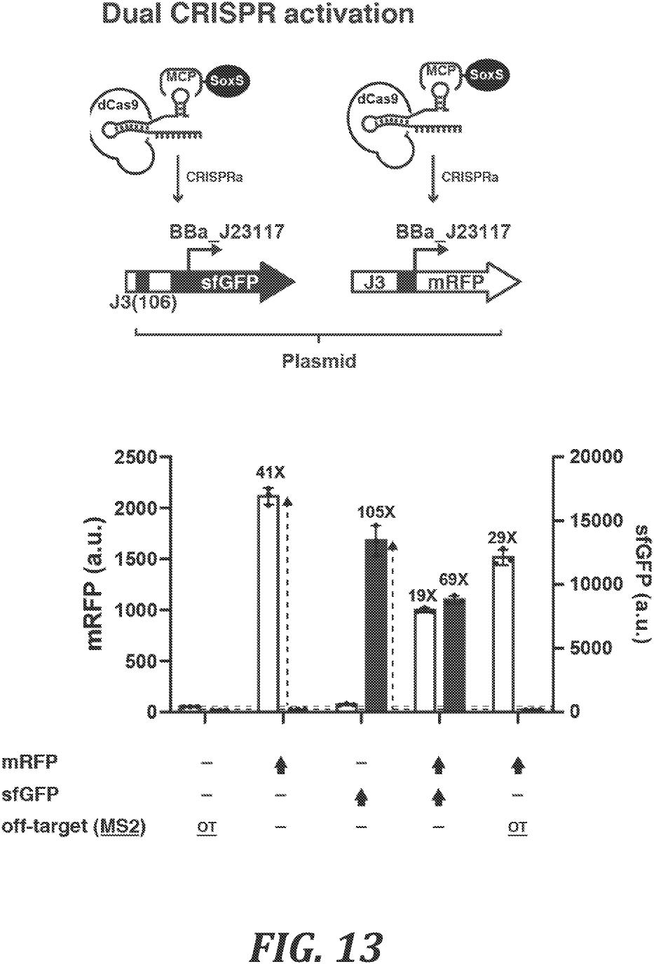

To determine if CRISPRa can be used to activate multiple genes simultaneously, a dual-reporter plasmid with weakly expressed mRFP (J3-BBa_J23117-mRFP) and weakly expressed sfGFP (J3(106)-BBa_J23117-sfGFP) was constructed. A dual scRNA cassette was inserted into this plasmid with scRNAs that target mRFP and sfGFP for activation and delivered it to a P. putida strain with integrated dCas9/MCP-SoxS. Simultaneous activation of mRFP (19-fold) and sfGFP (69-fold) (FIG. 13) was observed. As seen with simultaneous CRISPRa/CRISPRi, the CRISPRa effects with dual activation were weaker than those observed if each reporter was targeted individually (41-fold activation for mRFP and 105-fold activation for sfGFP), consistent with the idea that competition for dCas9 among multiple species of sgRNA/scRNA may be an issue for multi-gene programs (Huang et al., 2020. Programmable CRISPR-Cas transcriptional activation in bacteria. Mol. Syst. Biol. 16, e9427). Simultaneous CRISPRa at multiple genes using the weak mRFP/strong sfGFP reporter described above was observed; the strong sfGFP could be activated a further 2-3-fold when activated by an upstream scRNA (FIG. 4).

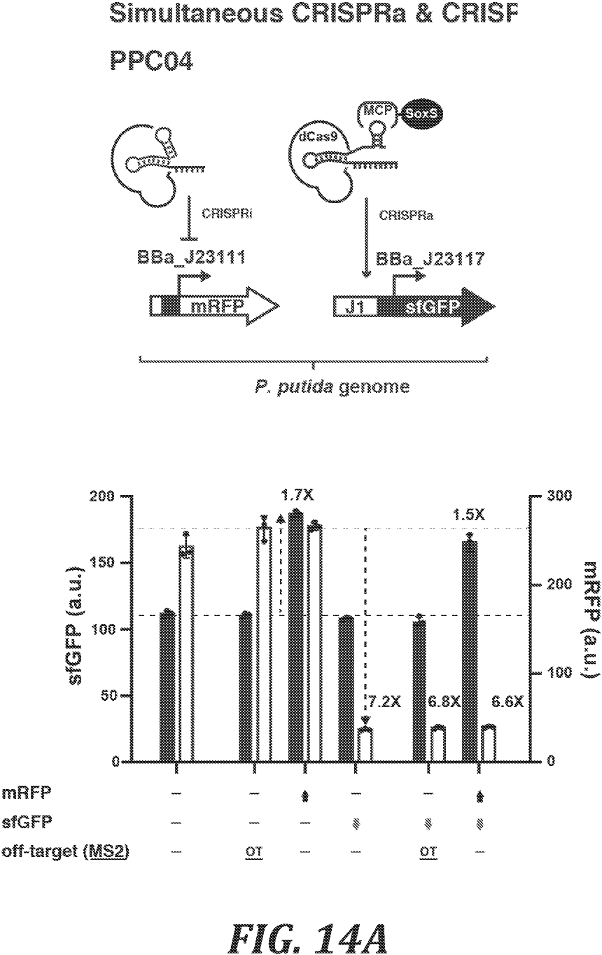

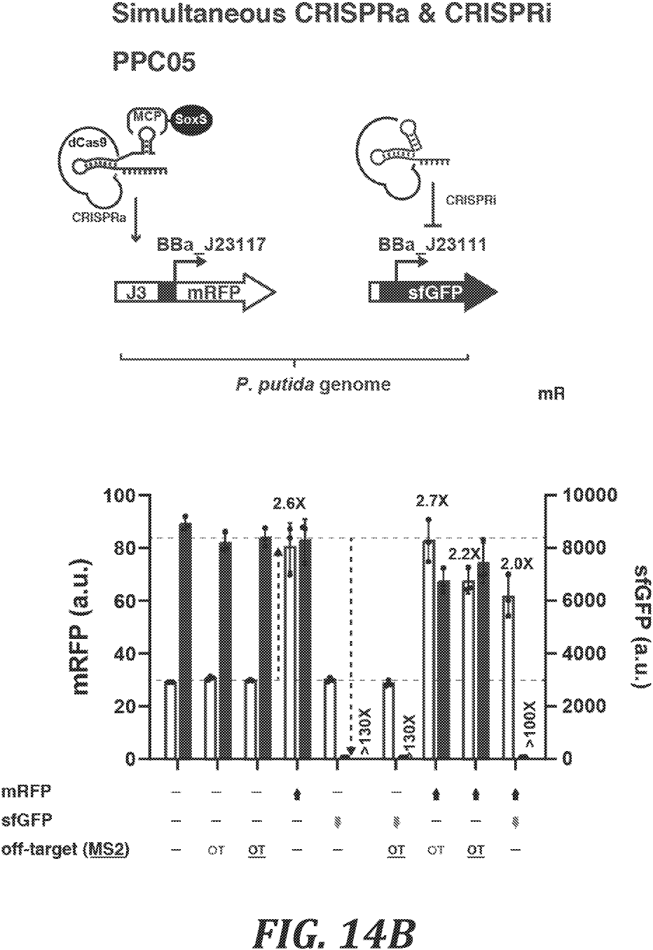

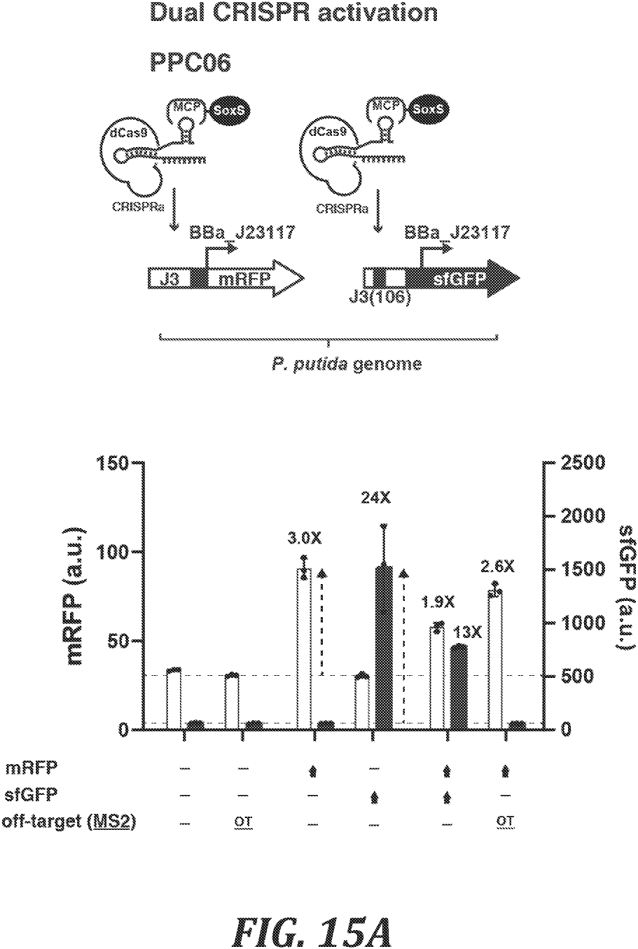

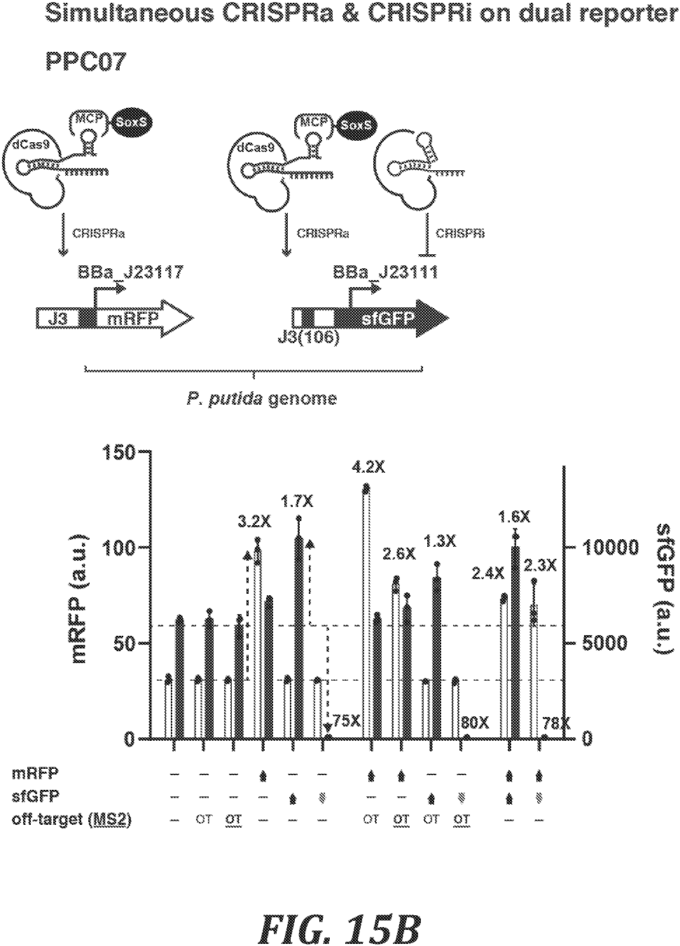

Additionally, simultaneous CRISPRa/CRISPRi and dual CRISPRa on multi-gene reporters with integrated genomic reporters were also demonstrated. The general trends were similar to what is observed with plasmid-based reporters (FIGS. 14A-14B and FIGS. 15A-15B), but the magnitudes of the effects were smaller, likely due to the lower copy number of the reporter gene. The ability to activate genomically-integrated heterologous reporters suggests that CRISPRa may be effective at endogenous genomic targets in P. putida.

### Example 17

### CRISPRa on P. Putida Endogenous Promoters

To determine if CRISPRa can activate endogenous promoters, a set of endogenous genes with appropriate upstream scRNA target sites was identified. Thousands of reported TSSs for P. putida (D′Arrigo et al., 2016. Genome-wide mapping of transcription start sites yields novel insights into the primary transcriptome of Pseudomonas putida. Environ. Microbiol. 18, 3466-3481) were analyzed and ten promoters with potentially activatable target sites located at the proper distance from the TSS were selected. Specifically, NGG protospacer adjacent motifs (PAMs), which are required for recognition of Sp-dCas9/guide-RNA complex (Qi et al., 2013. Repurposing CRISPR as an RNA-guided platform for sequence-specific control of gene expression. Cell 152, 1173-83), at distances corresponding to the J105-J112 target sites (FIG. 2B) with ± 2 bp flexibility (FIG. 2C) were identified. For each endogenous promoter, a reporter cassette with the promoter, flanking sequences, and an mRFP reporter gene were constructed following a strategy previously described for an E. coli endogenous promoter library (Zaslaver et al., 2006. A comprehensive library of fluorescent transcriptional reporters for Escherichia coli. Nat. Methods 3, 623-628). On-target or off-target scRNAs were introduced into the reporter plasmid and delivered to a P. putida strain with integrated dCas9/MCP-SoxS.

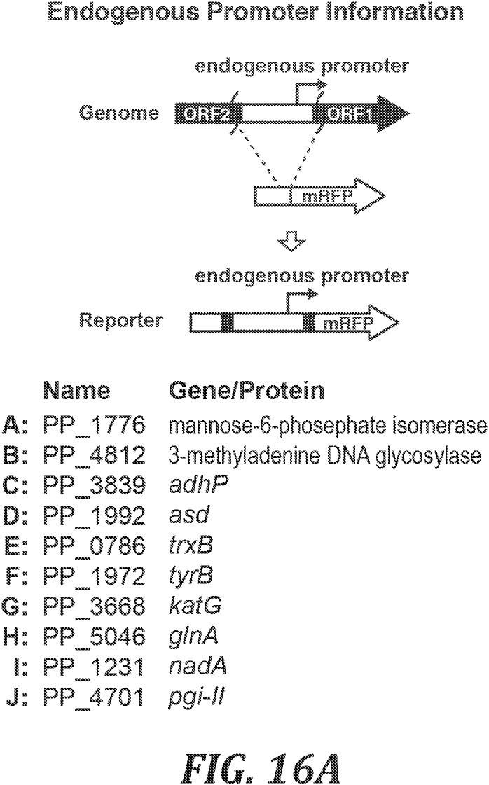

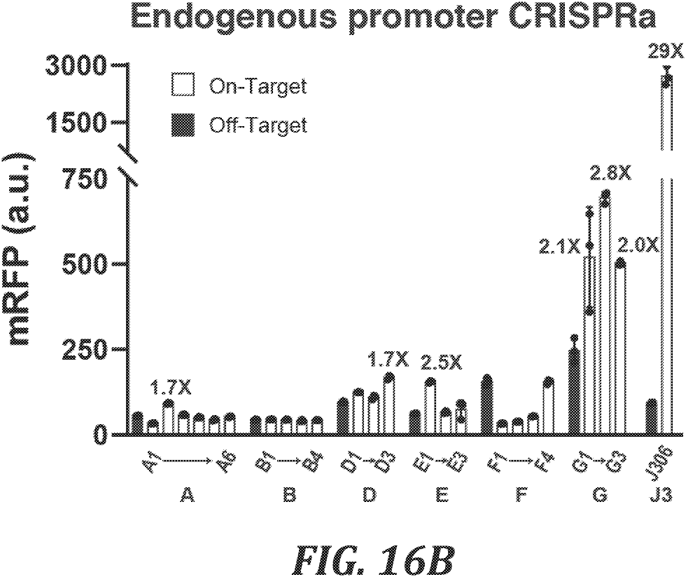

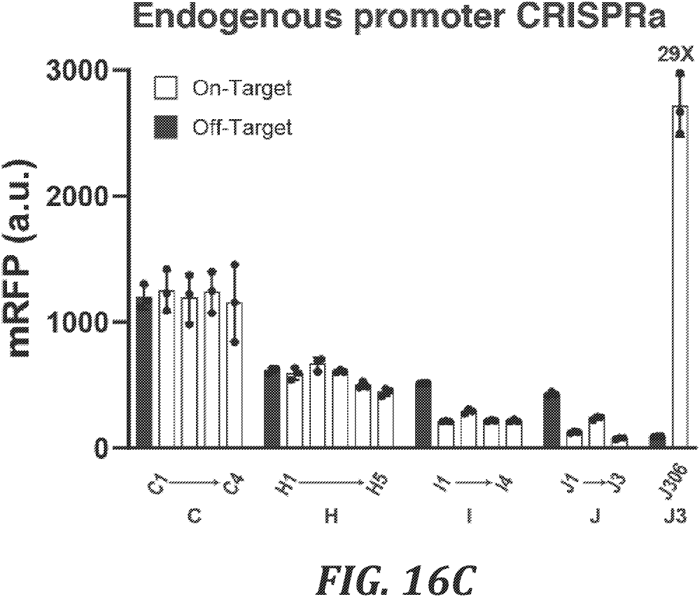

> 1.5-fold activation at 4 of the 10 promoters tested, with the highest fold-activation (2.8-fold) from scRNA G2 targeting katG (PP_3668) promoter was observed (FIG. 5A & FIG. 16B). The magnitudes of fold-activation from endogenous promoters are significantly lower than those under control of synthetic heterologous promoters (up to 40-fold and 100-fold for mRFP and sfGFP, respectively) (Table 6). Although higher fold-activation values may be desirable for future applications, relatively modest effects can still be physiologically significant. For example, external stresses can produce a wide range of expression changes in stress-responsive genes in P. putida. While some changes are quite large, others are in the 2-fold to 5-fold range (Bojanovič et al., 2017. Global transcriptional responses to osmotic, oxidative, and imipenem stress conditions in Pseudomonas putida. Appl. Environ. Microbiol. AEM.03236-16; Molina-Santiago et al., 2017. Global transcriptional response of solvent-sensitive and solvent-tolerant Pseudomonas putida strains exposed to toluene. Environ. Microbiol. 19, 645-658). Tools to perturb endogenous gene expression in this range may still be effective for modulating bacterial physiology and redirecting metabolic flux. Further, the ability to combine endogenous gene activation with heterologous gene activation and CRISPRi repression enables access to a vastly expanded space of gene expression programs compared to other synthetic gene regulatory methods.

This success rate and the magnitude of gene activation at endogenous targets in P. putida was similar to that observed previously in E. coli (Fontana et al., 2020a. Effective CRISPRa-mediated control of gene expression in bacteria must overcome strict target site requirements. Nat. Commun. 11, 1618). To predictably activate any endogenous gene, it will be necessary to further elucidate the rules for effective CRISPRa. Accurate annotations of TSSs and PAM-flexible dCas9 variants to precisely target the optimal distance upstream of the endogenous gene may improve activation (Fontana et al., 2020a. Effective CRISPRa-mediated control of gene expression in bacteria must overcome strict target site requirements. Nat. Commun. 11, 1618). Alternative bacterial activation domains are also available with different properties (Ho et al., 2020. Programmable CRISPR-Cas transcriptional activation in bacteria. Mol. Syst. Biol. 16, e9427; Liu et al., 2019. Engineered CRISPRa enables programmable eukaryote-like gene activation in bacteria. Nat. Commun. 10, 3693), and it may be possible to combine multiple activators as has been previously reported in eukaryotic systems (Chavez et al., 2015. Highly efficient Cas9-mediated transcriptional programming. Nat Methods 12, 326-8; Konermann et al., 2015. Genome-scale transcriptional activation by an engineered CRISPR-Cas9 complex. Nature 517, 583-8).

### Example 18

### Tunability of CRISPRa and CRISPRi With Inducible Promoter

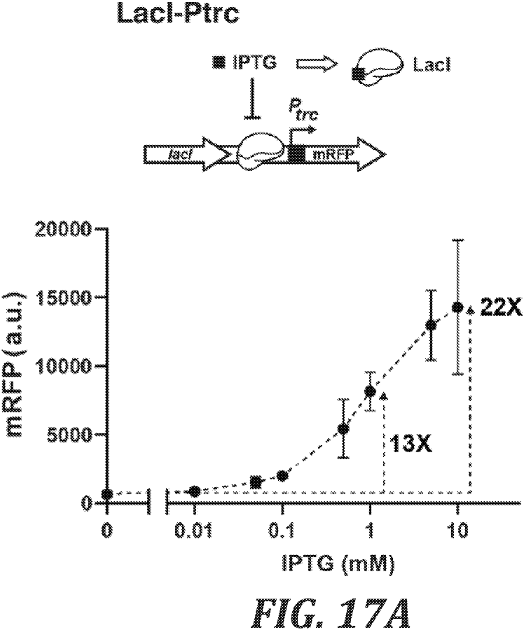

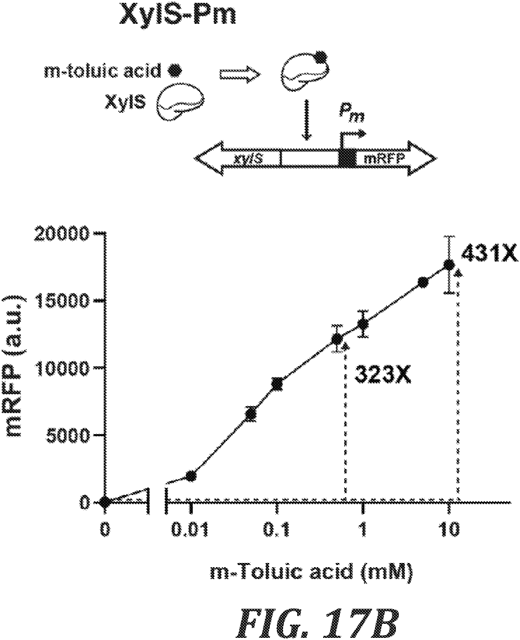

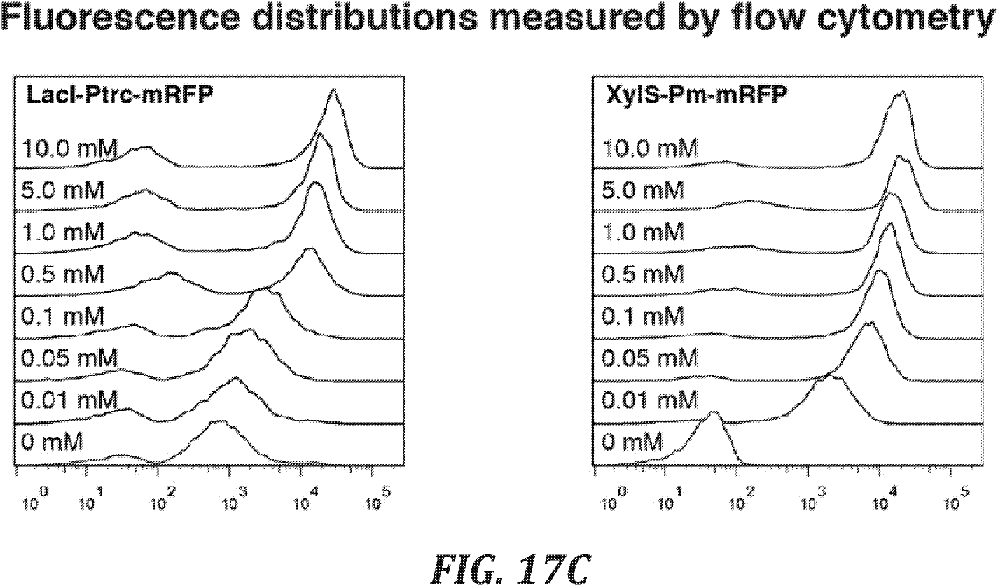

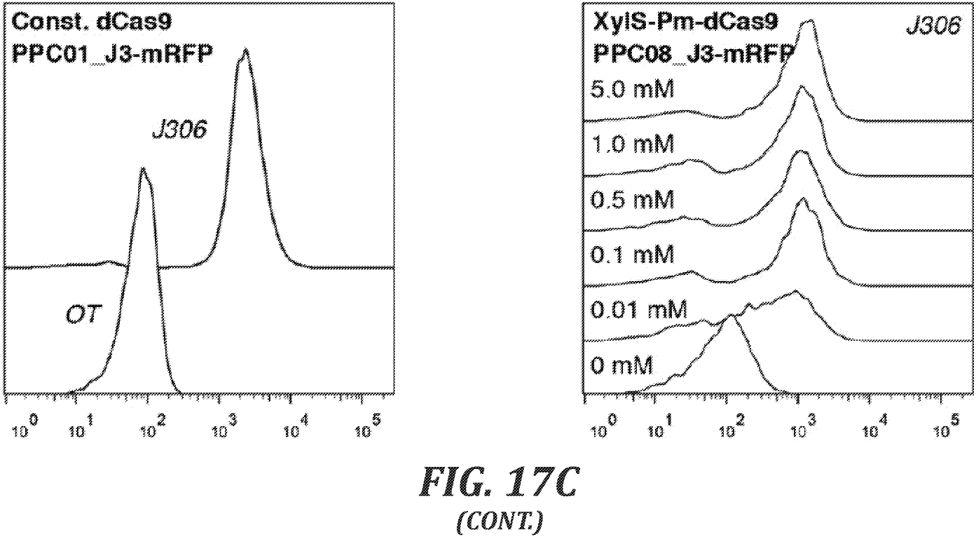

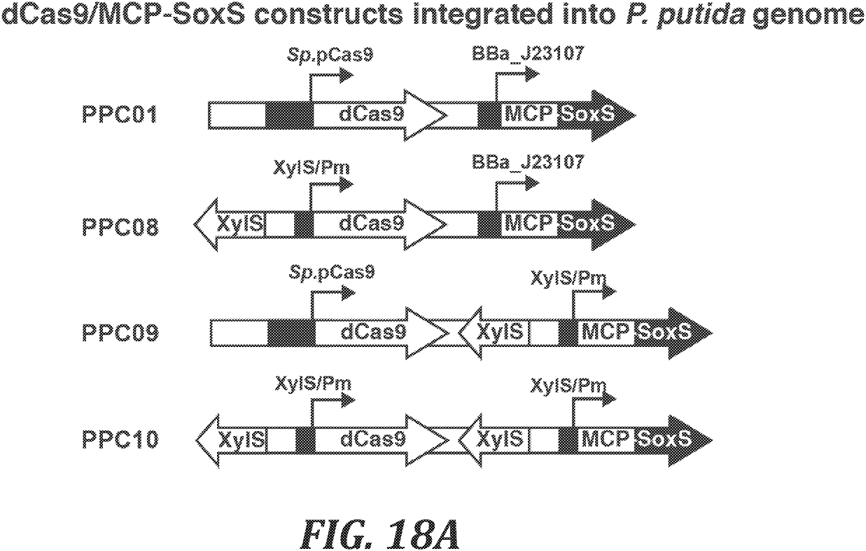

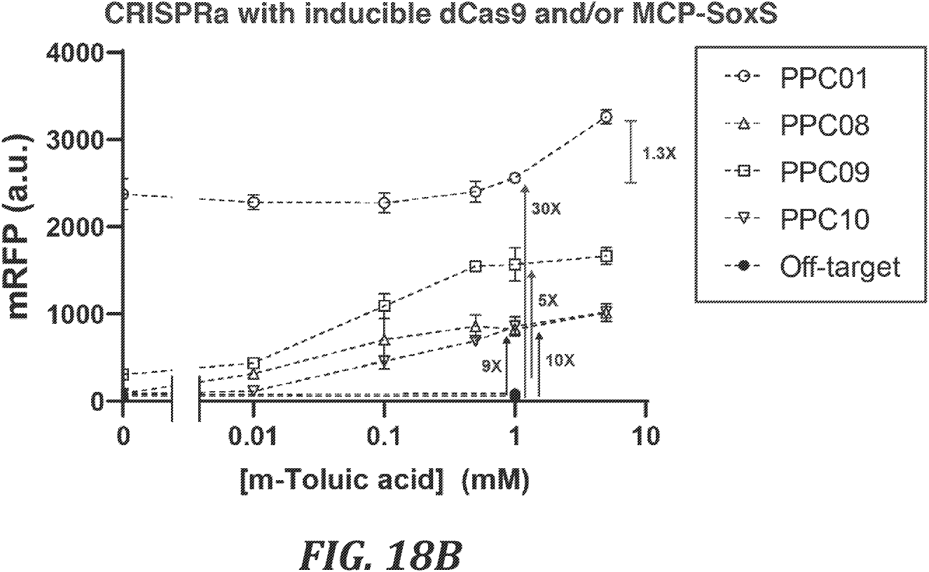

To tune expression levels with CRISPRa and CRISPRi, the CRISPR system components were placed under the control of a small-molecule inducible promoter. dCas9 and/or MCP-SoxS were expressed using XylS-Pm, an inducible promoter system from the P. putida mt-2 toluene degradation pathway (Wirth et al., 2019. Accelerated genome engineering of Pseudomonas putida by I-SceI-mediated recombination and CRISPR-Cas9 counterselection. Microb Biotechnol). XylS-Pm provides a higher dynamic range compared to the widely-used LacI-Ptrc system (FIGS. 17A-17B). Strains with inducible dCas9, inducible MCP-SoxS, or double-inducible dCas9/MCP-SoxS (PPC08-PPC 10) were constructed. A weak J3-BBa_J23117-mRFP reporter, induced with m-toluic acid (0-5 mM), was utilized for these experiments. In all three inducible strains assessed, tunable gene activation as a function of inducer concentration was observed (FIG. 18B). This approach will enable tunable and dynamically-regulatable expression control for further applications in metabolic engineering.

Using a strong reporter (J3-BBa_J23110-mRFP) that can be either activated or repressed, it was demonstrated that the extent of CRISPRa or CRISPRi could be tuned with different inducer levels. A reporter with either an activating scRNA or a repressing sgRNA was delivered to the inducible dCas9 strain (PPC08) and 3-fold activation with CRISPRa or 7-fold repression with CRISPRi at 1 mM m-toluic acid (FIG. 5B) was observed. This result suggests another potential strategy for improving the dynamic range of activation from heterologous genes. By targeting CRISPRi and CRISPRa to the same locus, we may be able to obtain lower basal expression and higher induced expression. Such a strategy would require expression of only the sgRNA for repression in the off state and only the scRNA for activation in the on state, which could potentially be achieved with orthogonal induction systems or with multi-layer CRISPR circuits.

### Example 19

### Biopterin Pathway Activation

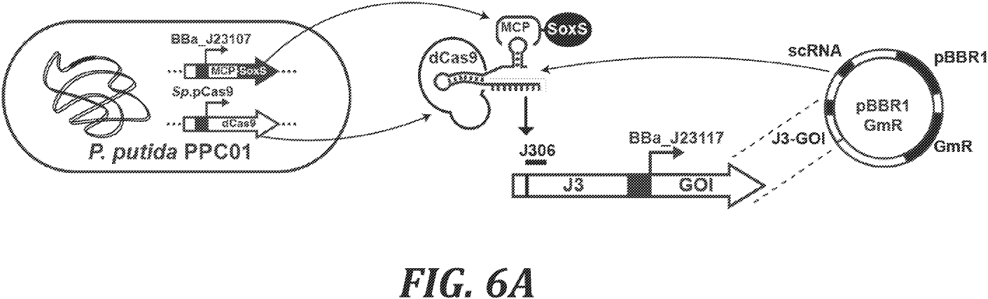

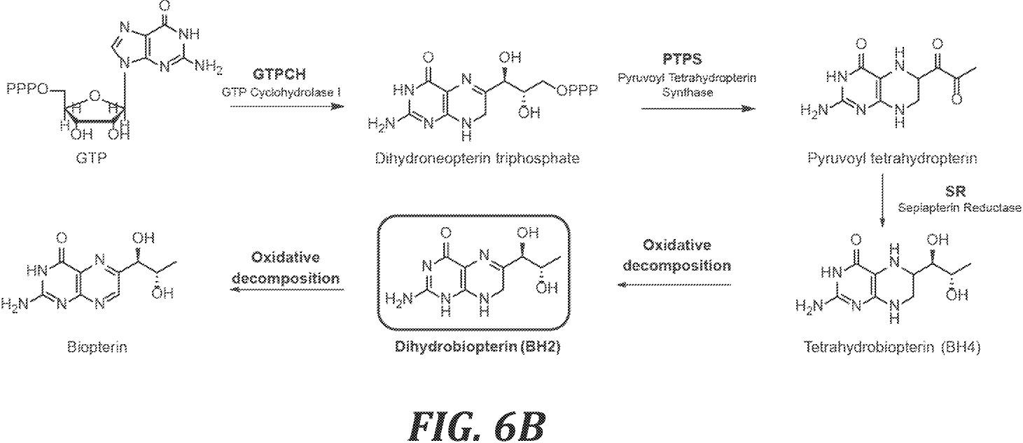

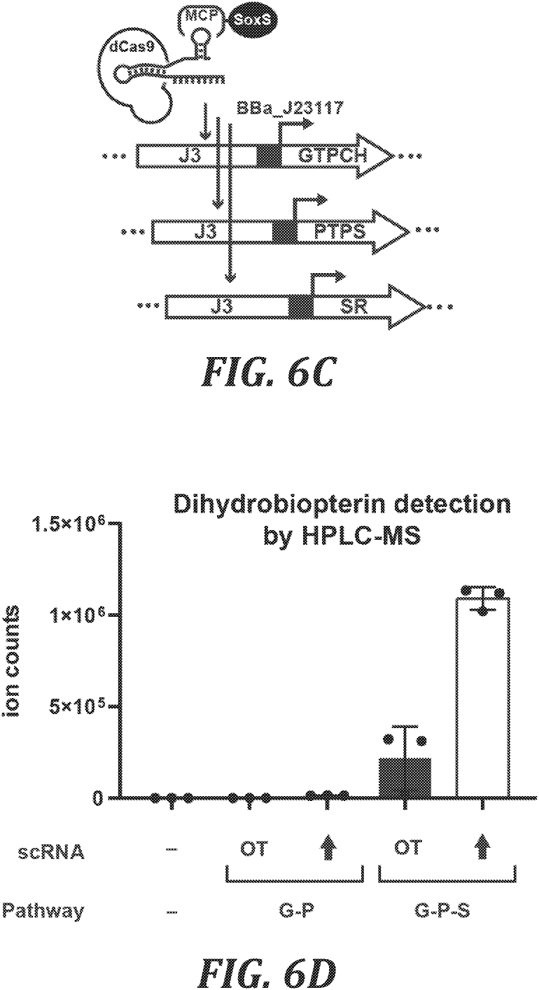

By characterizing the promoter features necessary for effective CRISPRa in P. putida, application of CRISPRa for metabolic pathway engineering was tested. The J3-BBa_J23117 promoter described in the previous section was used to place genes of interest under the control of a CRISPRa system. In a strain with integrated dCas9/MCP-SoxS (PPC01), transcriptional units controlled by J3-BBa_J23117 can be activated by the cognate J306 scRNA (FIG. 6A). Using this approach, it was demonstrated that CRISPRa can be used to switch on two different heterologous biosynthesis pathways, one for tetrahydrobiopterin (BH4) production with multiple transcriptional units activated by the same scRNA and one for mevalonate production as a multi-gene transcriptional unit under a single promoter.

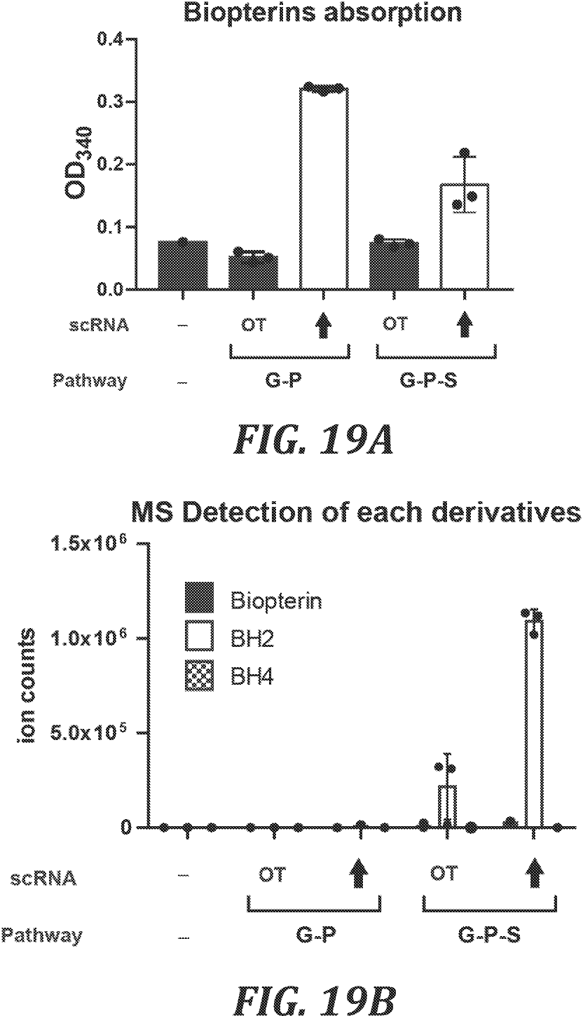

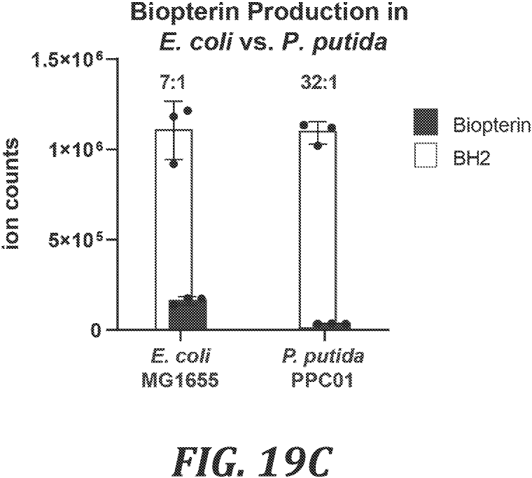

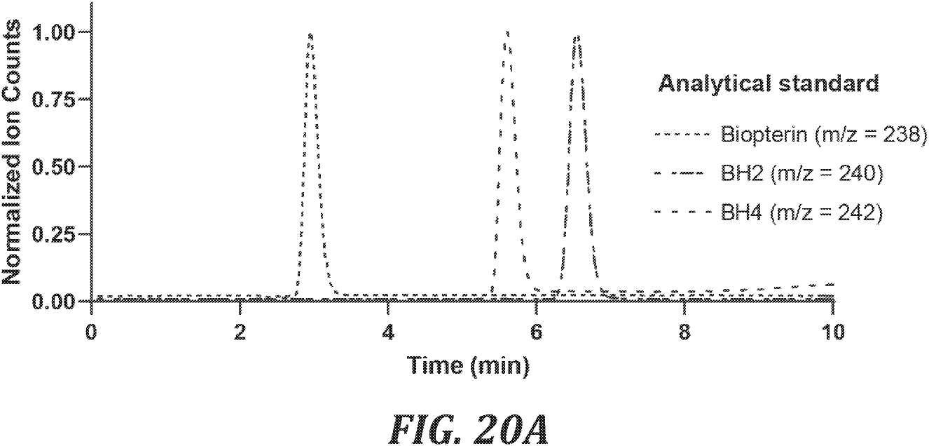

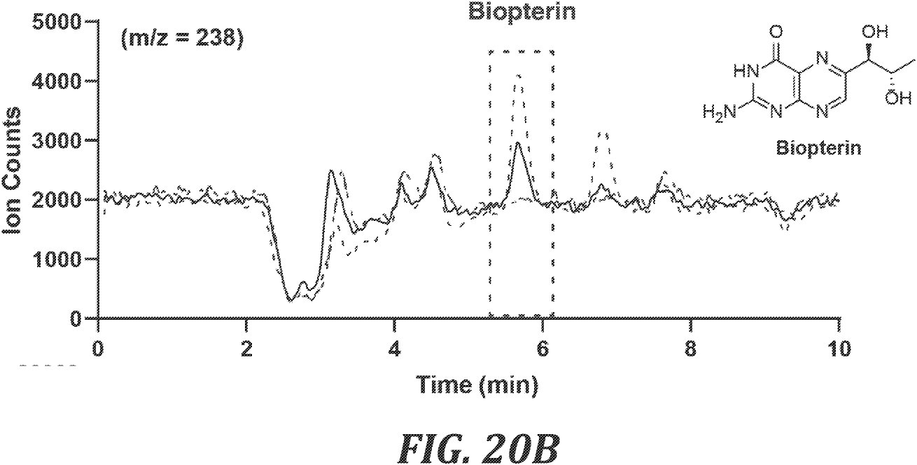

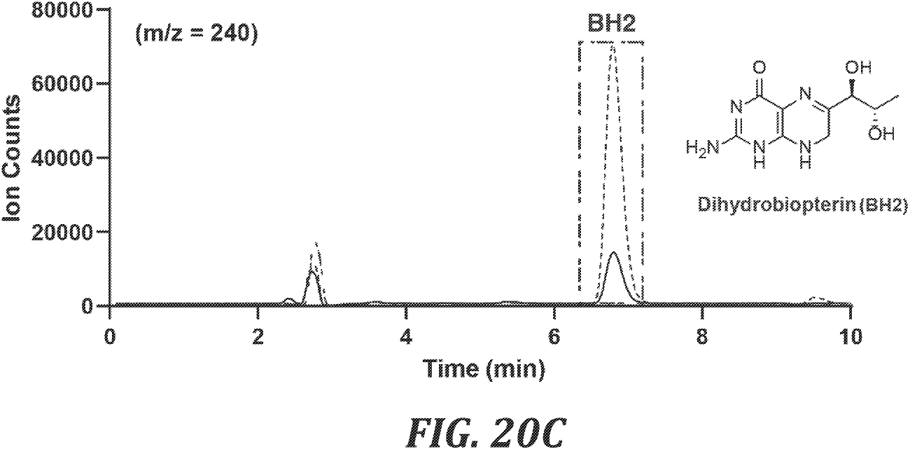

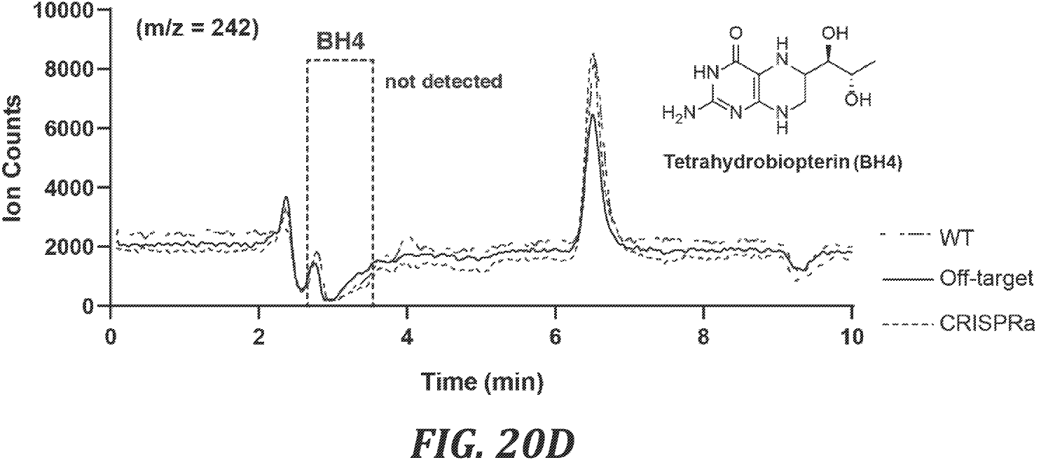

BH4 is an important cofactor in aromatic amino acid biosynthesis that can be produced from a three-enzyme pathway (FIG. 6B). BH4 has been previously produced in yeast using the E. coli GTPCH enzyme and the M. alpina PTPS and SR enzymes (Ehrenworth et al., 2015. Pterin-Dependent Mono-oxidation for the Microbial Synthesis of a Modified Monoterpene Indole Alkaloid. ACS Synth. Biol. 4, 1295-1307; Trenchard et al., 2015. De novo production of the key branch point benzylisoquinoline alkaloid reticuline in yeast. Metab. Eng. 31, 74-83). the gtpch gene from E. coli MG1655 and ptps/sr genes from M. alpina that were codon-optimized for expression in E. coli were used. Each gene was placed under control of the J3-BBa_J23117 promoter in a P. putida compatible plasmid (FIG. 6C). Because BH4 can be readily oxidized by atmospheric oxygen into dihydrobiopterin (BH2) and then biopterin in yeast (Ehrenworth et al., 2015. Pterin-Dependent Mono-oxidation for the Microbial Synthesis of a Modified Monoterpene Indole Alkaloid. ACS Synth. Biol. 4, 1295-1307), an initial screen for pathway output by absorbance at 340 nm, which reports on BH2 and biopterin was performed. A significant increase in OD₃₄₀ when the pathway was switched on with the cognate scRNA was observed (FIG. 19A). Subsequent analysis by HPLC-MS to identify specific parental ions confirmed that BH2 is the major product (FIG. 6D, FIG. 19B and FIG. 20). BH2 was also detected in the off-target scRNA sample (FIG. 6D), likely due to basal expression of the biopterin pathway enzymes. When the last gene in the pathway (sr) was omitted, no biopterin derivatives were detected by HPLC-MS, confirming that the full pathway is necessary for heterologous biopterin production (FIG. 6D and FIGS. 19A-19B). Thus, biopterin pathway activation by CRISPRa was able to significantly increase heterologous production. In some metabolic engineering applications, basal production may be problematic and pathway promoters may need to be modified to minimize leaky expression of the heterologous pathway genes. In future experiments, the CRISPRa system can be used to test whether product titers can be further optimized by independently activating biopterin pathway genes with orthogonal scRNAs and tuning their expression to different levels.

The major product of the biopterin pathway in P. putida is BH2, in contrast to S. cerevisiae where fully oxidized biopterin is the major product (Ehrenworth et al., 2015. Pterin-Dependent Mono-oxidation for the Microbial Synthesis of a Modified Monoterpene Indole Alkaloid. ACS Synth. Biol. 4, 1295-1307). The finding that BH2 is the major product suggests that the reducing potential of P. putida prevented BH2 from further oxidation. In E. coli, BH2 is the major product but the ratio of BH2:biopterin is significantly lower than in P. putida (FIG. 19C). Even though the fully reduced BH4, which is the desired product, was not observed in our system, the low biopterin level in P. putida suggests that its reducing power is advantageous for biosynthesis of oxidation-sensitive compounds.

### Example 20

### Mevalonate Pathway Activation

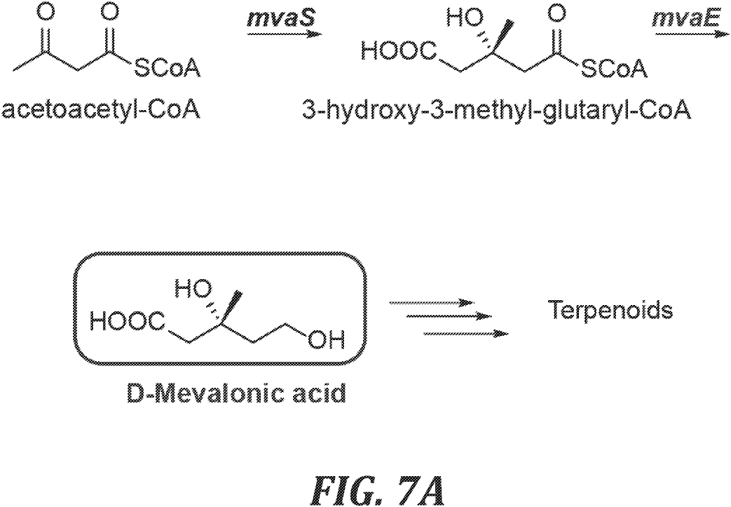

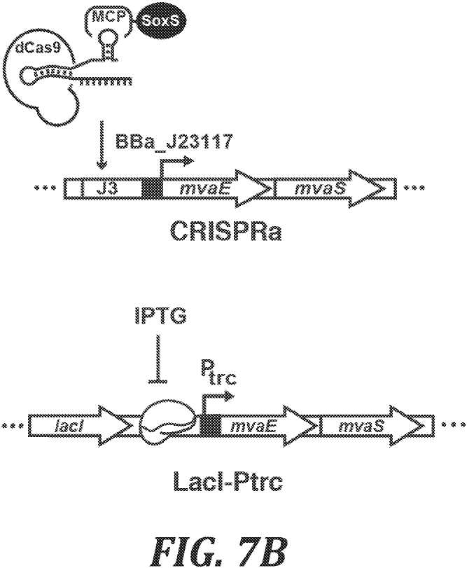

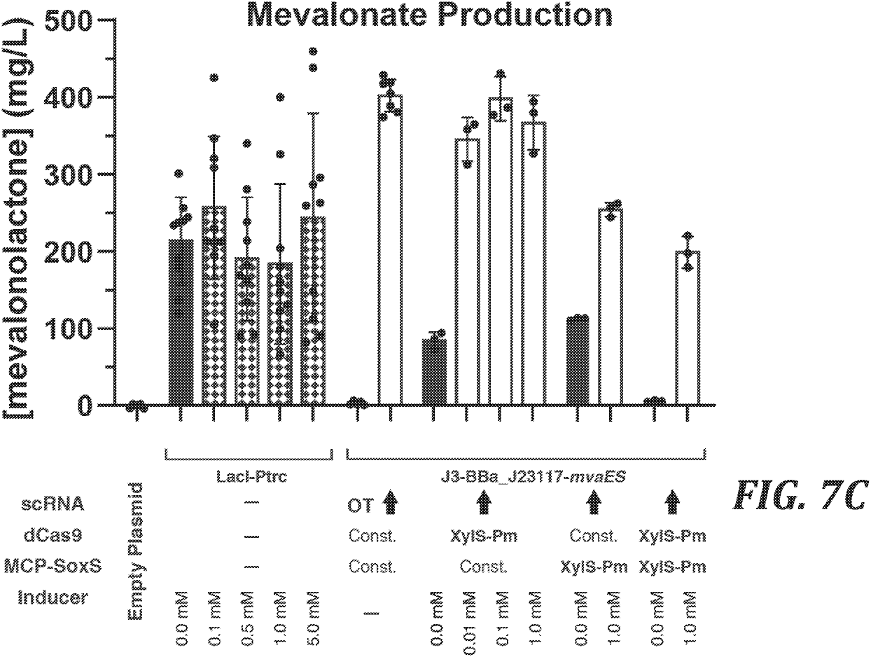

Next, it was determined whether CRISPRa could be used to produce mevalonic acid, a precursor to terpenoid natural products including fine chemicals, biofuels, and therapeutics (Anthony et al., 2009. Optimization of the mevalonate-based isoprenoid biosynthetic pathway in Escherichia coli for production of the anti-malarial drug precursor amorpha-4,11-diene. Metab. Eng. 11, 13-19; Jervis et al., 2019. Machine Learning of Designed Translational Control Allows Predictive Pathway Optimization in Escherichia coli. ACS Synth. Biol. 8, 127-136; Peralta-Yahya et al., 2011. Identification and microbial production of a terpene-based advanced biofuel. Nat. Commun. 2, 483). Mevalonate has previously been produced in P. putida using two genes, mvaE and mvaS, expressed in a single operon under the control of LacI-Ptrc (FIG. 7A) (Kim et al., 2019. CRISPR interference-mediated gene regulation in Pseudomonas putida KT2440. Microb Biotechnol). The mvaES operon was placed under the control of J3-BBa_J23 117 synthetic promoter (FIG. 7B). The constitutively-active CRISPRa-regulated mevalonate production strain was cultured side-by-side with the LacI-Ptrc regulated mvaES strain as a control. It was observed that the CRISPRa strain yielded 402 ± 21 mg/L mevalonate, which is similar to the highest mevalonate titer of 459 mg/L obtained with LacI-Ptrc after IPTG induction (FIG. 7C). The CRISPRa-regulated mvaES operon enables tight control of mevalonate production, with basal mevalonate production from the off-target CRISPRa control strain indistinguishable from the empty plasmid control (FIG. 7C). In contrast, the uninduced LacI-Ptrc strain produced mevalonate levels up to 214 ± 57 mg/L and yielded highly variable mevalonate levels in every IPTG concentration (ranging from 66 to 459 mg/L). A highly variable IPTG-induced mRFP expression was also observed, suggesting that expression from the LacI-Ptrc promoter may be unstable in P. putida (FIG. 17C). Taken together, these results demonstrate that one can effectively activate multi-gene biosynthesis pathways using a single operon (>40-fold increase in mevalonate biosynthesis, FIG. 7C) or with each enzyme produced from a separate transcriptional unit with its own CRISPRa-responsive promoter (5-fold increase in BH2 production, FIG. 6).

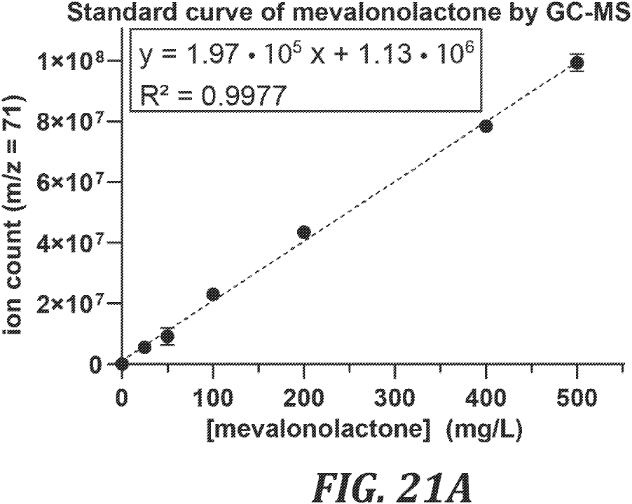

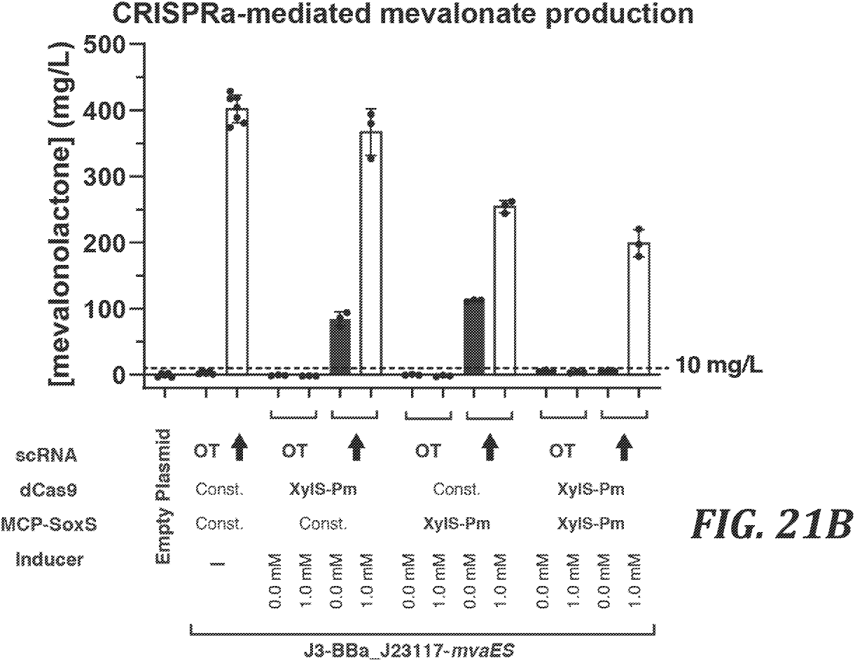

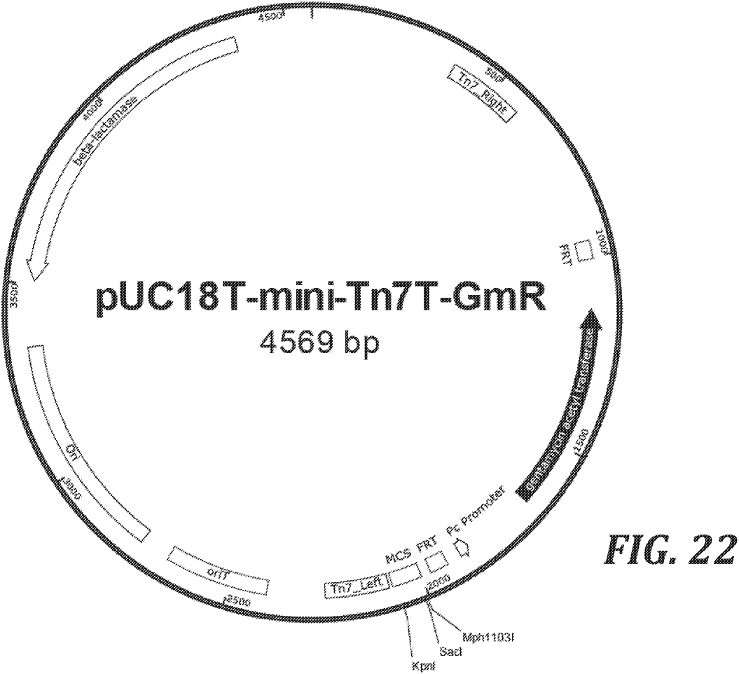

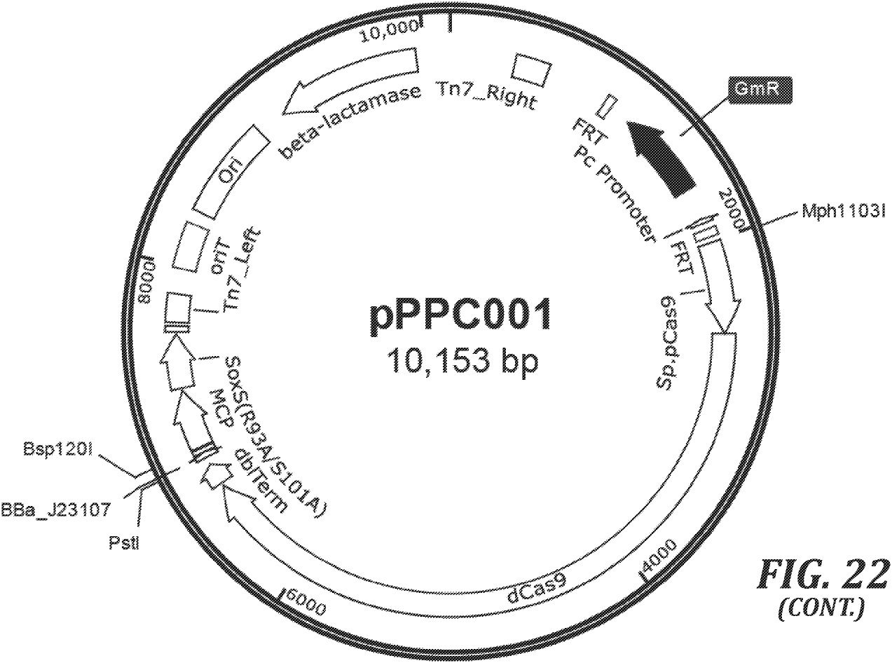

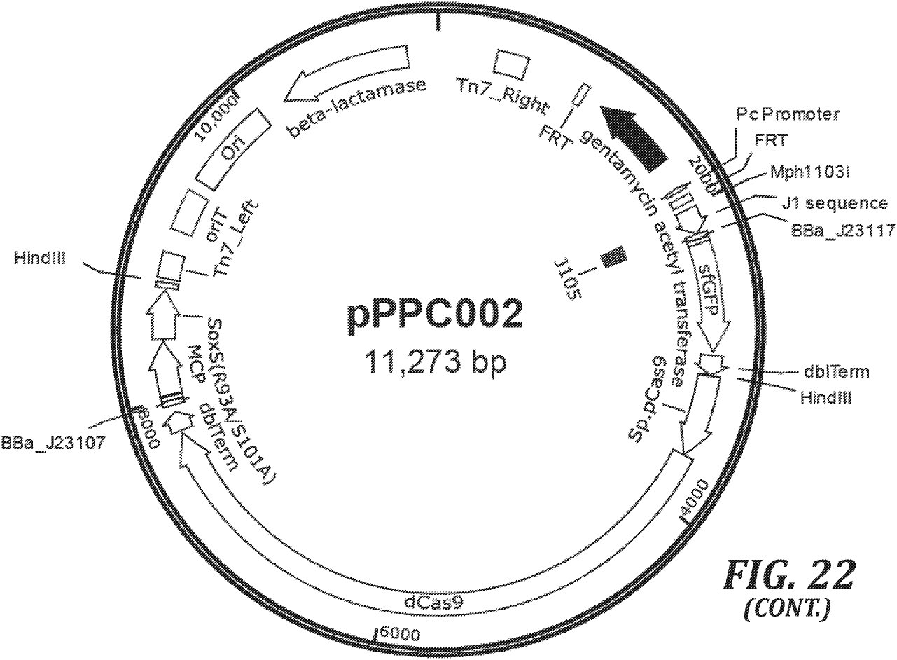

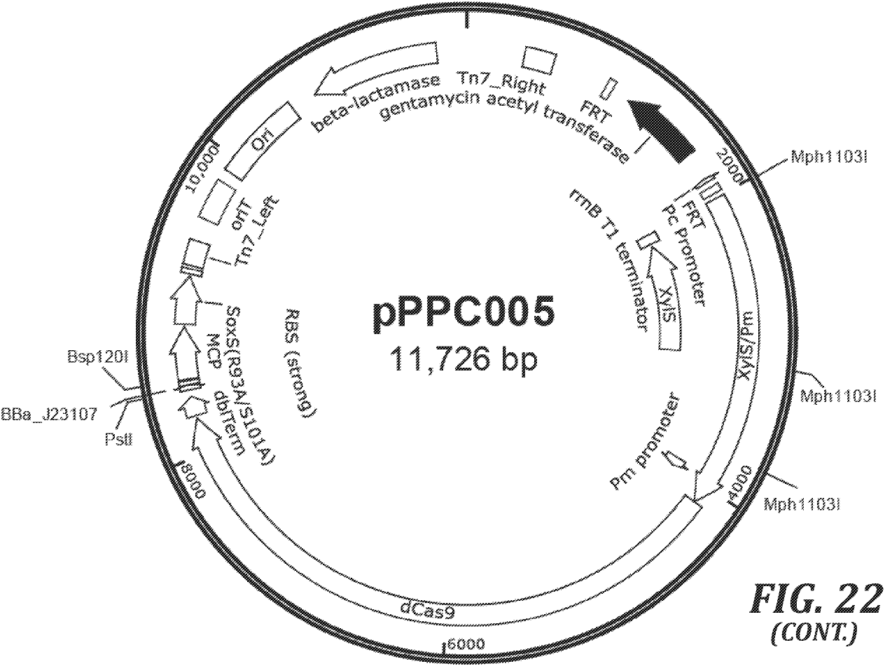

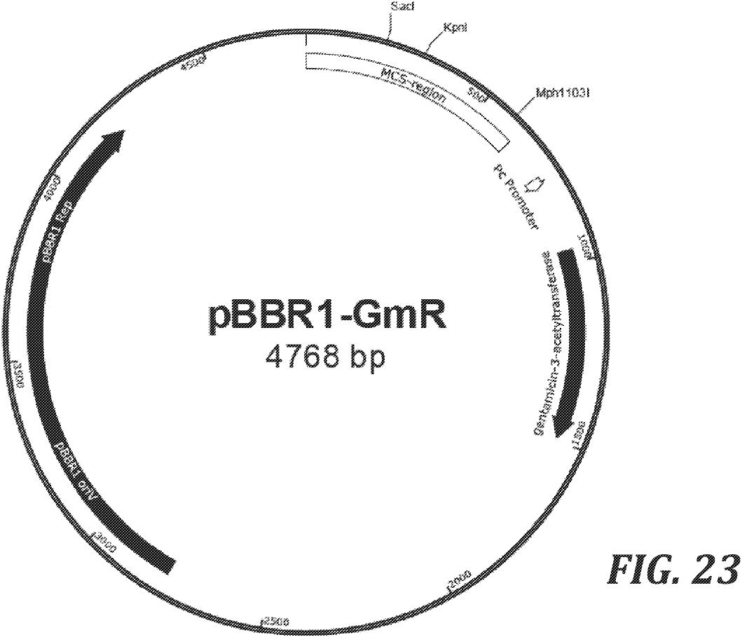

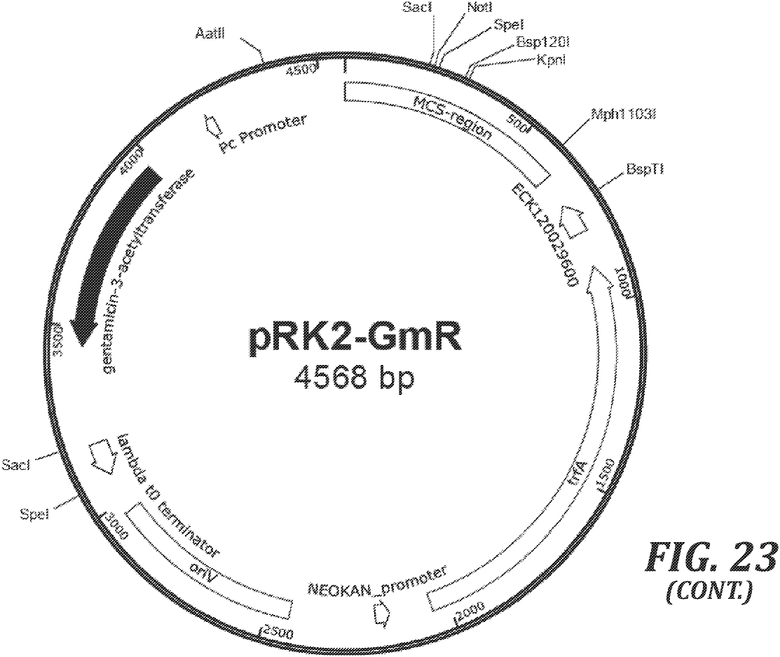

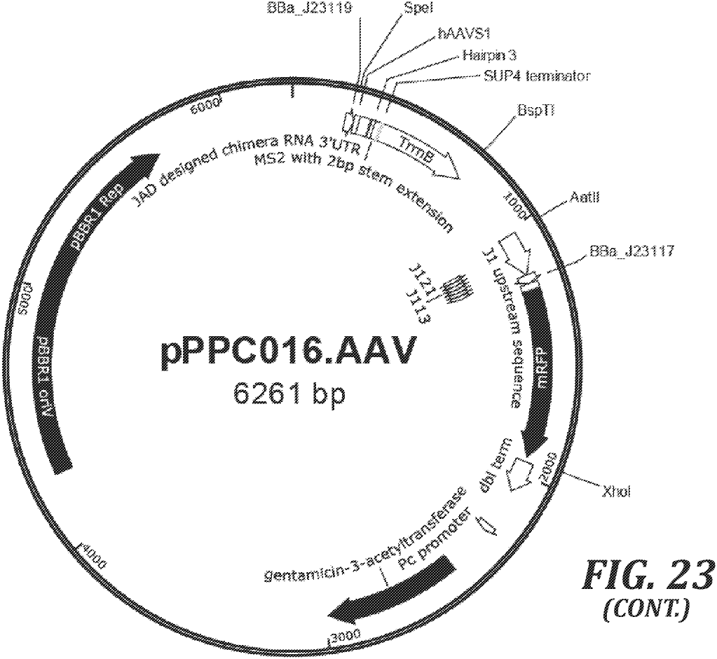

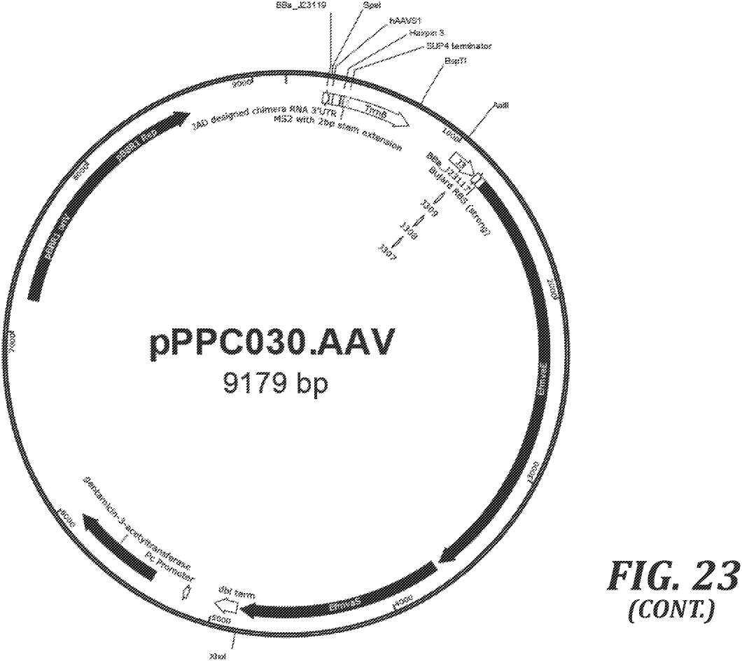

To determine if an inducible CRISPRa system could effectively regulate mevalonate production, a strain with toluic acid-inducible CRISPRa machinery (dCas9, MCP-SoxS, or both) was tested. In the absence of inducer 84 ± 11 mg/L mevalonate from the inducible dCas9 strain was observed. With inducer added to this strain (0.01 to 1.0 mM), a similar mevalonate level to that with constitutively expressed dCas9 was observed (345 to 397 mg/L and 402 ± 21 mg/L, respectively) (FIG. 7C). The inducible MCP-SoxS strain appeared to be leaky in the absence of inducer (112 ± 2 mg/L) and gave a lower mevalonate titer when induced (254 ± 9 mg/L). The double-inducible strain, with both dCas9 and MCP-SoxS controlled by XylS-Pm, had no significant leaky production in the absence of inducer but yielded the lowest mevalonate titer (199 ± 20 mg/L). The off-target scRNA yielded a level of mevalonate indistinguishable from the empty plasmid controls (less than 10 mg/L in FIG. 21B). The inducible CRISPRa system provides an additional layer of control that can be switched on at different growth phases and could be coupled with an inducible CRISPRi system for multi-gene programs with both activation and repression. Compared to the LacI-Ptrc regulated mvaES strain, which showed significant leaky production, the inducible dCas9 CRISPRa-regulated mvaES strain had minimal leakage and could provide advantages in situations where leaky metabolic gene expression could be toxic or burdensome to the cell.

### Example 21

### Increasing Number of gRNA in pACA Production

To validate the ability of CRISPRa to modulate multiple genetic constructs, functionality of CRISPRa in pACA production with increasing number of scRNA (3 to 6 scRNAs with J306, J506, and J606 as the first set of 3 scRNAs) was tested. The additional scRNAs (J106, hAAV, and J206 as the 4^{th} to 6^{th} scRNA, respectively) are off--target, i.e., these have no target anywhere on the plasmid or on P. putida genome. Two expression strategies: A) express pACA pathway and scRNAs on plasmid; or B) move pACA pathway to the genome but keep scRNAs on the plasmid, were tested. When pACA pathway was delivered on the plasmid, a minimal decrease in pACA level where production at 6 scRNAs equal to ~75% of pACA production at 3 scRNAs (FIG. 34A), was observed. However, when the pACA pathway was moved to the genome, pACA production decreased by half when the number of scRNAs increased from 3 to 6 (FIG. 34B). These data suggested that expression system optimization is needed to sustain CRISPRa efficiency with increasing number of scRNAs. Since additional scRNAs can be tolerated in the plasmid system, they can be repurposed for endogenous gene manipulation to further leverage chemical production by redirecting the flux to desired pathway, e.g., chorismate pathway upregulation.

### Example 22

### CRISPRa in Acinetobacter Baylyi ADP1

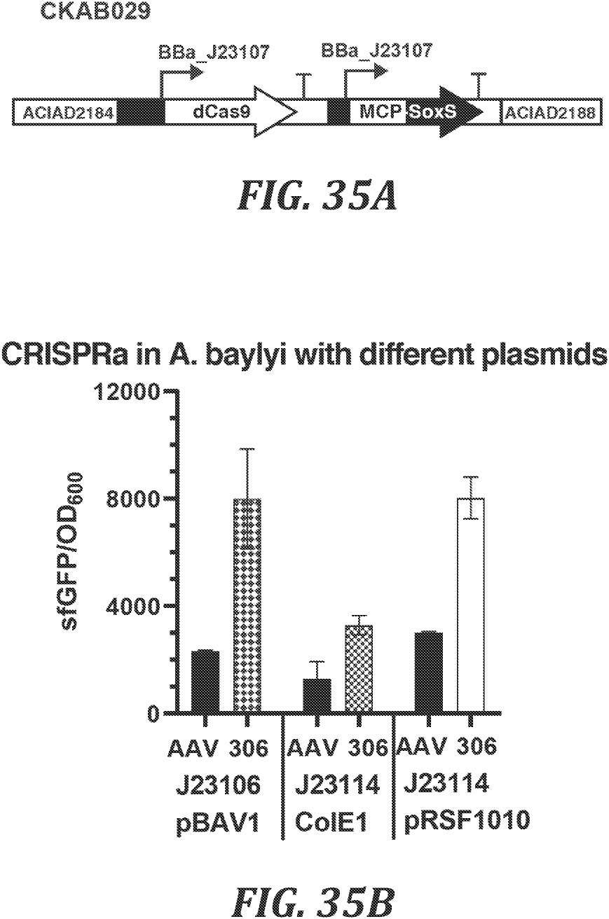

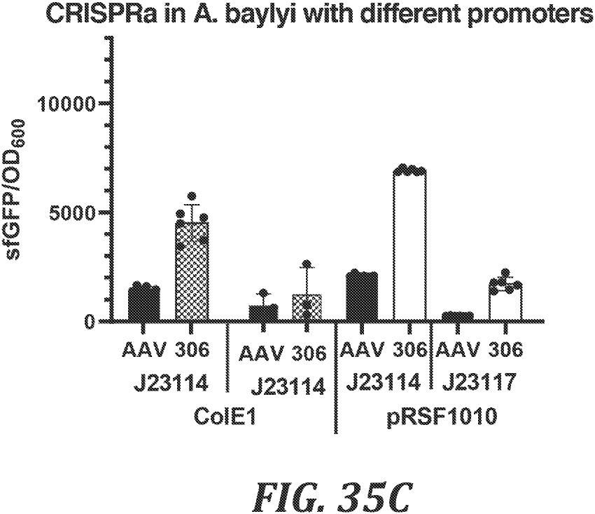

Since CRISPRa was shown to be functional both in E. coli and P. putida, it was contemplated that CRISPRa will be functional in broad range of bacteria. Acinetobacter baylyi ADP1 which has been reported for its ability to use lignocellulosic biomass was selected to demonstrate the transferability/portability of CRISPRa. Inspired by P. putida CRISPRa portability studies, showing that dCas9 and activator expression on the genome is more reliable, ADP1 was engineered into CRISPR enabled strain (CKAB029, FIG. 35A), which can consistently activate heterologous gene. CRISPRa was tested on different plasmid replicon and promoter strengths. Like E. coli and P. putida, CRISPRa in A. baylyi yielded higher fold-change at weak basal expression level (FIGS. 35B-35C). In combination with the previously reported CRISPRi in A. baylyi (Biggs et al. Development of a genetic toolset for the highly engineerable and metabolically versatile Acinetobacter baylyi ADP1. Nucleic Acids Res. 2020 1;48(9):5169-5182.), similar genetic manipulation by CRISPRa/i hold a potential to be applied in A. baylyi and other bacteria.

### Example 23

### PspF CRISPRa in P. Putida and Simultaneous Functionality With SoxS

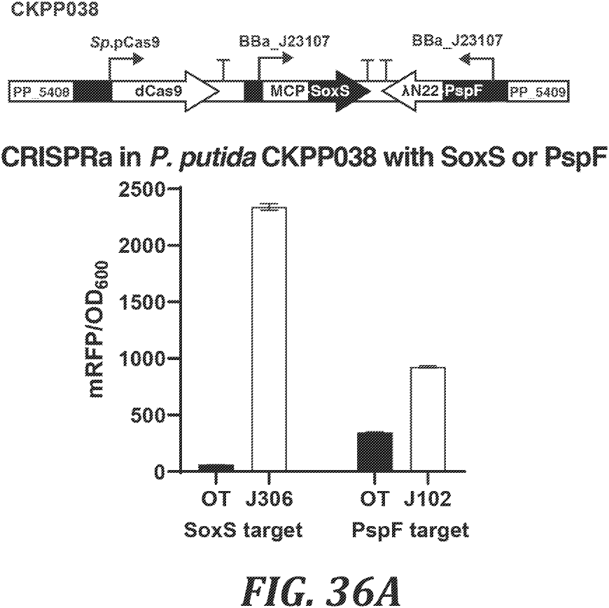

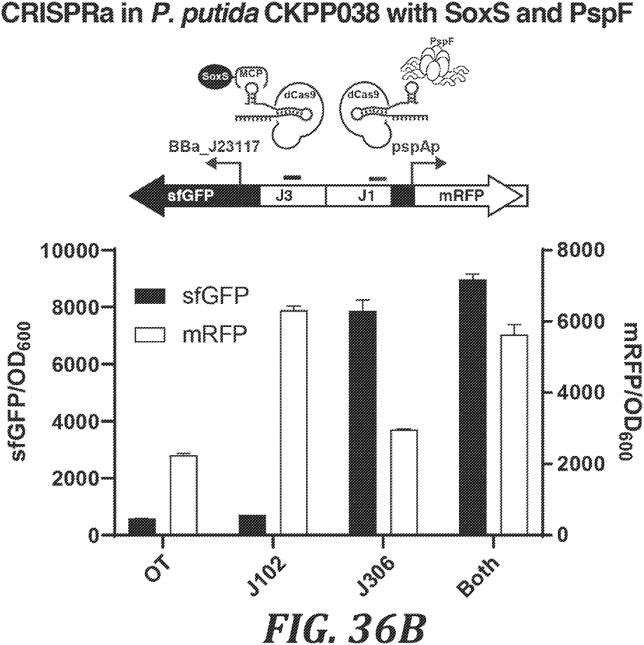

Apart from SoxS, multiple CRISPRa systems have been recently reported (Dong C et al. Synthetic CRISPR-Cas gene activators for transcriptional reprogramming in bacteria. Nat Commun. 2018 ;9(1):2489., Liu Y et al. Engineered CRISPRa enables programmable eukaryote-like gene activation in bacteria. Nat Commun. 2019 ;10(1):3693., Ho et al. Programmable CRISPR-Cas transcriptional activation in bacteria. Mol Syst Biol. 2020 Jul;16(7):e9427. Among these systems, PspF-mediated CRISPRa is the most distinct from that of SoxS as it works on sigma54 promoters instead of sigma70 family (Liu Y et al. Engineered CRISPRa enables programmable eukaryote-like gene activation in bacteria. Nat Commun. 2019 ;10(1):3693.). Here, it was demonstrated that PspF-mediated CRISPRa is functional in P. putida. PspF-AN22 was integrated into dCas9/MCP-SoxS bearing strain to enable PspF CRISPRa (CKPP038, FIG. 36A). This strain is functional for both SoxS-CRISPRa and PspF-CRISPRa (FIG. 36B). Since PspF-CRISPRa was developed with a different RNA recruitment strategy based on BoxB hairpin, it is postulated to function independently to that of MS2 used in SoxS system. To show the orthogonal programmability of two CRISPRa systems, a dual fluorescent reporter was used to demonstrate programmability of CRISPRa with scRNA hairpin (FIG. 36C). These results further validate portability of multiple bacterial CRISPRa systems to P. putida and other organisms.

### Example 24

### PAM-Flexible CRISPRa With Engineered dCas9

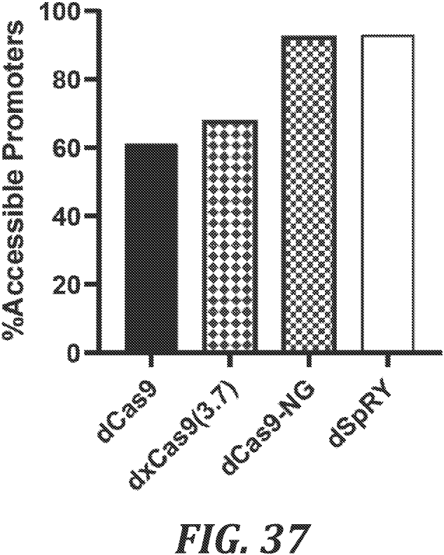

Key challenge of CRISPR-Cas9 system is the availability of PAM at the proper position. To bypass the PAM requirement, engineered dCas9 proteins with expanded PAM sequences have been used instead of the original dCas9 (Fontana J. et al. Effective CRISPRa-mediated control of gene expression in bacteria must overcome strict target site requirements. Nat Commun. 2020;11(1):1618. The ability to expand the targetable sites further with dxCas9-NG and dSpRY variants has been recently reported (Kiattisewee C et al. Expanding the Scope of Bacterial CRISPR Activation with PAM-Flexible dCas9 Variants. ACS Synth Biol. 2022, 4103-4112.) (FIG. 37).

In this work, the inventors ported a CRISPRa system from E. coli to P. putida successfully. The expression methods of dCas9, MCP-SoxS, and scRNA were optimized in P. putida and criteria for effective CRISPRa target sites in P. putida were defined. Based on the data and the methods disclosed herein, it is contemplated that a similar process of optimizing expression systems will enable effective CRISPRa-regulated gene expression in a wide range of bacterial species to enable complex CRISPR-based transcriptional programming in other industrially relevant microbes.

As reported previously in E. coli and in many eukaryotic systems, CRISPRa and CRISPRi can be used to target multiple genes simultaneously for activation or repression. Further, the CRISPRa system can be induced with small molecules, which will enable dynamic control of heterologous pathway activation. In P. putida, CRISPRa was applied to metabolic pathway engineering for tetrahydrobiopterin and mevalonate biosynthesis, providing proof-of-concept that CRISPRa-mediated gene regulation can be used to activate heterologous biosynthetic pathways.

Based on the present disclosure, the inventors contemplate an inducible CRISPR-Cas transcriptional control system to enable the rapid exploration of large combinatorial spaces of gene expression levels. A key advantage of CRISPR-Cas-mediated control is that, in principle, each gene of interest can be targeted by an orthogonal guide RNA and its expression level can be independently tuned. Endogenous genes can be targeted in this manner for both activation and repression to redirect metabolic flux towards the desired pathway precursors and activate heterologous pathways in a controlled manner to achieve optimal expression levels to maximize the production of desired biosynthetic products. The present disclosure therefore contemplates that the design principles disclosed herein can be used to rewire metabolic networks to enable more efficient biosynthetic production pathways for valuable chemical products.

TABLE 2

Description of bacterial strains and plasmids used in the present disclosure

| | | | |
|---|---|---|---|
| Figures | Strains | Plasmid | scRNA target (or sgRNA) |
| 1C | KT2440, PPC01 | pPPC010 + pPPC014, pPPC018, pPPC016 | hAAVS1, J109 |
| 2B | PPC02 | pPPC008 | hAAVS1, J101-121 |
| 2C | PPC02, PPC03.1-12 | pPPC008 | hAAVS1, J106 |
| 2D | PPC01 | pPPC016, pPPC020, pPPC020(106), pPPC016(306) | hAAVS1, J106, J306 |
| 3A | PPC01 | pPPC020, pPPC021.J231XX (10 promoters) | hAAVS1, J306 |
| 3B | PPC01 | pPPC022.5PS | J306 |
| 3C | PPC01, MG1655 | pPPC023.5PSN (PS1 to PS5), co-transform with pCD442 in E. coli | hAAVS1, J306 |
| 3D | PPC01, MG1655 | pPPC016, pPPC020, pPPC021.J231XX, pPPC023.5PSN E. coli plasmids according to (Fontana et al., 2020) | hAAVS1, J106, J306 |
| 4 | PPC01 | pPPC024 | hAAVS1, J306, jfGFP1, J306-jfGFP1, J106, J306-J106 |
| 5A | PPC01 | pPPC026.XN | hAAVS1, AN-JN as listed in Table 5 |
| 5B | PPC01, PPC08 | pPPC021.J23110 | hAAVS1, J306, RR2 |
| 6D | PPC01 | pPPC027, pPPC028 | hAAVS1, J306 |
| 7C | KT2440, PPC01, PPC08, PPC09, PPC10 | pBBR1-GmR, pPPC029, pPPC030 | hAAVS1, J306 |
| 8 | KT2440 | pPPC012, pPPC013, pPPC014, pPPC015, pPPC016, pPPC017, pPPC018, pPPC019 | hAAVS1 |
| 9A | KT2440, PPC01 | pPPC010 + pPPC014, pPPC011 + pPPC016, pPPC019, pPPC018, pPPC017, pPPC016 | hAAVS1, J109 |
| 9B | KT2440, PPC01 | pPPC010 + pPPC014, pPPC011 + pPPC016, pPPC016, pPPC017, pPPC016 + pRK2-KmR | hAAVS1 |
| 10 | PPC01 | pPPC016, pPPC017, pPPC018, pPPC019, pBBR1-GmR, pBBR1-KmR, pRK2-GmR, pRK2-KmR | hAAVS1, J109 |
| 11 | PPC01 | pPPC016, pPPC020 | hAAVS1, J106, J107, J108, J109, J306, J307, J308, J309 |
| 12A | KT2440, PPC01 | pPPC022.5PS | J306 |
| 12B | PPC01, MG1655 | pPPC016, pPPC020, pPPC021.J231XX, | hAAVS1, J106, J306 |
| | | pPPC023.5PSN E. coli plasmids according to (Fontana et al., 2020) | |
| 13 | PPC01 | pPPC025 | hAAVS1, J306, J106, J306-J106, J306-hAAVS1 |
| 14A | PPC04 | pBBR1-KmR, pPPC009 | hAAVS1, J106, RR2, hAAVS1-RR2, J106-RR2 |
| 14B | PPC05 | pBBR1-GmR, pPPC008 | hAAVS1 (sgRNA), hAAVS1, J306, jfGFP1, hAAVS1-jfGFP1, J306-hAAVS1 (sgRNA), J306-hAAVS1, J306-jfGFP1 |
| 14A | PPC06 | pBBR1-GmR, pPPC008 | hAAVS1, J306, J106, J306-J106, J306-hAAVS1 |
| 15B | PPC07 | pBBR1-GmR, pPPC008 | hAAVS1(sgRNA), hAAVS1, J306, J106, jfGFP1, J306-hAAVS1(sgRNA), J306-hAAVS1, hAAVS1 (sgRNA)-J106, hAAVS1-jfGFP1, J306-J106, J306-jfGFP1 |
| 16 | PPC01 | pPPC026.XN | hAAVS1, AN-JN as listed in Table 5 |
| 17A | KT2440 | pCK241 | |
| 17B | KT2440 | pCK243 | |
| 17C | KT2440, PPC01, PPC08 | pCK241, pCK243, pPPC020 | hAAVS1, J306 |
| 18 | PPC01, PPC08, PPC09, PPC10 | pPPC020 | hAAVS1, J306 |
| 19A, 19B | PPC01 | pPPC027, pPPC028 | hAAVS1, J306 |
| 19C | PPC01, MG1655 | pPPC027, pCD442, pCD581, pCK015 | hAAVS1, J306 |
| 20 | KT2440, PPC01 | pPPC027 | hAAVS1, J306 |
| 21 | KT2440, PPC01, PPC08, PPC09, PPC10 | pBBR1-GmR, pPPC030 | hAAVS1, J306 |
| 26 | E. coli MG1655 | pJF229B, pJF234.X | hAAVS1, J306, J206, J306+J206 |
| 27 | CKPP002 (PPC01) | pIDFP003.117.X, pCK425.At-pal | J306, J506, J606 |
| 28 | CKPP002, IFPP002 | pIDFP003.117.X, pCK425.At-pal | J306, J506, J606 |
| 29 | CKPP002, IFPP002, IFPP008 | pCK440.X, pCK443.X, pCK439.X, pCK537.N | hAAVS1, J306, J506, J606 |
| 30 | CKPP002, IFPP002, IFPP008 | pCK440.X, pCK443.X, pCK439.X, pCK537.N | hAAVS1, J306, J506, J606 |
| 31A | CKPP002 | pCK365.J231XX | hAAVS1, J306 |
| 31AB | CKPP002 | pCK190.N | hAAVS1, J306.N |
| 31E | CKPP002 | pCK422, pCK537.N | hAAVS1, J306, J506, J606 |
| 32B | CKPP002 | pIDFP003.117.X, pCK425.X, pCK426.X | hAAVS1, J306, J506, J606 |
| 32D | CKPP002, IFPP002 | pIDFP003.117.X, pCK425.X, pCK440.X, pCK439.X | hAAVS1, J306, J506, J606 |
| 33A | CKPP002 | pCK343.P.X | hAAVS1, F4, N3, 02, P1, Q2, R4, S4, T2, U1, W2 |
| 33B | CKPP002 | pCK348.P.X | hAAVS1, a2, β1, γ2, δ2, ε1 |
| 33C | CKPP002, PPCO5 | pPPC024.jfGFP, pPPC008.jfGFP1 | hAAVS1, jfGFP1 |
| 34C | CKPP002 | pCK509, pCK511, pCK683, pCK684 | J306, J506, J606, J106, hAAVS1, J206 |
| 34D | IFPP008 | pCK537.N | J306, J506, J606, J106, hAAVS1, J206 |
| 35B | CKAB029 | pCK396.X, pCK681.X, pCK682.X | hAAVS1, J306 |
| 35C | CKAB029 | pCK681.X, pCK682.X | hAAVS1, J306 |
| 36A | CKPP038 | pPPC020.X, pCK279.X | hAAVS1, J306, J102 |
| 36B | CKPP038 | pCK729.X | hAAVS1, J102, J306, J102+J306 |

TABLE 3

Description of biological parts in exemplary plasmids used in the P. putida toolbox

| | | | | |
|---|---|---|---|---|
| Integration plasmid | Backbone | Mph1103I | SacI/KpnI | HindIII |
| pPPC001 | pUC18T-miniTn7T-Gm | | Sp.pCas9-dCas9/BBa_J23107-MCP-SoxS | |
| pPPC002 | pUC18T-miniTn7T-Gm | | J1-BBa_J23117-sfGFP | Sp.pCas9-dCas9/BBa_J23107-MCP-SoxS |
| pPPC003.N | pUC18T-miniTn7T-Gm | | J1(+N)-BBa_J23117-sfGFP | Sp.pCas9-dCas9/BBa_J23107-MCP-SoxS |
| pPPC004 | pUC18T-miniTn7T-Gm | BBa_J23111-mRFP | J1-BBa_J23117-sfGFP | Sp.pCas9-dCas9/BBa_J23107-MCP-SoxS |
| pPPC005 | pUC18T-miniTn7T-Gm | | XylS-Pm-dCas9/BBa_J23107-MCP-SoxS | |
| pPPC006 | pUC18T-miniTn7T-Gm | | Sp.pCas9-dCas9/XylS-Pm-MCP-SoxS | |
| pPPC007 | pUC18T-miniTn7T-Gm | | XylS-Pm-dCas9/XylS-Pm-MCP-SoxS | |
| Integration plasmid | Backbone | Integration site | Gene Island | |
| pGNW2-ppl | pGNW2 | pp1 | | |
| pPPC031 | pGNW2 | pp1 | J3-BBa_J23117-mRFP | |
| pGNW2-pp2 | pGNW2 | pp2 | | |
| pPPC032 | pGNW2 | pp2 | BBa_J23111-sfGFP | |
| pPPC033 | pGNW2 | pp2 | J3(106)-BBa_J23117-sfGFP | |
| pPPC034 | pGNW2 | pp2 | J3(106)-BBa_J23111-sfGFP | |
| Replicable plasmid | Backbone | SacI/KpnI (NotI/Bsp120I) | Mph1103I (Aatll/Xhol or KpnI/XhoI) | scRNA or sgRNA |
| pPPC008 | pBBR1-GmR | scRNA, sgRNA, scRNA/sgRNAs | | hAAVS1, J101-121, J306, jfGFP1, hAAVS1(sgRNA), hAAVS1-jfGFP1, J306-hAAVS1(sgRNA), 1306-hAAVS1, J306-jfGFP1, J306-J106, hAAVS1(sgRNA)-J106 |
| pPPC009 | pBBR1-KmR | scRNA, sgRNA, scRNA/sgRNAs | | hAAVS1, J106, RR2, hAAVS1-RR2, J106-RR2 |
| pPPC010 | pBBR1-KmR | scRNA | Sp.pCas9-dCas9/BBa_J23107-MCP-SoxS | hAAVS1, J109, |
| pPPC011 | pRK2-KmR | | Sp.pCas9-dCas9/BBa_J23107-MCP-SoxS | |
| pPPC012 | pBBR1-GmR | | J1-BBa_J23117-mRFP | |
| pPPC013 | pBBR1-KmR | | J1-BBa_J23117-mRFP | |
| pPPC014 | pRK2-GmR | | J1-BBa_J23117-mRFP | |
| pPPC015 | pRK2-KmR | | J1-BBa_J23117-mRFP | |
| pPPC016 | pBBR1-GmR | scRNA | J1-BBa_J23117-mRFP | HAAVS1, J106,J107, J108, J109 |
| pPPC016(3 06) | pBBR1-GmR | scRNA | J1-BBa_J23117-mRFP (swap J106 to J306) | hAAVS1, J306 |
| pPPC017 | pBBR1-KmR | scRNA | J1-BBa_J23117-mRFP | hAAVS1, J109 |
| pPPC018 | pRK2-GmR | scRNA | J1-BBa_J23117-mRFP | hAAVS1, J109 |
| pPPC019 | pRK2-KmR | scRNA | J1-BBa_J23117-mRFP | hAAVS1, J109 |
| pPPC020 | pBBR1-GmR | scRNA | J3-BBa_J23117-mRFP | hAAVS1, J306, J307, J308, J309 |
| pPPC020(1 06) | pBBR1-GmR | scRNA | J1-BBa_J23117-mRFP (swap J306 to J106) | hAAVS1, J106 |
| pPPC021.J2 31XX | pBBR1-GmR | scRNA | J3-BBa_J231XX-mRFP where BBa_J231XX refers to either of J23109, J23113, J23114, J23115, J23107, J23105, J23106, J23108, J23111 | hAAVS1, J306 |
| pPPC021.J2 3110 | pBBR1-GmR | scRNA | J3-BBa_J23110-mRFP | hAAVS1, J306, RR2 |
| pPPC022.5PS | pBBR1-GmR | scRNA | J3(random-5PS)-BBa_J23117-mRFP | J306 |
| pPPC023.5PSN | pBBR1-GmR | scRNA | J3(SPSN)-BBa J23117-mRFP | hAAVS1, J306 |
| pPPC024 | pBBR1-GmR | scRNA | J3(106)-BBa_J23111-sfGFP_J3-BBa_J23117-mRFP | hAAVS1, J306, jfGFP1, J306-jfGFP1, J106, J306-J106 |
| pPPC025 | pBBR1-GmR | scRNA | J3(106)-BBa_J23117-sfGFP_J3-BBa_J23117-mRFP | hAAVS1, J306, J106, J306-J106, J306-hAAVS1 |
| pPPC026.XN | pBBR1-GmR | scRNA | PP_NNNN-mRFP where PP_NNNN refers to 10 endogenous promoters as listed in DNA Sequences | hAAVVS1, AN-JN as listed in Table 5 |
| pPPC027 | pBBR1-GmR | scRNA | J3-BBa_J23117-GTPCH, J3-BBa_J23117-PTPS, J3-BBa_J23117-SR | hAAVS1, J306 |
| pPPC028 | pBBR1-GmR | scRNA | J3-BBa_J23117-GTPCH, J3-BBa_J23117-PTPS | hAAVS1, J306 |
| pPPC029 | pBBR1-GmR | | LacI-Ptrc-mvaE-mvaS | |
| pPPC030 | pBBR1-GmR | scRNA | J3-BBa_J23117-mvaE-mvaS | hAAVS1, J306 |
| pCK241 | pBBR1-GmR | | LacI-Ptrc-mRFP | |
| pCK243 | pBBR1-GmR | | XylS-Pm-mRFP | |
| pCK255 | pBBR1-GmR | | I-Scel_sacB | |

TABLE 4

Primers used for cloning

| | | |
|---|---|---|
| Name | Sequence | Descriptive name |
| SEQ ID NO: 1 oCDP057 | ATTCGATCATGCATGTTACGAAATCATCCTGTGGAGCTT | pPPC001_Sp.pCas9_F |
| SEQ ID NO: 2 oCDP058 | CAAGGCCTTCGCGAGGCGAAAAAACCCCGCCG | pPPC001_BBa_B1002_R |
| SEQ ID NO: 3 oCDP003 | attcgatcatgcatgCTGCAGGCCTACGGTATCCACCGG | pPPC002_J1 |
| SEQ ID NO: 4 oCDP002 | caaggccttcgcgagAAGCTTtataaacgcagaaaggcccac | pPPC002_dblTerm_R |
| SEQ ID NO: 5 oCDP021 | tgcgtttataAAGCTTTTACGAAATCATCCTGTGGAGC | pPPC002_Sp.pCas9_F |
| SEQ ID NO: 6 oCDP022 | ccttcgcgagAAGCTTgcgaaaaaaccccgccg | pPPC002_BBa_B1102_R |
| SEQ ID NO: 7 oCDP061 | AAGCTAATTCGATCatgcatttgacggctagctcagtcc | pPPC003_BBa_J23111_F |
| SEQ ID NO: 8 oCDP062 | CGTAGGCCTGCAGcatataaacgcagaaaggcccacc | pPPC003_dblTerm_F |
| SEQ ID | gaagctaattcgatcatgcatgatttgtcctactcaggagagcg | pPPC005_XylS_F |
| NO: 9 oCK257 | | |
| SEQ ID NO: 10 oCK258 | attgagtatttcttatccatatgtttttcctcc | pPPC005_Pm_R |
| SEQ ID NO: 11 oCK259 | cggtttgcgtattgggcgcaatttgtcctactcaggagagcg | pPPC006_XylS_F |
| SEQ ID NO: 12 oCK260 | ACGTCTTCGCTACTCGCCATatgtttttcctcctaaccgcg | pPPC006_Pm_R |
| SEQ ID NO: 13 oCK101 | ccagctggcaattccgacgtcgtcgaatttgctttcgaatttctgc | pRK2-GmR_MCS_F |
| SEQ ID NO: 14 oCK102 | aagaccggcggtcttaagttttttggctgaagaattcgcaaatattatacgcaaggcgac | pRK2-GmR_MCS_R |
| SEQ ID NO: 15 oCK085 | aacaggagtccaagcgcatggaagccatcacaaacg | pRK2-KmR_KmR_F |
| SEQ ID NO: 16 oCK086_short | gacgtcggaattgccagctggg | pRK2-KmR_KmR_R |
| SEQ ID NO: 17 | tatagggcgaattggagctcTTGACAGCTAGCTCAGTCC | pPPC008_scRNA_F |
| oCDP023 | | |
| SEQ ID NO: 18 oCDP008 | gggaacaaaagctggTCTAGCTTAAGAGTTCACCGACAAACAACAGATA | pPPC008_scRNA_R |
| SEQ ID NO: 19 oCDP056 | TCGGTGAACTCTTAAcGGATCCTTGACAGCTAGCTCAGTCCTAGG | 2nd-gRNA_F |
| SEQ ID NO: 20 oCDP051 | gctggTCTAGCTTAAGAGTTCACCGACAAACAACAGAT | 2nd-gRNA_R |
| SEQ ID NO: 21 oCK079 | GCTCAGTCCTAGGTATAATACTAGT | change-gRNA_F |
| SEQ ID NO: 22 oCK287 | TAGGTATAATACTAGTNNNNNNNNNNNNNNNNNNNNGTTTTAGAGCTAGAAATAGCAAGT | new-gRNA_F |
| SEQ ID NO: 23 oCK077 | ttgcgtattgggcgcaTTACGAAATCATCCTGTGGAGCTT | pPPC010_Sp.pCas9_F |
| SEQ ID NO: 24 oCK078 | attacaacagtttttagcgaaaaaaccccgccg | pPPC010_Sp.pCas9_R |
| SEQ ID NO: 25 oCK097 | ttgcgtattgggcgcatggcaattccgacgtc | pPPC012_J1_F |
| SEQ ID NO: 26 oCK098 | attacaacagtttttaTATAAACGCAGAAAGGCCC | pPPC012-dblTerm_R |
| SEQ ID NO: 27 oCK237 | GCGTTCTGGACACAATTGGGTTCCACCGGATACCTCCGGACttgacagctagctcagtcc | pPPC0_16_J106-to-J306_F |
| SEQ ID NO: 28 oCK279 | TTGTGTCCAGAACGCTCCGTAGGACACCGCAGGATACCTGAGGTCGCCCG | pPPC016_J106-to-J306_R |
| SEQ ID NO: 29 oCK130 | ccaccgcggtggcggccgcTTGACAGCTAGCTCAGTCCTAG | pPPC018_gRNA_F |
| SEQ ID NO: 30 oCK146 | agctgggtaccgggcccAGTTCACCGACAAACAACAGATA | pPPC018_gRNA_R |
| SEQ ID NO: 31 oJF365 | GCGGTTACCAAAGGCGTCCTCGTCGTCTTGAAGTTGCG | pPPC020_J306-to-J106_F |
| SEQ ID NO: 32 oJF366 | GCCTTTGGTAACCGCAGGAGAAGTGAGGAGACGAGC | pPPC020_J306-to-J106_R |
| SEQ ID NO: 33 oCK177 | ttgggcgcatggcaattccg | pPPC021_J3_F |
| SEQ ID NO: 34 | GCCTGGagatccttactcga | pPPC021_dblTerm_R |
| oCK219 | | |
| SEQ ID NO: 35 oJF447 | GCGTTCTGGACACAANNNNNNNNNNNNNNNNNNNNNNNNNNttgacagctagctcagtcc | pPPC022_BBa_J23117_F |
| SEQ ID NO: 36 oJF448 | TTGTGTCCAGAACGCTCCGTAGGAGAAG | pPPC022_J3_R |
| SEQ ID NO: 37 oCK084 | cggtgcttaaaaactcgagtaaggatctCCAGG | pPPC022_dblTerm_F |
| SEQ ID NO: 38 oBT110 | gctagcactatacctaggactgagctagccgtcaa | pPPC024_BBa_J23111_R |
| SEQ ID NO: 39 oBT072 | CAGTCCTAGGTATAGTGCTAGCGAATTCATTAAAGAGGAG | pPPC024_BBa_J23111-RBS_F |
| SEQ ID NO: 40 oCK383 | ATGATCGCAAATGCTgAGTACTtataaacgcagaaaggcccacccg | pPPC024_dblT_R |
| SEQ ID NO: 41 oCDP059 | TTAAAGAGGAGAAAGGTACCATGGCGAGTAGCGAAGACG | pPPC026_mRFP_F |
| SEQ ID NO: 42 oCK251 | tgcgcccaatacgcaaaccg | pPPC026_MCS_R |
| SEQ ID NO: 43 oCK253 | cggtttgcgtattgggcgcagttctcagggctcgccgaga | pPPC026_PP_1776-A_F |
| SEQ ID NO: 44 oCK354 | GGTACCTTTCTCCTCTTTAATGAATTCtgatactggccacagacggct | pPPC026_PP_1776-A_R |
| SEQ ID NO: 45 oCK169 | gcgcatggcaattccgatatcAGCATTT | pPPC027_J3_F |
| SEQ ID NO: 46 oCK170 | GGagatccttactcgagtttTTATTCGTCGTAGAAATCAATGTGG | pPPC027_SR_R |
| SEQ ID NO: 47 oCK073 | ccagctggcaattccgacgtc | pPPC029_LacI_F |
| SEQ ID NO: 48 oCK072_S hort | agatccttactcgagtttttaattacgatagctacgcacgg | pPPC029_mvaS_R |
| SEQ ID NO: 49 oCDP046 | TTAAAGAGGAGAAAGGTACCatgaaaaccgtggtgattattgatg | pPPC030_mvaE_F |
| SEQ ID NO: 50 oCK325 | tgcctgcaggtcgactctagatgaccgacctgatcgaagtgaagac | pGNW2-pp1_HR1_F |
| SEQ ID | atggcggATGCATgggctcggttctctactggcg | pGNW2-pp1_HR1_R |
| NO: 51 oCK326 | | |
| SEQ ID NO: 52 oCK327 | cgagcccATGCATccgccattacgatctgacttgcc | pGNW2-pp1_HR2_F |
| SEQ ID NO: 53 oCK328 | ataacagggtaatctgaatttcacttcactcgggaaaaatcagggg | pGNW2-pp1_HR2_R |
| SEQ ID NO: 54 oCK344 | ccagtagagaaccgagcccAgcatggcaattccgacgtc | pPPC031_J3_F |
| SEQ ID NO: 55 oCK345 | agtcagatcgtaatggcggATATAAACGCAGAAAGGCCC | pPPC031_dblT_R |
| SEQ ID NO: 56 oCK369 | tgcctgcaggtcgactctagaccaggatgaataccttaaggacgcc | pGNW2-pp2_HR1_F |
| SEQ ID NO: 57 oCK370 | acaccttATGCATgcgcgtgatgcgctgcacac | pGNW2-pp2_HR1_R |
| SEQ ID NO: 58 | cacgcgcATGCATaaggtgtattcccccggcattca | pGNW2-pp2_HR2_F |
| oCK371 | | |
| SEQ ID NO: 59 oCK372 | ataacagggtaatctgaatttccgctcggcgcgtgatccgc | pGNW2-pp2_HR2_R |
| SEQ ID NO: 60 oCK373 | agcgcatcacgcgcATGCATttgacggctagctcagtcctaggt | pPPC032_BBa_J23111_F |
| SEQ ID NO: 61 oCK374 | cgggggaatacaccttAGTACTtataaacgcagaaaggcccacccg | pPPC032_dblT_R |
| SEQ ID NO: 62 oCK384 | cagcgcatcacgcgcATGCATAGCATTTGCGATCATTCACGCAGC | pPPC033_J3_F |
| SEQ ID NO: 63 oCDP004 | GGTACCTTTCTCCTCTTTAATGAATTC | RBS_R |
| SEQ ID NO: 64 oCD292 | GAATTCATTAAAGAGGAGAAAGGTACC | RBS_F |
| SEQ ID NO: 65 oCD281 | ATGGCGAGTAGCGAAGACGT | mRFP_F |
| SEQ ID NO: 66 oCK320 | TTCGCTACTCGCCATGGTACCTTTCTCCTCTTTAATGAATTCtgaaattgttatccgctc | Ptrc_RBS_R |
| SEQ ID NO: 67 oCK259 | cggtttgcgtattgggcgcaatttgtcctactcaggagagcg | XylS-Pm_F |
| SEQ ID NO: 68 oCK277 | GGTACCTTTCTCCTCTTTAATGAATTCttgcataaagcctaaggggtaggccttactaga | Pm_RBS_R |

TABLE 5

Target sequences of scRNA and sgRNA

| Name | Sequence | Target Promoter/Gene | Target Strand | Distance to TSS |
|---|---|---|---|---|
| SEQ ID NO: 69 sgRNA (20bp spacer is underlined) | NNNNNNNNNNNNNNNNNNNNGTTTTAG AGCTAGAAATAGCAAGTTAAAATAAGGC TAGTCCGTTATCAACTTGAAAAAGTGGC ACCGAGTCGGTGCTTTTTTT | | | |
| SEQ ID NO: 70 scRNA_1 | NNNNNNNNNNNNNNNNNNNNGTTTTAG AGCTAGAAATAGCAAGTTAAAATAAGGC TAGTCCGTTATCAACTTGAAAAAGTGGC | | | |
| xMS2.b2 (20bp spacer and MS2 hairpin are underline 4 | ACATGAGGATCACCCATGTGCTTTTTTT | | | |
| SEQ ID NO: 71 hAAVSI | GGGGCCACTAGGGACAGGAT | Off-target | N.A. | N.A. |
| SEQ ID NO: 72 RR2 | TGGAACCGTACTGGAACTGC | mRFP (CRISPRi) | Template | 185 from ATG |
| SEQ ID NO: 73 jfGFP1 | CATCTAATTCAACAAGAATT | sfGFP (CRISPRi) | Template | 38 from ATG |
| SEQ ID NO: 74 J101 | TGGGTTCCACCGGATACCTC | J1 | Non-template | 40 |
| SEQ ID NO: 75 J102 | AGGTATCCGGTGGAACCCAA | J1 | Template | 61 |
| SEQ ID NO: 76 | AGGCGTCCTTTGGGTTCCAC | J1 | Non-template | 50 |
| J103 | | | | |
| SEQ ID NO: 77 J104 | TGGAACCCAAAGGACGCCTT | J1 | Template | 71 |
| SEQ ID NO: 78 J105 | CGGTTACCAAAGGCGTCCTT | J1 | Non-template | 60 |
| SEQ ID NO: 79 J106 | AGGACGCCTTTGGTAACCGC | J1, J3(106) | Template | 81 |
| SEQ ID NO: 80 J107 | CGGTGTCCTGCGGTTACCAA | J1 | Non-template | 70 |
| SEQ ID NO: 81 J108 | TGGTAACCGCAGGACACCGC | J1 | Template | 91 |
| SEQ ID NO: 82 J109 | AGGTATCCTGCGGTGTCCTG | J1 | Non-template | 80 |
| SEQ ID NO: 83 J110 | AGGACACCGCAGGATACCTG | J1 | Template | 101 |
| SEQ ID NO: 84 | GGGCGACCTCAGGTATCCTG | J1 | Non-template | 90 |
| J111 | | | | |
| SEQ ID NO: 85 J112 | AGGATACCTGAGGTCGCCCG | J1 | Template | 111 |
| SEQ ID NO: 86 J113 | GGGCCACCACGGGCGACCTC | J1 | Non-template | 100 |
| SEQ ID NO: 87 J114 | AGGTCGCCCGTGGTGGCCCA | J1 | Template | 121 |
| SEQ ID NO: 88 J115 | TGGTGACCATGGGCCACCAC | J1 | Non-template | 110 |
| SEQ ID NO: 89 J116 | TGGTGGCCCATGGTCACCAT | J1 | Template | 131 |
| SEQ ID NO: 90 J117 | GGGTGACCTATGGTGACCAT | J1 | Non-template | 120 |
| SEQ ID NO: 91 J118 | TGGTCACCATAGGTCACCCT | J1 | Template | 141 |
| SEQ ID NO: 92 | TGGTTGCCAAGGGTGACCTA | J1 | Non-template | 130 |
| J119 | | | | |
| SEQ ID NO: 93 J120 | AGGTCACCCTTGGCAACCAA | J1 | Template | 151 |
| SEQ ID NO: 94 J121 | AGGACACCTTTGGTTGCCAA | J1 | Non-template | 140 |
| SEQ ID NO: 95 J306 | TTGTGTCCAGAACGCTCCGT | J3, J1(306) | Template | 81 |
| SEQ ID NO: 96 J307 | ACTTCTCCTACGGAGCGTTC | J3 | Non-template | 70 |
| SEQ ID NO: 97 J308 | AACGCTCCGTAGGAGAAGTG | J3 | Template | 91 |
| SEQ ID NO: 98 J309 | TCGTCTCCTCACTTCTCCTA | J3 | Non-template | 80 |
| SEQ ID NO: 99 A1 | TTCATGTAGCTTGTCCCCCG | PP_1776-A | Template | 82 |
| SEQ ID NO: 100 | CCGACTGAAGATGCGCTCTC | PP_1776-A | Non-template | 92 |
| A2 | | | | |
| SEQ ID NO: 101 A3 | ATGCGCTCTCTGGCGCTCCT | PP_1776-A | Non-template | 82 |
| SEQ ID NO: 102 A4 | TGCGCTCTCTGGCGCTCCTC | PP_1776-A | Non-template | 81 |
| SEQ ID NO: 103 A5 | GCGCTCTCTGGCGCTCCTCG | PP_1776-A | Non-template | 80 |
| SEQ ID NO: 104 A6 | CGCTCTCTGGCGCTCCTCGG | PP_1776-A | Non-template | 79 |
| SEQ ID NO: 105 B1 | ACTGGGATTTGTGTAGGAGC | PP_4812-B | Non-template | 92 |
| SEQ ID NO: 106 B2 | GGGTTTACCCGCGAAAGGGC | PP_4812-B | Non-template | 72 |
| SEQ ID NO: 107 B3 | GGCAGTGCCGGCCCTTTCGC | PP_4812-B | Template | 82 |
| SEQ ID NO: 108 | TGGCAGTGCCGGCCCTTTCG | PP_4812-B | Template | 81 |
| B4 | | | | |
| SEQ ID NO: 109 C1 | AATGCGTGGTCGCTTAATCC | PP_3839-C | Non-template | 82 |
| SEQ ID NO: 110 C2 | ATGCGTGGTCGCTTAATCCT | PP_3839-C | Non-template | 81 |
| SEQ ID NO: 111 C3 | TAATCCTGGGTTAACCGGAC | PP_3839-C | Non-template | 68 |
| SEQ ID NO: 112 C4 | TGCGCCGGTCCGGTTAACCC | PP_3839-C | Template | 81 |
| SEQ ID NO: 113 D1 | GGCCCCTGCGCTGCGCTCCG | PP_1992-D | Non-template | 72 |
| SEQ ID NO: 114 D2 | CAGCGCAGGGGCCGGATGAT | PP_1992-D | Template | 99 |
| SEQ ID NO: 115 D3 | CCGGAGCGCAGCGCAGGGGC | PP_1992-D | Template | 91 |
| SEQ ID NO: 116 | TATCGATGAAATCGCAGCAT | PP_0786-E | Non-template | 92 |
| E1 | | | | |
| SEQ ID NO: 117 E2 | CAGCATAGGCGATGCCTATG | PP_0786-E | Non-template | 78 |
| SEQ ID NO: 118 E3 | CCTTAGACAATCCACCTCAT | PP_0786-E | Template | 81 |
| SEQ ID NO: 119 F1 | AAAGCTGCGCCAGAGTGTCG | PP_1972-F | Non-template | 69 |
| SEQ ID NO: 120 F2 | ACACTCTGGCGCAGCTTTTG | PP_1972-F | Template | 89 |
| SEQ ID NO: 121 F3 | GACACTCTGGCGCAGCTTTT | PP_1972-F | Template | 90 |
| SEQ ID NO: 122 F4 | CGACACTCTGGCGCAGCTTT | PP_1972-F | Template | 91 |
| SEQ ID NO: 123 G1 | GGCGTCCTGGGCAAAGGGTA | PP_3668-G | Non-template | 91 |
| SEQ ID NO: 124 | CTGTGTATTGAAGCATGGCG | PP_3668-G | Non-template | 68 |
| G2 | | | | |
| SEQ ID NO: 125 G3 | CACAGCCATACCCTTTGCCC | PP_3668-G | Template | 103 |
| SEQ ID NO: 126 H1 | TATCCCACCCTCGCCATTTT | PP_5046-H | Non-template | 88 |
| SEQ ID NO: 127 H2 | CCCTCGCCATTTTCGGGCAC | PP_5046-H | Non-template | 81 |
| SEQ ID NO: 128 H3 | TGCCCGAAAATGGCGAGGGT | PP_5046-H | Template | 102 |
| SEQ ID NO: 129 H4 | GTGCCCGAAAATGGCGAGGG | PP_5046-H | Template | 101 |
| SEQ ID NO: 130 H5 | TGCATGCCAGTGCCCGAAAA | PP_5046-H | Template | 92 |
| SEQ ID NO: 131 11 | GGTTTTTGTAGTGCTTGTGC | PP_1231-I | Template | 101 |
| SEQ ID NO: 132 | GGGTTTTTGTAGTGCTTGTG | PP_1231-I | Template | 100 |
| I2 | | | | |
| SEQ ID NO: 133 I3 | CAATCCAGCGATTACTAAAG | PP_1231-I | Template | 80 |
| SEQ ID NO: 134 I4 | ACAATCCAGCGATTACTAAA | PP_1231-I | Template | 79 |
| SEQ ID NO: 135 J1 | TGGGTATGGCAGGGGGATTT | PP_4701-J | Template | 118 |
| SEQ ID NO: 136 J2 | GTGCTGGGAATGGGTATGGC | PP_4701-J | Template | 108 |
| SEQ ID NO: 137 J3 | CCACGTGCTGGGAATGGGTA | PP_4701-J | Template | 104 |

TABLE 6

Summary of CRISPRa-mediated fold-changes in gene expression and metabolite production

| | | | | |
|---|---|---|---|---|
| Description | Reporter gene/ Metabolic genes | scRNA | *Fold-change | Figure |
| Activation of plasmid-bourne mRFP with J1 promoter using a 2-plasmid system | J1-mRFP (2-plasmid) | J109 | 1.6-fold | FIG. 1C |
| Activation of plasmid-bourne mRFP with J1 promoter using a strain with integrated dCas9/MCP-SoxS (PPC01) | J1-mRFP (pBBR1-GmR) | J109 | 5-fold | FIG. 1C |
| Activation of plasmid-bourne mRFP with J3 promoter | J3-mRFP (pBBR1-GmR) | J306 | 34-fold | FIG. 2D |
| Dual activation of mRFP and sfGFP reporters (plasmid-bourne) | J3-mRFP (pBBR1-GmR) | J306 | 41-fold | FIG. 13 |
| | J3(106)-sfGFP (pBBR1-GmR) | J106 | 105-fold | FIG. 13 |
| Dual activation of mRFP and sfGFP reporters (genomically-integrated) | J3-mRFP (integrated) | J306 | 3-fold | FIG. 15A |
| | J3(106)-sfGFP (integrated) | J106 | 24-fold | FIG. 15A |
| Activation of plasmid-borne endogenous promoter reporter constructs (mRFP) | PP_1776-A-mRFP (pBBR1-GmR) | A2 | 1.7-fold | FIG. 5A |
| | PP_1992-D-mRFP (pBBR1-GmR) | D3 | 1.7-fold | FIG. 5A |
| | PP_0786-E-mRFP (pBBR1-GmR) | E1 | 2.5-fold | FIG. 5A |
| | PP_3668-G-mRFP (pBBR1-GmR) | G2 | 2.8-fold | FIG. 5A |
| Metabolite production upon activation of biopterin pathway genes | J3-GTPCH, J3-PTPS, J3-SR (pBBR1-GmR) | J306 | 5-fold | FIG. 6D |
| Metabolite production upon | 13-mvaES (pBBR1-GmR) | J306 | >40-fold | FIG. 7B |
| activation of mevalonate pathway genes | | | | |

*Fold-change values were calculated relative to a strain with an off-target scRNA. The J1 and J3 promoters shown in this table contain the BBa_J23117 minimal promoter.

TABLE 7

Additional DNA sequences used in the present disclosure

| | |
|---|---|
| SEQ ID NO: 138 >aroG* (RBS was underlined) | GAATTCAAAAGATCTAAATAACCTAAACGAGAGGAAAGAATAATGAAT TATCAGAACGACGATTTACGCATCAAAGAAATCAAAGAGTTACTTCCTC CTGTCGCATTGCTGGAAAAATTCCCCGCTACTGAAAATGCCGCGAATAC GGTTGCCCATGCCCGAAAAGCGATCCATAAGATCCTGAAAGGTAATGA TGATCGCCTGTTGGTTGTGATTGGCCCATGCTCAATTCATGATCCTGTCG CGGCAAAAGAGTATGCCACTCGCTTGCTGGCGCTGCGTGAAGAGCTGA AAGATGAGCTGGAAATCGTAATGCGCGTCTATTTTGAAAAGCCGCGTA CCACGGTGGGCTGGAAAGGGCTGATTAACGATCCGCATATGGATAATA GCTTCCAGATCAACGACGGTCTGCGTATAGCCCGTAAATTGCTGCTTGA TATTAACGACAGCGGTCTGCCAGCGGCAGGTGAGTTTCTCAACATGATC ACCCCACAATATCTCGCTGACCTGATGAGCTGGGGCGCAATTGGCGCA CGTACCACCGAATCGCAGGTGCACCGCGAACTGGCATCAGGGCTTTCTT GTCCGGTCGGCTTCAAAAATGGCACCGACGGTACGATTAAAGTGGCTA TCGATGCCATTAATGCCGCCGGTGCGCCGCACTGCTTCCTGTCCGTAAC GAAATGGGGGCATTCGGCGATTGTGAATACCAGCGGTAACGGCGATTG CCATATCATTCTGCGCGGCGGTAAAGAGCCTAACTACAGCGCGAAGCA CGTTGCTGAAGTGAAAGAAGGGCTGAACAAAGCAGGCCTGCCAGCACA GGTGATGATCGATTTCAGCCATGCTAACTCGTCCAAACAATTCAAAAAG CAGATGGATGTTTGTGCTGACGTTTGCCAGCAGATTGCCGGTGGCGAAA AGGCCATTATTGGCGTGATGGTGGAAAGCCATCTGGTGGAAGGCAATC AGAGCCTGGAGAGCGGGGAGCCGCTGGCCTACGGTAAGAGCATCACCG |
| | ATGCCTGCATCGGCTGGGAAGATACCGATGCTCTGTTACGTCAACTGGC GAATGCAGTAAAAGCGCGTCGCGGGTAA |
| SEQ ID NO: 139 >aroL (RBS was underlined) | GGATCTAAAGGAGGCCATCCATGACACAACCTCTTTTTCTGATCGGGCC TCGGGGCTGTGGTAAAACAACGGTCGGAATGGCCCTTGCCGATTCGCTT AACCGTCGGTTTGTCGATACCGATCAGTGGTTGCAATCACAGCTCAATA TGACGGTCGCGGAGATCGTCGAAAGGGAAGAGTGGGCGGGATTTCGCG CCAGAGAAACGGCGGCGCTGGAAGCGGTAACTGCGCCATCCACCGTTA TCGCTACAGGCGGCGGCATTATTCTGACGGAATTTAATCGTCACTTCAT GCAAAATAACGGGATCGTGGTTTATTTGTGTGCGCCAGTATCAGTCCTG GTTAACCGACTGCAAGCTGCACCGGAAGAAGATTTACGGCCAACCTTA ACGGGAAAACCGCTGAGCGAAGAAGTTCAGGAAGTGCTGGAAGAACG CGATGCGCTATATCGCGAAGTTGCGCATATTATCATCGACGCAACAAAC GAACCCAGCCAGGTGATTTCTGAAATTCGCAGCGCCCTGGCACAGACG ATCAATTGTTAA |
| SEQ ID NO: 140 >papA (RBS was underlined) | GAATTCAAAAGATCTAACTGGTAATTTGAGGAGGTAATTTATGAAGAT CCTGCTGATCGATAACTTTGATAGCTTCACCCAGAATATTGCCCAGTAT CTGTATGAAGTTACCGGTATTTGTGCCGATATTGTTACCAATACCGTGA CCTATGAACATCTGCAAATCGAACAGTATGATGCCGTTGTTCTGAGCCC TGGTCCGGGTCATCCGGGTGAATATCTGGATTTTGGTGTTTGTGGTCAG GTGATTCTGCATAGTCCGGTTCCGCTGCTGGGTATTTGTCTGGGTCATC AGGGTATTGCACAGTTTTTAGGTGGCACCGTTGGTCATGCACCGACACC GGTTCATGGTTATCGTAGCAAAATTACCCATAGCGGTAGCGGTCTGTTT CGTGATCTGCCGGAACAGTTTGAAGTTGTTCGTTATCATAGCCTGATGT GTACCCATCTGCCGCAAGAACTGCGTTGTACCGCATGGACCGAAGAGG GTGTTGTTATGGCAATTGAACATGAAAGCCGTCCGATTTGGGGTGTTCA GTTTCATCCGGAAAGCATTGATAGCGAATATGGTCATGCCCTGCTGAGC AACTTTATTGGTATGGCCATCGAACATAATGGCAATCATCGTACCAGCG CAACCCAGAATCCGGATGCAAGCGCAAGCGCCAATGAACATTATCGTG CAGTTGGTGGTCTGCTGAATATGCAGCTGGCCTATCGTACCTATCCGGG TCCGTTTGATCCGCTGGCACTGTTTACCCAGCGTTATGCACAGGATCAT |
| | CATGCATTTTGGCTGGATAGCGAAAAAAGCGAACGTCCGAATGCACGT TATAGCATTATGGGTAGTGGTCAGGCACAGGGTAGCATTCGTCTGACAT ATGATGTTAATAGCGAAAGTCTGACCCTGGCAGGTCCGAAAGGTAGCC GTATTGTGACCGGTGATTTTTTCACCCTGTTTAGCCAGATTGTTGAAAG CGTTAATGTTGCAGTTCCGCAGTATCTGCCGTTTGAGTTTAAAGGTGGT TTTGTGGGTTATATGGGCTATGAACTGAAAGCACTGACCGGTGGTAATA AAGTGTATCGTAGCGGTCAGCCGGATGCAGGTTTTATGTTTGCACCGCA TTTTTTTGTGTTCGATCATCACGATCAGACCGTGTATGAGTGCATGATTA GCGCAACCGGTCAGAGTCCGCAGTGGCCTCAGCTGCTGACCAGCATGA CCACACTGAATAATGCAACCGATCGTCGTCCGTTTGTTCCTGGTGCAGT TGATGAACTGGAACTGAGCCTGGAAGATGGTCCGGATGATTATATTCGT AAAGTTAAACAGAGCCTGCAGTATATTACCGATGGTGAAAGCTATGAA ATCTGTCTGACCAATCGTGCACGTATGAGCTATAGCGGTGAACCGCTGG CAGCATATCGTCGTATGCGTGAAGCATCACCGGTTCCGTATGGTGCATA TCTGTGTTTTGATAGTTTTAGCGTTCTGAGCGCAAGTCCGGAAACCTTTC TGCGTATTGATGAAGGTGGTCTGATTGAATCACGTCCGATTAAAGGCAC CCGTGCGCGTAGCAAAGATCCGAGCGAAGATCAGCGTCTGCGTAGCGA TCTGCAGGCAAGCACCAAAGATCGTGCAGAAAATCTGATGATTGTTGA TCTGGTTCGCCATGATCTGAATCAGGTTTGTCGTAGTGGTAGCGTTCAT GTTCCGCATATTTTTGCAGTTGAAAGCTTTAGCAGCGTTCATCAGCTGG TTAGCACCGTTCGTGGTCATCTGCGTAATGATATTAGCACCATGGAAGC AATTCGTGCCTGTTTTCCTGGTGGTAGTATGACAGGTGCACCGAAAAAA CGTACCATGGAAATTATTGATGGTCTGGAAACCTGTGCACGTGGTGTTT ATAGTGGTGCATTAGGTTGGATTAGCTTTAGCGGTAGTGCAGAACTGAG CATTGTTATTCGTACCGCAGTGCTGCATAAACAGCAGGCAGAATTTGGT ATTGGTGGTGCAATTGTTGCACATAGCGATCCGAATGAAGAGCTTGAA GAGACGCTGGTCAAAGCCAGCGTGCCTTATTACAGTTTTTACGCAGGGA GCGAAAAATGA |
| SEQ ID NO: 141 >papB (RBS was underlined) | ATAGTAATCAGTAAGGAGATAAAGAATGAACATGACCGAACATCGTCA TATGAGCCCGACCACACCGAGCGCAATTCTGCAGCCGCAGCGTGATCA GCTGGATCGTATTAACAATCATCTGGTTGATCTGCTGGGTGAACGTATG |
| | AGCGTTTGTATGGATATTGCAGAACTGAAAGCAGCACATGATATTCCG ATGATGCAGCCTCAGCGCATTGTTCAGGTTCTGGATCAGCTGAAAGATA AAAGCAGTACCGTTGGTCTGCGTCCGGATTATGTTCAGAGCGIIIIIAA ACTGATCATCGAGGAAACCTGCATCCAAGAAGAACAGCTGATTCAGCG TCGTCGTAATCAGGGTCAGCGTAGCTAA |
| SEQ ID NO: 142 >papC (RBS was underlined) | CGTAAATATAAGGAGGTCAAACATGAATACCAATACCGTTGTGGTTTTA GGTGGTGCAGGTCTGATTGGTAGCATGATTAGCCGTATTCTGAAACAGT ATGGTTATTTTGTTCGTGTGGTTGATCGTCGTCCGGCAGAATTTGAATGT GAATATCATGAAATGGACGTGACCAAACCGTTTAATGATACCGGTGCA GTTTTTCGTAATGCAACCGCAGTTGTTTTTGCACTGCCGGAAAGCGTTG CAGTTAGCGCAATTCCGTGGGTTACCACCTTTCTGAGCAGCGAAGTTGT TCTGATTCCGACCTGTAGCGTTCAGGGTCCGTTCTATAAAGCACTGAAA GCAGCAGCACCGCGTCAGCCGTTTGTTGGTGTTAATCCGATGTTTAGCC CGAAACTGAGCGTGCAGGGTCGTAGCGTTGCCGTTTGTGTTGAAGATAC CCAGGCAGCACAGACCTTTATTGAACGTCATCTGATGGAAGCCGGTAT GAAAATTCGTCGTATGACCCCGAGCGCACATGATGAACTGATGGCACT GTGTCAGGCACTGCCGCATGCAGCAATTTTAGGTTTTGGTATGGCACTG GCAAAAAGCAGCGTTGATATGGATATTGTTGCAGAAGTTATGCCTCCGC CTATGCGTACCATGATGGCCCTGCTGAGTCGTATTCTGGTTAATCCGCC TGAAGTTTATTGGGATATTCAGCTGGAAAATGATCAGGCAACCGCACA GCGTGATGCACTGGTTCATGGTCTGGAACGTCTGCAAGAAAATATTGTG GAACAGGATTATGAGCGCTTCAAAAGCGATCTGCAGAGCGTTAGCACC GCACTGGGTAAACGTCTGAATGCGGGTGCAGTTGATTGTCAGCACCTGT TTAGCCTGCTGAATTAA |
| SEQ ID NO: 143 >Rg-pal (RBS was underlined) | ATCGGGCTGCCCCAGCCAATCACACCATACCATAAGGAGCAITTTTTAT GGCGCCAAGCGTCGACTCCATCGCCACGTCCGTGGCCAACAGCTTGAG CAACGGACTGGCTGGCGATCTGCGGAAGAAAACCAGCGGCGCGGGAA GCCTGCTCCCGACAACCGAGACTACCCAGATCGACATCGTCGAGCGGA TTCTCGCCGATGCCGGTGCTACTGACCAGATCAAGTTGGATGGCTATAC CTTAACGCTCGGCGATGTGGTGGGCGCCGCGCGGCGAGGGAGGACCGT |
| | TAAAGTCGCCGACTCGCCGCAGATTCGCGAAAAGATCGATGCGTCAGT GGAGTTTCTGCGCACTCAACTGGACAACAGTGTGTACGGCGTGACCAC CGGCTTCGGCGGCTCGGCCGACACCCGCACCGAAGACGCCATTTCGCT GCAAAAGGCGCTGCTGGAGCACCAGCTGTGCGGCGTTTTGCCAACCTC GATGGACGGCTTCGCCTTGGGGCGTGGTCTGGAGAACTCGTTGCCACTG GAAGTCGTGCGGGGCGCCATGACCATCAGAGTCAATAGCCTGACCCGC GGCCATTCGGCTGTCCGTATCGTCGTCCTGGAAGCCCTGACCAACTTCT TGAACCACGGCATCACCCCGATCGTGCCGCTGCGCGGGACCATCAGCG CGTCGGGCGACTTGAGCCCGCTCAGTTACATCGCTGCGTCGATCACCGG GCATCCGGACAGCAAGGTGCATGTAGACGGGCAAATCATGAGCGCCCA AGAAGCTATTGCACTGAAAGGCCTGCAACCCGTCGTTCTTGGCCCGAA GGAAGGCTTGGGCCTGGTCAACGGCACCGCCGTGTCGGCCTCCATGGC CACGCTCGCCCTGACCGACGCCCACGTGCTCAGCCTGCTCGCGCAGGCC AATACCGCACTGACGGTGGAAGCAATGGTGGGCCATGCCGGCTCATTC CACCCGTTCCTGCATGATGTGACTCGCCCGCACCCCACCCAGATCGAGG TGGCCCGTAACATTCGCACGCTGCTGGAGGGCAGCAAGTATGCCGTAC ACCACGAAACCGAAGTAAAAGTGAAAGACGACGAGGGTATCCTTCGCC AGGACCGCTACCCGCTTCGCTGCTCGCCTCAGTGGCTGGGTCCGCTGGT GAGCGACATGATCCACGCGCACAGCGTGCTGTCACTGGAAGCAGGCCA GAGTACGACCGACAACCCGCTGATTGACCTAGAAAACAAGATGACCCA TCATGGTGGTGCCTTCATGGCCAGCAGCGTGGGTAACACCATGGAGAA GACCCGGCTGGCCGTAGCACTGATGGGCAAGGTGTCGTTCACCCAATT GACAGAAATGCTGAACGCTGGCATGAACCGCGCCCTGCCCAGCTGCCT GGCAGCCGAGGACCCGTCACTGAGTTACCACTGCAAGGGCCTTGATAT CGCCGCCGCAGCCTACACCAGCGAGCTGGGCCACCTGGCCAATCCTGT CAGCACGCATGTGCAGCCGGCCGAGATGGGTAACCAGGCAATCAATTC CCTGGCGCTCATATCTGCCCGCCGTACAGCCGAGGCCAACGACGTGCTG TCTTTGCTGCTTGCAACTCACTTGTACTGTGTACTGCAAGCCGTTGATCT GCGTGCGATGGAATTTGAACACACCAAGGAGTTCGAGCCGATGGTCAC CGATTTGCTAAAACAGCACTTCGGCGCCCTGGCCACAGCGGACGTGGA GGACAAGGTGCGCAAGAGCATCTACAAGCGCTTACAGCAGAACAATTC CTACGACCTGGAGCAACGCTGGCACGACACCTTCAGCGTAGCGACCGG |
| | TGCGGTCGTCGAGGCGCTGGCCGGCAACGAAGTGTCTCTGGCCTCCCTG AACGCCTGGAAGGTGGCGTGCGCCGAAAAAGCTATCGCCCTGACCCGT ACCGTGCGCGACAGCTTCTGGGCTGCGCCTAGCTCGGCCTCGCCGGCGC TCAAGTACCTGTCACCACGGACCCGCATCCTGTACAGCTTTGTCCGCGA AGATGTGGGCGTCAAGGCCAGGCGCGGTGATGTTTATCTCGGCAAGCA GGAAGTGACGATCGGTACGAACGTGTCCCGTATCTATGAGGCCATCAA AGACGGGCGCATTGCACCCGTGCTGGTCAAAATGATGGCATAA |
| SEQ ID NO: 144 >PspF-λN22 (RBS is underlined) | GAATTCATTAAAGAGGAGAAAGGTACCatggggcccatggcagaatacaaagataatttact tggtgaggcgaacagctttctcgaagtgctggaacaggtttcgcatctcgcaccgctggacaaaccggtgctcatcatc ggcgaacgcggcaccggtaaagagctgattgccagccgcctgcattatctctcctcccgttggcaagggccgtttattt cccttaactgcgcggcgttaaatgaaaatctgctggattccgaactgtttggtcacgaagcgggggcgtttaccggtgc gcaaaaacgtcatccagggagatttgaacgtgccgacggcggtacgctatttcttgatgaactcgctacggcaccgat gatggtgcaggagaaattattgcgcgtgattgagtacggtgaactggagcgcgttggcggcagccaaccattgcaggt gaatgtgcggttggtatgcgcgacgaatgccgatctcccggcgatggtcaatgaaggcacttttcgcgctgacctgctc gaccgactggcttttgatgttgtacaactgccaccactgcgcgagcgcgaaagcgacataatgttgatggcagaatact ttgccatccagatgtgtcgggaaatcaagctgcctctgttcccggggtttacggagcgcgccagagaaacattgctgaa ttatcgttggccgggaaatattcgtgaattgaaaaacgtggtggaacgttcagtgtatcgccacggcaccagcgattatc cgcttgatgacatcattattgatccctttaaacggcgtccgcctgaagacgctatcgccgtttcagaaaccacctcgcttc caacactgccgctggatttacgtgagtttcagatgcagcaggaaaaagagttgctgcaactcagtttgcaaATGAA TGCACGCACACGCCGCCGCGAACGTCGCGCAGAGAAACAGGCTCAATG GAAAGCAGCAAATTAA |
| SEQ ID NO: 145 Promoter >pspAp (TSS is underlined) | ataaaaaattggcacgcaaattgtattaacagttcA |
| SEQ ID NO: 146 2xBoxB scRNA (BoxB hairpins are underlined) | NNNNNNNNNNNNNNNNNNNNGTTTGAGAGCTAGGGCCCTGAAGAAGG GCCCTAGCAAGTTCAAATAAGGCTAGTCCGTTATCAACTTGGGCCCTGA AGAAGGGCCCAAGTGGCACCGAGTCGGTGCTTTTTTT |
| SEQ ID NO: 147 >Sp.pCas9-dCas9-dblTerm (Promoter and terminator sequence encoding protein is bolded) | TTACGAAATCATCCTGTGGAGCTTAGTAGGTTTAGCAAGATGGCAGCGC CTAAATGTAGAATGATAAAAGGATTAAGAGATTAATTTCCCTAAAAAT GATAAAACAAGCGTTTTGAAAGCGCTTGTTTTTTTGGTTTGCAGTCAGA GTAGAATAGAAGTATCAAAAAAAGCACCGACTCGGTGCCACTTTTTCA AGTTGATAACGGACTAGCCTTATTTTAACTTGCTATGCTGTTTTGAATG GTTCCAACAAGATTATTTTATAACTTTTATAACAAATAATCAAGGAGAA ATTCAAAGAAATTTATCAGCCATAAAACAATACTTAATACTATAGAATG ATAACAAAATAAACTACTTTTTAAAAGAATTTTGTGTTATAATCTATTT ATTATTAAGTATTGGGTAATATTTTTTGAAGAGATATTTTGAAAAAGAA AAATTAAAGCATATTAAACTAATTTCGGAGGTCATTAAAACTATTATTG AAATCATCAAACTCATTATGGATTTAATTTAAACTTTTTATTTTAGGAG GCAAAAATGGATAAGAAATACTCAATAGGCTTAGcTATCGGCACAA ATAGCGTCGGATGGGCGGTGATCACTGATGAATATAAGGTTCCGTC TAAAAAGTTCAAGGTTCTGGGAAATACAGACCGCCACAGTATCAAA AAAAATCTTATAGGGGCTCTTTTATTTGACAGTGGAGAGACAGCGG AAGCGACTCGTCTCAAACGGACAGCTCGTAGAAGGTATACACGTCG GAAGAATCGTATTTGTTATCTACAGGAGATTTTTTCAAATGAGATG GCGAAAGTAGATGATAGTTTCTTTCATCGACTTGAAGAGTCTTTTTT GGTGGAAGAAGACAAGAAGCATGAACGTCATCCTATTTTTGGAAAT ATAGTAGATGAAGTTGCT TATCATGAGAAATATCCAACTATCTATCATCTGCGAAAAAAATTGG TAGATTCTACTGATAAAGCGGATTTGCGCTTAATCTATTTGGCCTTA GCGCATATGATTAAGTTTCGTGGTCATTTTTTGATTGAGGGAGATT TAAATCCTGATAATAGTGATGTGGACAAACTATTTATCCAGTTGGT ACAAACCTACAATCAATTATTTGAAGAAAACCCTATTAACGCAAGT GGAGTAGATGCTAAAGCGATTCTTTCTGCACGATTGAGTAAATCAA GACGATTAGAAAATCTCATTGCTCAGCTCCCCGGTGAGAAGAAAAA TGGCTTATTTGGGAATCTCATTGCTTTGTCATTGGGTTTGACCCCTA ATTTTAAATCAAATTTTGATTTGGCAGAAGATGCTAAATTACAGCTT TCAAAAGATACTTACGATGATGATTTAGATAATTTATTGGCGCAAA TTGGAGATCAATATGCTGATTTGTTTTTGGCAGCTAAGAATTTATCA |
| | GATGCTATTTTACTTTCAGATATCCTAAG AGTAAATACTGAAATAACTAAGGCTCCCCTATCAGCTTCAATGATT AAACGCTACGATGAACATCATCAAGACTTGACTCTTTTAAAAGCTT TAGTTCGACAACAACTTCCAGAAAAGTATAAAGAAATCTTTTTTGAT CAATCAAAAAACGGATATGCAGGTTATATTGATGGGGGAGCTAGCC AAGAAGAATTTTATAAATTTATCAAACCAATTTTAGAAAAAATGGAT GGTACTGAGGAATTATTGGTGAAACTAAATCGTGAAGATTTGCTGC GCAAGCAACGGACCTTTGACAACGGCTCTATTCCCCATCAAATTCA CTTGGGTGAGCTGCATGCTATTTTGAGAAGACAAGAAGACTTTTAT CCATTTTTAAAAGACAATCGTGAGAAGATTGAAAAAATCTTGACTT TTCGAATTCCTTATTATGTTGGTCCATTGGCGCGTGGCAATAGTCG TTTTGCATGGATGACTCGGAAGTCTGAAGAAACAATTACCCCATGG AATTTTGAAGAAGTTGTCGATAAAGGTGCTTCAGCTCAATCATTTA TTGAACGCATGACAAACTTTGATAAAAATCTTCCAAATGAAAAAGT ACTACCAAAACATAGTTTGCTTTATGAGTATTTTACGGTTTATAACG AATTGACAAAGGTCAAATATGTTACTGAAGGAATGCGAAAACCAGC ATTTCTTTCAGGTGAACAGAAGAAAGCCATTGTTGATTTACTCTTCA AAACAAATCGAAAAGTAACCGTTAAGCAATTAAAAGAAGATTATTT CAAAAAAATAGAATGTTTTGATAGTGTTGAAATTTCAGGAGTTGAA GATAGATTTAATGCTTCATTAGGTACCTACCATGATTTGCTAAAAAT TATTAAAGATAAAGATTTTTTGGATAATGAAGAAAATGAAGATATC TTAGAGGATATTGTTTTAACATTGACCTTATTTGAAGATAGGGAGA TGATTGAGGAAAGACTTAAAACATATGCTCACCTCTTTGATGATAA GGTGATGAAACAGCTTAAACGTCGCCGTTATACTGGTTGGGGACGT TTGTCTCGAAAATTGATTAATGGTATTAGGGATAAGCAATCTGGCA AAACAATATTAGATTTTTTGAAATCAGATGGTTTTGCCAATCGCAAT TTTATGCAGCTGATCCATGATGATAGTTTGACATTTAAAGAAGACA TTCAAAAAGCACAAGTGTCTGGACAAGGCGATAGTTTACATGAACA TATTGCAAATTTAGCTGGTAGCCCTGCTATTAAAAAAGGTATTTTAC AGACTGTAAAAGTTGTTGATGAATTGGTCAAAGTAATGGGGCGGCA TAAGCCAGAAAATATCGTTATTGAAATGGCACGTGAAAATCAGACA ACTCAAAAGGGCCAGAAAAATTCGCGAGAGCGTATGAAACGAATC |
| | GAAGAAGGTATCAAAGAATTAGGAAGTCAGATTCTTAAAGAGCATC CTGTTGAAAATACTCAATTGCAAAATGAAAAGCTCTATCTCTATTAT CTCCAAAATGGAAGAGACATGTATGTGGACCAAGAATTAGATATTA ATCGTTTAAGTGATTATGATGTCGATgcCATTGTTCCACAAAGTTTC CTTAAAGACGATTCAATAGACAATAAGGTCTTAACGCGTTCTGATA AAAATCGTGGTAAATCGGATAACGTTCCAAGTGAAGAAGTAGTCAA AAAGATGAAAAACTATTGGAGACAACTTCTAAACGCCAAGTTAATC ACTCAACGTAAGTTTGATAATTTAACGAAAGCTGAACGTGGAGGTT TGAGTGAACTTGATAAAGCTGGTTTTATCAAACGCCAATTGGTTGA AACTCGCCAAATCACTAAGCATGTGGCACAAATTTTGGATAGTCGC ATGAATACTAAATACGATGAAAATGATAAACTTATTCGAGAGGTTA AAGTGATTACCTTAAAATCTAAATTAGTTTCTGACTTCCGAAAAGAT TTCCAATTCTATAAAGTACGTGAGATTAACAATTACCATCATGCCCA TGATGCGTATCTAAATGCCGTCGTTGGAACTGCTTTGATTAAGAAA TATCCAAAACTTGAATCGGAGTTTGTCTATGGTGATTATAAAGTTTA TGATGTTCGTAAAATGATTGCTAAGTCTGAGCAAGAAATAGGCAAA GCAACCGCAAAATATTTCTTTTACTCTAATATCATGAACTTCTTCAA AACAGAAATTACACTTGCAAATGGAGAGATTCGCAAACGCCCTCTA ATCGAAACTAATGGGGAAACTGGAGAAATTGTCTGGGATAAAGGG CGAGATTTTGCCACAGTGCGCAAAGTATTGTCCATGCCCCAAGTCA ATATTGTCAAGAAAACAGAAGTACAGACAGGCGGATTCTCCAAGGA GTCAATTTTACCAAAAAGAAATTCGGACAAGCTTATTGCTCGTAAA AAAGACTGGGATCCAAAAAAATATGGTGGTTTTGATAGTCCAACGG TAGCTTATTCAGTCCTAGTGGTTGCTAAGGTGGAAAAAGGGAAATC GAAGAAGTTAAAATCCGTTAAAGAGTTACTAGGGATCACAATTATG GAAAGAAGTTCCTTTGAAAAAAATCCGATTGACTTTTTAGAAGCTA AAGGATATAAGGAAGTTAAAAAAGACTTAATCATTAAACTACCTAA ATATAGTCTTTTTGAGTTAGAAAACGGTCGTAAACGGATGCTGGCT AGTGCCGGAGAATTACAAAAAGGAAATGAGCTGGCTCTGCCAAGC AAATATGTGAATTTTTTATATTTAGCTAGTCATTATGAAAAGTTGAA GGGTAGTCCAGAAGATAACGAACAAAAACAATTGTTTGTGGAGCAG CATAAGCATTATTTAGATGAGATTATTGAGCAAATCAGTGAATTTTC |
| | TAAGCGTGTTATTTTAGCAGATGCCAATTTAGATAAAGTTCTTAGT GCATATAACAAACATAGAGACAAACCAATACGTGAACAAGCAGAAA ATATTATTCATTTATTTACGTTGACGAATCTTGGAGCTCCCGCTGCT TTTAAATATTTTGATACAACAATTGATCGTAAACGATATACGTCTAC AAAAGAAGTTTTAGATGCCACTCTTATCCATCAATCCATCACTGGT CTTTATGAAACACGCATTGATTTGAGTCAGCTAGGAGGTGACTAAC TCGAGTAAGGATCTCCAGGCATCAAATAAAACGAAAGGCTCAGTCG AAAGACTGGGCCTTTCGTTTTATCTGTTGTTTGTCGGTGAACGCTC TCTACTAGAGTCACACTGGCTCACCTTCGGG TGGGCCTTTCTGCGTTTATA |
| SEQ ID NO: 148 >BBa J23107-MCP-GGGGS linker-SoxS (R93A. S101A) (Promoter is underlined. sequence encoding protein is bolded) | TTTACGGCTAGCTCAGCCCTAGGTATTATGCTAGCGAATTCATTAAAGA GGAGAAAGGTACCATGGGGCCCGCTTCTAACTTTACTCAGTTCGTTC TCGTCGACAATGGCGGAACTGGCGACGTGACTGTCGCCCCAAGCA ACTTCGCTAACGGGATCGCTGAATGGATCAGCTCTAACTCGCGTTC ACAGGCTTACAAAGTAACCTGTAGCGTTCGTCAGAGCTCTGCGCAG AATCGCAAATACACCATCAAAGTCGAGGTGCCTAAAGGCGCCTGGC GTTCGTACTTAAATATGGAACTAACCATTCCAATTTTCGCCACGAAT TCCGACTGCGAGCTTATTGTTAAGGCAATGCAAGGTCTCCTAAAAG ATGGAAACCCGATTCCCTCAGCAATCGCAGCAAACTCCGGCATCTA CGGTGGCGGAGGTAGCATGTCCCATCAGAAAATTATTCAGGATCTT ATCGCATGGATTGACGAGCATATTGACCAGCCGCTTAACATTGATG TAGTCGCAAAAAAATCAGGCTATTCAAAGTGGTACTTGCAACGAAT GTTCCGCACGGTGACGCATCAGACGCTTGGCGATTACATTCGCCAA CGCCGCCTGTTACTGGCCGCCGTTGAGTTGCGCACCACCGAGCGT CCGATTTTTGATATCGCAATGGACCTGGGTTATGTCTCGCAGCAGA CCTTCTCCCGCGTTTTCGCGCGGCAGTTTGATCGCACTCCCGCGGA TTATCGCCACCGCCTGTAAGCGGCCGCCACGCAAAAAACCCCGCTTC GGCGGGGTTTTTTCGC |
| SEQ ID NO: 149 >XylS-Pm-CDS | ATTTGTCCTACTCAGGAGAGCGTTCACCGACAAACAACAGATAAAACG AAAGGCCCAGTCTTTCGACTGAGCTTTTCGTTTTATTTGATGCCTTTAAT TAAACGTTCGTAATCAAGCCACTTCCTTTTTGCATTGACGCAGGGTGTC |
| (Promoter is underlined, sequence encoding protein is bolded) | GGAAGGCAACTCGCCGAACGCGCTCCTATAGTTTTCAGCGAAGCGTCC CAAATGTAAGAAGCCGTAGTCTAGGGCTATCTCAGTTATACTACGCACA TTGGCACTGGGATCGTTCAAGCAGGCGCGGATGCTTTCGAGCTTGCGGT TGCGGATGTAGTTCTTCGGCGTGGTGCCGGCGTGCTTCTCGAACAAATT GTAGAGCGAGCGTGGACTCATCATCGCCAGCTCCGCTAACCGCTCAAG GCTGATATTCCGTTTGAGATTCTCCTCAATGAATTGAACGACTCGCTCG AAAGACGGGTTACCTTTGCTGAAAATTTCACGGCTGACATTGCTGCCCA GCATTTCGAGCAGCTTGGAAGCGATGATCCCCGCATAGTGCTCTTGGAC CCGAGGCATCGACTTTGTATGTTCCGCTTCGTCACAAACTAACCCGAGT AGATTGATAAAGCCATCGAGTTGCTGGAGATTGTGTCGCGCGGCGAAA CGGATACCCTCCCTCGGCTTGTGCCAATTGTTGTCACTGCACGCCCGAT CAAGGACCACTGAGGGCAATTTAACGATAAATTTCTCGCAATCTTCTGA ATAGGTCAGGTCGGCTTGGTCATCCGGATTGAGCAGCAATAGTTCGCCC GGCGCAAAATAGTGCTCCTGGCCATGGCCACGCCACAGGCAATGGCCT TTGAGTATTATTTGCAGATGATAACAGGTTTCTAATCCAGGCGAGATTA CCCTCACGCTACCGCCGTAGCTGATTCGACACAGATCGAGGCATCC GAAGATTCTGTGGTGCAGCCTGCCTGCCGGGCGCCCGCCCTTGGGCAG GCGAATAGAGTGCGTACCGACATACTGGTTAACATAATCGGAGACTGC ATAGGGCTCGGCGTGGACGAAGATCTGACTTTTCTCGTTCAATAAGCAA AAATCCATAGTTCACGGTTCTCTTATTTTAATGTGGGCTGCTTGGTGTGA TGTAGAAAGGCGCCAAGTCGATGAAAATGCATCTCGACGTGATGCGTA TACGGGTTACCCCCATTGCCACGTTGCGCCATCCTTTTTGCAATCAGTG ACCACTTTTCCAAGCAAAAATAACGCCAAGCAGAACGAAGACGTTCTT TTTAAGAAGCGAGAACACCAGAAGTTCGTGCTGTCGGGGCATGGG GCGACGAATTGGCGGATAAAGGGGATCTGCTGGATATTACGGCCTTTTT AAAGACCGTAAAGAAAAATAAGCACAAGTTTTATCCGGCCTTTATTCA CATTCTTGCCCGCCTGATGAATGCTCATCCGTAATTACGTATGGCAATG AAAGACGGTGAGCTGGTGATATGGGATAGTGTTCACCCTTGTTACACCG TTTTCCATGAGCAAACTGAAACGTTTTCATCGCTCTGGAGTGAATACCA CGACGATTTCCGGCAGTTTCTACACATATATTCGCAAGATGTGGCGTGT TACGGTGAAAACCGCCTATTTCCCTAAAGGGTTTATTGAGAATATGTTT TTCGTCTCAGCCAATCCCTGGGTGAGTTTCACCAGTTTTGATTTAAACGT |
| | GGCCAATATGGACAACTTCTTCGCCCCCGTTTTCACCATGCATGGGAAT TAGCTTGATCTGACCAACGACCGGTAGCGGAGCTATCCAACGGCGGTA TACCAGGAAAACACACAGCAGGTACATCAGAACAGTACCATGACTGAA GAACAAATAGTTTTTTCCTGATCCATAAAGCAGAACGGCCTGCTCCATG ACAAATCTGGCTCCCCAACTAATGCCCCATGCAGCCAGCATAACCAGC ATAAAGTGCAGTGTCCGGTTTGATAGGGATAAGTCCAGCCTTGCAAGA AGCGGATACAGGAGTGCAAAAAATGGCTATCTCTAGTAAGGCCTACCC CTTAGGCTTTATGCAACAGAAACAATAATAATGGAGTCATGACCATGC CTAGGCCGCGGTTAGGAGGAAAAACATATG |
| SEQ ID NO: 150 >J1-BBa J23117-sfGFP (Promoter is underlined. sequence encoding protein is bolded) | GCCTACGGTATCCACCGGAGACCTATGGCAGCCTCCGGCCGCCATAGG ACACCTTTGGTTGCCAAGGGTGACCTATGGTGACCATGGGCCACCACG GGCGACCTCAGGTATCCTGCGGTGTCCTGCGGTTACCAAAGGCGTCCTT TGGGTTCCACCGGATACCTCCGGACTTGACAGCTAGCTCAGTCCTAGGG ATTGTGCTAGCGAATTCATTAAAGAGGAGAAAGGTACCATGAGCAAA GGAGAAGAACTTTTCACTGGAGTTGTCCCAATTCTTGTTGAATTAG ATGGTGATGTTAATGGGCACAAATTTTCTGTCCGTGGAGAGGGTGA AGGTGATGCTACAAACGGAAAACTCACCCTTAAATTTATTTGCACT ACTGGAAAACTACCTGTTCCGTGGCCAACACTTGTCACTACTCTGA CCTATGGTGTTCAATGCTTTTCCCGTTATCCGGATCACATGAAACG GCATGACTTTTTCAAGAGTGCCATGCCCGAAGGTTATGTACAGGAA CGCACTATATCTTTCAAAGATGACGGGACCTACAAGACGCGTGCTG AAGTCAAGTTTGAAGGTGATACCCTTGTTAATCGTATCGAGTTAAA GGGTATTGATTTTAAAGAAGATGGAAACATTCTTGGACACAAACTC GAGTACAACTTTAACTCACACAATGTATACATCACGGCAGACAAAC AAAAGAATGGAATCAAAGCTAACTTCAAAATTCGCCACAACGTTGA AGATGGTTCCGTTCAACTAGCAGACCATTATCAACAAAATACTCCA ATTGGCGATGGCCCTGTCCTTTTACCAGACAACCATTACCTGTCGA CACAATCTGTCCTTTCGAAAGATCCCAACGAAAAGCGTGACCACAT GGTCCTTCTTGAGTTTGTAACTGCTGCTGGGATTACACA TGGCATGGATGAGCTCTACAAATAA |
| SEQ ID NO: 151 >J3-BBa J23111-mRFP (Promoter is underlined, sequence encoding protein is bolded) | AGCATTTGCGATCATTCACGCAGCGCTTATTCAGTTGCTCACTGCGATGTCAT AATCATCGCTACGAGCTGTGAAAGATGCATAAAGCTCGTACGACGCGTTCGCT CGTCTCCTCACTTCTCCTACGGAGCGTTCTGGACACAACGTCGTCTTGAAGTT GCGATTATAGATTGACGGCTAGCTCAGTCCTAGGTATAGTGCTAGCGAATTCA TTAAAGAGGAGAAAGGTACCATGGCGAGTAGCGAAGACGTTATCAAAGAGTTC ATGCGTTTCAAAGTTCGTATGGAAGGTTCCGTTAACGGTCACGAGTTCGAAAT CGAAGGTGAAGGTGAAGGTCGTCCGTACGAAGGTACCCAGACCGCTAAACTGA AAGTTACCAAAGGTGGTCCGCTGCCGTTCGCTTGGGACATCCTGTCCCCGCAG TTCCAGTACGGTTCCAAAGCTTACGTTAAACACCCGGCTGACATCCCGGACTA CCTGAAACTGTCCTTCCCGGAAGGTTTCAAATGGGAACGTGTTATGAACTTCG AAGACGGTGGTGTTGTTACCGTTACCCAGGACTCCTCCCTGCAAGACGGTGAG TTCATCTACAAAGTTAAACTGCGTGGTACCAACTTCCCGTCCGACGGTCCGGT TATGCAGAAAAAAACCATGGGTTGGGAAGCTTCCACCGAACGTATGTACCCGG AAGACGGTGCTCTGAAAGGTGAAATCAAAATGCGTCTGAAACTGAAAGACGGT GGTCACTACGACGCTGAAGTTAAAACCACCTACATGGCTAAAAAACCGGTTCA GCTGCCGGGTGCTTACAAAACCGACATCAAACTGGACATCACCTCCCACAAC GAAGACTACACCATCGTTGAACAGTACGAACGTGCTGAAGGTCGTCACTCCAC CGGTGCTTAA |
| SEQ ID NO: 152 J3-BBa J23117-EcGTPCH (Promoter is underlined. sequence encoding protein is bolded) | AGCATTTGCGATCATTCACGCAGCGCTTATTCAGTTGCTCACTGCGATGTCAT AATCATCGCTACGAGCTGTGAAAGATGCATAAAGCTCGTACGACGCGTTCGCT CGTCTCCTCACTTCTCCTACGGAGCGTTCTGGACACAACGTCGTCTTGAAGTT GCGATTATAGATTGACAGCTAGCTCAGTCCTAGGGATTGTGCTAGCGAATTCA TTAAAGAGGAGAAAGGTACCATGCATCACCATCACCATCACCCATCACTCAGT AAAGAAGCGGCCCTGGTTCATGAAGCGTTAGTTGCGCGAGGACTGGAAACACC GCTGCGCCCGCCCGTGCATGAAATGGATAACGAAACGCGCAAAAGCCTTATTG CTGGTCATATGACCGAAATCATGCAGCTGCTGAATCTCGACCTGGCTGATGAC AGTTTGATGGAAACGCCGCATCGCATCGCTAAAATGTATGTCGATGAAATTTT CTCCGGTCTGGATTACGCCAATTTCCCGAAAATCACCCTCATTGAAAACAAAA TGAAGGTCGATGAAATGGTCACCGTGCGCGATATCACTCTGACCAGCACCTGT GAACACCATTTTGTTACCATCGATGGCAAAGCGACGGTGGCCTATATCCCGAA AGATTCGGTGATCGGTCTGTCAAAAATTAACCGCATTGTGCAGTTCTTTGCCC |
| | AGCGTCCGCAGGTGCAGGAACGTCTGACGCAGCAAATTCTTATTGCGCTACAA ACGCTGCTGGGCACCAATAACGTGGCTGTCTCGATCGACGCGGTGCATTACTG CGTGAAGGCGCGTGGCATCCGCGATGCAACCAGTGCCACGACAACGACCTCT CTTGGTGGATTGTTCAAATCCAGTCAGAATACGCGCCACGAGTTTCTGCGCGC TGTGCGTCATCACAACTAA |
| SEQ ID NO: 153 J3-BBa J23117-MaPTPS (Promoter is underlined, sequence encoding protein is bolded) | AGCATTTGCGATCATTCACGCAGCGCTTATTCAGTTGCTCACTGCGATGTCAT AATCATCGCTACGAGCTGTGAAAGATGCATAAAGCTCGTACGACGCGTTCGCT CGTCTCCTCACTTCTCCTACGGAGCGTTCTGGACACAACGTCGTCTTGAAGTT GCGATTATAGATTGACAGCTAGCTCAGTCCTAGGGATTGTGCTAGCGAATTCA TTAAAGAGGAGAAAGGTACCATGCATCACCACCATCACCATACGTCCTCAACT CCAGTTAGAACTGCTTACGTTACCAGGATCGAACACTTCTCCGCTGCGCACAG ATTGAACTCCGTCCACCTCTCGCCTGCTGAGAACGTCAAGCTCTTCGGTAAGT GCAACCACACTTCCGGTCACGGTCACAACTACAAGGTCGAGGTGACCATCAAG GGTCAGATCAACCCACAATCCGGCATGGTCATCAACATCACCGATCTTAAGAA GACTTTGCAAGTCGCTGTCATGGACCCTTGTGACCATAGAAACTTGGATATAG ACGTCCCATACTTCGAGTCCAGACCCTCCACTACTGAGAACCTCGCTGTCTTC TTGTGGGAGAATATCAAGAGCCACTTGCCACCTTCCGACGCGTACGATTTGTA CGAGATCAAGTTGCACGAAACCGACAAGAACGTTGTCGTTTACAGAGGTGAAT AA |
| SEQ ID NO: 154 J3-BBa J23117-MaSR (Promoter is underlined. sequence encoding start codon and termination codon are bolded) | AGCATTTGCGATCATTCACGCAGCGCTTATTCAGTTGCTCACTGCGATGTCAT AATCATCGCTACGAGCTGTGAAAGATGCATAAAGCTCGTACGACGCGTTCGCT CGTCTCCTCACTTCTCCTACGGAGCGTTCTGGACACAACGTCGTCTTGAAGTT GCGATTATAGATTGACAGCTAGCTCAGTCCTAGGGATTGTGCTAGCGAATTCA TTAAAGAGGAGAAAGGTACCATGCATCACCATCACCACCATAGCAGTAAAGAA CATCATTTGGTTATTATTAACGGTGTTAATAGAGGTTTCGGGCACTCCGTCGC GTTGGATTACATAAGACACTCAGGTGCTCACGCGGTGTCCTTTGTCTTGGTTG GTAGAACCCAGCATTCCTTGGAGCAAGTCTTAACGGAGCTGCACGAGGCTGCA TCCCACGCTGGTGTCGTCTTCAAGGGTGTCGTTGTGTCCGAGGTCGACCTGGC TCACTTGAACTCCCTCGACTCCAACCTCGCGAGGATACAGTCCGCCGCCGCTG ACCTAAGAGACGAGGCGGCGCAAAGCACCAGAACTATCACTAAGTCGGTCCTC |
| | TTCAACAACGCGGGTAGCTTGGGTGACTTGTCCAAGACTGTTAAGGAGTTCAC CTGGCAAGAGGCTCGTTCCTACCTCGATTTCAACGTCGTGTCCCTCGTTGGTT TGTGCTCCATGTTCTTGAAGGATACCCTCGAAGCATTCCCAAAGGAACAATAC CCAGATCATAGAACTGTGGTCGTGTCCATCTCTTCCCTATTAGCTGTTCAGGC TTTCCCAAACTGGGGTTTGTACGCTGCTGGTAAGGCAGCTAGAGATAGACTA TTAGGTGTTATTGCTCTCGAAGAAGCAGCTAATAACGTAAAGACCTTGAACTA CGCTCCAGGTCCATTGGATAACGAAATGCAGGCTGACGTCCGCAGAACTTTGG GTGATAAGGAACAACTGAAGATCTACGACGACATGCATAAGTCTGGTT CCTTGGTGAAGATGGAGGACTCCTCTAGAAAGTTGATTCATTTGTTAAAGGCT GACACCTTCACCTCCGGTGGCCAC ATTGATTTCTACGACGAATAA |
| SEQ ID NO: 155 > LacI-Ptrc-mvaE-mvaS (Promoter is underlined. sequence encoding protein is bolded) | CCAGCTGGCAATTCCGACGTCGACACCATCGAATGGTGCAAAACCTTTCGCGG TATGGCATGATAGCGCCCGGAAGAGAGTCAATTCAGGGTGGTGAATGTGAAAC CAGTAACGTTATACGATGTCGCAGAGTATGCCGGTGTCTCTTATCAGACCGTT TCCCGCGTGGTGAACCAGGCCAGCCACGTTTCTGCGAAAACGCGGGAAAAAGT GGAAGCGGCGATGGCGGAGCTGAATTACATTCCCAACCGCGTGGCACAACAAC TGGCGGGCAAACAGTCGTTGCTGATTGGCGTTGCCACCTCCAGTCTGGCCCTG CACGCGCCGTCGCAAATTGTCGCGGCGATTAAATCTCGCGCCGATCAACTGGG TGCCAGCGTGGTGGTGTCGATGGTAGAACGAAGCGGCGTCGAAGCCTGTAAAG CGGCGGTGCACAATCTTCTCGCGCAACGCGTCAGTGGGCTGATCATTAACTAT CCGCTGGATGACCAGGATGCCATTGCTGTGGAAGCTGCCTGCACTAATGTTCC GGCGTTATTTCTTGATGTCTCTGACCAGACACCCATCAACAGTATTATTTTCT CCCATGAAGACGGTACGCGACTGGGCGTGGAGCATCTGGTCGCATTGGGTCAC CAGCAAATCGCGCTGTTAGCGGGCCCATTAAGTTCTGTCTCGGCGCGTCTGCG TCTGGCTGGCTGGCATAAATATCTCACTCGCAATCAAATTCAGCCGATAGCGG AACGGGAAGGCGACTGGAGTGCCATGTCCGGTTTTCAACAAACCATGCAAATG CTGAATGAGGGCATCGTTCCCACTGCGATGCTGGTTGCCAACGATCAGATGG CGCTGGGCGCAATGCGCGCCATTACCGAGTCCGGGCTGCGCGTTGGTGCGGAT ATCTCGGTAGTGGGATACGACGATACCGAAGACAGCTCATGTTATATCCCGCC GTTAACCACCATCAAACAGGATTTTCGCCTGCTGGGGCAAACCAGCGTGGACC GCTTGCTGCAACTCTCTCAGGGCCAGGCGGTGAAGGGCAATCAGCTGTTGCCC GTCTCACTGGTGAAAAGAAAAACCACCCTGGCGCCCAATACGCAAACCGCCTC |
| | TCCCCGCGCGTTGGCCGATTCATTAATGCAGCTGGCACGACAGGTTTCCCGAC TGGAAAGCGGGCAGTGAGCGCAACGCAATTAATGTAAGTTAGCGCGAATTGAT CTGGTTTGACAGCTTATCATCGACTGCACGGTGCACCAATGCTTCTGGCGTCA GGCAGCCATCGGAAGCTGTGGTATGGCTGTGCAGGTCGTAAATCACTGCATAA TTCGTGTCGCTCAAGGCGCACTCCCGTTCTGGATAATGTTTTTTGCGCCGACA TCATAACGGTTCTGGCAAATATTCTGAAATGAGCTGTTGACAATTAATCATCC GGCTCGTATAATGTGTGGAATTGTGAGCGGATAACAATTTCAGAATTCAAAAG ATCTTTTAAGGACGAAACGTACATATGAAAACCGTGGTGATTATTGATGCACT GCGTACCCCGATTGGTAAATACAAAGGTAGCCTGAGCCAGGTTAGCGCAGTTG ATCTGGGCACCCATGTTACCACCCAGCTGCTGAAACGTCATAGCACCATTAGC GAAGAAATTGATCAGGTGATTTTTGGCAATGTTCTGCAGGCAGGTAATGGTC AGAATCCGGCACGTCAGATTGCAATTAATAGCGGTCTGAGCCATGAAATTCCG GCAATGACCGTTAATGAAGTTTGTGGTAGCGGTATGAAAGCAGTTATTCTGGC AAAACAGCTGATCCAGCTGGGCGAAGCCGAAGTTCTGATTGCCGGTGGTATTG AAAATATGAGCCAGGCACCGAAACTGCAGCGTTTCAATTATGAAACCGAAAGC TATGATGCACCGTTTAGCAGCATGATGTATGATGGTCTGACCGATGCATTTAG CGGTCAGGCAATGGGTCTGACAGCAGAAAATGTTGCAGAAAAATATCATGTGA CCCGTGAAGAACAGGATCAGTTTAGCGTTCATAGCCAGCTGAAAGCAGCACAG GCACAGGCCGAAGGTATTTTCGCAGATGAAATTGCACCGCTGGAAGTTAGCGG CACCCTGGTTGAAAAAGATGAAGGTATTCGTCCGAATAGCAGCGTTGAAAAAC TGGGTACACTGAAAACGGTGTTTAAAGAAGATGGCACCGTTACCGCAGGCAAT GCAAGTACCATTAATGATGGTGCAAGCGCACTGATTATTGCCAGCCAAGAATA TGCCGAAGCACATGGTCTGCCGTATCTGGCAATTATTCGTGATAGCGTTGAAG TTGGTATTGATCCGGCATATATGGGTATTAGCCCGATTAAAGCAATTCAGAAA CTGCTGGCACGTAATCAGCTGACCACCGAAGAAATCGACCTGTACGAAATTAA TGAAGCATTTGCCGCAACCAGCATTGTTGTTCAGCGTGAACTGGCACTGCCGG AAGAAAAAGTTAACATTTATGGCGGTGGCATCAGCCTGGGTCATGCAATTGG TGCAACCGGTGCACGTCTGCTGACCAGCCTGAGCTATCAGCTGAATCAGAAAG AGAAAAAATACGGCGTTGCAAGCCTGTGTATTGGTGGTGGCCTGGGTCTGGCA ATGCTGCTGGAACGCCCTCAACAGAAAAAAAACAGCCGTTTTTATCAGATGAG TCCGGAAGAACGTCTGGCCAGCCTGCTGAATGAAGGTCAGATTAGCGCAGATC CAAAAAAGAATTTGAAAACACCGCACTGAGCAGCCAGATTGCCAACCACATGA |
| | TTGAAAATCAGATCAGCGAAACCGAAGTGCCGATGGGTGTTGGTCTGCATCTG ACCGTGGATGAAACGGATTATCTGGTTCCGATGGCAACCGAAGAACCGAGCGT TATTGCAGCCCTGAGCAATGGTGCAAAAATTGCACAGGGCTTTAAAACCGTGA ATCAGCAGCGTCTGATGCGTGGTCAGATTGTTTTTTATGATGTTGCCGATGCA GAAAGCCTGATTGATGAACTGCAGGTTCGTGAAACAGAAATTTTCCAGCAGGC AGAACTGAGTTATCCGAGCATTGTTAAACGCGGTGGTGGTCTGCGTGATCTGC AGTATCGTGCATTTGATGAAAGTTTTGTTAGCGTGGATTTTCTGGTGGATGTT AAAGACGCAATGGGTGCCAATATTGTTAATGCAATGCTGGAAGGTGTTGCCGA ACTGTTTCGTGAATGGTTTGCAGAACAAAAAATCCTGTTTAGCATCCTGAGTA ACTATGCCACCGAAAGCGTTGTTACCATGAAAACAGCAATTCCGGTTAGCCGT CTGAGCAAAGGTAGTAATGGTCGTGAAATTGCCGAAAAAATTGTTCTGGCA AGCCGTTATGCCAGCCTGGATCCGTATCGTGCCGTTACCCATAATAAAGGTAT TATGAATGGCATTGAAGCAGTTGTGCTGGCCACCGGTAATGATACCCGTGCAG TTAGCGCAAGCTGTCATGCATTTGCAGTTAAAGAAGGTCGTTATCAGGGTCTG ACCAGCTGGACCCTGGATGGTGAGCAGCTGATTGGTGAAATTAGCGTTCCGCT GGCACTGGCAACCGTTGGTGGTGCCACCAAAGTTCTGCCGAAAAGCCAGGCAG CAGCCGATCTGCTGGCAGTTACCGATGCAAAAGAACTGAGCCGTGTTGTTGCA GCAGTTGGTCTGGCACAGAATCTGGCAGCACTGCGTGCACTGGTTAGCGAAGG CATTCAGAAAGGTCACATGGCACTGCAGGCACGTTCACTGGCCATGACCGTGG GTGCGACCGGTAAAGAAGTTGAAGCCGTTGCACAGCAACTGAAACGCCAGAAA ACAATGAATCAGGATCGTGCCCTGGCAATTCTGAATGATCTGCGTAAACAGTA ATGATTAGCGACAAAATATGAGGAGTGCAAAAAATGACCATTGGCATCGACAA AATCAGCTTTTTTGTTCCGCCTTACTATATCGACATGACCGCACTGGCCGAAG CACGTAATGTTGATCCGGGTAAATTTCATATTGGTATTGGTCAGGATCAGATG GCCGTTAATCCGATTAGCCAGGATATTGTTACCTTTGCAGCAAATGCAGCAGA AGCAATTCTGACCAAAGAAGATAAAGAAGCCATCGATATGGTTATTGTTGGCA CCGAAAGCAGCATTGATGAAAGCAAAGCAGCCGCAGTTGTTCTGCATCGTCT GATGGGTATTCAGCCGTTTGCACGTAGCTTTGAAATTAAAGAAGGTTGTTACG GCGCAACCGCAGGTCTGCAGCTGGCAAAAAATCATGTTGCACTGCATCCGGAT AAAAAAGTTCTGGTTGTTGCAGCAGATATCGCCAAATATGGTCTGAATAGCGG TGGTGAACCGACCCAGGGTGCCGGTGCAGTTGCAATGCTGGTTGCAAGCGAAC CGCGTATTCTGGCACTGAAAGAGGATAATGTTATGCTGACGCAGGATATCTAT |
| | GATTTTTGGCGTCCGACCGGTCATCCGTATCCGATGGTTGATGGTCCGCTGAG CAATGAAACCTATATTCAGAGCTTTGCACAGGTGTGGGATGAACATAAAAAAC GTACCGGTCTGGATTTCGCAGATTATGATGCACTGGCCTTTCATATTCCGTAT ACCAAAATGGGTAAAAAAGCACTGCTGGCGAAAATTAGCGATCAGACCGAAGC CGAACAAGAACGTATCCTGGCACGTTATGAAGAAAGCATTATCTATAGCCGTC GTGTGGGTAATCTGTATACCGGTAGCCTGTATCTGGGTCTGATTAGCCTGCTG GAAAATGCAACCACCCTGACCGCTGGTAATCAGATTGGTCTGTTTAGCTATGG TAGCGGTGCCGTTGCAGAATTCTTTACCGGTGAACTGGTTGCAGGTTATCAGA ATCATCTGCAGAAAGAAACCCATCTGGCCCTGCTGGATAATCGTACCGAACTG AGCATTGCAGAATATGAAGCAATGTTTGCAGAAACCCTGGATACCGATATTGA TCAGACCCTGGAAGACGAATTAAAATATAGCATTAGCGCCATTAATAACACC GTGCGTAGCTATCGTAATTAA |
| SEQ ID NO: 156 >J3-BBa J23117-mvaE-mvaS (Promoter is underlined. sequence encoding protein is bolded) | AGCATTTGCGATCATTCACGCAGCGCTTATTCAGTTGCTCACTGCGATGTCAT AATCATCGCTACGAGCTGTGAAAGATGCATAAAGCTCGTACGACGCGTTCGCT CGTCTCCTCACTTCTCCTACGGAGCGTTCTGGACACAACGTCGTCTTGAAGTT GCGATTATAGATTGACGGCTAGCTCAGTCCTAGGTATAGTGCTAGCGAATTCA TTAAAGAGGAGAAAGGTACCATGAAAACCGTGGTGATTATTGATGCACTGCGT ACCCCGATTGGTAAATACAAAGGTAGCCTGAGCCAGGTTAGCGCAGTTGATCT GGGCACCCATGTTACCACCCAGCTGCTGAAACGTCATAGCACCATTAGCGAAG AAATTGATCAGGTGATTTTTGGCAATGTTCTGCAGGCAGGTAATGGTCAGAAT CCGGCACGTCAGATTGCAATTAATAGCGGTCTGAGCCATGAAATTCCGGCAAT GACCGTTAATGAAGTTTGTGGTAGCGGTATGAAAGCAGTTATTCTGGCAAAAC AGCTGATCCAGCTGGGCGAAGCCGAAGTTCTGATTGCCGGTGGTATTGAAAAT ATGAGCCAGGCACCGAAACTGCAGCGTTTCAATTATGAAACCGAAAGCTATGA TGCACCGTTTAGCAGCATGATGTATGATGGTCTGACCGATGCATTTAGCGGTC AGGCAATGGGTCTGACAGCAGAAAATGTTGCAGAAAAATATCATGTGACCCGT GAAGAACAGGATCAGTTTAGCGTTCATAGCCAGCTGAAAGCAGCACAGGCACA GGCCGAAGGTATTTTCGCAGATGAAATTGCACCGCTGGAAGTTAGCGGCACC CTGGTTGAAAAAGATGAAGGTATTCGTCCGAATAGCAGCGTTGAAAAACTGGG TACACTGAAAACGGTGTTTAAAGAAGATGGCACCGTTACCGCAGGCAATGCAA GTACCATTAATGATGGTGCAAGCGCACTGATTATTGCCAGCCAAGAATATGCC |
| | GAAGCACATGGTCTGCCGTATCTGGCAATTATTCGTGATAGCGTTGAAGTTGG TATTGATCCGGCATATATGGGTATTAGCCCGATTAAAGCAATTCAGAAACTGC TGGCACGTAATCAGCTGACCACCGAAGAAATCGACCTGTACGAAATTAATGAA GCATTTGCCGCAACCAGCATTGTTGTTCAGCGTGAACTGGCACTGCCGGAAGA AAAAGTTAACATTTATGGCGGTGGCATCAGCCTGGGTCATGCAATTGGTGCAA CCGGTGCACGTCTGCTGACCAGCCTGAGCTATCAGCTGAATCAGAAAGAGAAA AAATACGGCGTTGCAAGCCTGTGTATTGGTGGTGGCCTGGGTCTGGCAATGCT GCTGGAACGCCCTCAACAGAAAAAAAACAGCCGTTTTTATCAGATGAGTCCGG AAGAACGTCTGGCCAGCCTGCTGAATGAAGGTCAGATTAGCGCAGATACCAAA AAAGAATTTGAAAACACCGCACTGAGCAGCCAGATTGCCAACCACATGATTGA AAATCAGATCAGCGAAACCGAAGTGCCGATGGGTGTTGGTCTGCATCTGACCG TGGATGAAACGGATTATCTGGTTCCGATGGCAACCGAAGAACCGAGCGTTATT GCAGCCCTGAGCAATGGTGCAAAAATTGCACAGGGCTTTAAAACCGTGAATC AGCAGCGTCTGATGCGTGGTCAGATTGTTTTTTATGATGTTGCCGATGCAGAA AGCCTGATTGATGAACTGCAGGTTCGTGAAACAGAAATTTTCCAGCAGGCAGA ACTGAGTTATCCGAGCATTGTTAAACGCGGTGGTGGTCTGCGTGATCTGCAGT ATCGTGCATTTGATGAAAGTTTTGTTAGCGTGGATTTTCTGGTGGATGTTAAA GACGCAATGGGTGCCAATATTGTTAATGCAATGCTGGAAGGTGTTGCCGAACT GTTTCGTGAATGGTTTGCAGAACAAAAAATCCTGTTTAGCATCCTGAGTAACT ATGCCACCGAAAGCGTTGTTACCATGAAAACAGCAATTCCGGTTAGCCGTCTG AGCAAAGGTAGTAATGGTCGTGAAATTGCCGAAAAAATTGTTCTGGCAAGCCG TTATGCCAGCCTGGATCCGTATCGTGCCGTTACCCATAATAAAGGTATTATGA ATGGCATTGAAGCAGTTGTGCTGGCCACCGGTAATGATACCCGTGCAGTTAGC GCAAGCTGTCATGCATTTGCAGTTAAAGAAGGTCGTTATCAGGGTCTGACCAG CTGGACCCTGGATGGTGAGCAGCTGATTGGTGAAATTAGCGTTCCGCTGGCAC TGGCAACCGTTGGTGGTGCCACCAAAGTTCTGCCGAAAAGCCAGGCAGCAGCC GATCTGCTGGCAGTTACCGATGCAAAAGAACTGAGCCGTGTTGTTGCAGCAGT TGGTCTGGCACAGAATCTGGCAGCACTGCGTGCACTGGTTAGCGAAGGCATTC AGAAAGGTCACATGGCACTGCAGGCACGTTCACTGGCCATGACCGTGGGTGCG ACCGGTAAAGAAGTTGAAGCCGTTGCACAGCAACTGAAACGCCAGAAAACAAT GAATCAGGATCGTGCCCTGGCAATTCTGAATGATCTGCGTAAACAGTAATGAT AGCGACAAAATATGAGGAGTGCAAAAAATGACCATTGGCATCGACAAAATCAG |
| | CTTTTTTGTTCCGCCTTACTATATCGACATGACCGCACTGGCCGAAGCACGTA ATGTTGATCCGGGTAAATTTCATATTGGTATTGGTCAGGATCAGATGGCCGTT AATCCGATTAGCCAGGATATTGTTACCTTTGCAGCAAATGCAGCAGAAGCAAT TCTGACCAAAGAAGATAAAGAAGCCATCGATATGGTTATTGTTGGCACCGAAA GCAGCATTGATGAAAGCAAAGCAGCCGCAGTTGTTCTGCATCGTCTGATGGGT ATTCAGCCGTTTGCACGTAGCTTTGAAATTAAAGAAGGTTGTTACGGCGCAAC CGCAGGTCTGCAGCTGGCAAAAAATCATGTTGCACTGCATCCGGATAAAAAAG TTCTGGTTGTTGCAGCAGATATCGCCAAATATGGTCTGAATAGCGGTGGTGAA CCGACCCAGGGTGCCGGTGCAGTTGCAATGCTGGTTGCAAGCGAACCGCGTAT TCTGGCACTGAAAGAGGATAATGTTATGCTGACGCAGGATATCTATGATTTTT GGCGTCCGACCGGTCATCCGTATCCGATGGTTGATGGTCCGCTGAGCAATGAA ACCTATATTCAGAGCTTTGCACAGGTGTGGGATGAACATAAAAAACGTACCGG TCTGGATTTCGCAGATTATGATGCACTGGCCTTTCATATTCCGTATACCAAAA TGGGTAAAAAAGCACTGCTGGCGAAAATTAGCGATCAGACCGAAGCCGAACAA GAACGTATCCTGGCACGTTATGAAGAAAGCATTATCTATAGCCGTCGTGTGGG TAATCTGTATACCGGTAGCCTGTATCTGGGTCTGATTAGCCTGCTGGAAAATG CAACCACCCTGACCGCTGGTAATCAGATTGGTCTGTTTAGCTATGGTAGCGGT GCCGTTGCAGAATTCTTTACCGGTGAACTGGTTGCAGGTTATCAGAATCATCT GCAGAAAGAAACCCATCTGGCCCTGCTGGATAATCGTACCGAACTGAGCATTG CAGAATATGAAGCAATGTTTGCAGAAACCCTGGATACCGATATTGATCAGACC CTGGAAGACGAATTAAAATATAGCATTAGCGCCATTAATAACACCGTGCGTAG CTATCGTAATTAA |
| SEQ ID NO: 157 >dblTerm | TAAAAACTCGAGTAAGGATCTCCAGGCATCAAATAAAACGAAAGGCTCAGTCGAA AGACTGGGCCTTTCGTTTTATCTGTTGTTTGTCGGTGAACGCTCTCTACTAGAGT CACACTGGCTCACCTTCGGGTGGGCCTTTCTGCGTTTATA |
| SEQ ID NO: 158 *>PP 1776-A-mRFP (Promoter is underlined. sequence encoding protein is bolded) | GTTCTCAGGGCTCGCCGAGAAACGCATAACCCATGCTTTGAGGTAATTATTCCTG AATAAAGCGGGTTGGCCATTGAACGTTCACGCGCGCAGTTGTCTCAAACCTGCCA TTTGAGTTTCGCCGCCCGACGGTGCAGTTGCTAAAACGGCGGTTGAACAGCCGAC TGAAGATGCGCTCTCTGGCGCTCCTCGGGGGACAAGCTACATGAAAAAAACTCTG GTATTCTACGAGCCTCAGCTCGGGTCTGGAGCCTGGATGGCCAGGAATTGCCTCG CTGGGCGCGTATATTTATTGCTGCTGCACCCGACCGCAGCGGCTGTCAGATATTA |
| | AGATACATGCAGGTTTCCTGAGGTTTGAAACTTCAAGTGGCCGTTAAGGACTCAA ATATGGAATTGATCCCGGTAATTTTATCCGGTGGCGTTGGTAGCCGTCTGTGGCC AGTATCAGAATTCATTAAAGAGGAGAAAGGTACCATGGCGAGTAGCGAAGACGTT ATCAAAGAGTTCATGCGTTTCAAAGTTCGTATGGAAGGTTCCGTTAACGGTCACG AGTTCGAAATCGAAGGTGAAGGTGAAGGTCGTCCGTACGAAGGTACCCAGACCGC TAAACTGAAAGTTACCAAAGGTGGTCCGCTGCCGTTCGCTTGGGACATCCTGTCC CCGCAGTTCCAGTACGGTTCCAAAGCTTACGTTAAACACCCGGCTGACATCCCGG ACTACCTGAAACTGTCCTTCCCGGAAGGTTTCAAATGGGAACGTGTTATGAACTT CGAAGACGGTGGTGTTGTTACCGTTACCCAGGACTCCTCCCTGCAAGACGGTGAG TTCATCTACAAAGTTAAACTGCGTGGTACCAACTTCCCGTCCGACGGTCCGGTTA TGCAGAAAAAAACCATGGGTTGGGAAGCTTCCACCGAACGTATGTACCCGGAAGA CGGTGCTCTGAAAGGTGAAATCAAAATGCGTCTGAAACTGAAAGACGGTGGTCAC TACGACGCTGAAGTTAAAACCACCTACATGGCTAAAAAACCGGTTCAGCTGCCGG GTGCTTACAAAACCGACATCAAACTGGACATCACCTCCCACAACGAAGACTACAC CATCGTTGAACAGTACGAACGTGCTGAAGGTCGTCACTCCACCGGTGCTTAA |
| SEQ ID NO: 159 *>PP 4812-B (Promoter is underlined. sequence encoding start codon bolded) | GGAAGCCTCACGGGCAGCGCGACCCAAACGGGTCATATAGTCAAGAACGGACT CAGTCATGGGTTCGGTGTCTTGGCGAAGGGGAAATCGGCTGATTATAACTGCC GCGCAGGTGTACGCCCAGCGGCGGGTGGCGGATGGTAGAAAATGGATGGGGCA ATGTGTAGGAAAGATGTAACCGGGGTTATCGAGATTTCCATCTCCTGTCGCGG CCCTTTAGCGGGCGCGCCCGCTCCCACTGGGATTTGTGTAGGAGCGGGTTTAC CCGCGAAAGGGCCGGCACTGCCAACATCACCGTCCAATTCAGCCTCGATTAAG CCATTCATTGCTATCATCCCCGCCTCTCCAGCCACGAACTGCCCCGCATGCCA GCCCTGCCCGACAGCTTTTTCGACCGCGACGCCCAGACCCTGGCCAAGGCCCT GGAATTCATTAAAGAGGAGAAAGGTACCATG |
| SEQ ID NO: 160 *>PP 3839-C (Promoter is underlined. sequence encoding start codon is bolded) | AAATTCGTGGTCAAGCAGGTGATCGTGCTCGGGTTCGTGCAGGTCGTCTTCGTCG CGCATGCTGGCTCCTGGGATAATGGCAGGCCTGTATAGGCTGATCAGGGTGCCGG GTCAATGCGTGGTCGCTTAATCCTGGGTTAACCGGACCGGCGCAACCTGCAGGCT CTCCCTTCAAACGTCTTTTGCCCGCCTTCCATGGCGGGCTTTTTTATGACCCTGC GCACTTCGTTGCAGATGATGACAGCCTCATGACCTGACCTTCACGAAATGTCGAC ATCCGGATGGGCACACTGGCCCTGCTCCGTATGTTCTTGCAGCCCGCCGCATCCT TGCCGCGGGCTTTTCTTTTTTCCGCAAAGGCCAGCCAGGCATACGCAGGAATTTT GTGGAAGCGCCCACCTTGACCATGACCTGAAGCAGTTTTGCCTGGGCCGGCAAGG |
| | TCTATGCTATCCCGACGATCACCCAAGCTCACATCGGATAGACACGGAGGCTCTC ATGAAAGCTGCTGTCGTTGCACCAGGCCGTCGCGTGGACGTGATAGAGAAAAGCC TGCGCGAATTCATTAAAGAGGAGAAAGGTACCATG |
| SEQ ID NO: 161 *>PP 1992-D (Promoter is underlined. sequence encoding start codon is bolded) | GTACATGCTCATGTTTCGACGGATCGGGTCATCAAAGCCGCCAAAACCAAAAGAC CTTGAAAGGGATGGCCCTTACGGCGAGTCACTTTTTGTCAAACGCGACAAAAAGT AACCAAAAAACGCTGCGCTCCCATCATCCGGCCCCTGCGCTGCGCTCCGGGGTCC CCTCACTCCGGCCTTGCTCCCGGCAGGACCGCGCCGAAGGCCCCATCCTGGGGCC TCAGCGCTTGACGGGCATCCATGCCCGTCACCTGCCTCCGCAAGGCCTGCGTTCG GCCTCCTGAAGTCGCGAAGATCAAGATCAAGATCAAGATCAAGATCAAGATCAAG ATCAACAGCAACAGCAACAGCAATAGCTACAGCAATAGGCAACGCTGGGGGAGGC ACAGACAAAACCCTACAATCCGAGTAAAGTGCCGCCCCCGCCGTTTACAGCAAAA GGATCCCTTCCATGACACACCCTTTGGATATCGCCGTCGTCGGCGCCACCGGCAG CGTCGGTGAAGCCTTGGAATTCATTAAAGAGGAGAAAGGTACCATG |
| SEQ ID NO: 162 *>PP 0786-E (Promoter is underlined. sequence encoding start codon for protein is bolded) | CAGCACCAGTTTCAGGCCTTCGCGCAGGTATTGGGGGCCAGAGAGGACAGGGGCT TGCATCTAGAGACTCCGCAGAGAAGGAAAACGCGCTGACCTTACCGAGTTTGCCG CGCCGACGAAAGGCACGCCGTGGGCGGGAAACGGGATGTAACAAAAGCGCCCGGG GCTTGTTTATCGATGAAATCGCAGCATAGGCGATGCCTATGAGGTGGATTGTCTA AGGATATTTCCTTAATCTTTGCGACCTCGCTACAGTGCGCTCAACTTTTCGCTTT GCGGGCCTGCGCGTTTTAGCCTTCCCCAAGTGCTGCGTGGGTCCTTTTTAATTTC TTGGCTGGCGCAGCCGGTACACCGATGCCGGCCCTGCGGCCCGCTCGACAGGAGT TCGACATGTCTGAAGTACGTCATTCGCGCGTCATCATTCTCGGTTCCGGCCCTGC CGGTTACAGCGAATTCATTAAA GAGGAGAAAGGTACCATG |
| SEQ ID NO: 163 *>PP 1972-F (Promoter is underlined. sequence encoding start codon for protein is bolded) | GATAGCCTCGGGCTGGTCGCCAGCCGGCTGGAAACGTGTGACGAGCTGGAACTCG GACATGAAGGACCTCGCGGTGCAGCAGCTGTAAATTTAGCCAGTAGTCTATACCC AAATGCGCCCGTTCGGGGGCGCTTTCAAGGCACAGGTGTGGGCGCCTGCAACGGT GGACACGGCATTTAGACCAATGGTCGAAAAATATTTGCGCAAATAGCCCCAAAAG CTGCGCCAGAGTGTCGCGGTGACCGGTCGGTATCACTATACTGACTCCCCGTTTG TGCACCGCTTCAGTGCATTCGGCTGGAGCGTGTACGCCCTATCACACTCCAATCA GAGCCAAGGTAACAATGAGCCTGTTTTCCGCTGTCGAGCTGGCACCCCGCGACCC TATTCTGGGCCTCAACGAAGAATTCATTAAAGAGGAGAAAGGTACCATG |
| SEQ ID NO: 164 *>PP 3668-G | CAGGGCTTCGAACAGCACCAGCAGGTTCATGTCGACGCGGCGCAGGTCGTTGCGG TTCATGAGGGCTCGGCGTCCTGGGCAAAGGGTATGGCTGTGTATTGAAGCATGGC |
| (Promoter is underlined, sequence encoding start codon for protein is bolded) | GCGGGGCCTTGTGCCTTGTATGGATGTGCCGGCCTCATCGCGGGCTTGACCGCGA TGAGGCCGATACAGGCCTGCGCCTGACAGAGCCAGCCTATCAGGCTCCAACAGCC ACTCTATTAGACCTCTGCCCAAGCTCGGCTAGTCTTTCTCGTGGCCCCGCGAATT CCGCGACAGGGCAGCGCGTCAGCACCTGCGTGCAAGACCGTGCCCCCTCGCCGTG ACAGCTTCGCAAGCCCAGTGTACACCTGATGAGGGGTAGTACGAGCCCACCCGCT CGGCTGAAAGAACACTGGCATAGACCGGAAATCTGGATAACCGACCCAAAGGTAC CCGCAGATGTCGAACGAATCGAAATGCCCGTTCCATCAAACCGCAGGTGGCGGCA CCACCAACCGTGAATTCATTAAAGAGGAGAAAGGTACCATG |
| SEQ ID NO: 165 *>PP 5046-H (Promoter is underlined. sequence encoding start codon for protein is bolded) | CTTGCGCACCGGCCGGCTTTTGATGGTGATTTCTGGGAAGACTTTGACGATAAGT TTCATTGGTTAACAGCGCGCGCAGGGCCTGCCGAAAATGAGGGGCGCGAATTATA TCGGAAATTGCTCAGGATTTGACCAACTTTTGATCAGAAGCTTTGAGATAAATGC AGAGGCCAGGTTTTGCAGCGCCTGTTCCGGCCCTTTCGCGGGTAAACCCGCGCCT ACAGGCGGTGCAAAGCCCGTAGGAGCGGGTTTACCCGCGAAGAGGCCCTAGAACC TGGCACACCACTCACGCCACGCACCCTATTGGTGCGACACGATCAAAAAACGCAT CGTAAGGGTGCACTTTCACCCGCTAACCCAACGCCAAATGCACGCAAACGCCCCC TTTTATCCCACCCTCGCCATTTTCGGGCACTGGCATGCAATTTGCTCCCTTGTGA GGCAGGTAAGCTTGGCCGACTATCCGCGCCCGGCAACACCCTTTTTCCAGGGCAG CGGCCCACCGCGCTCTAGACCATCCGGAGGACAACATGTCGAAGTCGGTTCAACT CATCAAAGATCATGACGTCAAGTGGATTGATCTGCGTTTCGAATTCATTAAAGAG GAGAAAGGTACCATG |
| SEQ ID NO: 166 *>PP 1231-I (Promoter is underlined. sequence encoding start codon for protein is bolded) | GTTGAAGAATTTGCCATGTTCATGGCGGCGCATCTGGGGGCCCTGTCGCCGCAGG GGTGATGGTTTCTCCTGTTGCGGCCTCTTCGCGGGTGAACCCGCTCCTACGAGGA TTTCACAGTGTAGGGGCGGGTTCACCCGCGAAGGGGCCCGCACAAGCACTACAAA AACCCCTTTAGTAATCGCTGGATTGTCTGTAGCCTTCGGCCTCCGATAATAATCC GCGCCCGCAGAGGGCCGTGGCGGTCAACATGCCGTCCGGCACCCTTAGCCGATCT GCCAGGGCCGGGTGATACACTCGATTTGACGCGCAAGCGCGCCTGCAGGTCCAGC GATCATGACCCAGATTTCCGAACGCCTTTTGGTTCAGGCCCACCTCGACGCCAAG CAGCCCAACGAATTCATTAAAGAGGAGAAAGGTACCATG |
| SEQ ID NO: 167 *>PP 4701-J (Promoter is underlined. sequence | CTGGGTAACACGCGGTTGATCGACAACCTCTACCTGCATTTGGAAGAGAAGACCG CATAACGGTCTTGGGCTGCCTTGCAGCCCATTCGCGGGCAAGCCCGCTCCTGCAC GTTTACCTGTAGGGGCGGGCTTGCTTGCAATGCCCCCAAAATCC CCCTGCCATACCCATTCCCAGCACGTGGCCTTTGCCTATAATGGTGCCAGCCTGA |
| encoding start codon for protein is bolded) | ACCCGGCAACGACTGCCGTGTCCAAGCCCTCACCACGCACCAAGGGAACCCCGCG CAATGGCGTATTACCGTACACCCCACGATGTGACGGCCCTGCCCGCCTGGCAGGC GCTTCAGGAATTCATTAAAGAGGAGAAAGGTACCATG |
| SEQ ID NO: 168 >pBBR1-MCS | GTCGAATTTGCTTTCGAATTTCTGCCATTCATCCGCTTATTATCACTTATTCAGG CGTAGCAACCAGGCGTTTAAGGGCACCAATAACTGCCTTAAAAAAATTACGCCCC GCCCTGCCACTCATCGCAGTACGGCCTATTGGTTAAAAAATGAGCTGATTTAACA AAAATTTAACGCGAATTTTAACAAAATATTAACGCTTACAATTTCCATTCGCCAT TCAGGCTGCGCAACTGTTGGGAAGGGCGATCGGTGCGGGCCTCTTCGCTATTACG CCAGCTGGCGAAAGGGGGATGTGCTGCAAGGCGATTAAGTTGGGTAACGCCAGGG TTTTCCCAGTCACGACGTTGTAAAACGACGGCCAGTGAGCGCGCGTAATACGACT CACTATAGGGCGAATTGGAGCTCCACCGCGGTGGCGGCCGCTCTAGAACTAGTGG ATCCCCCGGGCTGCAGGAATTCGATATCAAGCTTATCGATACCGTCGACCTCGAG GGGGGGCCCGGTACCCAGCTTTTGTTCCCTTTAGTGAGGGTTAATTGCGCGCTTG GCGTAATCATGGTCATAGCTGTTTCCTGTGTGAAATTGTTATCCGCTCACAATTC CACACAACATACGAGCCGGAAGCATAAAGTGTAAAGCCTGGGGTGCCTAATGAGT GAGCTAACTCACATTAATTGCGTTGCGCTCACTGCCCGCTTTCCAGTCGGGAAAC CTGTCGTGCCAGCTGCATTAATGAATCGGCCAACGCGCGGGGAGAGGCGGTTTGC GTATTGGGCGCATGCATAAAAACTGTTGTAATTCATTAAGCATTCTGCCGACATG GAAGCCATCACAAACGGCATGATGAACCTGAATCGCCAGCGGCATCAGCACCTTG TCGCCTTGCGTATAATATTTGC |
| SEQ ID NO: 169 >pp1 HR1-J3-BBa J23117-mRFP1-dblT-pp1 HR2 (Promoter and terminator are underlined, sequence encoding protein is bolded) | ATGACCGACCTGATCGAAGTGAAGACGGCAGACCTGGTGGGCGAGGCGCTTGGGT GGGCCGTGGGCACGGCGGAAGGCCTGGACCTGTTCATGGCGCCGCCGGAGTACGG CAACCCACACCGAGTGTTCGCCCGCTACCAGGGCCAGGCCATCGAGCACACCAAG CGCTTCAACCCGTGGGAAGACTGGGCGGTTGGCGGGCCGATCATGCAGAAGCACA ACGTCAGCCTGCACTGCCCGCAGCCAGAGTGGGACTACTGGGCAGCCTGGATAAC CGATAACGGCAAGGACGTCGCCCAGGGCGCTGATCTGCCGTTGCCGGCGGCGTGC CGGGCCATAGTCGCCCACCAGCTCGGCGATACCGTCCAGGTGCCGAAGGAGCTGA TGCCATGACCGTGATCCTTCCCCTCGCCTACATGGCCTACCTGATCTACAGGGGG CTTCTCGGTGAGGGAGGCGCCTGCAAGCAAAGGGCACGACATGACCTGACGACAG CACGGCAAAAAACAAACTCGAAAGGATCATCCACAAGATCAAGCGCTGCCTGGCG CTATTCAAAAGCTCGAATGAATATGAGAGAGTCTAGGCCCACCCGCCGATTACGA AGGTCTTCGCTCGGAGCACACCCCAGACCAAGGCTCGACTCATAGTTTCGCTTGG |
| | TCTGGTGCTGTAAGCCTCTTCTACAATTCGGTCCCCGCTTTTGGAGTACACCCCG ATGAAGAGCTGCGTTTCGCCTGTCCGCGAAAGACGGGTTTGCACGTCGATACTCC TGCCGTCCTCAAGGATTTCGTCGTGATGACGAAGGTGAAGCGCTGGGTCTGCCCA GGTCCAGAATTTTTCGCCGCGACATCTCATATCAATCTCCTCTTACTTATCCCAG TAGGCGCGGTAAAGAGAGGGATAGATATCCATTTCGCTTAAATGCGACCGGTGGA AAATGATCGGCCCTAATCCTTGCTGATAGATATCAGCGGGACAGCGCCAGTAGAG AACCGAGCCCAGCATGGCAATTCCGACGTCAGCATTTGCGATCATTCACGCAGCG CTTATTCAGTTGCTCACTGCGATGTCATAATCATCGCTACGAGCTGTGAAAGATG CATAAAGCTCGTACGACGCGTTCGCTCGTCTCCTCACTTCTCCTACGGAGCGTTC TGGACACAACGTCGTCTTGAAGTTGCGATTATAGATTGACAGCTAGCTCAGTCCT AGGGATTGTGCTAGCGAATTCATTAAAGAGGAGAAAGGTACCATGGCGAGTAGCG AAGACGTTATCAAAGAGTTCATGCGTTTCAAAGTTCGTATGGAAGGTTCCGTTAA CGGTCACGAGTTCGAAATCGAAGGTGAAGGTGAAGGTCGTCCGTACGAAGGTACC CAGACCGCTAAACTGAAAGTTACCAAAGGTGGTCCGCTGCCGTTCGCTTGGGACA TCCTGTCCCCGCAGTTCCAGTACGGTTCCAAAGCTTACGTTAAACACCCGGCTGA CATCCCGGACTACCTGAAACTGTCCTTCCCGGAAGGTTTCAAATGGGAACGTGTT ATGAACTTCGAAGACGGTGGTGTTGTTACCGTTACCCAGGACTCCTCCCTGCAAG ACGGTGAGTTCATCTACAAAGTAAACTGCGTGGTACCAACTTCCCGTCCGACGGT CCGGTTATGCAGAAAAAAACCATGGGTTGGGAAGCTTCCACCGAACGTATGTACC CGGAAGACGGTGCTCTGAAAGGTGAAATCAAAATGCGTCTGAAACTGAAAGACGG TGGTCACTACGACGCTGAAGTTAAAACCACCTACATGGCTAAAAAACCGGTTCAG CTGCCGGGTGCTTACAAAACCGACATCAAACTGGACATCACCTCCCACAACGAAG ACTACACCATCGTTGAACAGTACGAACGTGCTGAAGGTCGTCACTCCACCGGTG CTTAAAAACTCGAGTAAGGATCTCCAGGCATCAAATAAAACGAAAGGCTCAGTCG AAAGACTGGGCCTTTCGTTTTATCTGTTGTTTGTCGGTGAACGCTCTCTACTAGA GTCACACTGGCTCACCTTCGGGTGGGCCTTTCTGCGTTTATATCCGCCATTACGA TCTGACTTGCCACTTTGGCCGTTATCCCTATCTCTGGTCATACGCACCTCTCAAT GCTCAGAAGTCCTGAGCTAAATTGAGTGTGCGCCTACAGAGACTAGAGAAGATTT ATATCAGACCGACGGGTACAAACGAAAAAATCAATAACCGGCTCCTGTCAGCACC TCGCAGAGAAAACAGATCGCAGATATTCAGCATGCGTAGCTGATGCTCGACCGAG ATCGCACCTGACATGCACCGCTGCGCGGCACATCACACTGAGTAGAGAGCGTTCC GCGACGCCGATGGCGTGAAGCGCGAGCTGGAAAACGTCGTGCCGTTCAAGGGCGC |
| | CTAACCCCTCCCCATACAACTCAAGCCCGCCGACATGCGCGGGCGAGGATTACCT ATGTCCAATTTCCTGACTCGCTGGCTCAAGCGCAAGAAGAAGCCCGGGCCGCGTC CAACACTGGCACCAACTGGATTTGCTCGCGGCCACAGCCCGGCAGTCGGCAGGCT AGATCCGATGCTTGATCCGCTCAACCCGTTGAGCCCCGTCAGCCCGTTGCATCCC GCCTACCAGGCCGACAGCTACGAACCACCGCGCAGCACCAGCAGTTCCTGCTCCA GCCGTGATTACAGCAGCTACGACTGCGGCAGCAGCTACTCGTCGAGCGACAGCAG CAGTTCCAGCGATAGCGGATCCAGCTCCAGCAGCTGCGACTGACCACCAACCTGC CGCCACCGGCGGCGTGGAGACCATCCCATGGAAACCGAAATCCTTTCGGACGAAG AGCTGGTGGCGATCACCGGCTACAAACCCCGGGCGTGGCAGCGCCGTTGGCTAAC AGAAAAAGGCTGGCACTTCGTCGAGAGCCGCGGCGGCCGGCCACTGGTTGGCCGC CAGTACGCCCGCCAGAAGCTCAGCGGCGTGGTGATCGACACCTTGCCGGTCGCAC CAGCCCCACCACCAACGCCCGCCTGGACCCCTGATTTTTCCCGAGTGAAGTGA |
| SEQ ID NO: 170 >pp2 HR1-J3(106)-BBa J23111-mRFP1-dblT-pp2 HR2 (Promoter and terminator are underlined. sequence encoding protein is bolded) ) | ACCAGGATGAATACCTTAAGGACGCCACCGGTAACCGGCGTTATTGGCCGGTC GCTTGCGTCAAGGTGGACCTTGAAGCATTGCGTCGCGCTCGTGACCAGCTGTG GGCTGAGGCCATGTTCTGCTACCAGGCCGGTGATATCTGGTGGGTGACCCGTG AGGAGGAAGAACTGTTCACTGCAGAGCAGGAAGAGCGCTTCGTGGTAGATGAA TGGGAGGGGCCGATCCTGAAATGGTTGGAGGAATCCCAGGCCGGCGAGACGGT CACCGGAAGCGAAGTGTTGGGGCAGGCATTGAACCTTGACCCTGGCCACTGGG GCAAGCCTGAGCAGATGCGGGTGGGATCGATCATGCACCGCCTAGGTTGGCGG CGTCGCAGGCTGGCTGCGCTGCCGAAGAGCGGTAAGCGCCCTTGGGCATATCA GAAGCCTGATGGTTGGGGGCGCAGCGCCTTGGAGCAGTCCACGCAGCCGAAGG AGGAGTGCTTTTGATCAAACACATAGATGAGATGCTGAAACTGTGGGCTCAAG AGCTCCATGCGCCGGAGCCCTGTCATTCGGCGGGCGGTGTTGGTAGCATGCTC GGCCTGTTGATCGAGTGCAAGGGTGACCTTGTGCGTGGCACCCGAGGCAGCAA GGTGCTACTGGACGAGTCTGCGGACATCGAGATCATTGTGAATAAGCATCTGG CACCGGAGCTCTACCTGGTGGTTCGTGAGCACTACTGTAACGCCGACAGCGAG CTGTACCAAAAGTACCGGCACTGTGGGTGTAGCCGGGATACCTATTACAAGCG CTTACATGAGGCGCACGTCTGTATCGCAGGCTTGCTCTTGGGGCGAGCGGCAT GATTCGCCATCCACGGTCCTACCGTCCCGCTGCCGTCCTGCCGCGTTTGATGC AGGTCAGCCTAGCTCAAGCCCGCGCAGGTCGCGGGCTGTCCCACCGTCCCACC |
| | GTACACACGAAGGCGCGCACATAGGCGTGTGCAGCGCATCACGCGCATGCATA GCATTTGCGATCATTCACGCAGCGCTTATTCAGTTGCTCACTGCGATGTCATA ATCATCGCTACGAGCTGTGAAAGATGCATAAAGCTCGTACGACGCGTTCGCTC GTCTCCTCACTTCTCCTGCGGTTACCAAAGGCGTCCTCGTCGTCTTGAAGTTG CGATTATAGATTGACGGCTAGCTCAGTCCTAGGTATAGTGCTAGCGAATTCAT TAAAGAGGAGAAAGGTACCATGAGCAAAGGAGAAGAACTTTTCACTGGAGTTG TCCCAATTCTTGTTGAATTAGATGGTGATGTTAATGGGCACAAATTTTCTGTC CGTGGAGAGGGTGAAGGTGATGCTACAAACGGAAAACTCACCCTTAAATTTAT TTGCACTACTGGAAAACTACCTGTTCCGTGGCCAACACTTGTCACTACTCTGA CCTATGGTGTTCAATGCTTTTCCCGTTATCCGGATCACATGAAACGGCATGAC TTTTTCAAGAGTGCCATGCCCGAAGGTTATGTACAGGAACGCACTATATCTTT CAAAGATGACGGGACCTACAAGACGCGTGCTGAAGTCAAGTTTGAAGGTGATA CCCTTGTTAATCGTATCGAGTTAAAGGGTATTGATTTTAAAGAAGATGGAAAC ATTCTTGGACACAAACTCGAGTACAACTTTAACTCACACAATGTATACATCAC GGCAGACAAACAAAAGAATGGAATCAAAGCTAACTTCAAAATTCGCCACAACG TTGAAGATGGTTCCGTTCAACTAGCAGACCATTATCAACAAAATACTCCAATT GGCGATGGCCCTGTCCTTTTACCAGACAACCATTACCTGTCGACACAATCTGT CCTTTCGAAAGATCCCAACGAAAAGCGTGACCACATGGTCCTTCTTGAGTTTG TAACTGCTGCTGGGATTACACATGGCATGGATGAGCTCTACAAATAAGGATCC AAACTCGAGTAAGGATCTCCAGGCATCAAATAAAACGAAAGGCTCAGTCGAAA GACTGGGCCTTTCGTTTTATCTGTTGTTTGTCGGTGAACGCTCTCTACTAGAG TCACACTGGCTCACCTTCGGGTGGGCCTTTCTGCGTTTATAAGTACTAAGGTG TATTCCCCCGGCATTCAACAGACATTCCCCGGACGTTTACCACTTATTGCCGG GTGGCATTAAAACTCGCTTGCTGCCACCGGAATCGACCTGTAAAAAGTACCCA TCTTCGAGACGTGCGGGCGCACAAAGCGGCCCGCCAAACACTGAAAACCCCGG CCCTGGCGCCGGGGTTTTTGCCTTTGGGGGATTCGATGAACAGCGAGCAGCAA GCGTTAGTTGAGATGCCAATCTGGTTGGTGATCTTCCTGTCCCTGGTCGGCGG GGTGTCAGGCGAGATGTGGCGGGCCGACATGGCCGGCGTTAGCGGCTGGTTCA TTTTCCGCCAGGTGCTGCTGCGCTCCGGTGCCTGCGTCGTATGCGGACTGTCG ACCATCATGCTGCTGTACTCGGCGGGCATGTCGATGTGGTCGGCCAGTGCCAT TGGTTGTCTCACTGCCACTGCCGGTGCGGATGTGGCCATAGGGTTGTACAAGC GTTGGGTCGCCAAGCGGCTGGGCGTCTGCGATGTCACGTCCCGTAGCGGCGAA |
| | CCTGGACAGTGACCCGATCGCCAGCCTCGGTGGGGTCGGGGACCCTGGCGATA TGGCCGGGTTACGGGGCAGGAAACCCGCGGCTCTTCGCTAGCGGACAGTTCGC CAGCTTACTGAAATTCAACCTGTTGAAATTGAAAGGTTGTTGGTTGAAATACC ATTGAAATGGAGGGCTCATGACGGATTCGAACTTCTTGTCAAAGAGCGCCTTC GCTGCTCGCATAGGGAGATCACCCAGCTACATCACCTGGTTGAAAGACAACGG CCGCCTGGTGCTTTCACCCGATGGAAAATTGGTGGATGTGCTGGCCACCGAGG CCAAGATTCAGGAGACAGCTGATCCGGCCAAAGCAGCCGTCGCGGCTCGGCAT GAAGAAAACCGCATCGAGCGGGACGTCCGGGCCCACATCCAGCCTAGCGCCGA CACACC TGCGGTGCAGCCAGCGGATCACGCGCCGAGCGGA |
| SEQ ID NO: 171 >**BBa J23109-5′-UTR (-35, -10 are underlined, TSS is bolded) | TTTACAGCTAGCTCAGTCCTAGGGACTGTGCTAGCGAATTCATTAAAGAGGAGA AAGGTACC |
| SEQ ID NO: 172 >**BBa_J23113 (-35, -10 are underlined, TSS is bolded) | CTGATGGCTAGCTCAGTCCTAGGGATTATGCTAGCG |
| SEQ ID NO: 173 >**BBa_J23117 (-35, -10 are underlined, TSS is bolded) | TTGACAGCTAGCTCAGTCCTAGGGATTGTGCTAGCG |
| SEQ ID NO: 174 >**BBa_J23114 | TTTATGGCTAGCTCAGTCCTAGGTACAATGCTAGCG |
| (-35, -10 are underlined, TSS is bolded) | |
| SEQ ID NO: 175 **BBa_J23115 (-35, -10 are underlined, TSS is bolded) | TTTATAGCTAGCTCAGCCCTTGGTACAATGCTAGCG |
| SEQ ID NO: 176 **BBa_J23107 (-35, -10 are underlined, TSS is bolded) | TTTACGGCTAGCTCAGCCCTAGGTATTATGCTAGCG |
| SEQ ID NO: 177 **BBa_J23105 (-35, -10 are underlined, TSS is bolded) | TTTACGGCTAGCTCAGTCCTAGGTACTATGCTAGCG |
| SEQ ID NO: 178 **BBa_J23106 (-35, -10 are underlined, TSS is bolded) | TTTACGGCTAGCTCAGTCCTAGGTATAGTGCTAGCG |
| SEQ ID NO: 179 **BBa_J23108 (-35, -10 are underlined, TSS is bolded) | CTGACAGCTAGCTCAGTCCTAGGTATAATGCTAGCG |
| SEQ ID NO: 180 **BBa_J23110 | TTTACGGCTAGCTCAGTCCTAGGTACAATGCTAGCG |
| (-35, -10 are underlined, TSS is bolded) | |
| SEQ ID NO: 181 >**BBa_J23111 (-35, -10 are underlined, TSS is bolded) | TTGACGGCTAGCTCAGTCCTAGGTATAGTGCTAGCG |
| SEQ ID NO: 182 **BBa_J23119 (-35, -10 are underlined, TSS is bolded) | TTGACAGCTAGCTCAGTCCTAGGTATAATGCTAGCG |
| SEQ ID NO: 183 5′-UTR (Ribosome-Binding-Site ) (Shine-Dalgarno sequence is underlined) | GAATTCATTAAAGAGGAGAAAGGTACC |
| SEQ ID NO: 184 5′ Proximal sequence: PS1 | CGAATATGACGTGTTGTTAATTTGGTT |
| SEQ ID NO: 185 5′ Proximal sequence: PS2 (same as J1) | TTGGGTTCCACCGGATACCTCCGGAC |
| SEQ ID NO: 186 5′ Proximal sequence: PS3 | GTCGTAAATAAGTAAGTCACTCCCAC |
| SEQ ID NO: 187 5′ Proximal sequence: PS4 | GTTGTCCTTCTAGTCGCCCATGACTC |
| SEQ ID NO: 188 | ACACCGACTACCCCTGCTGGGCCCAG |
| 5′ Proximal sequence: PS5 | |
| SEQ ID NO: 189 sgRNA/scRNA >BBa J23119(SpeI)-sgRNA-rrnBTerm (Promoter and terminator are underlined) | TTGACAGCTAGCTCAGTCCTAGGTATAATACTAGTNNNNNNNNNNNNNNNNNNNN GTTTTAGAGCTAGAAATAGCAAGTTAAAATAAGGCTAGTCCGTTATCAACTTGAA AAAGTGGCACCGAGTCGGTGCTTTTTTTGAAGCTTGGGCCCGAACAAAAACTCAT CTCAGAAGAGGATCTGAATAGCGCCGTCGACCATCATCATCATCATCATTGAGTT TAAACGGTCTCCAGCTTGGCTGTTTTGGCGGATGAGAGAAGATTTTCAGCCTGAT ACAGATTAAATCAGAACGCAGAAGCGGTCTGATAAAACAGAATTTGCCTGGCGGC AGTAGCGCGGTGGTCCCACCTGACCCCATGCCGAACTCAGAAGTGAAACGCCGTA GCGCCGATGGTAGTGTGGGGTCTCCCCATGCGAGAGTAGGGAACTGCCAGGCATC AAATAAAACGAAAGGCTCAGTCGAAAGACTGGGCCTTTCGTTTTATCTGTTGTTT GTCGGTGAACT |
| SEQ ID NO: 190 sgRNA/scRNA >BBa 123119(SpeI)-scRNA 1xMS2.b2-rrnBTerm (Promoter and terminator are underlined. MS2 hairpin is italic) | TTGACAGCTAGCTCAGTCCTAGGTATAATACTAGTNNNNNNNNNNNNNNNNNN NNGTTTTAGAGCTAGAAATAGCAAGTTAAAATAAGGCTAGTCCGTTATCAACT TGAAAAAGTGGCACATGAGGATCACCCATGTGCTTTTTTTGAAGCTTGGGCCC GAACAAAAACTCATCTCAGAAGAGGATCTGAATAGCGCCGTCGACCATCATCA TCATCATCATTGAGTTTAAACGGTCTCCAGCTTGGCTGTTTTGGCGGATGAGA GAAGATTTTCAGCCTGATACAGATTAAATCAGAACGCAGAAGCGGTCTGATAA AACAGAATTTGCCTGGCGGCAGTAGCGCGGTGGTCCCACCTGACCCCATGCCG AACTCAGAAGTGAAACGCCGTAGCGCCGATGGTAGTGTGGGGTCTCCCCATGC GAGAGTAGGGAACTGCCAGGCATCAAATAAAACGAAAGGCTCAGTCGAAAGAC TGGGCCTTTCGTTTTATCTGTTGTTTGTCGGTGAACT |
| SEQ ID NO: 191 ***>PP_2329-N (Promoter is underlined. RBS site indicated in upper case. start codon is bolded) | qacqccqaqcaqcqqqtacqqqtaqcacaqqqtctqttcqqqcatqtqcccaa ccqctqaaatacaqqqqctqccttqcaqcccattcqcaqcacaaqqttqctct tgcaagcactgcagttgattcaagcgcttcgctcgacctgcaggaactgcctt gtgctgcgtatgggccaaacacaatgttgaatgtcecgtacccagegectaggege ttttcctgcacaggtctacaatcgccctcctcgcttttacatcgccgtccata ctqatqccqacctqcacqctacaccccctqccctaccaqcccqaccctqccqc ctattt cgccGAATTCATTAAAGAGGAGAAAGGTACCATG |
| SEQ ID NO: 192 ***>PP_1830-O (Promoter is underlined, RBS site indicated in upper case. start codon is bolded) | ggcggcggcctgggccaggccagtgcacagcaggaagagcaaggcgacaaggc gcgacatcatgcggtactacatggggaggtgtgggtttgacccaatgtttgeg ggaaggttctatgcagggcaggtctggccctttcgcgggtgaacccgctccta cgggggctgcacaccattggaacctggtggtgaccgtgtaggagcgggtttac ccgcgaagaggccagcacaggccacccaaccatctgtatactgctgctttcgt ccatttcaqqcaqaactcqcqqaqcqtccatqtccqqcaatacctacqqcaaq ctgttcactgtcaccaccgctggcgagagccatggcGAATTCATTAAAGAGGA GAAAGGTACCATG |
| SEQ ID NO: 193 ***>PP_4965-P (Promoter is underlined. RBS site indicated in upper case, start codon is bolded) | tttgcacagggctgccaatgtttcgcgttgctcggggatcgattgcgcacgca qattcatqqqqcqqcaqtctaqacaqqcatcqatqccctaqcaaqqccaatat caaaaaqttttqatattqqcqatqaqctqcaaqccqaaaqcctcaaqttacac gctttggggcgccccgagtcgcgctctgcttcttcagttgcggcttaaagccg gcgcttggcgcttaaatgtttgecccaagcgccccccagtgggcgaaaatggcc gcctttttcgtgacaccacctattcaagccccctaggagatcagcgatgccca qccqtcqtqaacqtqccaacqccattcqtqccctcaqcatqqatqccqtqcaa GAATTCATTAAAGAGGAGAAAGGTACCATG |
| SEQ ID NO: 194 ***>PP_2082-Q (Promoter is underlined, RBS site indicated in upper case. start codon is bolded) | gcccagggtttcggcagtgataccggtgccgtcggagatgaagaacgcggttc gtttcatttgcgatctgggccttaagctgatgacgattcttggatatgataag ttcqqtttqccqaatqcqqctqtcqacattctqccacatttqcaqcqcctatt ttccaggtacaggccacaaacgcccggccaagtcatagaagacaggcgggcgc cttttgagcttttccaacacagttagtggagagatcaccttggtagagtacgt agtttccctcgataagctcggcgtccatgatgtggagcatgtggggGAATTCA TTAAAGAGGAGAAAGGTACCATG |
| SEQ ID NO: 195 ***>2168-R (Promoter is underlined, RBS site indicated in upper case. start codon is bolded) | aaggcggctgaacccttggcggtgtttgaccaggccgtggagttgttgcaggg gcggtgacctcagagggagggaacctcgggcttgcttgcgactcgaatcttca cctttcgtacagccctgtgcgggccagcgggtccgcagggtttcggcggccct tqaqcqqctqccqqqqctcqqqtaatqtcqaqtaaatqcacaqqacaqaqcac gcccatgacctccaagctggaacaactcaagcagttcaccaccgtggtcgccg acaccggggacGAATTCATTAAAGAGGAGAAAGGTACCATG |
| SEQ ID NO: 196 ***>PP_5079-S (Promoter is underlined, RBS site indicated in upper case. start codon is bolded) | tacgagcgcgcagatgactgcgacggaagaccaccagaagaacttcagcagac gtatcaaggcttttcggtgtccaggttgggagtggattgcacgcaggtcccggt ggaccaaaaaacgctgggcattataagcatttttcgcctttccgggtgaccgg ccaqqctactcqtcqaqtcqatqqqtaqccctqqcccaqtqttqccqcqqqqt tgcgacaggtggctgcgcttggcagctgtaacagacagcaaaggagtcgcgat qtttqqacqcttcqqcaaqqatqccqqttcactcqtqqqqqtqqaaattacqc ccgacGAATTCATTAAAGAGGAGAAAGGTACCATG |
| SEQ ID NO: 197 ***>PP_4297-T (Promoter is underlined. RBS site indicated in upper case. start codon is bolded) | gcccgccaccagtgcggcattcaccaggctgacggcgactttcaggttaagaa cgttcatggaaaggtcctcttcttgttgtgagaagccctaagggcagttgaaa accqatcqaaaaaataqaacacaacqaqcqatatttttqtatacaatattttq aaacgatcgtatgcgatggcgcaaacctccgttttcacgggcgctctgacgaa aqccaqcttaqccqatqaaaacccattqacctaaqccqtcaqqcqtqaataca ctctgtcgcaaagcaagttgtatacaattacaaaatcgatgaggcacaaacca tgagcaaaatgagagcaatcgatgcagccgttctggtcatgecgecgtgaaggt gtagatGAATTCATTAARAGAGGAGAAAGGTACCATG |
| SEQ ID NO: 198 ***>PP₋1075-U (Promoter is underlined. RBS site indicated in upper case, start codon is bolded) | ataactqtqcacacqatqctcqqcacqqtqcttcttcaacaqqtctaqqqcqq qqqtcatqcaaqqqctccaqtcaqqqacqactcqqcqqctactttaqqacaat tcccacatcccaqccaccqcaccqcaatcqcqcqcqtctaqtacaqqqatcaq cagctgattctagcgaccatatcgtgactgaccgttcactttcgacctttgac atcaqcqtttcttqtctatattttttcqtttctqaataaqcqqtqcacttata aggtgcatttcctacatcacccggggtcaatggggatagacaccgggttttgc tgtcgagcgccacgcgcctcacaacaaaaaaaacgaggtcatacatgacgact gctctgcgccaacccacactgtccagccaatgcctggccgagtttctcggcac cgcccctgctcatctttteggtaccggctggtgecggtcaaggtggcg gcgccagcttcgggctttgggaaatcagtatcatctggggcgtgggcgtcagc atqqccatctacctcaccqccqqtatttccqqtqcqcacctqaacccaqccqt gagtatcgccctcacactgttcgcagggttcgacaagcgcaagctgcccttct acatgctggcccaggtatgcggcgcattctgtggcgcagcactggtctacacg ctgtacagcaatctgttcttcgatttcgaacaagcccacgccatgctgcgcgg tagcgaaggcagcctggagctggcctcggtgttctccacctacccgcacccgt cgctgtccaccagccaggegtttectggttgaagtggtgatcactgeccattcetg atqqccqtqatcatqqccctqaccqacqacaacaacqqcctqccqcqcqqcqc |
| | catggccccgctgctgatcggcctgctgattgcagtgattggcagcgccatgg gcccattgactggctttgcgatgaacccggcccgcgatttcgggccaaaactc atgaccttcctggccggttggggcgaaatcgccttcactggcgggcgggatat tccctatttcctggttccggtatttgcaccgatccttggcgcctgcttgggtg cagcgagctatcgcggcctgattgcacgcaacctgccaatggcgcccgccgcg acccctgagacaaatgacattcecgccagggcgatactcaageccaattgatgceca ccatcacgcacagccctgcccccctattcttgcaaggcctacgaccatgacag acacccaggataagaactacatcatcgccctggaccagggtaccaccagttcg GAATTCATTAAAGAGGAGAAAGGTACCATG |
| SEQ ID NO: 199 ***>PP_4169-W (Promoter is underlined. RBS site indicated in upper case. start codon is bolded) | accgccgagcagggcggtcaataacagcaggcgcgggtacgggggcgtgccag qqaacacqqaqcqqcctttqtttcaqqattqaaacaqqcatqqtaactqaccq tgggggttttgccagtgggtgtcgggcgtacgtcttgaaaagtaccgcgtggc qqtqctcqatctcqacqqctccqaaaatqccatqqcaaqtaccqcaaacqtcc agacactggttctgtagcaggcgagccgtataatggccgggtcgccacttaac ttttqqctttaatqqattqqatatqactqaacaqcaacctqttqcqqttctqq qcqqcqqcaqcttcqqcaccqccqtqqcaGAATTCATTAAAGAGGAGAAAGGT ACCATG |
| SEQ ID NO: 200 ***>PP_4823-α (Promoter is underlined. RBS site indicated in upper case, start codon is bolded ) | ttccgtgacggcatcgacaatgcagctaaagtgggtatcagcgccgtgatcca gccgggtggttcgatgcgtgatgctgaagtcatcgctgctgccgacgaggccg gcatcgcgatggtcttcactggcatgcgccacttccgccactaattacgcgga tcccgaggcaagtcgagatcctgtgggagcgggcttgcccgcgaatacgatgg tggattcaccgccgcattcgcgggcaagcccgctcccacagtgttcggcgcaa gccacgggatcacttgaatcgaggttttgacatgaaagttttgatcatcggca gcggtggccgtgagcacgccctggcctggaaagtcgcccaggacccacgcgtc gagaaagtcttcgttgccccgggcaacgccggtaccgccattgaagccaagtg cgagaacgtcgccatcgacgtgtgcgccctggagcaattggccgacttcgccg aqaaaaacqtcqacctqaccatcqtcqqcccqqaaqcaccqctqqttatcqqt gtggtcgacctgttccgcagccgcggcctggactgcttcggcccaaccaaggg cqcqqcccaqctqGAATTCATTAAAGAGGAGAAAGGTACCATG |
| SEQ ID NO: 201 ***>PP_4016- β (Promoter is underlined, RBS site indicated in upper case. start codon is bolded) | aacaatgcctcgaagatccgcgccctgctgctggccggcatccgcgccgcgcg actgtggcggcagctgggcgggcaccgttggcagctggtgttcagccggcgca agttgctgaacgaactgtacgacatgatgcgcagccccaactgacgggctggg cagcccgcttccacagggtctgccaccagatccaaattgggccgacctttggt cagccacccgacccaggcgcatttttcatgtatgatatgcgcccttccaaaag cctqactqtccqaqaacaccccatqcaqctttcttcqctcactqcqqtttccc ctqtaqacqqccqttatqccqqcaaaacccaqqccttqcqccccattttcaqc gaattcggcctgatccgtttccgcgccctggtcgaagtgcgctggctgcagcg cctqqccqcccacccqcaaatcqqcqaaqtqccqqcqttctccqccqaaqcca acqccctqctqqacaqcctqqccaccqatttcaaqctqqaqcacqccqaqcqc gtcaaggaaatcgagcgcaccaccaaccacgacgtcaaggccattgaatacct cctcGAATTCATTAAAGAGGAGAAAGGTACCATG |
| SEQ ID NO: 202 ***>PP_5155- γ (Promoter is underlined. RBS site indicated in upper case. and start codon is bolded) | qqcccaqtqcacaatqqcttqcacctqttctatcqttttqqqaaaqacqa tggcgctgggcgcaggcgggtaatgcttggtccagtccttgeccatacgct tcqaqqqaaaccqqqtcaqtcaqqaccttqccaqqqtcqacaaqqqtcat caqttcttcaataacaqcqqqqtqqqtcatcqctqqaactctcqacttat tcatqqtcaccctqaqcactcttcacctqtcqqqataaqctcaqattqtq tcqcqtatqctaqcataqccctcccqctqttcatqctaaqqctqccctcq cqcctqqcatcacccctcqccaatttatctccqqqatacaqqtttacqca qatqaqcaaqacttctctcqacaaqaqcaaqatccqqttccttcttcttq aaqqtqtqcaccaqaacqcqqtcqataccctcaaqqccqccqqctacacc aacatcgaatacctcactggttcgttgccggaageccgagctgaaggaaaa qatcqccqatqcccacttcatcqqcatccqttcqcqtacccaqctcaccq aaqaqatcttcqactqtqccaaqaaactqqtcqcaqttqqctqcttctqc atcqqcaccaaccaqqttqacctqqaaqctqcccqcqcqcqcqqtatcqc cGAATTCATTAAAGAGGAGAAAGGTACCATG |
| SEQ ID NO: 203 ***>PP_5128- δ | aacatcctqctqaccaqtqcccqccacctqqaccqcaacatqcaqctqct cqaqcqaqqcqqtcaqqqcccqqatcacccqqtacacccqqccatcqccq aaacccqctacatcaaaaqcatcacctqccqqttactqccaaacaqctqa |
| (Promoter is underlined, RBS site indicated in upper case. and start codon is bolded) | tatctqccaaaaaqqqccqcccaqcqqccctttcctqccqcaatcccccc qccaaccatccacatctqcattccctcaccqcqccaqcqqtqtaqaatcq qcctattcatcqccaqtcatccccqqcqqqtttatqaqctctqqtcaaqc acqcqqcqatcccqcqcqqtcttqqccccatccqtqccaatqqcaaccqq cctqcqqcqcaaaqqacaaqaqaaqctcactcccctatttqtqacctqat taaqccqccaqqaqtqtttcatqcctqattatcqttccaaqacttccacc caaqqccqcaacatqqccqqcqcccqtqccctqtqqcqcqccaccqqqat qaaqqacqaaqacttcaaqaaaccqatcatcqccatcqccaactcqttca cccagttcgtaccgggccatgtgcacctgaaggacctgggcccagctggtg gctcgcgaaatcgaacgcgccggtggecgtggccaaggaattcaacaccat cqcqqtcqatqacqqcatcqccatqqqccacqacqqcatqctqtactcqc tqccaaqccqcqaaatcattGAATTCATTAAAGAGGAGAAAGGTACCATG |
| SEQ ID NO: 204 ***>PP-4473- ε (Promoter is underlined. RBS site indicated in upper case. and start codon is bolded) | aaggctggcgatctgatgaaacaagccgctgcggeggtgggtggcaaggg cqqcqqccqtccqqacatqqcccaqqqtqqtqqcqtcqacqtcqctqccc tqqaccaqqccctqqcqctqqccqtqccattcqcaqaqcaqqqactttqa qatqaqqqqqtqqaqqqtctaqtqqccacacaqqccactcccqtccaccc cttcatqttqqattqttttttqqqqccctqtatqqqctqaqqcaccattq aaatqqcqttqatcqtacaqaaatttqqcqqcacctctqtcqqttccatc gagcggatcgagcaggtagccgaaaaggtcaagaaacaccgtgaagcggqg cgacgacctggtggttgtgctgtcggccatgagcggtgaaaccaatcgccc tgatcgacctggccaagcagatcaccgatcagccggttecctecgtgaactg gacgtgatcgtgtcgacgggtgagcaggtcaccattgccctgctgaccat qqccttqatcaaqcqtqqtqtqccaqcqqtqtcctacaccqqcaaccaqq tqGAATTCATTAAAGAGGAGAAAGGTACCATG |
| SEQ ID NO: 205 >J5 | AGCATTTGCGATCATTCACGCAGCGCTTATTCAGTTGCTCACTGCGATGTCATAA TCATCGCTACGAGCTGTGAAAGATGCATAAAGCTCGTACGACGCGTTCGCTCGT CTCCTCACTTCTCCTACGGAGCGTTCTGGACACAACGTCGTCTTGAAGTTGCGAT TATAGA |
| SEQ ID NO: 206 >J6 | TATACATCGCATCACTACACTATTGATTATCATTGTGTACGTAACGAGCTTGCAC AACGTGAAGTTCTTCGAGCACTTCAGCTCGCAACGTAAATGACAGTTGCTGTTA AGTGACGTGAATCCTTCAATGCTGCTCATGCTGCTGTCGTAAATAAGTAAGTCA CTCCCAC |
| SEQ ID NO: 207 >J3_LL (LL sequence was underlined) | AGCATTTGCGATCATTCACGCAGCGCTTATTCAGTTGCTCACTGCGATGTCATAA TCATCGCTACGAGCTGTGAAAGATGCATAAAGCTCGTACGACGCGTTCGCTCGT CTCCTCACTTCTCCTACGGAGCGTTCTGGACACAACCGGCCCCCCCCGCTGCCGC GGGCCG |
| SEQ ID NO: 208 >J5_LL (LL sequence was underlined) | TATACATCGCATCACTACACTATTGATTATCATTGTGTACGTAACGAGCTTGCAC AACGTGAAGTTCTTCGAGCACTTCAGCTCGCAACGTAAATGACAGTTGCTGTTA AGTGACGTGAATCCTTCAATGCTGCTCATGCTGCTCGCCGCCCGGCCCGTGCCC GCGCCCG |
| SEQ ID NO: 209 >J6_LL (LL sequence was underlined) | CTGCACGAGTTCGCTGTCGAGACAAGTCTCTTAGCGACGTATTACGAAGATCAC ATAGTCAGATGAAGCTATAGAGCACGACGCTAACGATTACGTCACGCTTGACAC AACAGTTTCGCTACCTAGTGCTCGCGCGACTGCGACCCGGCCCCCCCCGCTGCC GCGGGCCG |
| SEQ ID NO: 210 >>At-pal2 (RBS was underlined) | AACTGGTAATTTGAGGAGGTAATTTATGGATCAAATCGAAGCAATGTTGTGCGG CGGAGGAGAGAAGACAAAAGTGGCGGTTACTACGAAGACTTTGGCAGATCCAT TGAATTGGGGTTTAGCAGCGGATCAAATGAAAGGAAGTCATTTAGATGAAGTG AAGAAGATGGTCGAAGAGTATCGTAGACCAGTCGTGAATCTTGGCGGAGAAAC ACTGACGATCGGACAAGTTGCTGCCATCTCCACCGTAGGAGGCAGCGTTAAGG TTGAGTTAGCGGAGACTTCAAGAGCCGGTGTGAAAGCTAGCAGTGATTGGGTT ATGGAGAGCATGAACAAAGGTACTGACAGTTACGGAGTCACCACCGGCTTTGG TGCTACTTCTCACCGGAGAACCAAAAACGGCACCGCATTACAAACAGAACTCAT TAGATTTTTGAACGCCGGAATATTCGGAAACACGAAGGAGACATGTCACACACT GCCGCAATCCGCCACAAGAGCCGCCATGCTCGTCAGAGTCAACACTCTTCTCCA |
| | AGGATACTCCGGGATCCGATTCGAGATCCTCGAAGCGATTACAAGTCTCCTCAA CCACAACATCTCTCCGTCACTACCTCTCCGTGGAACCATTACCGCCTCCGGCGAT CTCGTTCCTCTCTCTTACATCGCCGGACTTCTCACCGGCCGTCCTAATTCCAAAGC CACCGGTCCCGACGGTGAATCGCTAACCGCGAAAGAAGCTTTTGAGAAAGCCG GAATCAGTACTGGATTCTTCGATTTACAACCTAAGGAAGGTTTAGCTCTCGTTAA TGGCACGGCGGTTGGATCTGGAATGGCGTCGATGGTTCTATTCGAAGCGAATG TCCAAGCGGTGTTAGCGGAGGTTTTATCAGCGATCTTCGCGGAGGTTATGAGCG GGAAACCTGAGTTTACCGATCATCTGACTCATCGTTTAAAACATCATCCCGGACA AATCGAAGCGGCGGCGATAATGGAGCACATACTCGACGGAAGCTCATACATGA AATTAGCTCAAAAGGTTCACGAGATGGATCCATTGCAGAAACCAAAACAAGATC GTTACGCTCTTCGTACATCTCCTCAATGGCTAGGTCCTCAAATTGAAGTAATCCG TCAAGCTACGAAATCGATAGAGCGTGAAATCAACTCCGTTAACGATAATCCGTT GATCGATGTTTCGAGGAACAAGGCGATTCACGGTGGTAACTTCCAAGGAACAC CAATCGGAGTTTCTATGGATAACACGAGATTGGCGATTGCTGCGATTGGGAAG CTAATGTTTGCTCAATTCTCTGAGCTTGTTAATGATTTCTACAACAATGGACTTCC TTCGAATCTAACTGCTTCGAGTAATCCAAGTTTGGATTATGGATTCAAAGGAGC AGAGATTGCTATGGCTTCTTATTGTTCTGAGCTTCAATACTTGGCTAATCCAGTC ACAAGCCATGTTCAATCAGCTGAGCAACATAATCAAGATGTGAACTCTCTTGGTT TGATCTCGTCTCGTAAAACATCTGAAGCTGTGGATATTCTTAAGCTAATGTCAAC AACGTTCCTTGTGGGGATATGTCAAGCTGTTGATTTGAGACATTTGGAGGAGAA TCTGAGACAAACTGTGAAGAACACAGTTTCTCAAGTTGCTAAGAAAGTGTTAAC CACTGGAATCAACGGTGAGTTACATCCGTCAAGGTTTTGCGAGAAGGACTTGCT TAAGGTTGTTGATCGTGAGCAAGTGTTCACGTATGTGGATGATCCTTGTAGCGC TACGTACCCGTTGATGCAGAGACTAAGACAAGTTATTGTTGATCACGCTTTGTCC AACGGTGAGACTGAGAAGAATGCAGTGACTTCGATCTTTCAAAAGATTGGAGC TTTTGAAGAGGAGCTTAAGGCTGTGCTTCCAAAGGAAGTTGAAGCGGCTAGAG CGGCTTATGGGAATGGAACTGCGCCGATTCCTAACCGGATTAAGGAATGTAGG TCGTATCCGTTGTATAGGTTCGTGAGGGAAGAGCTTGGAACGAAGTTGTTGACT GGAGAAAAGGTTGTGTCTCCGGGAGAGGAGTTTGATAAGGTCTTCACTGCTAT GTGTGAAGGTAAACTTATTGATCCGTTGATGGATTGTCTCAAGGAATGGAACGG AGCTCCGATTCCGATTTGCTAA |
| SEQ ID NO: 211 ECK120033736_termi nator | aacgcatgagAAAGCCCCCGGAAGATCACCTTCCGGGGGCTTTtttattgcgc |
| SEQ ID NO: 212 >PP_4715-V (promoter is underlined, RBS site indicated in upper case, and start codon is bolded) | accgaagcgctggcggggcggggtcgtgtgctgttgcgcaagtccggtaccgagccgttggtgcgggtca tggttgagggcgaggacgaaagccaggtgcgggcccatgctgaagcgctggccaaactggtcggcgaa gtttgtgtctgaaggcgcttgccagcgcagatctggttgggtaagatctgcgcccactttgaccgacgagg taaagcatgcgtcgccctatggtagctggtaactggaaaatgcacggtacccgcgctagcgtcgctGAA TTCATTAAAGAGGAGAAAGGTACCATG |
| SEQ ID NO:213 mTagBFP2>J5-BBa J23117-RBS-mTagBFP (Promoter is underlined. protein is bolded) | TATACATCGCATCACTACACTATTGATTATCATTGTGTACGTAACGAGCTTGC ACAACGTGAAGTTCTTCGAGCACTTCAGCTCGCAACGTAAATGACAGTTGCTG TTAAGTGACGTGAATCCTTCAATGCTGCTCATGCTGCTGTCGTAAATAAGTAA GTCACTCCCACttqacaqctaqctcaqtcctaqqqattqtqctaqcacccqtt ttttgggctaacaggaggaattaaccatggggagccaccatcaccatcaccat ggcagatctATGAGCGAGCTGATTAAGGAGAACATGCACATGAAGCTGTACAT GGAGGGCACCGTGGACAACCATCACTTCAAGTGCACATCCGAGGGCGAAGGCA AGCCCTACGAGGGCACCCAGACCATGAGAATCAAGGTGGTCGAGGGCGGCCCT CTCCCCTTCGCCTTCGACATCCTGGCTACTAGCTTCCTCTACGGCAGCAAGAC CTTCATCAACCACACCCAGGGCATCCCCGACTTCTTCAAGCAGTCCTTCCCTG AGGGCTTCACATGGGAGAGAGTCACCACATACGAAGACGGGGGCGTGCTGACC GCTACCCAGGACACCAGCCTCCAGGACGGCTGCCTCATCTACAACGTCAAGAT CAGAGGGGTGAACTTCACATCCAACGGCCCTGTGATGCAGAAGAAAACACTCG GCTGGGAGGCCTTCACCGAGACGCTGTACCCCGCTGACGGCGGCCTGGAAGGC AGAAACGACATGGCCCTGAAGCTCGTGGGCGGGAGCCATCTGATCGCAAACGC CAAGACCACATATAGATCCAAGAAACCCGCTAAGAACCTCAAGATGCCTGGCG TCTACTATGTGGACTACAGACTGGAAAGAATCAAGGAGGCCAACAACGAGACC TACGTCGAGCAGCACGAGGTGGCAGTGGCCAGATACTGCGACCTCCCTAGCAA ACTGGGGCACAAGCTTAATTAA |

*Endogenous promoters: Underlined sequences are 60bp from two ORF adjacent to the promoter. Bolded nucleotides are TSS according to (D′ Arrigo et al., 2016) or start codon of the gene or mRFP.
**Anderson Promoters: RNAP recognition site (-35 and -10 elements) of the Anderson promoter series are underlined. TSSs are indicated in bold at the predicted site. TSSs of the Anderson promoter series were characterized in (Kosuri et al., 2013); most TSSs are located immediately after the 35 bp sequence as expected. Some weak promoters have undefined TSSs due to low RNAseq signal and some of the TSSs reported in (Kosuri et al., 2013) are shifted downstream by 1 base when C is the first nucleotide of the 5′ -UTR. For all experiments reported in this manuscript, the first base of the 5′-UTR is G (see 5′-UTR sequence below) The promoters shown below appear in an order of ascending basal expression level in P. putida (weakest first).
***Endogenous promoters (shown in FIG. 33 ). Bold ATG is the coding sequence of the reporter gene.

TABLE 8

Additional scRNA spacer sequence

| | | | |
|---|---|---|---|
| Name | Sequence | Target Gene | Target Strand |
| (SEQ ID NO: 214) N3 | aagcgcttcgctcgacctgc | PP_2329 | Non-Template |
| (SEQ ID NO: 215) O2 | caatggtgtgcagcccccgt | PP_1830 | Template |
| (SEQ ID NO: 216) P1 | cttcagttgcggcttaaagc | PP_4965 | Non-Template |
| (SEQ ID NO: 217) Q2 | ggttcgtttcatttgcgatc | PP_2082 | Non-Template |
| (SEQ ID NO: 218) R4 | cggacccgctggcccgcaca | PP_2168 | Template |
| (SEQ ID NO: 219) S4 | gaacttcagcagacgtatca | PP_5083 | Non-Template |
| (SEQ ID NO: 220) T2 | cgtcagagcgcccgtgaaaa | PP_4297 | Template |
| (SEQ ID NO: 221) U1 | aatcgcgcgcgtctagtaca | PP_1075 | Non-Template |
| (SEQ ID NO: 222) W2 | tcttgaaaagtaccgcgtgg | PP_4169 | Non-Template |
| (SEQ ID NO: 223) α2 | GGCCGAAGCAGTCCAGGCCG | PP_4823 | Non-Template |
| (SEQ ID NO: 224) β1 | GGCCGAATTCGCTGAAAATG | PP_4016 | Non-Template |
| (SEQ ID NO: 225) γ2 | GGCACAGTCGAAGATCTCTT | PP_5155 | Non-Template |
| (SEQ ID NO: 226) δ2 | GATGCCGTCATCGACCGCGA | PP_5128 | Non-Template |
| (SEQ ID NO: 227) ε1 | GGCCAGGTCGATCAGGCGAT | PP_4473 | Non-Template |
| SEQ ID NO: 228 J506 | AGCAGCATGAGCAGCATTGA | J5 promoter | Non-Template |
| SEQ ID NO: 229 | GTCGCAGTCGCGCGAGCACT | J6 promoter | Non-Template |
| J606 | | | |

### Example 25

### Media and Chemicals

E. coli and P. putida culture and engineering were generally performed in LB media. Pseudomonas isolation agar (Difco) was used in the tri-parental mating for mini-Tn7 cloning (Choi, K.-H., Schweizer, H.P., 2006. mini-Tn7 insertion in bacteria with single attTn7 sites: example Pseudomonas aeruginosa. Nat. Protoc. 1, 153-161). Fluorescent protein reporter gene activation and metabolic engineering experiments were performed in EZ rich-defined media (Teknova) with 0.2% glucose as the carbon source, unless specified. Appropriate concentration of antibiotics were included for plasmid maintenance: 100 µg/mL for carbenicillin, 25 µg/mL for chloramphenicol, 30 µg/mL for gentamicin, 30 µg/mL for kanamycin. For two-plasmid transformations in this work, the antibiotic concentration was reduced by half to 15 µg/mL each of gentamicin and kanamycin. IPTG was prepared in water as a 1 M stock solution prior to use. m-Toluic acid was prepared as a 0.5 M stock solution in 50% DMSO/water. Biopterin, BH2, and BH4 (Cayman Chemical) were stored in DMSO and diluted into water prior to use. D,L-mevalonolactone (Sigma) was freshly prepared in ethanol as a 20 mg/mL solution before dilution in ethyl acetate.

### Example 26

### Plasmids Construction Strategy

Plasmids used in this study can be separated into genome integration plasmids and replicable plasmids. Integration plasmids were made based on pUC18T-mini-Tn7T-Gm. Replicable plasmids were constructed from pBBR1-MCS2 (named pBBR1-KmR in this study), pBBR1-MCS5 (named pBBR1-GmR in this study), and pRK2-AraE (Table 1). The AraE cassette from pRK2-AraE (bearing GmR marker) was replaced with multiple-cloning-site regions of pBBR1 to generate pRK2-GmR. pRK2-KmR was made by replacing the GmR marker and AraE cassette with KmR marker and its multiple-cloning-site from pBBR1-KmR. The CRISPRa components and genes of interest were incorporated into each backbone at the multiple-cloning-site region. The detailed methodology for construction of each backbone is provided below. Table 2 shows the list of strains and plasmids used in each figure. Plasmids descriptions are listed in Table 3.

MCP-SoxS(R93A/S101A) was used in this study and will be abbreviated as MCP-SoxS. Both dCas9 and MCP-SoxS were obtained from the pCD442 plasmid (Fontana, J., et al. 2020. Effective CRISPRa-mediated control of gene expression in bacteria must overcome strict target site requirements. Nat. Commun. 11, 1618). The 1xMS2 scRNA.b2 was used in this study with variable 20 bp target sequences (Dong, C., et al. 2018. Synthetic CRISPR-Cas gene activators for transcriptional reprogramming in bacteria. Nat Commun 9, 2489). The full sequence of sgRNA and scRNA are provided in the DNA sequences section. Any plasmid with sg/scRNA has different 20bp target sequences according to Table 3. sg/scRNA sequences were provided in Table 5.

### Example 27

### Integration Plasmids

### pPPC001 and pPPC005-007

For pPPC001, the dCas9/MCP-SoxS cassette was amplified from pCD442 and inserted into pUC 18T-miniTn7T-Gm with KpnI/SacI. For pPPC005, the dCas9/MCP-SoxS coding sequence was amplified from pCD442 and inserted together with XylS-Pm as a promoter of dCas9, amplified from pS448-CsR (Wirth et al., 2019. Accelerated genome engineering of Pseudomonas putida by I-SceI-mediated recombination and CRISPR-Cas9 counterselection. Microb Biotechnol. 13, 223-249). Further modification of the MCP-SoxS promoter was achieved by digestion of pPPC001/pPPC05 with PstI/Bsp120I and XylS-Pm was inserted into the corresponding site to give pPPC006/007, respectively. See FIG. 16A for representative plasmid maps.

### pPPC002-004

For integration plasmids with the reporter gene included, J1-BBa_J23117-sfGFP was amplified from pJF076Sa (Fontana et al., 2020) and inserted into the KpnI/SacI site of pUT18T-miniTn7T-Gm. Then, dCas9/MCP-SoxS was added to the HindIII site to give pPPC002. For pPPC003.N, the J1(+N)-BBa_J23117-sfGFP fragments were amplified from pJF155.1-12 (Fontana et al., 2020. Effective CRISPRa-mediated control of gene expression in bacteria must overcome strict target site requirements. Nat. Commun. 11, 1618) instead of pJF076Sa. In the case of pPPC004 with additional BBa_J23111-mRFP reporter, the corresponding reporter fragment was amplified from the pJF143.J3.J23111 (Fontana et al., 2020. Effective CRISPRa-mediated control of gene expression in bacteria must overcome strict target site requirements. Nat. Commun. 11, 1618), with BBa_J23111 promoter instead of a BBa_J23117, and inserted into pPPC002 at the Mph1103I cut site. Replicable plasmids

pRK2-GmR was made by digesting pRK2-AraE (containing GmR marker) with AatII/BspTI and the multiple-cloning-site (MCS) from pBBR1 was inserted into the pRK2 backbone. pRK2-KmR was made by digestion of pRK2-AraE with SacI/BspTI and insert KmR and MCS fragments from pBBR1-KmR. Then, the further modification of these plasmids followed the general manipulation at the MCS. See FIG. 17A for representative plasmid maps.

scRNA (or sgRNA) was inserted into the replicable plasmid at the SacI/KpnI site of the MCS. Then, the reporter fragment was inserted at the Mph1103I region. The dCas9/MCP-SoxS cassette was inserted into Mph1103I. For pRK2-GmR and pRK2-KmR, the scRNA fragment was amplified from pPPC013 and inserted into pRK2 backbones at NotI/Bsp120I site due to conflicting SacI/KpnI cut sites in the pRK2 backbone.

To change the scRNA target sequence, the existing scRNA cassette was excised with SpeI/BspTI and the new scRNA fragment was inserted. To express multiple scRNAs from the same plasmid, additional scRNA (or sgRNA) cassettes can be inserted at the BspTI site. To generate a new scRNA fragment, any existing scRNA construct can be amplified with a forward primer binding at the promoter region, oCK079_GCTCAGTCCTAGGTATAATACTAGT. To introduce a new 20 base target sequence, a forward primer with the same overhang can be used, oCK287_TAGGTATAATACTAGTNNNNNNNNNNNNNNNNNNNNGTTTTAGAGC TAGAAATAGCAAGT, where the variable 20nt in oCK287 can be replaced with the desired target sequence.

To insert J1-mRFP reporter cassettes, the PCR fragment was amplified from pJF076Sa (Fontana et al., 2020. Effective CRISPRa-mediated control of gene expression in bacteria must overcome strict target site requirements. Nat. Commun. 11, 1618) as a template and cloned into the Mph1103I site. The J3-mRFP variants were constructed in the same manner with pJF143.J3 (Fontana et al., 2020) as a PCR template. To insert other genes of interest under control of J1 or J3 promoters, several approaches are available. AatII was introduced upstream of J1 and J3 sequences. KpnI was added at the end of strong RBS and XhoI was added between the stop codon and terminator. The desired cassette can be cloned into the Mph1103I site directly or inserted at the aforementioned sites. Biopterin pathway genes were inserted with AatII/XhoI using pCK015 (J3-GTPCH-J3-PTPS-J3-SR) and pCK014 (J3-GTPCH-J3-PTPS) as templates for pPPC027-028. LacI-Ptrc was added into AatII/XhoI and mvaES was added into KpnI/XhoI using pMVA2RBS035 as a template for PCR to give pPPC029-030 respectively.

pCK014 and pCK015 were analogs of pPPC028 and pPPC027, respectively, in pSC101** origin for E. coli experiment which can be double transformed with pCK005.AAV and pCD581 (Fontana et al., 2020. Effective CRISPRa-mediated control of gene expression in bacteria must overcome strict target site requirements. Nat. Commun. 11, 1618). The gtpch gene was amplified from the E. coli MG1655 genome. ptps and sr from M. alpina were synthesized from GeneArt (Thermo-Fisher) with codon-optimization for expression in E. coli using Gene Designer (Atum). Each J3-CDS was individually added into the reporter cassette. Then, an additional J3-CDS construct was inserted into the existing one at the EcoRV site (altered from AatII due to the presence of cut-site in sr gene) to get pCK014 and pCK015.

### Example 28

### Changing scRNA Target Sequence of J1 or J3

To alter the scRNA 20 base target sequence, a single-fragment PCR was used to change the existing 20 bp target of J106 in pPPC016 to the desired J306 using oCK237/oCK279 (Table 4). Then, the fragment was treated with DpnI, gel purified, and circularized with Infusion. The same method was used for converting J306 to J106 with oJF365 /oJF366.

### Example 29

### Construction of 5’-Proximal Sequence Library (pPPC022)

To generate a library of different 26bp sequences upstream of a minimal promoter, a fragment with randomized 26bp region (5′-PS-BBa_J23117-mRFP) was constructed with the oJF447 (SEQ ID NO: 35) and oCK219 (SEQ ID NO: 34) primers (Table 4). pPPC020 bearing J306 scRNA was linearized by PCR with oJF448 (SEQ ID NO: 36)/oCK084 (SEQ ID NO: 37) and treated with DpnI to remove the parent vector. Then the linearized pPPC016 backbone fragment and a randomized 26bp library fragment were assembled with Infusion.

### Example 30

### Construction of 5’-roximal Sequence Variants Characterized in E. Coli (pPPC023)

pPPC023 was constructed similar to pPPC022 as described above. Five oJF447 (SEQ ID NO: 35) variants with known 26bp sequences (provided in the DNA Sequences section) were used to generate 5′-PSN-BBa_J23117-mRFP fragments (PS1 to PS5) for insertion into the linearized backbone.

### Example 31

### Construction of Dual Reporter Plasmids (pPPC024-025, pPPC031-034)

For the plasmid-based dual reporter for multi-gene CRISPRa with two strongly expressed fluorescent reporters, a J3(106)-BBa_J23117-sfGFP cassette was inserted at the AatII site of J3-BBa ₋J23117-mRFP (pPPC020) to generate pPPC024. The plasmid-based dual reporter for CRISPRi/a with weakly expressed mRFP and strongly expressed sfGFP was constructed by delivering J3(106)-BBa_J23111-sfGFP to pPPC020 to generate pPPC025. Multiple sgRNA/scRNA cassettes were delivered as described above in the Replicable plasmids section.

The genomically-integrated dual reporter strains were constructed by sequentially integrating separate mRFP and sfGFP reporters at different genomic sites. Plasmids, pGNW2-ppl and pGNW2-pp2 were constructed from pGNW2 (Wirth et al., 2019. Accelerated genome engineering of Pseudomonas putida by I-SceI-mediated recombination and CRISPR-Cas9 counterselection. Microb Biotechnol) by addition of prophage 1 (pp 1) or prophage2 (pp2) regions into the XbaI/EcoRI site. Flanking homology sites (HR1 and HR2) were separated by an Mph1103I site for insertion of the desired heterologous gene. J3-BBa_J23117-mRFP was inserted into pGNW2-ppl at the Mph1103I site to construct pPPC031. sfGFP constructs with different promoters were cloned into pGNW2-pp2 at the Mph1103I site to generate pPPC032-034.

### Example 32

### Construction of Endogenous Promoter Reporter (pPPC026)

The J3-BBa-J23117 reporter (pPPC020) was modified into an endogenous promoter reporter by replacing the J3-BBa_J23117 promoter with an intergenic region from each gene of interest. The intergenic region contained 60 bases from the ORF of interest on the 3′ end. On the 5′ end, the intergenic region extended 60 bp into the next upstream ORF, following a previously reported strategy (Zaslaver et al., 2006. A comprehensive library of fluorescent transcriptional reporters for Escherichia coli. Nat. Methods 3, 623-628). The mRFP cassette, along with its original strong RBS, was included downstream of the 60 bp fragment of the ORF of interest. Complete sequences are provided below.

### Example 33

### CaCl₂ Chemically Competent Cell Preparation

The chemically competent cell preparation was adapted from a prior method (Zhao et al., 2013. [CaCl₂-heat shock preparation of competent cells of three Pseudomonas strains and related transformation conditions]. Ying Yong Sheng Tai Xue Bao 24, 788-94). From an overnight culture seeded from a single colony of a P. putida strain in LB, the cell suspension was 100-fold diluted into 50 mL LB without antibiotic in a 250 mL Erlenmeyer flask. The culture was incubated at 30° C. to OD600 = 0.8 - 1.0, transferred to 2 × 50 mL conical tubes, and placed on ice for 5 minutes. The cell suspension was centrifuged at 4° C. for 10 min at 5000 rpm. After discarding the supernatant, the cells were washed with an ice-cold solution of 50 mM MgCl₂ + 10 mM CaCl₂ twice. The final pellets were resuspended in 15% glycerol + 100 mM CaCl₂ solution to give chemically competent cells. The competent cells can be stored at -80° C. for a month with negligible loss of activity.

For transformation, 50 ng of a P. putida compatible plasmid was added to 100 µL of CaCl₂ chemically competent cells in a 1.5 mL microcentrifuge tube. Cells were mixed gently and incubated on ice for 30 minutes. The incubated competent cells were subjected to heat-shock at 42° C. for 3 minutes and cooled on ice for another 5 minutes. Then, 900 µL of LB was added to the competent cells and cultures were shaken at 30° C. for 1.5 hours. The outgrowth competent cells were spun down at 10000 rcf, room temperature for 1 minute. After discarding ~900 µL of supernatant, cells were resuspended in residual media for plating on a pre-warmed agar plate with appropriate antibiotic selection.

### Example 34

### Biopterin Quantification by HPLC-MS

The LC-MS quantification was adapted from the prior method (Ehrenworth et al., 2015. Pterin-Dependent Mono-oxidation for the Microbial Synthesis of a Modified Monoterpene Indole Alkaloid. ACS Synth. Biol. 4, 1295-1307). LC-MS analysis was completed using an Agilent 1100/1260 series system equipped with a 1260 ALS autosampler and a 6120 Single Quadrupole LC-MS with a Poroshell 120 SB-Aq 3.0 mm × 100 mm × 2.7 µm column and an electrospray ion source. LC conditions: solvent A-150 mM acetic acid with 0.1% formic acid; solvent B-methanol with 0.1% formic acid. Gradient: 4 min ramp from 95%:5%:0.2 (A:B:flow rate in mL/min) to 70%:30%:0.2, 6 min ramp to 40%:60%:0.2, 2 min ramp to 2%:98%:0.2, 2 min ramp to 2%:98%:0.5, 4 min at 2%:98%:0.5, 1 min ramp to 95%:5%:0.5, 7 min at 95%:5%:0.5, and 1.5 min post time. MS acquisition (positive ion mode) included 25% scan from m/z 100-600, 25% scan from m/z 230-260, 25% scan from m/z 145-165, and 25% selective ion monitoring (SIM) for BH4 (m/z 242.1), dihydrobiopterin (m/z 240.1), and biopterin (m/z 238.1). Retention times were determined using commercially available standards (BH4, BH2, and biopterin from Cayman Chemical).

### Example 35

### Determination of P. Putida Growth Rate

Single colonies from LB plates were inoculated in 500 µL of EZ-RDM (Teknova) supplemented with the appropriate antibiotics and grown in 96-deep-well plates at 30° C. with shaking overnight. From the overnight cultures, OD600 of each replicate was measured in a 1-cm cuvette, then diluted to OD600 = 0.1 (30-50 fold dilution) and 200 µL of each diluted culture were grown in flat bottom microplate at 30° C. in a Biotek Synergy HTX plate reader for 16 hours with continuous slow orbital shaking.

### Example 36

As described in the proceeding examples, CRISPRa can be ported to P. putida when accompanied by select optimizations, e.g., optimizations to the expression of dCas9, MCP-SoxS, and scRNA. The CRISPRa system was successfully targeted to endogenous targets. Multiple genes can be activated simultaneously by targeting multiple promoters or by targeting a single promoter in a multi-gene operon. Ultimately, the disclosed approach can be used for chemical productions in P. putida.

This example describes implementation of additional modifications to P. putida to facilitate production of p-aminocinnamic acid (pACA), as determined HPLC chromatography.

Initial attempts in E. coli demonstrated difficulty to produce pACA from glucose. P. putida possess resistance towards aromatic compounds in general and, thus, may serve as an advantageous platform for production of various products. For example, growth resistance experiments demonstrated P. putida can tolerate a higher concentration of pACA.

To optimize the production of pACA in P. putida, certain changes were determined to be significantly beneficial. In one instance, change the key enzyme, i.e., from At-PAL (from plant, Arabinobsis thaliana) to Rg-PAL (from yeast, Rhodotorula glutinis), led to significant improvement. Additionally, a change to the expression system was beneficial. The expression system was changed from a two-plasmid system to a one-plasmid system, which led to significant improvement in production. However, this change comes with a tradeoff of plasmid burden due to a large plasmid size. Another optimization was to implement a genetically integrated system. However, it is not routine to simply place a heterologous cassette onto the genome. Instead, several rounds of optimization on the minimal promoter are necessary to achieve sufficient base expression to be CRISPR-activatable to relevant levels, and also to avoid overexpression when activated, leading to instability. It was determined that a stable integration that led to stable production of pACA. For CRISPRa-controlled expression, an optimization of scRNA expression level is also necessary

### Example 37

### Culture and HPLC Methods

Constitutive dCas9 and MCP-SoxS were previously integrated into P. putida KT2440 to make CKPP002. This strain, or its derivative IFPP006 with integrated papABC and aroGL, was transformed by electroporation with a pBBR1 plasmid containing either scRNAs only or pathway genes and scRNAs. Two-plasmid production strains including the additional pRK2 plasmid were doubly transformed in series, using competent cells containing the papABC/aroGL plasmid. A control strain for standard curve diluent was transformed with similar plasmids not containing pathway genes. Single colonies were picked in triplicate and used to inoculate 2 mL of MOPS EZ-Rich defined media (Teknova), supplemented with appropriate antibiotics, in 14 mL polypropylene culture tubes. Cultures were grown at 30° C. and shaken at 200 rpm for 24 hours.

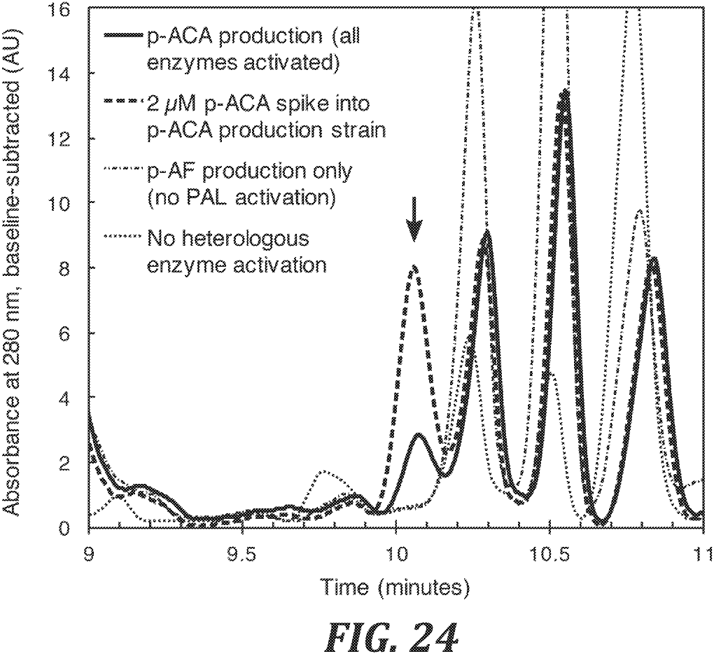

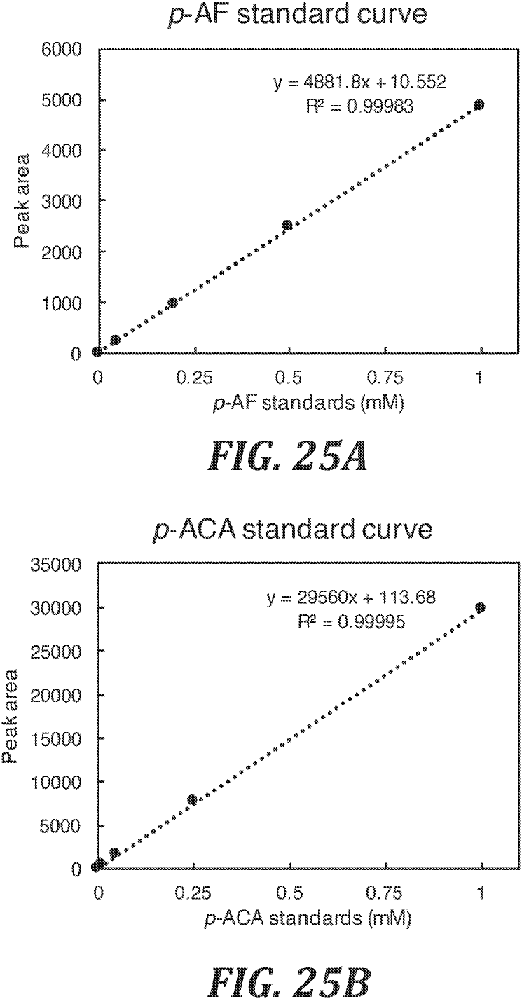

Culture supernatants were filtered by centrifuging at 14000 g for 20 minutes using an Amicon® Ultracel-10 centrifuge filter (Millipore). Filtered supernatants were supplemented with 0.2% trifluoroacetic acid (TFA) and assessed using an Agilent HPLC with a diode array detector set at 210 nm. p-AF, p-ACA, and other components were separated using a ZORBAX Eclipse Plus phenyl-hexyl column (Agilent) with water plus 0.2% TFA as solvent A and methanol plus 0.2% TFA as solvent B. See FIG. 24. The mobile phase gradient was as follows: 100% solvent A at 1 mL/min from 0 to 4 min, ratio increased to 95% solvent B and 5% solvent A at 1 mL/min from 4 to 18 min, 87 100% solvent A at 1 mL/min from 18 to 22 min. Sample concentrations were determined by interpolation from standard curves ranging from about 10 µM to 1 mM. p-AF, p-ACA, and pABA for standard curves were obtained from Santa Cruz Biotechnology. See FIG. 25.

### Example 38

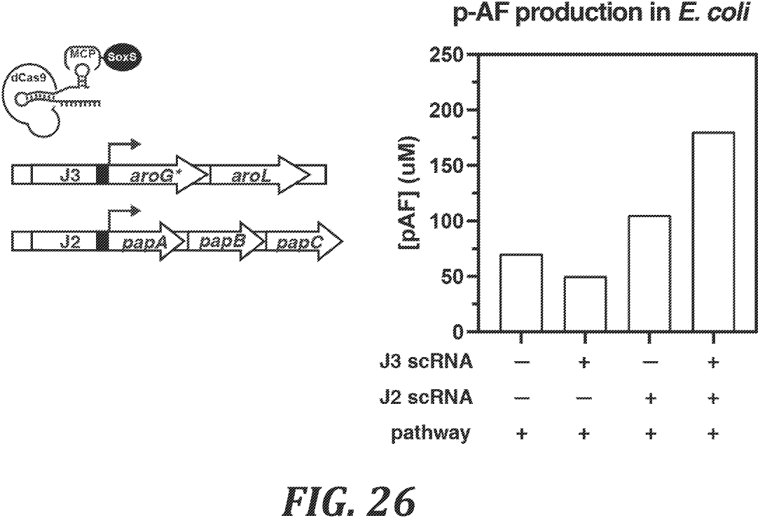

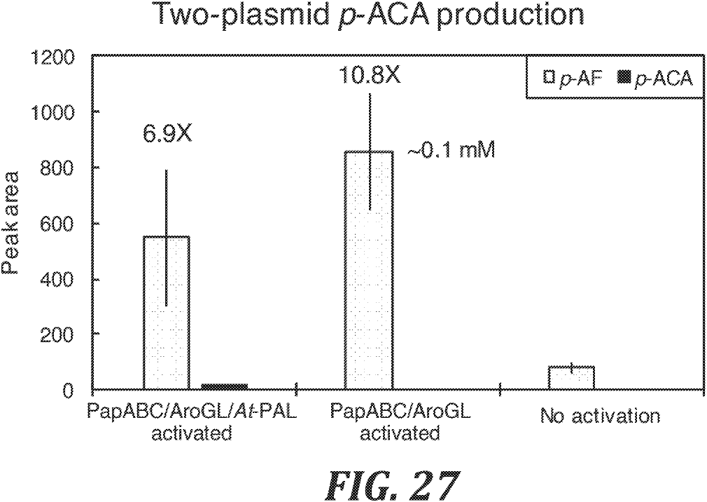

In E. coli, p-AF production can occur, but p-ACA is toxic. See, e.g., FIG. 26. In contrast, P. putida exhibits resistance to p-ACA. See FIG. 27. Prior to optimization, P. putida demonstrates a small increase in p-ACA production. See FIGS. 24, 27, and FIG. 32B, columns 3 and 4.

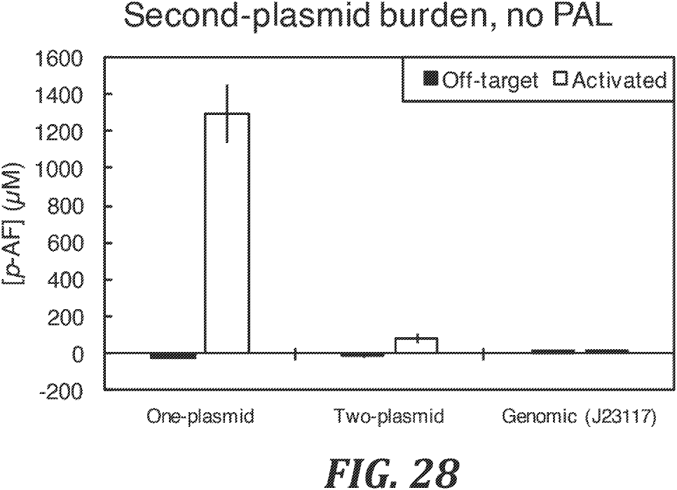

Various optimizations were implemented. FIG. 32B illustrates p-ACA production using R. glutinis PAL incorporating distinct optimizations. Expression and burden optimizations were made. See, e.g., FIG. 32D for p-ACA production differences in two-plasmid vs. one-big-plasmid. There were initial concerns about big-plasmid size, but tight replicates suggest stability for expression. Results for p-AF production in two-plasmid vs. one-plasmid are illustrated in FIG. 28. FIG. 32D provides comparison of optimal integrated strain (PapABC+AroGL integrated, RgPAL plasmid-based) with fully plasmid-based strains. It is noted that care must be taken with expression levels (promoter strengths) when integrating. Specifically, the promoter strengths must be increased relative to plasmid-based promoters, but not too strongly. To illustrate, see FIG. 29, which provides an exemplary instance of incorporating too strong of a promoter (e.g., 105 is good; 110 is too strong). Colony PCR suggests that the integrated 110-PapABC/AroGL is getting kicked out in most of the population. Once we get the entire pathway integrated on 105 promoters, p-ACA production looks pretty good; but the number of scRNAs and their expression level has big effects on production and should also be optimized. See FIG. 30.

While illustrative embodiments have been illustrated and described, it will be appreciated that various changes can be made therein without departing from the spirit and scope of the invention.

1. An engineered bacterium comprising genetic elements supporting programmable transcriptional activation and/or repression.

2. The engineered bacterium of claim 1, wherein the genetic elements comprise at least one heterologous nucleic acid construct comprising a first nucleic acid sequence encoding an endonuclease that lacks endonuclease activity.

3. The engineered bacterium of claim 2, wherein the endonuclease is dCas9, dCas12, dCasX, dCasPhi, dCas3 (Cascade), and the like.

4. The engineered bacterium of claim 2, wherein the at least one heterologous nucleic acid construct comprises a second nucleic acid sequence encoding a transcriptional activator.

5. The engineered bacterium of claim 4, wherein the transcriptional activator comprises an RNA-binding protein (RBP) fused to an effector domain, wherein the effector domain is selected from SoxS, TetD, PspF, AsiA, N-terminus of RpoA (aNTD), and SoxS-family activators.

6. The engineered bacterium of claim 5, wherein the RNA-binding protein is selected from MCP, PCP, Com, LambdaN22Plus, and Qbeta.

7. The engineered bacterium of claim 5, wherein the SoxS is engineered to reduce or abolish DNA-binding capacity.

8. The engineered bacterium of claim 7, wherein the SoxS is engineered to comprise a mutation, optionally wherein the mutation at R93 and/or S101, and optionally wherein the mutation comprises R93A and/or S101A.

9. The engineered bacterium of claim 2, wherein the at least one heterologous nucleic acid construct comprises a third nucleic acid sequence encoding a scaffold RNA (scRNA).

10. The engineered bacterium of claim 9, wherein the scRNA comprises a 3′ MS2 hairpin loop that interacts with a transcriptional activator.

11. The engineered bacterium of claim 9, wherein the scRNA comprises a 5′ domain comprising a guide sequence that hybridizes to a target sequence.

12. The engineered bacterium of claim 11, wherein the target sequence is proximal to a PAM and/or a promoter sequence of an endogenous gene of the engineered bacterium.

13. The engineered bacterium of claim 11, wherein the at least one heterologous nucleic acid construct comprises a fourth nucleic acid sequence comprising an open reading frame of a gene of interest operatively linked to a promoter sequence and/or a PAM sequence, and wherein the target sequence is proximal to the promoter sequence and/or the PAM sequence.

14. The engineered bacterium of claim 13, wherein the at least one heterologous nucleic acid construct comprises the first, second, third, and fourth sequences distributed in any combination on two vectors.

15. The engineered bacterium of claim 13, wherein the at least one heterologous nucleic acid construct comprises the first, second, third, and fourth sequences distributed on a single vector.

16. The engineered bacterium of claim 15, wherein the vector is optionally pBBR1, pRK2, pRSF1010, pBAV1, and the like, or derived from pBBR1, pRK2, pRSF1010, pBAV1, and the like.

17. The engineered bacterium of claim 13, wherein the at least one heterologous nucleic acid construct is integrated into the genome of the engineered bacterium.

18. The engineered bacterium of claim 13, wherein the first, second, third, and fourth sequences each comprise or are operatively linked to a promoter operable in the engineered bacterium.

19. The engineered bacterium of claim 13, wherein the engineered bacterium is Pseudomonas putida or Acinetobacter baylyi.

20. The engineered bacterium of claim 13, wherein the engineered bacterium is Pseudomonas putida, and wherein the target sequence is between about 60 to about 120 bases upstream (5′) of a transcriptional start site (TSS) of the endogenous gene or open reading frame.

21. The engineered bacterium of claim 13, wherein the target sequence is about 15 to about 25 bases upstream (5′) of a transcriptional start site (TSS) of the endogenous gene or open reading frame.

22. The engineered bacterium of claim 21, wherein the target sequence corresponds with the J1, J3, J5, or J6 promoter, or portions thereof.

23. The engineered bacterium of claim 20, wherein the promoter sequence resides in the intervening sequence between the target sequence and the transcriptional start site (TSS) of the endogenous genes or open reading frame.

24. The engineered bacterium of claim 23, wherein the promoter sequence is a synthetic 5′-upstream sequence containing appropriate NGG PAM at an optimal position, wherein the optimal position is selected from about 75 to 85 nucleotides, about 78 to 83 nucleotides, and about 81 nucleotides upstream of the TSS.

25. The engineered bacterium of claim 20, wherein the genetic elements are under control of a small-molecule inducible promoter, and wherein the small molecule inducer is selected from m-toluic acid, salicylic acid, benzoic acid, and related compounds.

26. The engineered bacterium of claim 25, wherein the small-molecule inducible promoter is XylS/Pm, derived from P. putida mt-2.

27. The engineered bacterium of claim 13, wherein the open reading frame encodes gene product that results in production of an aromatic compound.

28. The engineered bacterium of claim 27, wherein the bacterium is engineered to produce p-aminophenylalanine (p-AF) or p-aminocinnamic acid (p-ACA).

29. The engineered bacterium of claim 28, wherein the bacterium comprises an open reading frame encoding PAL, optionally wherein the PAL is derived from Arabinobsis thaliana or Rhodotorula glutinis.

30. The engineered bacterium of claim 28, wherein the bacterium comprises an open reading frame encoding PapABC, and optionally, wherein the open reading frame encoding PapABC is derived from Pseudomonas fluorescens.

31. The engineered bacterium of claim 28, wherein the bacterium comprises an open reading frame encoding AroGL, and optionally wherein the open reading frame encoding AroGL is derived from E. coli.

32. The engineered bacterium of claim 27, wherein the bacterium is engineered to produce tetrahydrobiopterin (BH4) or derivatives thereof.

33. The engineered bacterium of claim 32, wherein the bacterium comprises an open reading frame encoding GTPCH.

34. The engineered bacterium of claim 33, wherein the open reading frame encoding GTPCH is derived from E. coli.

35. The engineered bacterium of claim 32, wherein the bacterium comprises an open reading frame encoding PTPS/SR.

36. The engineered bacterium of claim 35, wherein the open reading frame encoding PTPS/SR is derived from M. alpina.

37. A system for production of aromatic compounds or compounds with aromatic metabolites or intermediates, comprising an engineered bacterium comprising genetic elements supporting programmable transcriptional activation and/or repression and a growth medium.

38. A method of producing aromatic compounds or compounds with aromatic metabolites or intermediates, comprising:
providing an engineered bacterium comprising genetic elements supporting programmable transcriptional activation and/or repression; and a suitable substrate permitting production of the compounds.

39. The method of claim 38, wherein the compound is p-AF, and/or p-ACA and the substrate is selected from glucose, glycerol, p-coumaric acid, and other substrates from lignocellulosic biomass.
